(19) United States
(12) Patent Application Publication
(10) Pub. No.: US 2009/0298187 A1
(43) Pub. Date: Dec. 3, 2009

(54) COMPOSITIONS, METHODS, AND KITS USING SYNTHETIC PROBES FOR DETERMINING THE PRESENCE OF A TARGET NUCLEIC ACID

(75) Inventors: Irina NAZARENKO, Gaithersburg, MD (US); Dominic O'NEIL, Gaithersburg, MD (US)

Correspondence Address: BAKER DONELSON BEARMAN, CALDWELL & BERKOWITZ, PC, 555 ELEVENTH STREET, NW, SIXTH FLOOR, WASHINGTON, DC, 20004, US

(73) Assignee: QIAGEN GAITHERSBURG, INC., Gaithersburg, MD (US)

(21) Appl. No.: 12/426,076

(22) Filed: Apr. 17, 2009

Related U.S. Application Data
(60) Provisional application No. 61/045,952, filed on Apr. 17, 2008.
(60) Provisional application No. 61/113,841, filed on Nov. 12, 2008.
(60) Provisional application No. 61/147,862, filed on Jan. 28, 2009.

(51) Int. Cl. G01N 33/53; C07H 21/04

(52) U.S. Cl. 436/94; 536/24.32

(57) ABSTRACT

Compositions, methods, and kits are provided for determining the presence of a target nucleic acid in a sample using synthetic probes.

### RELATED APPLICATIONS

This application claims priority to U.S. provisional applications: 61/045,952 (filed on Apr. 17, 2008; 61/113,841 (filed on Nov. 12, 2008); and 61/147,862 (filed on Jan. 28, 2009), all of which are herein incorporated in their entirety.

### FIELD OF THE INVENTION

The present invention relates to compositions, methods, and kits using synthetic probes for determining the presence of a target nucleic acid in a biological sample.

### BACKGROUND OF THE INVENTION

The detection and characterization of specific nucleic acid sequences and sequence changes have been utilized to detect the presence of viral or bacterial nucleic acid sequences indicative of an infection, the presence of variants or alleles of mammalian genes associated with disease and cancers, and the identification of the source of nucleic acids found in forensic samples, as well as in paternity determinations.

For example, the RNA or DNA for many microorganisms and viruses have been isolated and sequenced. Nucleic acid probes have been examined for a large number of infections. Detectable nucleic acid sequences that hybridize to complementary RNA or DNA sequences in a test sample have been previously utilized. Detection of the probe indicates the presence of a particular nucleic acid sequence in the test sample for which the probe is specific. In addition to aiding scientific research, DNA or RNA probes can be used to detect the presence of viruses and microorganisms such as bacteria, yeast and protozoa as well as genetic mutations linked to specific disorders in patient samples. Nucleic acid hybridization probes have the advantages of high sensitivity and specificity over other detection methods and do not require a viable organism. Hybridization probes can be labeled, for example with a radioactive substance that can be easily detected.

As nucleic acid sequence data for genes from humans and pathogenic organisms accumulates, the demand for fast, cost-effective, and easy-to-use tests increases. It would be desirable to provide novel and effective methods, compositions, and kits for determining a target nucleic acid in a sample.

### SUMMARY OF THE INVENTION

In one aspect, the present invention provides a method for determining the presence of a target nucleic acid in a sample. The method comprises:

a) contacting one or more polynucleotide probes with the sample under a hybridization condition sufficient for the one or more polynucleotide probes to hybridize to the target nucleic acid in the sample to form double-stranded nucleic acid hybrids, wherein the one or more polynucleotide probes does not hybridize to a variant of the target nucleic acid; and

b) detecting the double-stranded nucleic acid hybrids, wherein detecting comprises contacting the double-stranded nucleic acid hybrids with a first anti-hybrid antibody that is immunospecific to double-stranded nucleic acid hybrids, whereby detection of the double-stranded nucleic acid hybrids determines the target nucleic acid in the sample.

In another aspect of the invention, the hybridization of the nucleic acids and detection of the double-stranded nucleic acid hybrids are performed at the same time.

In a further aspect of the invention, after the double-stranded nucleic acid hybrids are contacted with a first anti-hybrid antibody that is immunospecific to double-stranded nucleic acid hybrids, a second anti-hybrid antibody is added to detect the double-stranded nucleic acid hybrids whereby detection of the double-stranded nucleic acid hybrids by these second anti-hybrid antibodies determines the presence of target nucleic acid in the sample.

In another aspect of the invention, synthetic RNA probes corresponding to more than one HPV type are used to detect for the presence of HPV infection.

In certain embodiments, the detecting further comprises providing a second anti-hybrid antibody that is immunospecific to double-stranded nucleic acid hybrids, wherein the second anti-hybrid antibody is detectably labeled.

In certain embodiments, the at least one probe and the anti-hybrid antibody are added in the same step.

The target nucleic acid is may be an HPV nucleic acid and in certain embodiments, it is a high risk HPV type and the variant is a low risk type or another high risk type HPV nucleic acid. In certain embodiments, the hrHPV type is 16, 18 and/or 45.

In certain embodiments the one or more polynucleotide probes consist essentially of a sequence or a complement thereof selected from the group consisting of SEQ ID NOs: 1-2026.

The present invention provides for a method of determining the presence of an HPV target nucleic acid in a sample wherein if the target nucleic acid is HPV 16, the one or more polynucleotide probes is a set of nucleic acid probes comprising at least one nucleic acid sequence chosen from the group consisting of: SEQ ID NOs: 1-162.

When the target nucleic acid is HPV 18, the one or more polynucleotide probes is a set of nucleic acid probes comprising at least one nucleic acid sequence chosen from the group consisting of: SEQ ID NOs: 163-309.

When the target nucleic acid is HPV 45, the one or more polynucleotide probes is a set of nucleic acid probes comprising at least one nucleic acid sequence chosen from the group consisting of: SEQ ID NOs: 842-974.

When the target nucleic acid is HPV 31, the one or more polynucleotide probes is a set of nucleic acid probes comprising at least one nucleic acid sequence chosen from the group consisting of: SEQ ID NOs: 310-454.

When the target nucleic acid is HPV 33, the one or more polynucleotide probes is a set of nucleic acid probes comprising at least one nucleic acid sequence chosen from the group consisting of: SEQ ID NOs: 455-579.

When the target nucleic acid is HPV 35, the one or more polynucleotide probes is a set of nucleic acid probes comprising at least one nucleic acid sequence chosen from the group consisting of: SEQ ID NOs: 580-722.

When the target nucleic acid is HPV 39, the one or more polynucleotide probes is a set of nucleic acid probes comprising at least one nucleic acid sequence chosen from the group consisting of: SEQ ID NOs: 723-841.

When the target nucleic acid is HPV 51, the one or more polynucleotide probes is a set of nucleic acid probes comprising at least one nucleic acid sequence chosen from the group consisting of: SEQ ID NOs: 975-1120.

When the target nucleic acid is HPV 52, the one or more polynucleotide probes is a set of nucleic acid probes comprising at least one nucleic acid sequence chosen from the group consisting of: SEQ ID NOs: 1121-1252.

When the target nucleic acid is HPV 56, the one or more polynucleotide probes is a set of nucleic acid probes comprising at least one nucleic acid sequence chosen from the group consisting of: SEQ ID NOs: 1253-1367.

When the target nucleic acid is HPV 58, the one or more polynucleotide probes is a set of nucleic acid probes comprising at least one nucleic acid sequence chosen from the group consisting of: SEQ ID NOs: 1368-1497.

When the target nucleic acid is HPV 59, the one or more polynucleotide probes is a set of nucleic acid probes comprising at least one nucleic acid sequence chosen from the group consisting of: SEQ ID NOs: 1498-1646.

When the target nucleic acid is HPV 66, the one or more polynucleotide probes is a set of nucleic acid probes comprising at least one nucleic acid sequence chosen from the group consisting of: SEQ ID NOs: 1647-1767.

When the target nucleic acid is HPV 68, the one or more polynucleotide probes is a set of nucleic acid probes comprising at least one nucleic acid sequence chosen from the group consisting of: SEQ ID NOs: 1768-1875.

When the target nucleic acid is HPV 82, the one or more polynucleotide probes is a set of nucleic acid probes comprising at least one nucleic acid sequence chosen from the group consisting of: SEQ ID NOs: 1876-2026.

In certain embodiments, the one or more polynucleotide probes comprises the whole set of probes for that HPV type provided herein. In certain embodiments, the one or more polynucleotide probes consists essentially of or consists of the whole set of probes for that HPV type provided herein.

The present invention further provides probe sets of SEQ ID NO: 1-162 (HPV 16); 163-309(HPV 18); 842-974(HPV 45); 310-454(HPV 31); 455-579(HPV 33); 580-722(HPV 35); 723-841(HPV 39); 975-1120(HPV 51); 1121-1252(HPV 52); 1253-1367(HPV 56); 1368-1497(HPV 58); 1498-1646(HPV 59); 1647-1767(HPV 66); 1768-1875(HPV 68); and 1876-2026(HPV 82).

The present invention further provides probe sets of SEQ ID NO: 1-161 (HPV 16); 163-299 (HPV 18); and 842-968 (HPV 45). In certain embodiments the one or more polynucleotide probes is a mixture of probe sets comprising the probes set forth in SEQ ID NO: 1-2026.

In certain embodiments the one or more polynucleotide probes is a mixture of probe sets comprising the probes set forth in SEQ ID NO: 1-19, 21-23, 25-53, 55-65, 67-71, 73-92, 94-116, 118-130, 132-241, 244-274, 276, 277, 279, 280, 282-849, 851-893, 895-917, 919-929, 931, 933-936, 938-2026.

In certain embodiments the hybridization is performed at about 45 to about 55° C.

The present invention also provides kits comprising any one of the probes disclosed herein from SEQ ID NO: 1-2026. In certain embodiments the kits comprise the probes set forth from the group consisting of SEQ ID NO: 1-162 (HPV 16); 163-309(HPV 18); 842-974(HPV 45); 310-454(HPV 31); 455-579(HPV 33); 580-722(HPV 35); 723-841(HPV 39); 975-1120(HPV 51); 1121-1252(HPV 52); 1253-1367(HPV 56); 1368-1497(HPV 58); 1498-1646(HPV 59); 1647-1767(HPV 66); 1768-1875(HPV 68); and 1876-2026(HPV 82). In another embodiment, the kit comprises the probes set forth in SEQ ID NO: 1-161 (HPV 16); 163-299 (HPV 18); and 842-968 (HPV 45). In another embodiment, the kit comprises the probes set forth in SEQ ID NO: 1-2026. In yet another embodiment, the kit comprises the 2,007 probes set forth in SEQ ID NO: 1-19, 21-23, 25-53, 55-65, 67-71, 73-92, 94-116, 118-130, 132-241, 244-274, 276, 277, 279, 280, 282-849, 851-893, 895-917, 919-929, 931, 933-936, 938-2026. Advantages and benefits of the present invention will be apparent to one skilled in the art from reading this specification.

### BRIEF DESCRIPTION OF THE FIGURES

FIG. 1a shows the sequence conservation across 20 HPV genomes.

FIG. 1b shows location of RNA probes along HPV18 genome.

FIG. 2 shows performance of RNA probes specific for HPVs 16, 18, 31, or 45.

FIG. 3 shows detection of 5,000 copies of HPV18 plasmid with synRNA coverage of 3.7 Kb. synRNA=((1.5 kb coverage; 30mers) or (3.7 kb coverage; 25mers)) (1.34 nM

FIG. 4 shows that increasing the concentration of synRNA increased sensitivity of detection.

FIG. 5 shows that 50mer synRNA gave higher signal than 25mer synRNA; synRNA=0.5 kb of coverage; 25 or 50mers (concentrations listed above; at about 40 min hybridization (about 50° C.

FIG. 6 shows the effect of contiguous synRNA coverage on sensitivity of detection; 40 min hybridization (50° C.; synRNA=1.5 kb of coverage; 30 mers (2.24 nM.

FIG. 7 shows HPV16 and HPV18 detection with synRNA is comparable; 55° C. hybridization; synRNA=3.7 kb (coverage for HPV 18) or 3.175 kb (coverage for HPV 16); 25 mers (1.34 nM.

FIG. 8 shows comparison of synRNA prepared by different chemistries.

FIG. 9 shows hybridization of synRNAs at different temperatures; synRNA=3.7 kb of coverage; 25mers (1.34 nM.

FIG. 10 shows detection in the presence or absence of exogenous RNase A.

FIG. 11 shows sensitivity of detection.

FIG. 12 shows amplification time course.

FIG. 13 shows enhancing sensitivity by increasing target amplification.

FIG. 14 shows specificity.

FIG. 15 represents another embodiment of a method in accordance with the present invention.

FIG. 16 shows that diluting the sample collected in PreservCyt® with a suitable collection medium (“DCM”—Digene Collection Medium) enhances the signal.

FIG. 17 shows that synRNA probes have the same signal and dynamic range as the full length probes.

FIG. 18 shows that synRNA probes detected all specific targets (15 hrHPV target nucleic acids) with robust S/N and low variability.

FIG. 19 shows that even with 108 copies of low-risk HPV mixed with 108 copies of positive control, the mixture of 2,007 hrHPV probes were specific enough not to provide a positive signal for the low risk HPV types and were still able to provide a strong signal for the positive control.

FIGS. 20A and B shows that decreasing hybridization temperature increases the detection signal where the biological sample containing the target nucleic acid has been collected in PreverveCyt®.

### DETAILED DESCRIPTION

The present inventors have discovered novel methods, compositions, and kits using synthetic probes for determining the presence of a target nucleic acid in a biological sample. The present invention also provides synthetic probes useful for detecting a target nucleic acid in a sample. The present invention includes use of novel detection methods, compositions, and kits for, among other uses, clinical diagnostic purposes, including but not limited to the detection and identification of pathogenic organisms.

In one aspect, the present invention provides a method for determining the presence of a target nucleic acid in a sample, the method comprising:

a) contacting one or more polynucleotide probes with the sample under a hybridization condition sufficient for the one or more polynucleotide probes to hybridize to the target nucleic acid in the sample to form double-stranded nucleic acid hybrids, wherein the one or more polynucleotide probes does not hybridize to a variant of the target nucleic acid; and

b) detecting the double-stranded nucleic acid hybrids, wherein detecting comprises contacting the double-stranded nucleic acid hybrids with a first anti-hybrid antibody that is immunospecific to double-stranded nucleic acid hybrids, whereby detection of the double-stranded nucleic acid hybrids determines the target nucleic acid in the sample.

The sample includes, without limitation, a specimen or culture (e.g. microbiological and viral cultures) including biological and environmental samples. Biological samples may be from an animal, including a human, fluid, solid (e.g., stool) or tissue, as well as liquid and solid food and feed products and ingredients such as dairy items, vegetables, meat and meat by-products, and waste. Environmental samples include environmental material such as surface matter, soil, water and industrial samples, as well as samples obtained from food and dairy processing instruments, apparatus, equipment, utensils, disposable and non-disposable items. Particularly preferred are biological samples including, but not limited to cervical samples (e.g., a sample obtained from a cervical swab), blood, saliva, cerebral spinal fluid, pleural fluid, milk, lymph, sputum and semen. The sample may comprise a single- or double-stranded nucleic acid molecule, which includes the target nucleic acid and may be prepared for hybridization analysis by a variety of methods known in the art, e.g., using proteinase K/SDS, chaotropic salts, or the like. These examples are not to be construed as limiting the sample types applicable to the present invention.

For example, a sample such as blood or an exfoliated cervical cell specimen can be collected and subjected to alkaline pH to denature the target nucleic acid and, if necessary, nick the nucleic acid that may be present in the sample. The treated, or hydrolyzed, nucleic acids can then be subjected to hybridization with a probe or group of probes diluted in a neutralizing buffer.

In certain embodiments, the sample is an exfoliated cell sample, such as an exfoliated cervical cell sample. The sample can be collected with a chemically inert collection device such as, but not limited to, a dacron tipped swab, cotton swap, cervical brush, etc. The sample and collection device can be stored in a transport medium that preserves nucleic acids and inhibits nucleases, for example in a transport medium comprising a chaotropic salt solution, a detergent solution such as sodium dodecyl sulfate (SDS), preferably 0.5% SDS, or a chelating agent solution such as ethylenediaminetetraacetic acid (EDTA), preferably 100 mM, to prevent degradation of nucleic acids prior to analysis. In certain embodiments, the sample is a cervical cell sample and in this situation, both the cell sample and the collection device are stored in the chaotropic salt solution provided as the Sample Transport Medium™ in the digene Hybrid Capture® 2 High-Risk HPV DNA Test® kit (Qiagen Gaithersburg, Inc., Gaithersburg, Md.). Alternatively, the sample can be collected and stored in a base hydrolysis solution, for example.

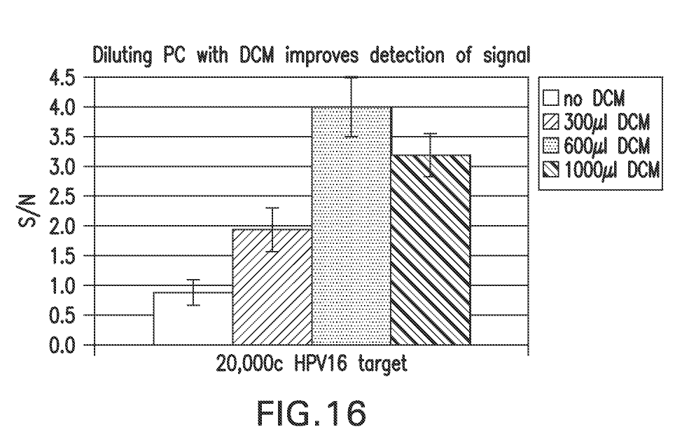

The sample may be collected and stored in a liquid based cytology collection medium such as, but not limited to, PreservCyt® and Surepath™. When such collection mediums are used (methanol based), it is preferable that the sample is diluted prior to performing methods of the present invention relating to detecting at target nucleic acid to obtain a stronger detection signal. A suitable solution is one that dilutes the methanol concentration, but still allows the rest of the reaction to proceed (i.e. allows hybridization of the probe to the target nucleic acid, allows binding of the hybrid capture antibody to the DNA:RNA, etc.). A useful solution is a collection medium comprising NP-40, sodium deoxycholate, Tris-HCl, EDTA, NaCl and sodium azide. In certain embodiments, the medium comprises or consists essentially of 1% NP-40, 0.25% sodium deoxycholate, 50 mM Tris-HCl, 25 mM EDTA, 150 mM NaCl and 0.09% sodium azide. This medium is often referred to herein and in the figures as Digene Collection Medium or DCM. FIG. 16 shows that diluting a methanol based collection medium, such as PreserveCyt® (or noted as “PC” in the figure) with a suitable solution such as DCM, produces a stronger signal and as such signals and hence detection of a target nucleic acid can be obtained even when the target nucleic acid has been collected in a relatively large volume of solution (i.e. >1 ml). Preferably the methanol based collection medium or PreserveCyt® is diluted in the following ratios of PC to DCM:

| Amount of | Amount of Digene |
|---|---|
| PreserveCyt ® | Collection Medium |
| (PC) in ml | (DCM) in μl |
| 1 | about 100 to about 1500 |
| 1 | about 200 to about 1300 |
| 1 | about 300 to about 1200 |
| 1 | about 400 to about 1100 |
| 1 | about 500 to about 1000 |
| 1 | about 600 to about 1000 |
| 1 | about 600 to about 900 |
| 1 | about 600 to about 800 |

In other embodiments 1 ml of PC is diluted with at least 200 μl of DCM, in other embodiments, 1 ml of PC is diluted with at least 300 μl of DCM, and in other embodiments, 1 ml of PC is diluted with at least 500 μl of DCM. In certain embodiments, 1 ml of PC is diluted with at least 500 DCM but no more than 1000 μl DCM. By diluting the PC containing the biological sample, the methods of the present invention are able to provide results and detect a target nucleic acid from a relative large sample volume (i.e. a biological sample collected in ≧1 ml).

If the nucleic acids to be determined are present in blood, a blood sample can be collected with a syringe, for example, and the serum separated by conventional methods. Preferably, serum is incubated for approximately 20 minutes at approximately 65° C. with a protease, such as proteinase K prior to a base treatment.

In some embodiments, the sample is treated with a base, or hydrolyzed, to render the target nucleic acid accessible to hybridization. Nucleic acids can be denatured and, if necessary, nicked by incubating the sample and collection device, if present, in 0.1 to 2.0 M base at about 20 to about 100° C. for 5 to 120 minutes. Preferably, treatment is achieved with 0.2 to 0.8 M NaOH, or a similar base such as KOH, at 60-70° C. for 30 to 60 minutes. Most preferably, the sample and swab are incubated in 0.415 M NaOH for 65° C. for 45 minutes. Approximately one volume of sample can be treated with about one-half volume of base, also referred to herein as the hydrolysis reagent. The pH will typically be about 13. This basic pH will both nick and denature a majority of the nucleic acid in the specimen. In addition, base treatment can disrupt interactions between peptides and nucleic acids to improve accessibility of the target nucleic acid and degrade protein. Base treatment effectively homogenizes the specimen to ensure reproducibility of analysis results for a given sample. Base treatment also can reduce the viscosity of the sample to increase kinetics, homogenize the sample, and reduce background by destroying any existing DNA-RNA or RNA-RNA hybrids in the sample. Base treatment also can help inactivate enzymes such as RNAases that may be present in the sample.

The variant of the target nucleic acid includes genetic variants of the target. A variant includes polymorphisms, mutants, derivatives, modified, altered, or the like forms of the target nucleic acid. By way of example with respect to a human papillomavirus (HPV), variants include the various types. Thus, for example, wherein the target nucleic acid corresponds to HPV type 18 nucleic acid, the variant can be a corresponding nucleic acid sequence of a type of HPV other than type 18.

In one embodiment, the target nucleic acid is an HPV nucleic acid. In another embodiment, the HPV nucleic acid is HPV DNA of an HPV type. In some embodiments, the HPV type is HPV 18, wherein the variant is nucleic acid of a type selected from the group consisting of: HPV 1, 2, 3, 4, 5, 6, 8, 11, 13, 16, 26, 30, 31, 33, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

In other embodiments, the HPV type is HPV 16, wherein the variant is nucleic acid of a type selected from the group consisting of: HPV 1, 2, 3, 4, 5, 6, 8, 11, 13, 18, 26, 30, 31, 33, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

In other embodiments, the HPV type is HPV 45, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 51, 52, 53, 54, 56, 58, 59, 61, 62, 66, 67, 68, 69, 70, 71, 72, 73, 74, 81, 82, 83, 84, and 89.

In other embodiments, the HPV type is HPV 31, wherein the variant is nucleic acid of a type selected from the group consisting of: HPV 1, 2, 3, 4, 5, 6, 8, 11, 13, 16, 18, 26, 30, 33, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

In other embodiments, the HPV type is HPV 33, wherein the variant is nucleic acid of a type selected from the group consisting of: HPV 1, 2, 3, 4, 5, 6, 8, 11, 13, 16, 18, 26, 30, 31, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

In other embodiments, the HPV type is HPV 35, wherein the variant is nucleic acid of a type selected from the group consisting of: HPV 1, 2, 3, 4, 5, 6, 8, 11, 13, 16, 18, 26, 30, 31, 33, 34, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

In other embodiments, the HPV type is HPV 39, wherein the variant is nucleic acid of a type selected from the group consisting of: HPV 1, 2, 3, 4, 5, 6, 8, 11, 13, 16, 18, 26, 30, 31, 33, 34, 35, 40, 42, 43, 44, 45, 51, 52, 53, 54, 56, 58, 59, 61, 62, 66, 67, 68, 69, 70, 71, 72, 73, 74, 81, 82, 83, 84, and 89.

In other embodiments, the HPV type is HPV 51, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 52, 53, 54, 56, 58, 59, 61, 62, 66, 67, 68, 69, 70, 71, 72, 73, 74, 81, 82, 83, 84, and 89.

In other embodiments, the HPV type is HPV 52, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 53, 54, 56, 58, 59, 61, 62, 66, 67, 68, 69, 70, 71, 72, 73, 74, 81, 82, 83, 84, and 89.

In other embodiments, the HPV type is HPV 56, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 52, 53, 54, 58, 59, 61, 62, 66, 67, 68, 69, 70, 71, 72, 73, 74, 81, 82, 83, 84, and 89.

In other embodiments, the HPV type is HPV 58, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 52, 53, 54, 56, 59, 61, 62, 66, 67, 68, 69, 70, 71, 72, 73, 74, 81, 82, 83, 84, and 89.

In other embodiments, the HPV type is HPV 59, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 52, 53, 54, 56, 58, 61, 62, 66, 67, 68, 69, 70, 71, 72, 73, 74, 81, 82, 83, 84, and 89.

In other embodiments, the HPV type is HPV 66, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 52, 53, 54, 56, 58, 59, 61, 62, 67, 68, 69, 70, 71, 72, 73, 74, 81, 82, 83, 84, and 89.

In other embodiments, the HPV type is HPV 68, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 52, 53, 54, 56, 58, 59, 61, 62, 66, 67, 69, 70, 71, 72, 73, 74, 81, 82, 83, 84, and 89.

In other embodiments, the HPV type is HPV 82, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 45, 51, 52, 53, 54, 56, 58, 59, 61, 62, 66, 67, 68, 69, 70, 71, 72, 73, 74, 81, 83, 84, and 89.

In other embodiments, the HPV type is HPV 16, 18 and 45, wherein the variant is nucleic acid of a low risk HPV type.

In other embodiments, the HPV type is a high risk HPV type (hrHPV), wherein the variant is nucleic acid of a low risk HPV type.

In other embodiments, the HPV type is 16, 18, 31, 33, 35, 39, 45, 51, 52, 56, 58, 59, 66, 68, and 82 wherein the wherein the variant is nucleic acid of a low risk HPV type (such as 1, 2, 3, 4, 5, 6, 8, 11, 13, 26, 30, 34, 53, 54, 61, 62, 67, 69, 70, 71, 72, 73, 74, 81, 83, 84, and 89).

Thus, the present invention provides methods, compositions, and kit for determining a target nucleic acid in a sample. The sample can be collected with a chemically inert device and optionally treated with a base or other denaturing solution. The sample is incubated with one or more polynucleotide probes that are specific for the target nucleic acid but not for any other member of the population (i.e. will not bind to a variant). For example, the target nucleic acid to be determined can be an oncogenic or non-oncogenic HPV DNA sequence, HBV DNA sequence, Gonorrhea DNA, Chlamydia DNA, or other pathogen DNA or RNA. The target nucleic acid may be from cells for the detection of cancer.

In one embodiment, the target nucleic acid is an HPV nucleic acid, wherein the target and the variant nucleic acids correspond to an HPV high risk or low risk type. HPV types characterized as low risk and high risk are known to one of ordinary skill in the art. Presently HPV types 16, 18, 31, 33, 35, 39, 45, 51, 52, 56, 58, 59, 66, 68, and 82 are considered hrHPVs and HPV types 1, 2, 3, 4, 5, 6, 8, 11, 13, 26, 30, 34, 53, 54, 61, 62, 67, 69, 70, 71, 72, 73, 74, 81, 83, 84, and 89 are considered low risk HPVs.

Thus, for example, the target nucleic acid to be determined can be nucleic acid of a microorganism such as, e.g. a disease-causing pathogen, preferably a virus or bacteria, preferably HPV, however, the invention is not restricted thereto and the description following is merely illustrated by reference to determining an HPV DNA in a sample.

### Polynucleotide Probes (“Synprobes”)

In accordance with the present invention, one or more polynucleotide probes are contacted with the sample under conditions sufficient for the one or more polynucleotide probes to hybridize to the target nucleic acid in the sample to form double-stranded nucleic acid hybrids. In certain embodiments, the target nucleic acid is DNA and the probes are RNA. In certain embodiments the RNA probes are short probes as opposed to full length transcribed RNA probes. These short probes are often referred to herein as synthetic RNA probes or “synRNA.”

In certain embodiments, sets of polynucleotide probes are used (i.e. more than one probe). For example, if the target nucleic acid to be detected is HPV 16, a set of probes designed to specifically (i.e. only) bind to HPV 16 as opposed to binding to other HPV types is used. In certain embodiments a set of probes is used to ensure coverage of about 3-4 kb of the target nucleic acid, which ensures a strong, readable signal. In certain embodiments, detection of HPV 16 using the methods of the present invention may use a probe set comprising all of the HPV 16 probes disclosed herein (see Table 1). In other embodiments, a set of probes designed to specifically bind to another HPV type is used. For example, for HPV 18, the set of probes comprises the probes disclosed in Table 2, for HPV 45—the set of probes comprises the probes disclosed in Table 3; for HPV 31—the set of probes comprises the probes disclosed in Table 4; for HPV 33—the set of probes comprises the probes disclosed in Table 5; for HPV 35—the set of probes comprises the probes disclosed in Table 6; for HPV 39—the set of probes comprises the probes disclosed in Table 7; for HPV 51—the set of probes comprises the probes disclosed in Table 8; for HPV 52—the set of probes comprises the probes disclosed in Table 9; for HPV 56—the set of probes comprises the probes disclosed in Table 10; for HPV 58—the set of probes comprises the probes disclosed in Table 11; for HPV 59—the set of probes comprises the probes disclosed in Table 12; for HPV 66—the set of probes comprises the probes disclosed in Table 13; for HPV 68—the set of probes comprises the probes disclosed in Table 14; for HPV 15—the set of probes comprises the probes disclosed in Table 15.

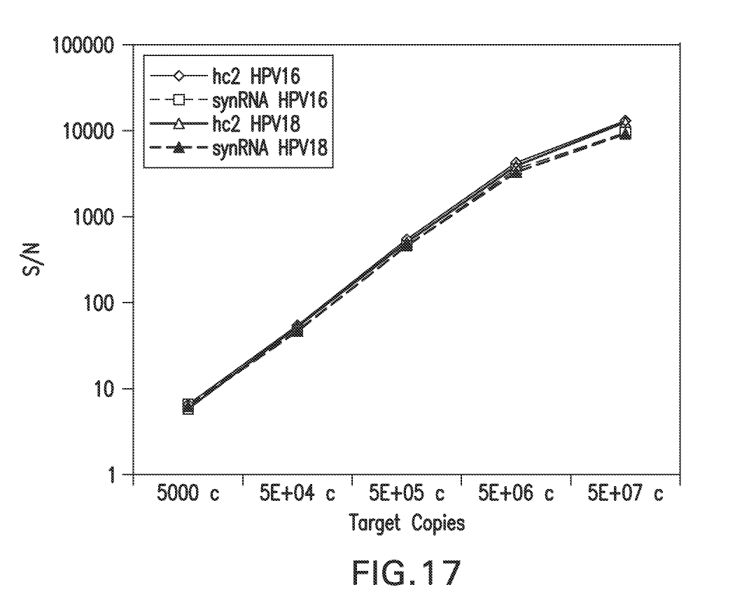

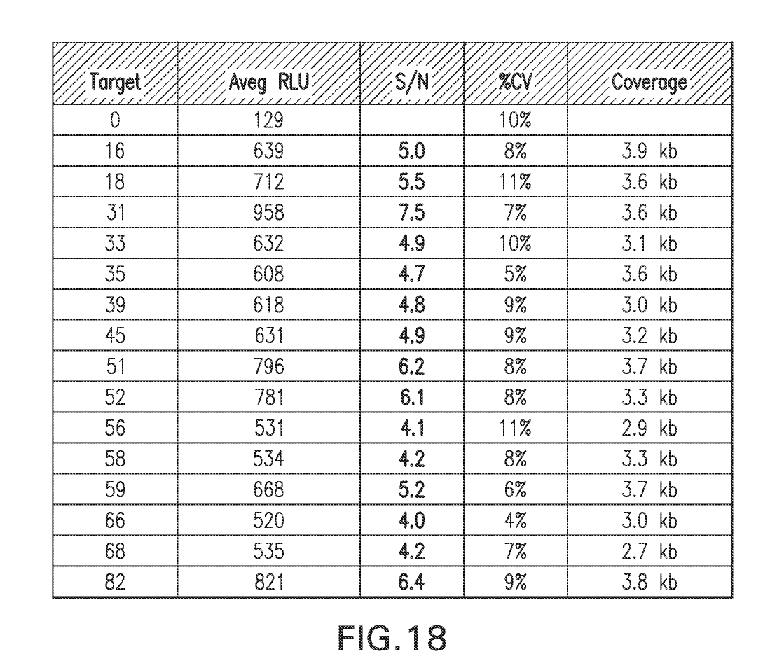

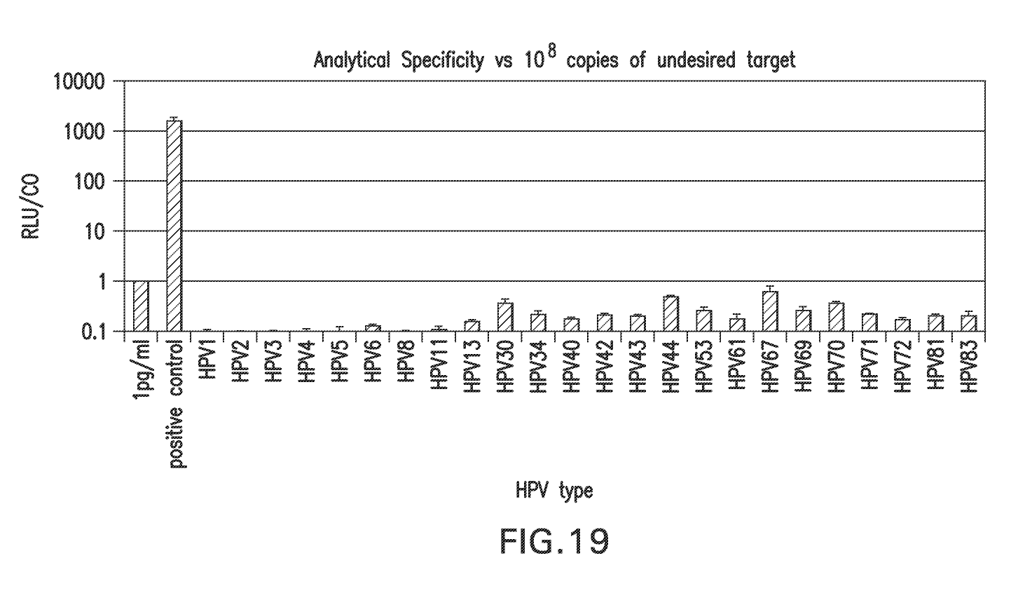

In certain embodiments a probe mixture comprising multiple sets of probes is used to simultaneously screen for any one of a mixture of desired target nucleic acids. For example, it may be desirable to screen a biological sample for the presence of any hrHPV type. In such a situation, a probe mixture of some, and in some cases, all of the probes provided in Tables 1-15 are used. For example, a probe mixture can be designed to provide a probe set for every high risk HPV (hrHPV) so one test can be run to identify whether the sample had any hrHPV target nucleic acid. For example, a probe mixture of 2,007 type-specific probes for the detection of 15 hrHPV types was used and was able to detect 5,000 copies/assay of each target genome (see FIGS. 17 and 18). FIG. 17 shows that the synthetic probes have the same signal and dynamic range as traditional full length probes. FIG. 19 provides the results of an analytical specificity test, which shows a good signal for the positive control having 108 copies, whereas the low risk HPV types had a signal below the cutoff, even when they were present at 108 copies. Thus, FIGS. 17-19 show that the methods of the present utilizing the synthetic RNA probes (“synRNA”) of the invention provide analytical specificity and are equivalent in limit of detection and dynamic range to full-length transcribed probes and do not suffer any loss of sensitivity with clinical samples. The probes of the present invention enable sensitive detection of a set of target genomes, while also achieving excellent specificity against even very similar related species. For example, the methods of the invention using the synprobes are able to distinguish HPV 67 from HPV 52 and 58 (HPV67 is greater than 72% identical to HPV 52 and 56). See FIG. 19.

If a positive signal is obtained in the example above, it may then be desirable to further test the sample to identify the actual hrHPV type target nucleic acid present. In such a situation, the sample would be further tested with one probe specific for the HPV type or a set of probes for the specific HPV type. For example, if one were testing the sample to determine whether the sample contained an HPV 16 target nucleic acid, then at least one probe from Table 1 (HPV 16 probes) would be used, or alternatively the entire set of probes from Table 1 would be used to increase the signal strength. Alternatively, it may be desirable to test for certain hrHPV types such as HPV 16, 18 and 45 and not necessarily test for each individual hrHPV types. In this situation, the mixture of probes would employ at least one probe from the HPV 16, 18 and 45 probe sets (or alternatively, all of the probes from the 16, 18 and 45 HPV probe sets are used).

The one or more polynucleotide probes are designed so that they do not hybridize to a variant of the target nucleic acid under the hybridization conditions utilized. The number of different polynucleotide probes employed per set can depend on the desired sensitivity. Higher coverage of the nucleic acid target with the corresponding polynucleotide probes can provide a stronger signal (as there will be more DNA-RNA hybrids for the antibodies to bind).

In one embodiment, the method further comprises determining the one or more polynucleotide probes, wherein determining comprises identifying a contiguous nucleotide sequence of the target nucleic acid, wherein the contiguous nucleotide sequence is not present in the variant. By way of example, relatively short regions (e.g., about 25mers) of the HPV genome with sufficient sequence specificity can be determined to provide the one or more polynucleotide probes for HPV type-specific hybridization.

Thus, depending on the target nucleic acid of interest, and the corresponding variant(s), the one or more polynucleotide probes can be prepared to have lengths sufficient to provide target-specific hybridization. In some embodiments, the one or more polynucleotide probes each have a length of at least about 15 nucleotides, illustratively, about 15 to about 1000, about 20 to about 800, about 30 to about 400, about 40 to about 200, about 50 to about 100, about 20 to about 60, about 20 to about 40, about 20 to about 20 and about 25 to about 30 nucleotides. In one embodiment, the one or more polynucleotide probes each have a length of about 25 to about 50 nucleotides. In certain embodiments, the probes have a length of 25 nucleotides. In certain embodiments, all of the probes in a set will have the same length, such as 25 nucleotides, and will have very similar melting temperatures to allow hybridization of all of the probes in the set under the same hybridization conditions.

Bioinformatics tools can be employed to determine the one or more polynucleotide probes. For example, Oligoarray 2.0, a software program that designs specific oligonucleotides can be utilized. Oligoarray 2.0 is described by Rouillard et al. Nucleic Acids Research, 31: 3057-3062 (2003), which is incorporated herein by reference. Oligoarray 2.0 is a program which combines the functionality of BLAST (Basic Local Alignment Search Tool) and Mfold (Genetics Computer Group, Madison, Wis.). BLAST, which implements the statistical matching theory by Karlin and Altschul (Proc. Natl. Acad. Sci. USA 87:2264 (1990); Proc. Natl. Acad. Sci. USA 90:5873 (1993), is a widely used program for rapidly detecting nucleotide sequences that match a given query sequence One of ordinary skill in the art can provide a database of sequences, which are to be checked against, for example HPV high risk and low risk types 1, 2, 3, 4, 5, 6, 8, 11, 13, 16, 26, 30, 31, 33, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89. The target sequence of interest, e.g. HPV 18, can then be BLASTed against that database to search for any regions of identity. Melting temperature (Tm) and % GC can then be computed for one or more polynucleotide probes of a specified length and compared to the parameters, after which secondary structure also can be examined. Once all parameters of interest are satisfied, cross hybridization can be checked with the Mfold package, using the similarity determined by BLAST. The various programs can be adapted to determine the one or more polynucleotide probes meeting the desired specificity requirements. For example, the parameters of the program can be set to prepare polynucleotides of 25 nt length, Tm range of 55-95° C., a GC range of 35-65%, and no secondary structure or cross-hybridization at 55° C. or below.

Accordingly in other aspects, the present invention utilizes bioinformatics to provide sequence information sufficient to design and/or prepare polynucleotide probes for determining the target in the sample.

In addition to using the synprobes in a method of the present invention, one aspect of the invention comprises the probes disclosed herein.

In one embodiment, the present invention provides an isolated polynucleotide for specific hybridization to HPV 16 consisting essentially of a sequence or a complement thereof selected from the group consisting of SEQ ID NOs: 1-162 (See Table 1). In some embodiments, the present invention provides a set of polynucleotides for specific hybridization to HPV 16, wherein the set comprises at least one polynucleotide consisting essentially of a sequence or a complement thereof selected from the group consisting of: SEQ ID NOs: 1-162. In some embodiments, the present invention provides a set of polynucleotides for specific hybridization to HPV 16, wherein the set comprises at least one polynucleotide consisting essentially of a sequence or a complement thereof selected from the group consisting of: SEQ ID NOs: 1-161. In certain embodiments, the methods of the present invention utilize a set of polynucleotide probes for specific hybridization to HPV 16 comprising SEQ ID NOs: 1-162. In certain embodiments, the methods of the present invention utilize a set of polynucleotide probes for specific hybridization to HPV 16 comprising SEQ ID NO: 1-19, 21-23, 25-53, 55-65, 67-71, 73-92, 94-116, 118-130, 132-162.

TABLE 1
Polyribonucleotide probes for
determining HPV 16 nucleic acid.

| SEQ | | |
|---|---|---|
| ID | | |
| NO: | Name | Sequence |
| 1 | HPV16_25_HR&LR_7866 | GGGUUACACAUUUACAAGCAACUUA |
| 2 | HPV16_25_HR&LR_7841 | ACAUGGGUGUGUGCAAACCGAUUUU |
| 3 | HPV16_25_HR&LR_7799 | CUGUGUAAAGGUUAGUCAUACAUUG |
| 4 | HPV16_25_HR&LR_7774 | AAUGUCACCCUAGUUCAUACAUGAA |
| 5 | HPV16_25_HR&LR_7749 | AGGUUUAAACUUCUAAGGCCAACUA |
| 6 | HPV16_25_HR&LR_7712 | GGCUUGUUUUAACUAACCUAAUUGC |
| 7 | HPV16_25_HR&LR_7676 | CAACGCCUUACAUACCGCUGUUAGG |
| 8 | HPV16_25_HR&LR_7629 | CUGAAUCACUAUGUACAUUGUGUCA |
| 9 | HPV16_25_HR&LR_7577 | GCACUGCUUGCCAACCAUUCCAUUG |
| 10 | HPV16_25_HR&LR_7552 | UGCCAAAUCCCUGUUUUCCUGACCU |
| 11 | HPV16_25_HR&LR_7527 | UUGUACGUUUCCUGCUUGCCAUGCG |
| 12 | HPV16_25_HR&LR_7502 | CUAUGUCAGCAACUAUGGUUUAAAC |
| 13 | HPV16_25_HR&LR_7433 | CCCAUUUUGUAGCUUCAACCGAAUU |
| 14 | HPV16_25_HR&LR_7408 | AUAUACUAUAUUUUGUAGCGCCAGG |
| 15 | HPV16_25_HR&LR_7371 | UAUAAACUAUAUUUGCUACAUCCUG |
| 16 | HPV16_25_HR&LR_7340 | CCUACUAAUUGUGUUGUGGUUAUUC |
| 17 | HPV16_25_HR&LR_7293 | GUGUAACUAUUGUGUCAUGCAACAU |
| 18 | HPV16_25_HR&LR_7250 | UGUAUGGUAUAAUAAACACGUGUGU |
| 19 | HPV16_25_HR&LR_7225 | AUAUUAAGUUGUAUGUGUGUUUGUA |
| 20 | HPV16_25_HR&LR_7201 | GUAUGUGCUUGUAUGUGCUUGUAAA |
| 21 | HPV16_25_HR&LR_7175 | UAGUGUUGUUUGUUGUGUAUAUGUU |
| 22 | HPV16_25_HR&LR_7150 | UGUAAGUAUUGUAUGUAUGUUGAAU |
| 23 | HPV16_25_HR&LR_7112 | AUCUACCUCUACAACUGCUAAACGC |
| 24 | HPV16_25_HR&LR_7087 | AACGAAAAGCUACACCCACCACCUC |
| 25 | HPV16_25_HR&LR_7061 | GGCCAAACCAAAAUUUACAUUAGGA |
| 26 | HPV16_25_HR&LR_6935 | AGCACCUAAAGAAGAUGAUCCCCUU |
| 27 | HPV16_25_HR&LR_6894 | UUUGUAACCCAGGCAAUUGCUUGUC |
| 28 | HPV16_25_HR&LR_6869 | AGGCACACUAGAAGAUACUUAUAGG |
| 29 | HPV16_25_HR&LR_6790 | CAGACGUUAUGACAUACAUACAUUC |
| 30 | HPV16_25_HR&LR_6675 | GCCAUAUCUACUUCAGAAACUACAU |
| 31 | HPV16_25_HR&LR_6541 | CUGAUGCCCAAAUAUUCAAUAAACC |
| 32 | HPV16_25_HR&LR_6496 | CCAGUUCAAAUUAUUUUCCUACACC |
| 33 | HPV16_25_HR&LR_6471 | GGCUCUGGGUCUACUGCAAAUUUAG |
| 34 | HPV16_25_HR&LR_6438 | GGUGAAAAUGUACCAGACGAUUUAU |
| 35 | HPV16_25_HR&LR_6350 | GUCAGAACCAUAUGGCGACAGCUUA |
| 36 | HPV16_25_HR&LR_6294 | GUUCCACUGGAUAUUUGUACAUCUA |
| 37 | HPV16_25_HR&LR_6192 | CCACCAUUAGAGUUAAUAAACACAG |
| 38 | HPV16_25_HR&LR_6165 | AAUGUUGCAGUAAAUCCAGGUGAUU |
| 39 | HPV16_25_HR&LR_6052 | CAGGUGUGGAUAAUAGAGAAUGUAU |
| 40 | HPV16_25_HR&LR_6022 | CAGAAAAUGCUAGUGCUUAUGCAGC |
| 41 | HPV16_25_HR&LR_5851 | UAUUUAGAAUACAUUUACCUGACCC |
| 42 | HPV16_25_HR&LR_5825 | UAAAGUAUCAGGAUUACAAUACAGG |
| 43 | HPV16_25_HR&LR_5800 | CUAACAAUAACAAAAUAUUAGUUCC |
| 44 | HPV16_25_HR&LR_5745 | GCAGGAACAUCCAGACUACUUGCAG |
| 45 | HPV16_25_HR&LR_5586 | GUUAUUACAUGUUACGAAAACGACG |
| 46 | HPV16_25_HR&LR_5546 | ACAAUUAUUGCUGAUGCAGGUGACU |
| 47 | HPV16_25_HR&LR_5521 | UAUAGUUCCAGGGUCUCCACAAUAU |
| 48 | HPV16_25_HR&LR_5496 | CUGACCAAGCUCCUUCAUUAAUUCC |
| 49 | HPV16_25_HR&LR_5469 | CAGGUCCUGAUAUACCCAUUAAUAU |
| 50 | HPV16_25_HR&LR_5442 | GUGGUGCAUACAAUAUUCCUUUAGU |
| 51 | HPV16_25_HR&LR_5406 | CAGGUUAUAUUCCUGCAAAUACAAC |
| 52 | HPV16_25_HR&LR_5381 | CCAUCUGUACCCUCUACAUCUUUAU |
| 53 | HPV16_25_HR&LR_5356 | UACAGAUACUUCUACAACCCCGGUA |
| 54 | HPV16_25_HR&LR_5336 | AUUUAUGCAGAUGACUUUAUUACAG |
| 55 | HPV16_25_HR&LR_5301 | CCUCACCUACUUCUAUUAAUAAUGG |
| 56 | HPV16_25_HR&LR_5276 | ACAUAUACUACCACUUCACAUGCAG |
| 57 | HPV16_25_HR&LR_5228 | ACUAUUGAUCCUGCAGAAGAAAUAG |
| 58 | HPV16_25_HR&LR_5182 | UGGAAAAUCUAUAGGUGCUAAGGUA |
| 59 | HPV16_25_HR&LR_5153 | GGUAAUAAACAAACACUACGUACUC |
| 60 | HPV16_25_HR&LR_5122 | UAGGCGUACUGGCAUUAGGUACAGU |
| 61 | HPV16_25_HR&LR_5051 | AAUAGUAUUAAUAUAGCUCCAGAUC |
| 62 | HPV16_25_HR&LR_5000 | GCAUAUGAAGGUAUAGAUGUGGAUA |
| 63 | HPV16_25_HR&LR_4965 | CCACUCCCACUAAACUUAUUACAUA |
| 64 | HPV16_25_HR&LR_4910 | GGAUUAUAUAGUCGCACAACACAAC |
| 65 | HPV16_25_HR&LR_4854 | CUAACACAGUAACUAGUAGCACACC |
| 66 | HPV16_25_HR&LR_4829 | GAUACAUUUAUUGUUAGCACAAACC |
| 67 | HPV16_25_HR&LR_4771 | GCAUUUUACACUUUCAUCAUCCACU |
| 68 | HPV16_25_HR&LR_4706 | CAUAAUAAUCCCACUUUCACUGACC |
| 69 | HPV16_25_HR&LR_4681 | UAAUACUGUUACUACUGUUACUACA |
| 70 | HPV16_25_HR&LR_4640 | ACUACUUCAACUGAUACCACACCUG |
| 71 | HPV16_25_HR&LR_4588 | UGCACCAACAUCUGUACCUUCCAUU |
| 72 | HPV16_25_HR&LR_4562 | GAAGAAACUAGUUUUAUUGAUGCUG |
| 73 | HPV16_25_HR&LR_4480 | UACAGAUACACUUGCUCCUGUAAGA |
| 74 | HPV16_25_HR&LR_4435 | CGGACGCACUGGGUAUAUUCCAUUG |
| 75 | HPV16_25_HR&LR_4369 | AUUACAAUAUGGAAGUAUGGGUGUA |
| 76 | HPV16_25_HR&LR_4275 | CGGCUACCCAACUUUAUAAAACAUG |
| 77 | HPV16_25_HR&LR_4232 | ACAAUGCGACACAAACGUUCUGCAA |
| 78 | HPV16_25_HR&LR_4131 | AAUUGUUGUAUACCAUAACUUACUA |
| 79 | HPV16_25_HR&LR_4103 | AUAUGUACAUAAUGUAAUUGUUACA |
| 80 | HPV16_25_HR&LR_4009 | CUCUGCGUUUAGGUGUUUUAUUGUA |
| 81 | HPV16_25_HR&LR_3984 | UAUUACUAUUGUGGAUAACAGCAGC |
| 82 | HPV16_25_HR&LR_3942 | UGCUUUUGUCUGUGUCUACAUACAC |
| 83 | HPV16_25_HR&LR_3866 | UGCAUCCACAACAUUACUGGCGUGC |
| 84 | HPV16_25_HR&LR_3824 | CAGUGUCUACUGGAUUUAUGUCUAU |
| 85 | HPV16_25_HR&LR_3765 | UGAUAGUGAAUGGCAACGUGACCAA |
| 86 | HPV16_25_HR&LR_3712 | CAUUGGACAGGACAUAAUGUAAAAC |
| 87 | HPV16_25_HR&LR_3686 | UGUAUACUGCAGUGUCGUCUACAUG |
| 88 | HPV16_25_HR&LR_3638 | CUAAUACUUUAAAAUGUUUAAGAUA |
| 89 | HPV16_25_HR&LR_3602 | GUAACACUACACCCAUAGUACAUUU |
| 90 | HPV16_25_HR&LR_3577 | CACAAAGGACGGAUUAACUGUAAUA |
| 91 | HPV16_25_HR&LR_3552 | AAUCCUCACUGCAUUUAACAGCUCA |
| 92 | HPV16_25_HR&LR_3520 | UUGUUGCACAGAGACUCAGUGGACA |
| 93 | HPV16_25_HR&LR_3495 | CGGAAACCCCUGCCACACCACUAAG |
| 94 | HPV16_25_HR&LR_3460 | ACGACUAUCCAGCGACCAAGAUCAG |
| 95 | HPV16_25_HR&LR_3417 | GACCCAUACCAAAGCCGUCGCCUUG |
| 96 | HPV16_25_HR&LR_3378 | UGAAAUUAUUAGGCAGCACUUGGCC |
| 97 | HPV16_25_HR&LR_3323 | GUCAGGUAAUAUUAUGUCCUACAUC |
| 98 | HPV16_25_HR&LR_3241 | GGAAUACGAACAUAUUUUGUGCAGU |
| 99 | HPV16_25_HR&LR_3201 | GGGUCAAGUUGACUAUUAUGGUUUA |
| 100 | HPV16_25_HR&LR_3176 | AAGAAGCAUCAGUAACUGUGGUAGA |
| 101 | HPV16_25_HR&LR_3145 | UAUACAAACUGGACACAUAUAUAUA |
| 102 | HPV16_25_HR&LR_3103 | GUGGAAGUGCAGUUUGAUGGAGACA |
| 103 | HPV16_25_HR&LR_3043 | GUUAGCCUUGAAGUGUAUUUAACUG |
| 104 | HPV16_25_HR&LR_3018 | UAAUGAAAAGUGGACAUUACAAGAC |
| 105 | HPV16_25_HR&LR_2974 | GAACUGCAACUAACGUUAGAAACAA |
| 106 | HPV16_25_HR&LR_2938 | CUGGCUGUAUCAAAGAAUAAAGCAU |
| 107 | HPV16_25_HR&LR_2890 | GCCAGAGAAAUGGGAUUUAAACAUA |
| 108 | HPV16_25_HR&LR_2863 | CGCCUAGAAUGUGCUAUUUAUUACA |
| 109 | HPV16_25_HR&LR_2828 | ACCUACGUGACCAUAUAGACUAUUG |
| 110 | HPV16_25_HR&LR_2794 | AAAAUACUAACACAUUAUGAAAAUG |
| 111 | HPV16_25_HR&LR_2630 | UAAUGAGUUUCCAUUUGACGAAAAC |
| 112 | HPV16_25_HR&LR_2602 | AUAAUAGAUUGGUGGUGUUUACAUU |
| 113 | HPV16_25_HR&LR_2555 | UACAUCUAACAUUAAUGCUGGUACA |
| 114 | HPV16_25_HR&LR_2501 | UAUGGAUGUAAAGCAUAGACCAUUG |
| 115 | HPV16_25_HR&LR_2444 | CUGUUGGAACUACAUAGAUGACAAU |
| 116 | HPV16_25_HR&LR_2345 | GCAAGGGUCUGUAAUAUGUUUUGUA |
| 117 | HPV16_25_HR&LR_2324 | UAUGAGUUUAAUGAAAUUUCUGCAA |
| 118 | HPV16_25_HR&LR_2282 | AUUACUAUAUGGUGCAGCUAACACA |
| 119 | HPV16_25_HR&LR_2171 | AGGUGAUUGGAAGCAAAUUGUUAUG |
| 120 | HPV16_25_HR&LR_2139 | AUAAAAUAUAGAUGUGAUAGGGUAG |
| 121 | HPV16_25_HR&LR_1957 | ACGAUAAUGACAUAGUAGACGAUAG |
| 122 | HPV16_25_HR&LR_1914 | AAUGAUUGUACAUUUGAAUUAUCAC |
| 123 | HPV16_25_HR&LR_1827 | UAUAAAACAGGUAUAUCAAAUAUUA |
| 124 | HPV16_25_HR&LR_1775 | UAUGAUGAUAGAGCCUCCAAAAUUG |
| 125 | HPV16_25_HR&LR_1750 | AACUAUUAUGUGUGUCUCCAAUGUG |
| 126 | HPV16_25_HR&LR_1676 | GGGAAUGGUUGUGUUACUAUUAGUA |
| 127 | HPV16_25_HR&LR_1584 | UUUGGACUUACACCCAGUAUAGCUG |
| 128 | HPV16_25_HR&LR_1559 | GUGUUGCGAUUGGUGUAUUGCUGCA |
| 129 | HPV16_25_HR&LR_1534 | GACCAUUUAAAAGUAAUAAAUCAAC |
| 130 | HPV16_25_HR&LR_1492 | AAUUUAAAGAGUUAUACGGGGUGAG |
| 131 | HPV16_25_HR&LR_1417 | CUAUAUGCCAAACACCACUUACAAA |
| 132 | HPV16_25_HR&LR_1364 | UUGCAGUCAGUACAGUAGUGGAAGU |
| 133 | HPV16_25_HR&LR_1331 | AUGUAGUCAGUAUAGUGGUGGAAGU |
| 134 | HPV16_25_HR&LR_1306 | AAGGGCGCCAUGAGACUGAAACACC |
| 135 | HPV16_25_HR&LR_1238 | AUUAUUUGAAAGCGAAGACAGCGGG |
| 136 | HPV16_25_HR&LR_1185 | CCUAGAUUAAAAGCUAUAUGUAUAG |
| 137 | HPV16_25_HR&LR_1150 | GUGAUAUUAGUGGAUGUGUAGACAA |
| 138 | HPV16_25_HR&LR_1101 | UAGAGAUGCAGUACAGGUUCUAAAA |
| 139 | HPV16_25_HR&LR_1076 | UUACUGCACAGGAAGCAAAACAACA |
| 140 | HPV16_25_HR&LR_1029 | UAAUGAUUAUUUAACACAGGCAGAA |
| 141 | HPV16_25_HR&LR_1004 | AUUUGGUAGAUUUUAUAGUAAAUGA |
| 142 | HPV16_25_HR&LR_984 | UGACAGUGAUACAGGUGAAGAUUUG |
| 143 | HPV16_25_HR&LR_848 | AGAAACCAUAAUCUACCAUGGCUGA |
| 144 | HPV16_25_HR&LR_790 | CGUACUUUGGAAGACCUGUUAAUGG |
| 145 | HPV16_25_HR&LR_732 | UUGUUGCAAGUGUGACUCUACGCUU |
| 146 | HPV16_25_HR&LR_702 | GGACAGAGCCCAUUACAAUAUUGUA |
| 147 | HPV16_25_HR&LR_569 | GAGAUACACCUACAUUGCAUGAAUA |
| 148 | HPV16_25_HR&LR_524 | AGAUCAUCAAGAACACGUAGAGAAA |
| 149 | HPV16_25_HR&LR_477 | UCCAUAAUAUAAGGGGUCGGUGGAC |
| 150 | HPV16_25_HR&LR_412 | UAUUAACUGUCAAAAGCCACUGUGU |
| 151 | HPV16_25_HR&LR_366 | UAGAACAGCAAUACAACAAACCGUU |
| 152 | HPV16_25_HR&LR_334 | ACAUUAUUGUUAUAGUUUGUAUGGA |
| 153 | HPV16_25_HR&LR_306 | AGUUUUAUUCUAAAAUUAGUGAGUA |
| 154 | HPV16_25_HR&LR_281 | UAUGCUGUAUGUGAUAAAUGUUUAA |
| 155 | HPV16_25_HR&LR_245 | CGGGAUUUAUGCAUAGUAUAUAGAG |
| 156 | HPV16_25_HR&LR_209 | CAGUUACUGCGACGUGAGGUAUAUG |
| 157 | HPV16_25_HR&LR_155 | GAGCUGCAAACAACUAUACAUGAUA |
| 158 | HPV16_25_HR&LR_130 | CAGAAAGUUACCACAGUUAUGCACA |
| 159 | HPV16_25_HR&LR_92 | AAGAGAACUGCAAUGUUUCAGGACC |
| 160 | HPV16_25_HR&LR_57 | CCGGUUAGUAUAAAAGCAGACAUUU |
| 161 | HPV16_25_HR&LR_18 | AUAAAACUAAGGGCGUAACCGAAAU |
| 162 | HPV16_7200 | UGUAUGUGCUUGUAUGUGCUUGUAA |

In one embodiment, the present invention provides an isolated polynucleotide for specific hybridization to HPV 18 consisting essentially of a sequence or a complement thereof selected from the group consisting of: SEQ ID NOs: 163-309 (See Table 2). In some embodiments, the present invention provides a set of polynucleotides for specific hybridization to HPV 18, wherein the set comprises at least one polynucleotide consisting essentially of a sequence or a complement thereof selected from the group consisting of: SEQ ID NOs: 163-309. In some embodiments, the present invention provides a set of polynucleotides for specific hybridization to HPV 18, wherein the set comprises at least one polynucleotide consisting essentially of a sequence or a complement thereof selected from the group consisting of: SEQ ID NOs: 163-299. In certain embodiments, the methods of the present invention utilize a set of polynucleotide probes for specific hybridization to HPV 18 comprising SEQ ID NOs: 163-309. In certain embodiments, the methods of the present invention utilize a set of polynucleotide probes for specific hybridization to HPV 18 comprising SEQ ID NO: 163-241, 244-274, 276, 277, 279, 280, 282-309.

TABLE 2

Polyribonucleotide probes for
determining HPV 18 nucleic acid.

| | | |
|---|---|---|
| SEQ ID NO: | Name | Sequence |

| | | |
|---|---|---|
| 163 | HPV18_25_HR&LR(−45)_7833 | UUGGGCAGCACAUACUAUACUUUUC |
| 164 | HPV18_25_HR&LR(−45)_7796 | UAAGCUGUGCAUACAUAGUUUAUGC |
| 165 | HPV18_25_HR&LR(−45)_7764 | CUGUCUACCCUUAACAUGAACUAUA |
| 166 | HPV18_25_HR&LR(−45)_7738 | GUACAACUACUUUCAUGUCCAACAU |
| 167 | HPV18_25_HR&LR(−45)_7658 | AUCCACUCCCUAAGUAAUAAAACUG |
| 168 | HPV18_25_HR&LR(−45)_7632 | GCUACAACAAUUGCUUGCAUAACUA |
| 169 | HPV18_25_HR&LR(−45)_7561 | UUGAACAAUUGGCGCGCCUCUUUGG |
| 170 | HPV18_25_HR&LR(−45)_7536 | CUUUUGGGCACUGCUCCUACAUAUU |
| 171 | HPV18_25_HR&LR(−45)_7501 | CAAUACAGUACGCUGGCACUAUUGC |
| 172 | HPV18_25_HR&LR(−45)_7476 | UGGCUUAUGUCUGUGGUUUUCUGCA |
| 173 | HPV18_25_HR&LR(−45)_7423 | CCAUUUUAUCCUACAAUCCUCCAUU |
| 174 | HPV18_25_HR&LR(−45)_7398 | UAUAAAACUGCACACCUUACAGCAU |
| 175 | HPV18_25_HR&LR(−45)_7370 | GGGCUAUAUAUUGUCCUGUAUUUCA |
| 176 | HPV18_25_HR&LR(−45)_7345 | GUUUGUGGUAUGGGUGUUGCUUGUU |
| 177 | HPV18_25_HR&LR(−45)_7320 | CCUAGUGAGUAACAACUGUAUUUGU |
| 178 | HPV18_25_HR&LR(−45)_7291 | UUGUGGUUCUGUGUGUUAUGUGGUU |
| 179 | HPV18_25_HR&LR(−45)_7249 | GUUACUAUAUUUGUUGGUAUGUGGC |
| 180 | HPV18_25_HR&LR(−45)_7211 | CAUUGUAUGGUAUGUAUGGUUGUUG |
| 181 | HPV18_25_HR&LR(−45)_7184 | CCUGUGUUUGUGUUUGUUGUAUGAU |
| 182 | HPV18_25_HR&LR(−45)_7123 | GUGCCAGGAAGUAAUAUGUGUGUGU |
| 183 | HPV18_25_HR&LR(−45)_7098 | AAACCUGCCAAGCGUGUGCGUGUAC |
| 184 | HPV18_25_HR&LR(−45)_7073 | UGCUCCAUCUGCCACUACGUCUUCU |
| 185 | HPV18_25_HR&LR(−45)_6982 | CUUUAGACUUAGAUCAAUAUCCCCU |
| 186 | HPV18_25_HR&LR(−45)_6911 | UGCACCGGCUGAAAAUAAGGAUCCC |
| 187 | HPV18_25_HR&LR(−45)_6876 | GUACAAUCUGUUGCUAUUACCUGUC |
| 188 | HPV18_25_HR&LR(−45)_6698 | GCAGUAUAGCAGACAUGUUGAGGAA |
| 189 | HPV18_25_HR&LR(−45)_6672 | GGGCAAUAUGAUGCUACCAAAUUUA |
| 190 | HPV18_25_HR&LR(−45)_6625 | CCAGUACCAAUUUAACAAUAUGUGC |
| 191 | HPV18_25_HR&LR(−45)_6482 | GUAUUCUCCCUCUCCAAGUGGCUCU |
| 192 | HPV18_25_HR&LR(−45)_6425 | GCCUCAAUCCUUAUAUAUUAAAGGC |
| 193 | HPV18_25_HR&LR(−45)_6254 | AGAUACUAAAUGUGAGGUACCAUUG |
| 194 | HPV18_25_HR&LR(−45)_6188 | CACAGUUUUGGAAGAUGGUGAUAUG |
| 195 | HPV18_25_HR&LR(−45)_6137 | UAAAUCGCGUCCUUUAUCACAGGGC |
| 196 | HPV18_25_HR&LR(−45)_6029 | UUCUGAGGACGUUAGGGACAAUGUG |
| 197 | HPV18_25_HR&LR(−45)_6004 | GUUCCCAUGCCGCCACGUCUAAUGU |
| 198 | HPV18_25_HR&LR(−45)_5766 | GUUCCUGCAGGUGGUGGCAAUAAGC |
| 199 | HPV18_25_HR&LR(−45)_5667 | GCAAGAGUUGUAAAUACCGAUGAUU |
| 200 | HPV18_25_HR&LR(−45)_5642 | CGUAUAUCUUCCACCUCCUUCUGUG |
| 201 | HPV18_25_HR&LR(−45)_5519 | CAGUAUAUUGGUAUACAUGGUACAC |
| 202 | HPV18_25_HR&LR(−45)_5487 | CCAUUGUAUCACCCACGGCCCCUGC |
| 203 | HPV18_25_HR&LR(−45)_5462 | UUACCAUCUACUACCUCUGUAUGGC |
| 204 | HPV18_25_HR&LR(−45)_5437 | UGUAUACACGGGUCCUGAUAUUACA |
| 205 | HPV18_25_HR&LR(−45)_5409 | UCCCUUUAACCUCCUCUUGGGAUGU |
| 206 | HPV18_25_HR&LR(−45)_5384 | GCCUCUUCCUAUAGUAAUGUAACGG |
| 207 | HPV18_25_HR&LR(−45)_5329 | AUCGCGUUCUACUACCUCCUUUGCA |
| 208 | HPV18_25_HR&LR(−45)_5304 | ACAUGGACCOUGOAGUGOCUGUACO |
| 209 | HPV18_25_HR&LR(−45)_5249 | CAGCCUUUAGUAUCUGCCACGGAGG |
| 210 | HPV18_25_HR&LR(−45)_5224 | ACCUUCCCCAGAAUAUAUUGAACUG |
| 211 | HPV18_25_HR&LR(−45)_5160 | UUACCCGCAGCGGUACACAAAUAGG |
| 212 | HPV18_25_HR&LR(−45)_5118 | GGACUGUUCGCUUUAGUAGAUUAGG |
| 213 | HPV18_25_HR&LR(−45)_5021 | GACACUACAUUAACAUUUGAUCCUC |
| 214 | HPV18_25_HR&LR(−45)_4971 | CACGUCCAUCCUCUUUAAUUACAUA |
| 215 | HPV18_25_HR&LR(−45)_4946 | UCAGUGGCUAACCCUGAGUUUCUUA |
| 216 | HPV18_25_HR&LR(−45)_4833 | UACAAACAUUUGCUUCUUCUGGUAC |
| 217 | HPV18_25_HR&LR(−45)_4737 | CGUCCAUUAUUGAAGUUCCACAAAC |
| 218 | HPV18_25_HR&LR(−45)_4701 | CCACAACCAAUUUUACCAAUCCUGC |
| 219 | HPV18_25_HR&LR(−45)_4676 | CCUUCGUCUACCUCUGUGUCUAUUU |
| 220 | HPV18_25_HR&LR(−45)_4634 | ACAUCUGCGGGUACAACUACACCUG |
| 221 | HPV18_25_HR&LR(−45)_4591 | UGCACCUAGGCCUACGUUUACUGGC |
| 222 | HPV18_25_HR&LR(−45)_4566 | AGGACUCCAGUGUGGUUACAUCAGG |
| 223 | HPV18_25_HR&LR(−45)_4483 | AGUGGUGGAUGUUGGUCCUACACGU |
| 224 | HPV18_25_HR&LR(−45)_4455 | ACAUUCCAUUGGGUGGGCGUUCCAA |
| 225 | HPV18_25_HR&LR(−45)_4375 | AUUGCAAUGGUCAAGCCUUGGUAUA |
| 226 | HPV18_25_HR&LR(−45)_4276 | GGCUUCGGUAACUGACUUAUAUAAA |
| 227 | HPV18_25_HR&LR(−45)_4234 | UAAUAAAAGUAUGGUAUCCCACCGU |
| 228 | HPV18_25_HR&LR(−45)_4113 | CCCAUGUUACUAUUGCAUAUACAUG |
| 229 | HPV18_25_HR&LR(−45)_4072 | CUGCCACAGCAUUCACAGUAUAUGU |
| 230 | HPV18_25_HR&LR(−45)_4047 | GUGUAUAUUGUGGUAAUAACGUCCC |
| 231 | HPV18_25_HR&LR(−45)_3971 | AUGCAUGUAUGUGUGCUGCCAUGUC |
| 232 | HPV18_25_HR&LR(−45)_3922 | GCUGUAGUACCAAUAUGUUAUCACU |
| 233 | HPV18_25_HR&LR(−45)_3888 | AUAUUGGUGGGAUACAUGACAAUGU |
| 234 | HPV18_25_HR&LR(−45)_3863 | UGUUGCAAUUCCAGAUAGUGUACAA |
| 235 | HPV18_25_HR&LR(−45)_3823 | CAUACCAUAGUGAAACACAAAGAAC |
| 236 | HPV18_25_HR&LR(−45)_3752 | CUAUAGAGAUAUAUCAUCCACCUGG |
| 237 | HPV18_25_HR&LR(−45)_3727 | ACAGAUUGCGAAAACAUAGCGACCA |
| 238 | HPV18_25_HR&LR(−45)_3647 | AAGACGGAAACUCUGUAGUGGUAAC |
| 239 | HPV18_25_HR&LR(−45)_3622 | CAGCUACACCUACAGGCAACAACAA |
| 240 | HPV18_25_HR&LR(−45)_3597 | GGACCUGUCAACCCACUUCUCGGUG |
| 241 | HPV18_25_HR&LR(−45)_3572 | UGGACUCGCGGAGAAGCAGCAUUGU |
| 242 | HPV18_25_HR&LR(−45)_3547 | CGGCUGCUACACGACCUGGACACUG |
| 243 | HPV18_25_HR&LR(−45)_3499 | AUUCCAGCACCGUGUCCGUGGGCAC |
| 244 | HPV18_25_HR&LR(−45)_3454 | CCGCUACUCAGCUUGUUAAACAGCU |
| 245 | HPV18_25_HR&LR(−45)_3382 | GGGAAGUACAUUUUGGGAAUAAUGU |
| 246 | HPV18_25_HR&LR(−45)_3315 | GAAGGGUACAACACGUUUUAUAUAG |
| 247 | HPV18_25_HR&LR(−45)_3269 | CAAAACCGCUACCUGUGUAAGUCAC |
| 248 | HPV18_25_HR&LR(−45)_3244 | AUAUGACUGAUGCAGGAACAUGGGA |
| 249 | HPV18_25_HR&LR(−45)_3219 | UAUGUAGCAUGGGACAGUGUGUAUU |
| 250 | HPV18_25_HR&LR(−45)_3168 | GGCCAAACAGUACAAGUAUAUUUUG |
| 251 | HPV18_25_HR&LR(−45)_3134 | GAAUACAGAACCUACUCACUGCUUU |
| 252 | HPV18_25_HR&LR(−45)_3080 | AAGUCGAUACAAAACCGAGGAUUGG |
| 253 | HPV18_25_HR&LR(−45)_2972 | ACAUGGCAUACAGACAUUAAACCAC |
| 254 | HPV18_25_HR&LR(−45)_2938 | GUUGGGAAAAUGCAAUAUUCUUUGC |
| 255 | HPV18_25_HR&LR(−45)_2903 | CAUAGACAGCCAAAUACAGUAUUGG |
| 256 | HPV18_25_HR&LR(−45)_2645 | GCAAAGGAUAAUAGAUGGCCAUAUU |
| 257 | HPV18_25_HR&LR(−45)_2612 | CCUCCAAUACUACUAACCACAAAUA |
| 258 | HPV18_25_HR&LR(−45)_2527 | CUUUGAUACCUAUAUGAGAAAUGCG |
| 259 | HPV18_25_HR&LR(−45)_2475 | CAGAUACUAAGGUGGCCAUGUUAGA |
| 260 | HPV18_25_HR&LR(−45)_2270 | CUGCGAUACCAACAAAUAGAGUUUA |
| 261 | HPV18_25_HR&LR(−45)_2202 | CACAGUGGAUACGAUUUAGAUGUUC |
| 262 | HPV18_25_HR&LR(−45)_2065 | UGAAUAUGCCUUAUUAGCAGACAGC |
| 263 | HPV18_25_HR&LR(−45)_2036 | GAGCUGACAGAUGAAAGCGAUAUGG |
| 264 | HPV18_25_HR&LR(−45)_1944 | CUGAGUGGAUACAAAGACUUACUAU |
| 265 | HPV18_25_HR&LR(−45)_1918 | UAUUAGUGAAGUAAUGGGAGACACA |
| 266 | HPV18_25_HR&LR(−45)_1829 | CACGUACCUGAAACUUGUAUGUUAA |
| 267 | HPV18_25_HR&LR(−45)_1802 | GUUGCUAAAGGUUUAAGUACGUUGU |
| 268 | HPV18_25_HR&LR(−45)_1777 | CAAAUGUGGUAAGAGUAGACUAACA |
| 269 | HPV18_25_HR&LR(−45)_1751 | GUAUUAAUAUUAGCCCUGUUGCGUU |
| 270 | HPV18_25_HR&LR(−45)_1726 | UCAAUGUCUAGACUGUAAAUGGGGA |
| 271 | HPV18_25_HR&LR(−45)_1572 | ACACAUAUGGGCUAUCAUUUACAGA |
| 272 | HPV18_25_HR&LR(−45)_1536 | ACAAUAAACAAGGAGCUAUGUUAGC |
| 273 | HPV18_25_HR&LR(−45)_1493 | CCACAAUGUACCAUAGCACAAUUAA |
| 274 | HPV18_25_HR&LR(−45)_1455 | ACGGUACAAGUGACAAUAGCAAUAU |
| 275 | HPV18_25_HR&LR(−45)_1429 | CACAGAGGGCAACAACAGCAGUGUA |
| 276 | HPV18_25HR&LR(−45)_1399 | CGGCAGUACGGAGGCUAUAGACAAC |
| 277 | HPV18_25HR&LR(−45)_1360 | AACUACAAAUGGCGAACAUGGCGGC |
| 278 | HPV18_25 HR&LR(−45)_1216 | GCGGCUGGAGGUGGAUACAGAGUUA |
| 279 | HPV18_25_HR&LR(−45)_1149 | CACAAGUGUUGCAUGUUUUAAAACG |
| 280 | HPV18_25HR&LR(−45)_1072 | ACAAGGAACAUUUUGUGAACAGGCA |
| 281 | HPV18_25HR&LR(−45)_959 | GGCUGGUUUUAUGUACAAGCUAUUG |
| 282 | HPV18_25HR&LR(−45)_885 | CGUGGUGUGCAUCCCAGCAGUAAGC |
| 283 | HPV18_25HR&LR(−45)_857 | UUUCUGAACACCCUGUCCUUUGUGU |
| 284 | HPV18_25HR&LR(−45)_816 | UAGAAAGCUCAGCAGACGACCUUCG |
| 285 | HPV18_25HR&LR(−45)_791 | UGUGAAGCCAGAAUUGAGCUAGUAG |
| 286 | HPV18_25HR&LR(−45)_695 | GAAGAAAACGAUGAAAUAGAUGGAG |
| 287 | HPV18_25HR&LR(−45)_670 | UCACGAGCAAUUAAGCGACUCAGAG |
| 288 | HPV18_25HR&LR(−45)_645 | AUGAAAUUCCGGUUGACCUUCUAUG |
| 289 | HPV18_25HR&LR(−45)_620 | AUUGUAUUGCAUUUAGAGCCCCAAA |
| 290 | HPV18_25HR&LR(−45)_589 | UAUGCAUGGACCUAAGGCAACAUUG |
| 291 | HPV18_25_HR&LR(−45)_554 | CCAACGACGCAGAGAAACACAAGUA |
| 292 | HPV18_25HR&LR(−45)_529 | GCAACCGAGCACGACAGGAACGACU |
| 293 | HPV18_25_HR&LR(−45)_489 | AACAUAGCUGGGCACUAUAGAGGCC |
| 294 | HPV18_25HR&LR(−45)_344 | UUAUUCAGACUCUGUGUAUGGAGAC |
| 295 | HPV18_25HR&LR(−45)_264 | GUGGUGUAUAGAGACAGUAUACCCC |
| 296 | HPV18_25HR&LR(−45)_216 | GUAUUGGAACUUACAGAGGUAUUUG |
| 297 | HPV18_25HR&LR(−45)_179 | GCAAGACAUAGAAAUAACCUGUGUA |
| 298 | HPV18_25HR&LR(−45)_154 | UGUGCACGGAACUGAACACUUCACU |
| 299 | HPV18_25HR&LR(−45)_92 | ACACCACAAUACUAUGGCGCGCUUU |
| 300 | HPV18_7601 | CCUGGUAUUAGUCAUUUUCCUGUCC |
| 301 | HPV18_6850 | CUAGUUUGGUGGAUACAUAUCGUUU |
| 302 | HPV18_5697 | ACUCCCACAAGCAUAUUUUAUCAUG |
| 303 | HPV18_5046 | GUAGUGAUGUUCCUGAUUCAGAUUU |
| 304 | HPV18_2877 | GACCACUAUGAAAAUGACAGUAAAG |
| 305 | HPV18_1298 | CUGUUUACAAUAUCAGAUAGUGGCU |
| 306 | HPV18_1241 | AGUCCACGGUUACAAGAAAUAUCUU |
| 307 | HPV18_739 | AGCCCGACGAGCCGAACCACAACGU |
| 308 | HPV18_405 | UUAUUAAUAAGGUGCCUGCGGUGCC |
| 309 | HPV18_289 | AUGCUGCAUGCCAUAAAUGUAUAGA |

In one embodiment, the present invention provides an isolated polynucleotide for specific hybridization to HPV 45 consisting essentially of a sequence or a complement thereof selected from the group consisting of: SEQ ID NOs: 842-974 (See Table 3). In some embodiments, the present invention provides a set of polynucleotides for specific hybridization to HPV 45, wherein the set comprises at least one polynucleotide consisting essentially of a sequence or a complement thereof selected from the group consisting of: SEQ ID NOs: 842-974. In some embodiments, the present invention provides a set of polynucleotides for specific hybridization to HPV 45, wherein the set comprises at least one polynucleotide consisting essentially of a sequence or a complement thereof selected from the group consisting of: SEQ ID NOs: 842-968. In certain embodiments, the methods of the present invention utilize a set of polynucleotide probes for specific hybridization to HPV 45 comprising SEQ ID NOs: 842-974. In certain embodiments, the methods of the present invention utilize a set of polynucleotide probes for specific hybridization to HPV 45 comprising SEQ ID NO: 842-849, 851-893, 895-917, 919-929, 931, 933-936, 938-974.

TABLE 3

Polyribonucleotide probes for
determining HPV 45 nucleic acid.

| | | |
|---|---|---|
| SEQ ID NO: | Name | Sequence |

| | | |
|---|---|---|
| 842 | HPV45_25_HR&LR(−18)_7834 | GGCCCUAUAACACAUACCUUUUCUU |
| 843 | HPV45_25_HR&LR(−18)_7754 | CCAACAAUCUGUCUACUUGUUACAU |
| 844 | HPV45_25_HR&LR(−18)_7726 | UAAUUGGCGUGUAGAACCACUUUCU |
| 845 | HPV45_25_HR&LR(−18)_7646 | GCACAACUGUAUCCACACCCUAUGU |
| 846 | HPV45_25_HR&LR(−18)_7552 | ACAUAGUUUAACCUACUGGCGCGCC |
| 847 | HPV45_25_HR&LR(−18)_7527 | CUAAACUGGCACAUUUACAACCCCU |
| 848 | HPV45_25_HR&LR(−18)_7495 | GUGGCUUAUAUGUGACCUUUUAAAC |
| 849 | HPV45_25_HR&LR(−18)_7440 | GCAUCCAUUUUACUUAUAAUCCUCC |
| 850 | HPV45_25_HR&LR(−18)_7385 | CUUUGUACCCUAUAUUCUUUCCUGU |
| 851 | HPV45_25_HR&LR(−18)_7322 | UAAUAGUGUUGUGUAGGGUUGCACC |
| 852 | HPV45_25_HR&LR(−18)_7282 | GGUGUUACUGUACAUAAUUGUGGUA |
| 853 | HPV45_25_HR&LR(−18)_7250 | GUGUAUGUAUGAAUGUGCCUUGUGG |
| 854 | HPV45_25_HR&LR(−18)_7225 | UACUGUAUUUUGUUUGUUUGCGUGC |
| 855 | HPV45_25_HR&LR(−18)_7106 | CAUCUAGGCCUGCCAAACGUGUACG |
| 856 | HPV45_25_HR&LR(−18)_7081 | GCUUCCACGUCUACUGCAUCUACUG |
| 857 | HPV45_25_HR&LR(−18)_7052 | CUACCAUAGGACCUCGUAAGCGUCC |
| 858 | HPV45_25_HR&LR(−18)_7027 | GUUCAGGCUGGGUUACGUCGUAGGC |
| 859 | HPV45_25_HR&LR(−18)_6911 | AUACUACACCUCCAGAAAAGCAGGA |
| 860 | HPV45_25_HR&LR(−18)_6885 | AUCAGUUGCUGUUACCUGUCAAAAG |
| 861 | HPV45_25_HR&LR(−18)_6697 | UUUAAGCAGUAUAGUAGACAUGUGG |
| 862 | HPV45_25_HR&LR(−18)_6672 | GCCAAGUACAUAUGACCCUACUAAG |
| 863 | HPV45_25_HR&LR(−18)_6505 | GGCUCUAUUAUUACUUCUGAUUCUC |
| 864 | HPV45_25_HR&LR(−18)_6479 | GUUGUGUGUAUUCCCCUUCUCCCAG |
| 865 | HPV45_25_HR&LR(−18)_6454 | GCUAAUAUGCGUGAAACCCCUGGCA |
| 866 | HPV45_25_HR&LR(−18)_6426 | UACGGACCUAUAUAUUAAAGGCACU |
| 867 | HPV45_25_HR&LR(−18)_6272 | CAUUAGACAUUUGUCAAUCCAUCUG |
| 868 | HPV45_25_HR&LR(−18)_6247 | UUGCAGGAUACAAAGUGCGAGGUUC |
| 869 | HPV45_25_HR&LR(−18)_6142 | GCACAAUUGCAACCUGGUGACUGUC |
| 870 | HPV45_25_HR&LR(−18)_6018 | AGCUGUUAUUACGCAGGAUGUUAGG |
| 871 | HPV45_25_HR&LR(−18)_5833 | GUAGCUUUACCCGAUCCUAAUAAAU |
| 872 | HPV45_25_HR&LR(−18)_5791 | GCUGUUCCUAAGGUAUCCGCAUAUC |
| 873 | HPV45_25_HR&LR(−18)_5766 | ACCUAAUGGUGCAGGUAAUAAACAG |
| 874 | HPV45_25_HR&LR(−18)_5741 | UAGGCAAUCCAUAUUUUAGGGUUGU |
| 875 | HPV45_25_HR&LR(−18)_5654 | CUUCUGUGGCCAGAGUUGUCAGCAC |
| 876 | HPV45_25_HR&LR(−18)_5534 | CACACAAUAUUAUUUAUGGCCAUGG |
| 877 | HPV45_25_HR&LR(−18)_5490 | UCUCCUACCAAUGCUUCCACCACCA |
| 878 | HPV45_25_HR&LR(−18)_5465 | CCAUACUCCUAUGUGGCCUAGUACA |
| 879 | HPV45_25_HR&LR(−18)_5437 | AUACUGGCCCGGACAUUAUAUUGCC |
| 880 | HPV45_25_HR&LR(−18)_5402 | AGUACCAUUAACAUCUGCAUGGGAU |
| 881 | HPV45_25_HR&LR(−18)_5372 | UACUGCUGCAUCCUCUUACAGUAAU |
| 882 | HPV45_25_HR&LR(−18)_5347 | CAAAGUAUUCCUUGACCAUGCCUUC |
| 883 | HPV45_25_HR&LR(−18)_5314 | CACCUAGCACUAUACACAAAUCAUU |
| 884 | HPV45_25_HR&LR(−18)_5289 | GACUUCCCACCUCCUGCGUCCACUA |
| 885 | HPV45_25_HR&LR(−18)_5254 | CUACAAAUGAUAGUGACCUGUUUGA |
| 886 | HPV45_25_HR&LR(−18)_5209 | CCAUUGCUGCUACAGAGGAAAUUGA |
| 887 | HPV45_25_HR&LR(−18)_5111 | CACUGUUAGAUUUAGUAGAUUGGGU |
| 888 | HPV45_25_HR&LR(−18)_5038 | CCAGUAAUGUUCCUGAUUCCGAUUU |
| 889 | HPV45_25_HR&LR(−18)_5013 | GACACCACACUAUCCUUUGAGCCUA |
| 890 | HPV45_25_HR&LR(−18)_4974 | UCGUUGGUUACAUUUGAUAAUCCAG |
| 891 | HPV45_25_HR&LR(−18)_4926 | AAUCAACAGGUCCGUGUGUCCACCU |
| 892 | HPV45_25_HR&LR(−18)_4837 | CAUCUUCUGGGUCAGGUACGGAACC |
| 893 | HPV45_25_HR&LR(−18)_4781 | UGGUACACCAACAUCGGGCAGCCAU |
| 894 | HPV45_25_HR&LR(−18)_4716 | GCAUUUUCUGAUCCCUCUAUUAUUG |
| 895 | HPV45_25_HR&LR(−18)_4679 | CUCUGUUUCUAUUUCGUCAACUAGU |
| 896 | HPV45_25_HR&LR(−18)_4654 | UGUUGGACAUCACACCUACCGUGGA |
| 897 | HPV45_25_HR&LR(−18)_4573 | UUGCCUCUGGUGCUCCGGUUCCCAC |
| 898 | HPV45_25_HR&LR(−18)_4463 | CAGGUCUAAUACUGUUGUGGAUGUU |
| 899 | HPV45_25_HR&LR(−18)_4367 | UUUACAGUGGUCUAGCCUUGGGAUA |
| 900 | HPV45_25_HR&LR(−18)_4224 | GUUUAAUAAACCAUGGUAUCCCACC |
| 901 | HPV45_25_HR&LR(−18)_4158 | AUACCUGUGAUGUGCAUGUUGUUGU |
| 902 | HPV45_25_HR&LR(−18)_4106 | GCAUGCUUUACACACCAUACAAUAA |
| 903 | HPV45_25_HR&LR(−18)_4053 | GCAUUUGCUGUAUACAUUUGUUGCU |
| 904 | HPV45_25_HR&LR(−18)_3989 | UGUGUGUGCUUUUGCUUGGUUGUUG |
| 905 | HPV45_25_HR&LR(−18)_3944 | GUGCCUUUAUGUGUGCUGCAAUGUC |
| 906 | HPV45_25_HR&LR(−18)_3857 | GGGAUACAUGACUAUAUGAAUCUGU |
| 907 | HPV45_25_HR&LR(−18)_3832 | UUCCUAACAGUGUACAAAUCUCGGU |
| 908 | HPV45_25_HR&LR(−18)_3717 | UACUCAGAAAUAUCCUCCACCUGGC |
| 909 | HPV45_25_HR&LR(−18)_3685 | UAAGAUAUAGGCUACGCAAAUAUGC |
| 910 | HPV45_25_HR&LR(−18)_3612 | AGAAGGAAAGUGUGUAGUGGUAACA |
| 911 | HPV45_25_HR&LR(−18)_3585 | CUGUGUUCAAGUACAAGUAACAACA |
| 912 | HPV45_25_HR&LR(−18)_3535 | UCACAGAGCAGCACCACGGACGUGU |
| 913 | HPV45_25_HR&LR(−18)_3492 | CACAUCCAGACGCCGGCUACUAAGC |
| 914 | HPV45_25_HR&LR(−18)_3429 | AGACAGCUACAACACGCCUCCACGU |
| 915 | HPV45_25_HR&LR(−18)_3325 | GAAAUAGUAAUACGUGGGAAGUACA |
| 916 | HPV45_25_HR&LR(−18)_3241 | GUGUUAGCUAUUGGGGUGUAUAUUA |
| 917 | HPV45_25_HR&LR(−18)_3216 | GGGAUAUGGGACAAAACAGCAGCAU |
| 918 | HPV45_25_HR&LR(−18)_3173 | GAACUAUGUAGUAUGGGACAGUAUA |
| 919 | HPV45_25_HR&LR(−18)_3134 | CGUGCACGUAUACUUUGAUGGCAAC |
| 920 | HPV45_25_HR&LR(−18)_3092 | GAAUACAGAACCGUCGCAGUGUUUU |
| 921 | HPV45_25_HR&LR(−18)_3039 | AGCAAGUAUAACAAUGAGGAAUGGA |
| 922 | HPV45_25_HR&LR(−18)_2918 | UACAGCAAGGGAACAUGGUAUUACC |
| 923 | HPV45_25_HR&LR(−18)_2883 | UGGCAACUUAUACGUUUGGAAAAUG |
| 924 | HPV45_25_HR&LR(−18)_2850 | GACAGUAAAGACAUAAACAGCCAAA |
| 925 | HPV45_25_HR&LR(−18)_2765 | GACGAUGAAGAUGCAGACACCGAAG |
| 926 | HPV45_25_HR&LR(−18)_2642 | ACGGUAUUUACAUUUCCACAUGCAU |
| 927 | HPV45_25_HR&LR(−18)_2586 | CAUCCAAUAUUGAUCCAGCAAAAGA |
| 928 | HPV45_25_HR&LR(−18)_2560 | GCUAAAAUGUCCUCCAAUCCUAUUA |
| 929 | HPV45_25_HR&LR(−18)_2431 | AGCAGAUACUAAGGUAGCCAUGUUG |
| 930 | HPV45_25_HR&LR(−18)_2358 | GUUUUAUACAUUUCCUACAAGGUGC |
| 931 | HPV45_25_HR&LR(−18)_2266 | GGCACUAAAGGAAUUUCUUAAAGGA |
| 932 | HPV45_25_HR&LR(−18)_1781 | UUGUUGCACGUACCUGAAACAUGUA |
| 933 | HPV45_25_HR&LR(−18)_1754 | CUAACUGUUGCAAAAGGCUUAAGCA |
| 934 | HPV45_25_HR&LR(−18)_1676 | GCCCAUAUCCAAUGUUUAGAUUGUA |
| 935 | HPV45_25_HR&LR(−18)_1599 | GGGUAAUGGCUAUAUUUGGAGUUAA |
| 936 | HPV45_25_HR&LR(−18)_1541 | CUGUCAUUUACGGAUUUGGUUAGAA |
| 937 | HPV45_25_HR&LR(−18)_1516 | GGCAGUAUUUAAAGACAUAUAUGGG |
| 938 | HPV45_25_HR&LR(−18)_1474 | AAAGGAGCUAUUACAAGCAAGUAAC |
| 939 | HPV45_25_HR&LR(−18)_1449 | AUCCGCAUUGCAGUAUUACAGAACU |
| 940 | HPV45_25_HR&LR(−18)_1424 | AGUAGUGACAAUGCAGAAAAUGUAG |
| 941 | HPV45_25_HR&LR(−18)_1399 | UAGUACACAAAGUAGUGGUGGGGAU |
| 942 | HPV45_25_HR&LR(−18)_1365 | UAAACACUAAUGCGGAAAAUGGCGG |
| 943 | HPV45_25_HR&LR(−18)_1338 | UGGAAGCUGCAGAGACUCAGGUAAC |
| 944 | HPV45_25_HR&LR(−18)_1242 | GUCCACGGUUACAAGAAAUUUCAUU |
| 945 | HPV45_25_HR&LR(−18)_1217 | CAGCUAAGUGUGGAUACGGAUCUAA |
| 946 | HPV45_25_HR&LR(−18)_1153 | GGUGUUGCAUCUUUUAAAACGAAAG |
| 947 | HPV45_25_HR&LR(−18)_1124 | CAUGCGCAGGAAGUUCAGAAUGAUG |
| 948 | HPV45_25_HR&LR(−18)_1072 | ACAAUUAUCCAUUUGUGAACAGGCA |
| 949 | HPV45_25_HR&LR(−18)_954 | GUAAUGGCUGGUUCUUUGUAGAAAC |
| 950 | HPV45_25_HR&LR(−18)_897 | CUAACCAAUAAUCUACAAUGGCGGA |
| 951 | HPV45_25_HR&LR(−18)_832 | GGACCUUAGAACACUACAGCAGCUG |
| 952 | HPV45_25_HR&LR(−18)_799 | CAGAAUUGAGCUUACAGUAGAGAGC |
| 953 | HPV45_25_HR&LR(−18)_649 | AGAUCCUGUUGACCUGUUGUGUUAC |
| 954 | HPV45_25_HR&LR(−18)_624 | UGCAUUUGGAACCUCAGAAUGAAUU |
| 955 | HPV45_25_HR&LR(−18)_596 | CCCCGGGAAACACUGCAAGAAAUUG |
| 956 | HPV45_25_HR&LR(−18)_570 | CAAGUAUAGCAAUAAGUAUGCAUGG |
| 957 | HPV45_25_HR&LR(−18)_536 | ACGGCAAGAAAGACUUCGCAGACGU |
| 958 | HPV45_25_HR&LR(−18)_511 | AGUGUAAUACAUGUUGUGACCAGGC |
| 959 | HPV45_25_HR&LR(−18)_486 | AGCAUAGCUGGACAGUACCGAGGGC |
| 960 | HPV45_25_HR&LR(−18)_461 | CCUUAAGGACAAACGAAGAUUUCAC |
| 961 | HPV45_25_HR&LR(−18)_348 | AACUCUGUAUAUGGAGAGACACUGG |
| 962 | HPV45_25_HR&LR(−18)_265 | UGUAUAGAGACUGUAUAGCAUAUGC |
| 963 | HPV45_25_HR&LR(−18)_218 | GGAACGCACAGAGGUAUAUCAAUUU |
| 964 | HPV45_25_HR&LR(−18)_188 | UAUUGCCUGUGUAUAUUGCAAAGCA |
| 965 | HPV45_25_HR&LR(−18)_163 | UGAAUACAUCACUACAAGACGUAUC |
| 966 | HPV45_25_HR&LR(−18)_138 | AAGCUACCAGAUUUGUGCACAGAAU |
| 967 | HPV45_25_HR&LR(−18)_113 | UGACGAUCCAAAGCAACGACCCUAC |
| 968 | HPV45_25_HR&LR(−18)_87 | AAAGUGCAUUACAGGAUGGCGCGCU |
| 969 | HPV45_7599 | CCUGGUAUUAGUCAUUUUCCUGUCC |
| 970 | HPV45_6860 | UGGUGGAUACAUAUCGUUUUGUGCA |
| 971 | HPV45_2617 | AUGGCCAUAUUUAGAAAGUAGGGUG |
| 972 | HPV45_1297 | GUUGUUUACAAUAUCAGAUAGUGGC |
| 973 | HPV45_733 | ACUACCAGCCCGACGAGCCGAACCA |
| 974 | HPV45_414 | UGCCUGCGGUGCCAGAAACCAUUGA |

In one embodiment, the present invention provides an isolated polynucleotide for specific hybridization to HPV 31 consisting essentially of a sequence or a complement thereof selected from the group consisting of: SEQ ID NOs: 310-454 (See Table 4). In some embodiments, the present invention provides a set of polynucleotides for specific hybridization to HPV 31, wherein the set comprises at least one polynucleotide consisting essentially of a sequence or a complement thereof selected from the group consisting of: SEQ ID NOs: 310-454. In certain embodiments, the methods of the present invention utilize a set of polynucleotide probes for specific hybridization to HPV 31 comprising SEQ ID NOs: 310-454.

TABLE 4
Polyribonucleotide probes for
determining HPV 31 nucleic acid.

| SEQ ID NO: | Name | Sequence |
|---|---|---|
| 310 | HPV31_7871 | GUUUUCGGUUACAGUUUUACAAGCA |
| 311 | HPV31_7799 | CCAAGGUUGUGUCAUGCAUUAUAAA |
| 312 | HPV31_7760 | CCUUGAUUGCAGUGCUGGCUUUUGC |
| 313 | HPV31_7709 | CCUACACACCUUAAACUGCUUUUAG |
| 314 | HPV31_7670 | UGUAGUUCAACUAUGUGUCAUGCAC |
| 315 | HPV31_7620 | CCAGUCCAACUUUGCAAUUAUACUA |
| 316 | HPV31_7595 | CUAACACACCUUGCCAACAUAUAAU |
| 317 | HPV31_7570 | AACAUUCUGGCUUGUAGUUUCCUGC |
| 318 | HPV31_7502 | CAUGCUAGUACAACUAUGCUGAUGC |
| 319 | HPV31_7462 | CAUUUUAAAUCCCUAACCGUUUUCG |
| 320 | HPV31_7437 | CUACUCCAUUUUGAUUUUAUGCAGC |
| 321 | HPV31_7396 | UAGUAAAAGUUGUACACCCGGUCCG |
| 322 | HPV31_7350 | CAAUAGUCAUGUACUUAUUUCUGCC |
| 323 | HPV31_7325 | UGUUCCUACUUGUUCCUGCUCCUCC |
| 324 | HPV31_7261 | GUUGUCCUUAUAUACACCCUAUUAG |
| 325 | HPV31_7232 | AUAUGUGUAUACCUGUGUGUGUUGU |
| 326 | HPV31_7111 | GCUGUAUUGUAUAUGUGUGUGUUUG |
| 327 | HPV31_7086 | UGUGUCUGUAUGUGUAUGUGCUUGU |
| 328 | HPV31_7024 | AUCUACCACUACACCAGCAAAACGU |
| 329 | HPV31_6984 | GUCCUAAAUUUAAAGCAGGUAAACG |
| 330 | HPV31_6860 | CCCAAGGAAGAUCCAUUUAAAGAUU |
| 331 | HPV31_6786 | CAGGUUCUUUGGAGGAUACCUAUAG |
| 332 | HPV31_6593 | GCAAUUGCAAACAGUGAUACUACAU |
| 333 | HPV31_6567 | GUAGUACCAAUAUGUCUGUUUGUGC |
| 334 | HPV31_6424 | AUACUUUCCUACACCUAGCGGCUCC |
| 335 | HPV31_6390 | GCUCCGGUUCAACAGCUACUUUAGC |
| 336 | HPV31_6358 | UGAAUCGGUCCCUACUGACUUAUAU |
| 337 | HPV31_6197 | GACACUAAAAGUAAUGUUCCUUUGG |
| 338 | HPV31_6089 | GCUAUUACCCCUGGUGAUUGUCCUC |
| 339 | HPV31_6017 | CAACUGUGUUUACUUGGUUGCAAAC |
| 340 | HPV31_5962 | CGGUGGUCCUGGCACUGAUAAUAGG |
| 341 | HPV31_5701 | UUCCAUACCUAAAUCUGACAAUCCU |
| 342 | HPV31_5666 | AGUGCUAGGCUGCUUACAGUAGGCC |
| 343 | HPV31_5640 | GAACCAACAUAUAUUAUCACGCAGG |
| 344 | HPV31_5596 | UGUCCCAGUGUCUAAAGUUGUAAGC |
| 345 | HPV31_5571 | GCGAGGCUACUGUCUACUUACCACC |
| 346 | HPV31_5440 | GCCCCUACAACGCCACAAGUGUCUA |
| 347 | HPV31_5415 | UACACAGGUUUUCCCAUUUCCUUUG |
| 348 | HPV31_5390 | CUGAUGUACCUAUAGAGCAUGCACC |
| 349 | HPV31_5364 | UUUUGACAUUCCCAUAUUUUCUGGG |
| 350 | HPV31_5337 | AAAUACCACUGUGCCACUAAGUACA |
| 351 | HPV31_5294 | CUGCUGUACAGUCCACAUCUGCUGU |
| 352 | HPV31_5258 | UGGAUACACCUGCCACACAUAAUGU |
| 353 | HPV31_5173 | AUGCAACCUUUAGGGGCGUCUGCAA |
| 354 | HPV31_5148 | UAAUCCUGCAGGUGAAAGUAUUGAA |
| 355 | HPV31_5097 | UGGUGCUACUAUUGGUGCAAGGGUG |
| 356 | HPV31_5072 | AUAAACAAACUUUGCGCACUCGUAG |
| 357 | HPV31_5046 | CACUGUUAGAUAUAGUAGACUAGGU |
| 358 | HPV31_4990 | CCCGACUUUCUAGAUAUUAUAGCAU |
| 359 | HPV31_4965 | UACAUCGCAUAAUAUAGCCCCUGAU |
| 360 | HPV31_4922 | CCUAUGAAACUGUAAAUGCUGAAGA |
| 361 | HPV31_4888 | GCUCCAAAACAGCUAAUUACAUAUG |
| 362 | HPV31_4841 | GUAAGGCUACACAACAAGUAAAAGU |
| 363 | HPV31_4782 | CAUAACAAGUAGCACACCCAUUCCA |
| 364 | HPV31_4688 | CAGGUCAUUUACUACUUUCAUCAUC |
| 365 | HPV31_4663 | CAGCCUCCUACACCUGCAGAAACAU |
| 366 | HPV31_4622 | GCACACAUGAAAAUCCUACUUUUAC |
| 367 | HPV31_4583 | CAGACACAACACCUGCAAUUUUAGA |
| 368 | HPV31_4558 | UCUGGGUUUGACAUUGCUACAACUG |
| 369 | HPV31_4533 | UCCUAUACCACACCCUCCUACAACA |
| 370 | HPV31_4508 | GAAUUGUUGAUGUUGGUGCCCCUGC |
| 371 | HPV31_4478 | CCUCUAUAGUAAGUCUUGUUGAAGA |
| 372 | HPV31_4442 | CACCAGUUAGCAUUGACCCUGUAGG |
| 373 | HPV31_4417 | UCUGAGGCAAGUAUACCUAUUAGAC |
| 374 | HPV31_4392 | UCUUAGUACACGUCCUUCUACAGUA |
| 375 | HPV31_4303 | AUAUUAAGGUAUGGUAGUAUGGGUG |
| 376 | HPV31_4255 | CCAUCAGACGUUAUACCUAAAAUAG |
| 377 | HPV31_4182 | ACGCUCUACAAAACGCACUAAACGU |
| 378 | HPV31_3967 | UUAUUGCAACCUCUCCAUUACGUUG |
| 379 | HPV31_3923 | GUCGGUAUAUGCAACACUACUAUUA |
| 380 | HPV31_3898 | UCAUACGUCCACUUGUGCUGUCUGU |
| 381 | HPV31_3873 | UGUGUGCUACUAUUUGUGUGUCUUG |
| 382 | HPV31_3789 | CAACAGGAUAUAUGACUAUUUAGCC |
| 383 | HPV31_3673 | UUGGACAUGUACAGAUGGAAAACAU |
| 384 | HPV31_3645 | UGUAUGAACAAGUGUCAUCUACAUG |
| 385 | HPV31_3561 | CUGCAACUACACCUAUAAUACACUU |
| 386 | HPV31_3536 | AACCAAACAAGGGCUGUCAGUUGUC |
| 387 | HPV31_3506 | UGUGGGGUUAUCAGUGCAGCUGCAU |
| 388 | HPV31_3428 | CCAAGAACAGAGCCAGAGCACAGAA |
| 389 | HPV31_3361 | GAAUUCCAAAACCUGCGCCUUGGGC |
| 390 | HPV31_3308 | UCCUUUGCUGGGAUUGUUACAAAGC |
| 391 | HPV31_3281 | GAAUCUGUAUUUAGCAGUGACGAAA |
| 392 | HPV31_3158 | GGCAUUUAUUAUGUACAUGAAGGAC |
| 393 | HPV31_3133 | UGUGGAAGGGCAAGUUAAUUGUAAG |
| 394 | HPV31_3108 | UAUGUAUAGAUGGCCAAUGUACUGU |
| 395 | HPV31_3073 | CACCAUGCAUUAUACUAACUGGAAA |
| 396 | HPV31_3046 | GGUGCAAUUUGAUGGUGAUGUACAC |
| 397 | HPV31_2988 | UUGAACUGUAUUUAACUGCACCUAC |
| 398 | HPV31_2963 | GACUGGACAAUGCAGCAAACAAGUC |
| 399 | HPV31_2897 | GCCUUACAAGCUAUUGAACUACAAA |
| 400 | HPV31_2870 | CCAGCGUUGUCAGUAUCAAAGGCCA |
| 401 | HPV31_2839 | GGGAAUACACAGUAUUAACCACCAG |
| 402 | HPV31_2783 | GACUAUUGGAAACAUAUUCGACUUG |
| 403 | HPV31_2698 | GACUCUUUCUCAACGUUUAAAUGUG |
| 404 | HPV31_2660 | UAAAUUUGCACGAGGAAGAGGACAA |
| 405 | HPV31_2520 | UGACAGAUGGCCAUACCUACAUAGC |
| 406 | HPV31_2430 | CCCUGUAUCUAUAGAUGUAAAGCAU |
| 407 | HPV31_2402 | AUUACCUACGAAAUGCACUAGAUGG |
| 408 | HPV31_2222 | UAAUACAUGGUGCACCUAAUACAGG |
| 409 | HPV31_2109 | AGGUGACUGGAGGGACAUAGUAAAG |
| 410 | HPV31_2084 | GUAGAUGUGACAAAGUUAGUGACGA |
| 411 | HPV31_1949 | CUGACAGUGAUAGUAAUGCAUGUGC |
| 412 | HPV31_1855 | GACACAACAUUUGAUUUGUCCCAAA |
| 413 | HPV31_1712 | GUAUGUUAAUUCAGCCACCCAAAUU |
| 414 | HPV31_1591 | UUACAAAGUUUAGCAUGUUCCUGGG |
| 415 | HPV31_1566 | GCAACCAUAUUGUUUGUAUUGCCAU |
| 416 | HPV31_1540 | GUUGCAGAAGGAUUUAAAACCCUAU |
| 417 | HPV31_1515 | AGCUGCGUUUGGAGUUACAGGUACA |
| 418 | HPV31_1490 | AAAGCACAUGUACUGAUUGGUGUGU |
| 419 | HPV31_1462 | GAACUAAUUAGGCCAUUUCAAAGCA |
| 420 | HPV31_1408 | GGUAAAGCUGCUAUGUUAGGUAAAU |
| 421 | HPV31_1369 | CCAACACGUAAUAUAUUGCAAGUGU |
| 422 | HPV31_1344 | ACAUAGUGAACGAGAGAAUGAAACU |
| 423 | HPV31_1319 | UAAGUUGUAAUGGUAGUGACGGGAC |
| 424 | HPV31_1294 | CAGGUAGAGGAGCAACAAACAACAU |
| 425 | HPV31_1269 | UGAAGUGGAAACGCAGCAGAUGGUA |
| 426 | HPV31_1233 | ACUCUUUGAACUUCCAGACAGCGGG |
| 427 | HPV31_1181 | CACGGUUAAAAGCUAUAUGCAUAGA |
| 428 | HPV31_1084 | GCGGAGGAACAUGCAGAGGCUGUGC |
| 429 | HPV31_994 | GAGGAUAUGGUUGACUUUAUUGACA |
| 430 | HPV31_965 | ACGAAAAUGAAGACAGUAGUGAUAC |
| 431 | HPV31_940 | CAGACAGGGGACAACAUUUCAGAGG |
| 432 | HPV31_907 | GGUUGGUUUUAUGUAGAAGCAGUAA |
| 433 | HPV31_848 | AGACUGUAACUACAAUGGCUGAUCC |
| 434 | HPV31_814 | CUCAUUUGGAAUCGUGUGCCCCAAC |
| 435 | HPV31_789 | GCAUAUUGCAAGAGCUGUUAAUGGG |
| 436 | HPV31_764 | GUACAGAGCACACAAGUAGAUAUUC |
| 437 | HPV31_727 | CUUUUGUUGUCAGUGUAAGUCUACA |
| 438 | HPV31_700 | GGACACAUCCAAUUACAAUAUCGUU |
| 439 | HPV31_662 | GAGGAUGUCAUAGACAGUCCAGCUG |
| 440 | HPV31_629 | UGUUAUGAGCAAUUACCCGACAGCU |
| 441 | HPV31_594 | UGUUAGAUUUGCAACCUGAGGCAAC |
| 442 | HPV31_569 | GAAACACCUACGUUGCAAGACUAUG |
| 443 | HPV31_535 | CUCGUACUGAAACCCAAGUGUAAAC |
| 444 | HPV31_510 | CGUUGCAUAGCAUGUUGGAGAAGAC |
| 445 | HPV31_478 | GAUUCCACAACAUAGGAGGAAGGUG |
| 446 | HPV31_340 | GGUAUAGAUAUAGUGUGUAUGGAAC |
| 447 | HPV31__287 | CGGAGUGUGUACAAAAUGUUUAAGA |
| 448 | HPV31_262 | UAGUAUAUAGGGACGACACACCACA |
| 449 | HPV31_220 | CAGAAACAGAGGUAUUAGAUUUUGC |
| 450 | HPV31_186 | AGAUUGAAUUGUGUCUACUGCAAAG |
| 451 | HPV31_161 | AUUGGAAAUACCCUACGAUGAACUA |
| 452 | HPV31_136 | GGAAAUUGCAUGAACUAAGCUCGGC |
| 453 | HPV31_89 | GUGCAAACCUACAGACGCCAUGUUC |
| 454 | HPV31_60 | CGGUUGGUAUAUAAAGCACAUAGUA |

In one embodiment, the present invention provides an isolated polynucleotide for specific hybridization to HPV 33 consisting essentially of a sequence or a complement thereof selected from the group consisting of: SEQ ID NOs: 455-579 (See Table 5). In some embodiments, the present invention provides a set of polynucleotides for specific hybridization to HPV 33, wherein the set comprises at least one polynucleotide consisting essentially of a sequence or a complement thereof selected from the group consisting of: SEQ ID NOs: 455-579. In certain embodiments, the methods of the present invention utilize a set of polynucleotide probes for specific hybridization to HPV 33 comprising SEQ ID NOs:455-579.

TABLE 5
Polyribonucleotide probes for
determining HPV 33 nucleic acid.

| SEQ ID NO: | Name | Sequence |
|---|---|---|
| 455 | HPV33_7867 | CCGUUUUAGGUCAUAUUGGUCAUUU |
| 456 | HPV33_7831 | UGAGUCACUACCUGUUUAUUACCAG |
| 457 | HPV33_7805 | GUAUGCCAAACUAUGCCUUGUAAAA |
| 458 | HPV33_7780 | CAGUUUUGGCUUACACAAUUGCUUU |
| 459 | HPV33_7680 | UCAUAUAUACAUGCAGUGCAAUUGC |
| 460 | HPV33_7655 | GUUUGUCUGUACUUGCUGCAUUGAC |
| 461 | HPV33_7630 | UUAAUCCUUUUCUUUCCUGCACUGU |
| 462 | HPV33_7605 | AUACCCUAUGACAUUGGCAGAACAG |
| 463 | HPV33_7576 | GUUUGUCUGUACUUGCUGCAUUGGC |
| 464 | HPV33_7551 | UUAAUCCUUUUCUUUCCUGCACUGU |
| 465 | HPV33_7526 | AUACCCUAUGACAUUGGCAGAACAG |
| 466 | HPV33_7465 | GUCCAUAUUGUACAAUUUCCUCCAU |
| 467 | HPV33_7425 | CCUACAUGUUUAGUAUUGCUUUACC |
| 468 | HPV33_7389 | CAAUGUACCUACCUUUAUUUCCCUA |
| 469 | HPV33_7364 | GUAUUGCUUGCCCUACCCUGCAUUG |
| 470 | HPV33_7339 | GGUGUACCUAUAUGAGUAAGGAGUU |
| 471 | HPV33_7228 | UGUACUUGUUUGUGUGCAUGUUCUA |
| 472 | HPV33_7129 | CUGUCUAUGUACUUUGUGUUGUUGU |
| 473 | HPV33_7051 | CACCCGCACAUCGUCUGCAAAACGC |
| 474 | HPV33_7016 | GCAAAACCUAAACUUAAACGUGCAG |
| 475 | HPV33_6914 | GGUAAAUAUACAUUUUGGGAAGUGG |
| 476 | HPV33_6804 | GUUUAACACCUCCUCCAUCUGCUAG |
| 477 | HPV33_6630 | CACAAGUAACUAGUGACAGUACAUA |
| 478 | HPV33_6490 | GGUUACUUCCGAAUCUCAGUUAUUU |
| 479 | HPV33_6434 | GGAACUACUGCCUCUAUUCAAAGCA |
| 480 | HPV33_6405 | UUCCCGAUGACCUGUACAUUAAAGG |
| 481 | HPV33_6380 | AGGGCUGGUACAUUAGGAGAGGCUG |
| 482 | HPV33_6135 | CUGCCAAUGAUUGUCCACCUUUAGA |
| 483 | HPV33_6109 | AGGUGUUGCUUGUACUAAUGCAGCA |
| 484 | HPV33_6063 | UAUGUUUACUUGGAUGUAAGCCUCC |
| 485 | HPV33_6004 | UGGACAACCGGGUGCUGAUAAUAGG |
| 486 | HPV33_5979 | ACACUGAAACCGGUAACAAGUAUCC |
| 487 | HPV33_5902 | UGUAGGCCUUGAAAUAGGUAGAGGG |
| 488 | HPV33_5839 | UAAAUUUGGAUUUCCUGACACCUCC |
| 489 | HPV33_5783 | CCCAAAGUAUCAGGCUUGCAAUAUA |
| 490 | HPV33_5521 | GCUGACUUUGUUUUACAUCCUAGUU |
| 491 | HPV33_5496 | UUUUGACACCAUUGUUGUAGACGGU |
| 492 | HPV33_5462 | CUAGCCCAUUUGUUCCUAUUUCGCC |
| 493 | HPV33_5412 | UACUCCUGUUAUGUCUGGCCCUGAU |
| 494 | HPV33_5375 | CCAGCAAUGUGUCUAUACCUUUAAA |
| 495 | HPV33_5349 | AUACAGUACGUUUGCAACAACACGU |
| 496 | HPV33_5324 | AUGUACACACCCCAAUGCAACACUC |
| 497 | HPV33_5299 | GAUGUUUAUGCUGACGAUGUGGAUA |
| 498 | HPV33_5249 | CUUUACAUGAUACUUCUACAUCGUC |
| 499 | HPV33_5219 | CCGUGCCAAAUGAACAAUAUGAAUU |
| 500 | HPV33_5194 | AGUCCUAUUGUGCCUUUAGACCACA |
| 501 | HPV33_5164 | GGAGCUAGAAUACAUUAUUAUCAGG |
| 502 | HPV33_5092 | CGUAGACAUACUGUGCGUUUUAGUA |
| 503 | HPV33_4993 | CCUGAAGACACAUUACAAUUUCAAC |
| 504 | HPV33_4888 | UUAUAUAGUCGCAAUACCCAACAGG |
| 505 | HPV33_4836 | UGUAACAUCAAGCACGCCCAUUCCA |
| 506 | HPV33_4811 | UUGUUGUUUCCACAGACAGUAGUAA |
| 507 | HPV33_4775 | GCACACAAAGUUAUGAAAACAUACC |
| 508 | HPV33_4742 | CUGGACAUUUUAUAUUUUCUUCCCC |
| 509 | HPV33_4715 | UACACCCUCCAGCGCCUGCAGAAGC |
| 510 | HPV33_4652 | GGGAGUCAUCUAUUCAAACUAUUUC |
| 511 | HPV33_4603 | ACUACAUCUGCAGAUACUACACCUG |
| 512 | HPV33_4568 | CCCCAUCUAUUCCUACACCAUCAGG |
| 513 | HPV33_4510 | GACUCGUCUAUAGUGUCAUUAAUAG |
| 514 | HPV33_4485 | UACUGUAGACACUGUUGGACCUUUA |
| 515 | HPV33_4460 | CCUUGCAGCCUAUACGUCCUCCGGU |
| 516 | HPV33_4435 | ACUGACCCACCUACAGCUGCAAUCC |
| 517 | HPV33_4317 | AGGAAGUACCAUAGCAGAUCAAAUU |
| 518 | HPV33_4119 | CAUGGUGGUGUUUUAACAUUGUUGU |
| 519 | HPV33_4060 | GCAUAUGACACAACAAGAGUAAUGU |
| 520 | HPV33_3969 | UUUGGGUGUUUGUGGGAUCUCCUUU |
| 521 | HPV33_3944 | UGGUUGCUGGUGUUGGUAUUGCUGC |
| 522 | HPV33_3773 | CUACUGUGCAAAUAAGUACUGGAUU |
| 523 | HPV33_3719 | CAUUUGUAACUGAACAGCAACAACA |
| 524 | HPV33_3646 | UAUAGUUCUAUGUCAUCCACCUGGC |
| 525 | HPV33_3555 | UAGUUCUAACGUUGCACCUAUAGUG |
| 526 | HPV33_3530 | GCACAAACAAGCAGCGGACUGUGUG |
| 527 | HPV33_3497 | UGGACAAUAGAACAGCACGUACUGC |
| 528 | HPV33_3463 | CCCCUUACAAAGCUGUUCUGUGCAG |
| 529 | HPV33_3408 | ACCACAAGCAGCGGCCAAACGACGA |
| 530 | HPV33_3380 | ACAUACAGACAGACAACGAUAACCG |
| 531 | HPV33_3338 | CGUCUAUAUCUAGCAACCAAAUAUC |
| 532 | HPV33_3185 | CUAUGGUUACAGGGAAAGUAGAUUA |
| 533 | HPV33_3135 | GGAUUAUACAAACUGGGGUGAAAUA |
| 534 | HPV33_3096 | AGUAACUGUGCAAUAUGACAAUGAC |
| 535 | HPV33_3008 | AUAGUACAAGCCAAUGGACAUUGCA |
| 536 | HPV33_2939 | CAUCAAAGACCAAAGCAUUUCAAGU |
| 537 | HPV33_2895 | GGGAUUUUCACAUUUAUGCCACCAG |
| 538 | HPV33_2867 | GUGCUUUAUUGUAUACAGCCAAACA |
| 539 | HPV33_2809 | GCUGAUAAAACUGAUUUACCAUCAC |
| 540 | HPV33_2654 | CCCAGUGUAUGCAAUAAAUGAUGAA |
| 541 | HPV33_2576 | CUCUAGAUGGCCAUAUUUACAUAGU |
| 542 | HPV33_2526 | UUAAAAUGUCCACCACUGCUUCUUA |
| 543 | HPV33_2454 | GAUGAUUACAUGAGAAAUGCGUUAG |
| 544 | HPV33_2419 | UAGAUGAUGUAACGCCAAUAAGUUG |
| 545 | HPV33_2269 | GCUGUAUGCUAAUUUGUGGACCAGC |
| 546 | HPV33_2174 | GAGACCAAUAGUACAGUUGUUAAGA |
| 547 | HPV33_2004 | GCAGAUUCAAAUAGUAAUGCUGCUG |
| 548 | HPV33_1951 | AUGAUAACGAGUUAACGGACGAUAG |
| 549 | HPV33_1795 | GGAGCCAAACAUGUGCAUUGUAUUG |
| 550 | HPV33_1763 | AACAUGUAUGGUUAUAGAGCCACCA |
| 551 | HPV33_1715 | CAGGUUAACAGUAGCAAAACUAAUG |
| 552 | HPV33_1567 | GUAUAACAGGAUAUGGAAUUAGUCC |
| 553 | HPV33_1496 | GGCCUAUGGAAUAAGUUUUAUGGAA |
| 554 | HPV33_1426 | CGUUGCAGGAAAUUAGUAAUGUUCU |
| 555 | HPV33_1395 | GAGACAAAUGUAGAUAGCUGUGAAA |
| 556 | HPV33_1345 | UAAAUGACUUAGAAUCUAGUGGGGU |
| 557 | HPV33_1320 | GAAAGUCAAAAUGGCGACACAAACU |
| 558 | HPV33_1295 | AACUCAGCAGAUGGUACAACAGGUA |
| 559 | HPV33_1183 | AUCGUGCUGCAAACCCGUGUAGAAC |
| 560 | HPV33_1154 | UUCACAAAGUGCUGCGGAGGACGUU |
| 561 | HPV33_1009 | GCACGGAUUUACUAGAGUUUAUAGA |
| 562 | HPV33_984 | GAGGAUGAAACAGCAGAUGACAGUG |
| 563 | HPV33_870 | UCAUCUACAAUGGCCGAUCCUGAAG |
| 564 | HPV33_830 | AGUGAAUAUUGUGUGCCCUACCUGU |
| 565 | HPV33_805 | CCAUACAGCAACUACUUAUGGGCAC |
| 566 | HPV33_780 | AACAGUACAGCAAGUGACCUACGAA |
| 567 | HPV33_742 | GUUGUCACACUUGUAACACCACAGU |
| 568 | HPV33_717 | ACAGCUGAUUACUACAUUGUAACCU |
| 569 | HPV33_617 | AUAUCCUGAACCAACUGACCUAUAC |
| 570 | HPV33_575 | GAGAGGACACAAGCCAACGUUAAAG |
| 571 | HPV33_539 | GUAGAGAAACUGCACUGUGACGUGU |
| 572 | HPV33_490 | AUUUCGGGUCGUUGGGCAGGGCGCU |
| 573 | HPV33_457 | CGACAUGUGGAUUUAAACAAACGAU |
| 574 | HPV33_424 | UGUCAAAGACCUUUGUGUCCUCAAG |
| 575 | HPV33_301 | CUGUGUUUGCGGUUCUUAUCUAAAA |
| 576 | HPV33_274 | GAGGGAAAUCCAUUUGGAAUAUGUA |
| 577 | HPV33_214 | CCUUUGCAACGAUCUGAGGUAUAUG |
| 578 | HPV33_183 | CAUUGAACUACAGUGCGUGGAAUGC |
| 579 | HPV33_103 | ACGACUAUGUUUCAAGACACUGAGG |

In one embodiment, the present invention provides an isolated polynucleotide for specific hybridization to HPV 35 consisting essentially of a sequence or a complement thereof selected from the group consisting of: SEQ ID NOs: 580-722 (See Table 6). In some embodiments, the present invention provides a set of polynucleotides for specific hybridization to HPV 35, wherein the set comprises at least one polynucleotide consisting essentially of a sequence or a complement thereof selected from the group consisting of: SEQ ID NOs:580-722. In certain embodiments, the methods of the present invention utilize a set of polynucleotide probes for specific hybridization to HPV 35 comprising SEQ ID NOs:580-722.

TABLE 6
Polyribonucleotide probes for
determining HPV 35 nucleic acid.

| SEQ ID NO: | Name | Sequence |
|---|---|---|
| 580 | HPV35_7767 | CACAUUGUUAUAUGCACACAGGUGU |
| 581 | HPV35_7737 | CAUGCAUGUAAAACAUUACUCACUG |
| 582 | HPV35_7711 | CACAUCCUGCCAACUUUAAGUUAAA |
| 583 | HPV35_7648 | CUAAAGGGCUUUAAUUGCACACCUU |
| 584 | HPV35_7606 | AACACUUGUAACAGUGCUUUUAGGC |
| 585 | HPV35_7549 | CUUGAUUCAUCUUGCAGUAUUAGUC |
| 586 | HPV35_7493 | GUGUUCCUGAUAUAUAUUGUUUGCC |
| 587 | HPV35_7424 | GGUUGCUGUUGGUAAGCUUUAUAUG |
| 588 | HPV35_7393 | GUGUCCUUUACAUUACCUUUCAACC |
| 589 | HPV35_7327 | GAGCUUACAUAAUUACAUGACAGCU |
| 590 | HPV35_7302 | UUGUAUGACUAUGGUGCACCGAUAU |
| 591 | HPV35_7261 | GCCAUAAAGUGAUGUGUGUGUUUAU |
| 592 | HPV35_7236 | GUACUUAGUGUGUAGUAGUUCAGUA |
| 593 | HPV35_7206 | UUGUGCAAUGUGUUGUACGUGGGUG |
| 594 | HPV35_7180 | CAUGGCGUGUAAAUGUGUGUAUAAU |
| 595 | HPV35_7155 | UGUUGUGGUGCCUGUUUGUGUUGUA |
| 596 | HPV35_7108 | CAUGUAUACUGUGUGUUAUGUGUUG |
| 597 | HPV35_7028 | GCGUGCAGCUCCAGCAUCUACAUCU |
| 598 | HPV35_6874 | CACCAAAACCUAAAGAUGAUCCAUU |
| 599 | HPV35_6825 | ACAUAUCGCUAUGUAACAUCACAGG |
| 600 | HPV35_6759 | AACCCGUCCAUUUUAGAGGAUUGGA |
| 601 | HPV35_6592 | GUACAAAUAUGUCUGUGUGUUCUGC |
| 602 | HPV35_6474 | GUAACCUCCGAUGCACAAAUAUUUA |
| 603 | HPV35_6439 | GUACUAGUUAUUUUCCUACUCCUAG |
| 604 | HPV35_6392 | AGUACCUGCAGACCUAUAUAUUAAG |
| 605 | HPV35_6296 | UUCUGAGCCAUAUGGAGAUAUGUUA |
| 606 | HPV35_6245 | CCUAGAUAUAUGCAGUUCCAUUUGC |
| 607 | HPV35_6141 | CCUUUGGAGUUACUAAACACUGUAC |
| 608 | HPV35_6115 | ACCAGGUAAAAGCAGGAGAAUGUCC |
| 609 | HPV35_6045 | CAAUUGUGUUUAAUAGGUUGUAGGC |
| 610 | HPV35_6006 | GAUAACAGGGAAUGCAUUUCUAUGG |
| 611 | HPV35_5981 | UGGUAACUCUGGUAACUCUGGUACA |
| 612 | HPV35_5877 | UGUACAGGAGUUGAAGUAGGUCGUG |
| 613 | HPV35_5849 | UCCCUGCCUCCAGCGUUUGGUUUGG |
| 614 | HPV35_5748 | AAAAUAGCAGUACCCAAGGUAUCUG |
| 615 | HPV35_5682 | GCAGGCAGUUCUAGGCUAUUAGCUG |
| 616 | HPV35_5589 | UCUAACGAAGCCACUGUCUACCUGC |
| 617 | HPV35_5465 | CCCACAGGUCCUAUAUAUUCUAUUA |
| 618 | HPV35_5440 | UAUUACUAACUCUGUACUACCGGUA |
| 619 | HPV35_5412 | GGCCAGACAUUGUAUUUAACUCUAA |
| 620 | HPV35_5387 | GGCUAUGAUAUUCCUAUAACAGCAG |
| 621 | HPV35_5354 | GUUCCUAGCAAUACUACUAUACCAU |
| 622 | HPV35_5274 | CUCCUAUAGAUACUGAGGAAGAUAU |
| 623 | HPV35_5223 | CACAUACCACUGUUUCAACAUCAUU |
| 624 | HPV35_5198 | UUACAACAUGUACCAUCCUCUUUAC |
| 625 | HPV35_5120 | AGUGGAAAAGCUAUAGGGGCACGGG |
| 626 | HPV35_5094 | GUAAUAAACGUACUAUGCAUACACG |
| 627 | HPV35_5050 | UGCACUAACAUCUAGGAAAGGCACU |
| 628 | HPV35_5024 | AUGGACAUUAUAGCUUUACAUAGGC |
| 629 | HPV35_4993 | GGAUAUUAGCUUAGCUCCGGAUCCU |
| 630 | HPV35_4967 | GAUACAACCUUACAAUUUGAGCAUG |
| 631 | HPV35_4909 | GACUUCUCCUGCAAAACUUAUUACA |
| 632 | HPV35_4857 | GAUUAUAUAGUAAAGGUACCCAGCA |
| 633 | HPV35_4800 | GCAAUAAUAUAACUAAUAGCACGCC |
| 634 | HPV35_4713 | CAGGUCAUUUUGUACUUUCAUCAUC |
| 635 | HPV35_4688 | CACCCACCCACGCCUGCAGAAACUU |
| 636 | HPV35_4634 | GUGACAUCCAUAAGUACACAUGAUA |
| 637 | HPV35_4605 | CUACAGAUACCACACCUGCUAUUUU |
| 638 | HPV35_4578 | CUACAACAGGUUUUACAAUAACCAC |
| 639 | HPV35_4553 | CCUGUUGUUACACCAAGGGUCCCAC |
| 640 | HPV35_4506 | CUAUAGUGUCAUUAGUAGAGGAAAC |
| 641 | HPV35_4481 | GACACAAUUGGCCCUUUAGAUUCUU |
| 642 | HPV35_4426 | GGCUGCCACAAACAUUCCUAUACGA |
| 643 | HPV35_4401 | UUCCACUGGGUACAACACCUCCAAC |
| 644 | HPV35_4376 | GGCACAGGUGGAAGAUCUGGAUAUG |
| 645 | HPV35_4233 | AACUAUAUCGUACUUGCAAAGCUGC |
| 646 | HPV35_4190 | CACAAAAGGUCUACAAAACGUGUUA |
| 647 | HPV35_4068 | GUAACAUGUGUGUAUGGUGGUUUUA |
| 648 | HPV35_4026 | GGCAGUACAGUAAUUGUAUACAAAC |
| 649 | HPV35_3999 | GAUGAUUAACGCUCAUGCACAAUAU |
| 650 | HPV35_3958 | CUACUUGCUUUUGUUGUUUCUUGCU |
| 651 | HPV35_3933 | ACUGUGGGUUACUGUAGCAACACCA |
| 652 | HPV35_3889 | CUAUCUGUGUCAUUAUACUCAGCAU |
| 653 | HPV35_3864 | GUGUCUGCUUGUACGUUCGCUAUUG |
| 654 | HPV35_3839 | UGUGCUUUUGUGUGCUUUUGUGCUU |
| 655 | HPV35_3807 | AGCUUCCAGUACUGUGUUGCUGUGC |
| 656 | HPV35_3760 | CACAGUUACAGUGUCUAAAGGAUAU |
| 657 | HPV35_3705 | CUUACACAACAGAAUAUCAAAGGGA |
| 658 | HPV35_3652 | AUGGAGAUGGACAUGUACAAACGAU |
| 659 | HPV35_3513 | ACUGCACAAACAAAGACCGGUGUGG |
| 660 | HPV35_3481 | CAGUGUUGACAGAGGGGUCUACUCU |
| 661 | HPV35_3456 | AGCGAGUGCGACUCAGUGCCGUGGA |
| 662 | HPV35_3431 | ACCGAGCUCCCCUACAACCCCACCA |
| 663 | HPV35_3399 | AGAAGACAAAUCACAAACGACUUCG |
| 664 | HPV35_3360 | CCCAUACCAAAGCCUGCUCCGUGGG |
| 665 | HPV35_3293 | UUUAGCAGCACAGAACUAUCCACUG |
| 666 | HPV35_3196 | UUAUGUUACUUUUAGGGAAGAGGCU |
| 667 | HPV35_3171 | AUGUGCAUCAGGGUGUAGAAACAUA |
| 668 | HPV35_3123 | GUAUAUGUACUGUUGUAAAGGGACU |
| 669 | HPV35_3047 | GAAGCACAAUUUGAUGGUGAUAAAC |
| 670 | HPV35_2946 | CAACUGAGUAUAGCACAGAGGACUG |
| 671 | HPV35_2890 | AAAAGCCAAAGCAAUGCAAGCAAUU |
| 672 | HPV35_2865 | AAGUGGUUCCAACGCAGGCCAUUUC |
| 673 | HPV35_2840 | AUGGGAAUUAAAACUCUUAACCACC |
| 674 | HPV35_2788 | GUAUUGGAAACUGAUUCGUCUUGAA |
| 675 | HPV35_2763 | GCACAUGUUUGUCUGAUCACAUACA |
| 676 | HPV35_2679 | AGAGGUCAAAGAAAAUGAUGGAGAC |
| 677 | HPV35_2648 | GGACGUGGUGCAGAUUAAAUUUGCA |
| 678 | HPV35_2551 | GUAGUGGUCUUUACAUUUCACAAUG |
| 679 | HPV35_2526 | CAGGUGGCCAUACUUACAUAGCAGG |
| 680 | HPV35_2386 | CCAUGUGGCAUAUAUAGACCAAUAU |
| 681 | HPV35_2338 | CAGCCAUUAUAUGAUGCCAAAAUAG |
| 682 | HPV35_2275 | CUAAUGCAUUUCUUACAAGGAGCUA |
| 683 | HPV35_2220 | UUGCAUACUAAUAUAUGGAGCACCA |
| 684 | HPV35_2147 | GAUAUCAACAAGUAGAUUUUGUGGC |
| 685 | HPV35_2075 | CACAGUGGAUUAAAAGGCGAUGUGC |
| 686 | HPV35_1955 | CAGAAACUAAUAGUAAUGCAUGUGC |
| 687 | HPV35_1791 | UAUUAGUGAGGUUGAUGGAGAAACA |
| 688 | HPV35_1744 | CGUAGUACCCCAGCUGCGUUAUAUU |
| 689 | HPV35_1698 | GCUAUGUAUUUCAGCUGCAAGUAUG |
| 690 | HPV35_1619 | GGGCUAUGGUAAUUCUAGCAUUAUU |
| 691 | HPV35_1559 | GUGUGGCGAACUUUAAACAUAUAAC |
| 692 | HPV35_1534 | GUGGCCGCAUUUGGAAUAGCCCCAA |
| 693 | HPV35_1391 | CAACGCGAGACAUAAUACAAAUACU |
| 694 | HPV35_1366 | AGCGAUGAAAGACAUGAUGAGACUC |
| 695 | HPV35_1341 | CAGUGGGGAUAGUAUAACCUCUAGU |
| 696 | HPV35_1316 | AUACAGUUGAACAAUGUAGUAUGGG |
| 697 | HPV35_1286 | UACACGAGAUACAACAGGUAGAGGG |
| 698 | HPV35_1237 | CGAUUAUUUGAACUACCAGACAGCG |
| 699 | HPV35_1136 | CUAGUAGUCCACUUAGCAGCGUGAG |
| 700 | HPV35_1101 | CAAAGAGGCUGUACAGGUCCUAAAA |
| 701 | HPV35_1051 | GAAACAGAGACAGCACAAGCAUUAU |
| 702 | HPV35_970 | GACGAAAAUGAAGAUGACUGUGACA |
| 703 | HPV35_945 | UAGACGUACGGGAUCCAGUGUAGAG |
| 704 | HPV35_858 | AUAAUCUACAAUGGCUGAUCCUGCA |
| 705 | HPV35_828 | AAUAGUGUGCCCCGGCUGUUCACAG |
| 706 | HPV35_781 | CACAUUGACAUACGUAAAUUGGAAG |
| 707 | HPV35_739 | UGUAAAUGUGAGGCGACACUACGUC |
| 708 | HPV35_703 | CCAGACACCUCCAAUUAUAAUAUUG |
| 709 | HPV35_669 | AGAUACUAUUGACGGUCCAGCUGGA |
| 710 | HPV35_592 | UAUGUUUUAGAUUUGGAACCCGAGG |
| 711 | HPV35_554 | GUGUAAUCAUGCAUGGAGAAAUAAC |
| 712 | HPV35_529 | GAAACCAACACGUAGAGAAACCGAG |
| 713 | HPV35_443 | CCAGUUGAAAAGCAAAGACAUUUAG |
| 714 | HPV35_350 | UAUAGUGUGUAUGGAGAAACGUUAG |
| 715 | HPV35_284 | CCAUAUGGAGUAUGCAUGAAAUGUU |
| 716 | HPV35_259 | GUGUAUAGUAUAUAGAGAAGGCCAG |
| 717 | HPV35_232 | GGUAUAUGACUUUGCAUGCUAUGAU |
| 718 | HPV35_207 | GCAAACAAGAAUUACAGCGGAGUGA |
| 719 | HPV35_163 | GGUAGAAGAAAGCAUCCAUGAAAUU |
| 720 | HPV35_131 | CGACCUUACAAACUGCAUGAUUUGU |
| 721 | HPV35_106 | CGGUAUGUUUCAGGACCCAGCUGAA |
| 722 | HPV35_46 | ACGGUUGCCAUAAAAGCAGAAGUGC |

In one embodiment, the present invention provides an isolated polynucleotide for specific hybridization to HPV 39 consisting essentially of a sequence or a complement thereof selected from the group consisting of SEQ ID NOs: 723-841 (See Table 7). In some embodiments, the present invention provides a set of polynucleotides for specific hybridization to HPV 39, wherein the set comprises at least one polynucleotide consisting essentially of a sequence or a complement thereof selected from the group consisting of: SEQ ID NOs: 723-841. In certain embodiments, the methods of the present invention utilize a set of polynucleotide probes for specific hybridization to HPV 39 comprising SEQ ID NOs: 723-841.

TABLE 7
Polyribonucleotide probes for
determining HPV 39 nucleic acid.

| SEQ ID NO: | Name | Sequence |
|---|---|---|
| 723 | HPV39_7780 | CACACAAUAGUUUAUGCAACCGAAA |
| 724 | HPV39_7735 | CAGGAAUGUGUCUUACAGUAUAAGU |
| 725 | HPV39_7692 | CUUGCUUAAUUAAAUAGUUGGCCUG |
| 726 | HPV39_7642 | CCACCCUAUGUAAUAAAACUGCUUU |
| 727 | HPV39_7617 | CAAUACUUUGGCAACAUCCAUAUCU |
| 728 | HPV39_7581 | CCUUAUUACUCAUCAUCCUGUCCAG |
| 729 | HPV39_7538 | UUCACCCUGCAUAGUUGGCACUGGU |
| 730 | HPV39_7429 | CAUUUUAUACUUCGCCAUUUUGUGG |
| 731 | HPV39_7349 | UCAUACAUAAUCUAUAUGCCCUACC |
| 732 | HPV39_7273 | AUGACAGUUUCAUGUGUGAUUGCAC |
| 733 | HPV39_7203 | CCUUAUGUGUUGAGUGUAUAUGUGU |
| 734 | HPV39_7173 | CCUUGUUAUGUGUGUGUAUGUUGUU |
| 735 | HPV39_7146 | CGUGUGUCUAAAUAAUGCAUGUGUA |
| 736 | HPV39_7111 | CUUCCUCGUCCUCAGCUACUAAACA |
| 737 | HPV39_7072 | GCCCUACUAUAGGUCCCCGAAAGCG |
| 738 | HPV39_7012 | UGGAACUUGAUCAAUUCCCUUUGGG |
| 739 | HPV39_6956 | AGAUCCAUAUGACGGUCUAAAGUUU |
| 740 | HPV39_6902 | CCUACAGUCUGCAGCCAUUACAUGU |
| 741 | HPV39_6877 | CCAGUUUGGUAGACACUUACAGAUA |
| 742 | HPV39_6851 | UUUUGCUGUAGCUCCUCCACCAUCU |
| 743 | HPV39_6824 | GAAUUCCUCUAUAUUGGACAAUUGG |
| 744 | HPV39_6696 | CCUUCUACAUAUGAUCCUUCUAAGU |
| 745 | HPV39_6671 | AUCUACCUCUAUAGAGUCUUCCAUA |
| 746 | HPV39_6511 | ACUGCCCCUCUCCCAGCGGUUCCAU |
| 747 | HPV39_6486 | CGUGCAAACCCCGGUAGUUCUGUAU |
| 748 | HPV39_6458 | CCAAUUGUAUAUUAAGGGCACAGAU |
| 749 | HPV39_6370 | ACAGUAUGUUCUUCUGUUUACGUAG |
| 750 | HPV39_6204 | GAACUAGUAAACACCCCUAUUGAGG |
| 751 | HPV39_6160 | CAUGCAAGCCCAAUAAUGUAUCUAC |
| 752 | HPV39_6039 | CCAUUUUCAUCAACCACCAAUAAGG |
| 753 | HPV39_5998 | GACACCCAUUAUAUAAUAGACAGGA |
| 754 | HPV39_5908 | CCUUAUAUAAUCCAGAAACACAACG |
| 755 | HPV39_5875 | CCGAUCCUAAUAAAUUCAGUAUUCC |
| 756 | HPV39_5850 | UAUAGGGUAUUUCGCGUGACAUUGC |
| 757 | HPV39_5792 | UAAAGUGGGUAUGAAUGGUGGUCGC |
| 758 | HPV39_5758 | GCUCUAGAUUAUUAACAGUAGGACA |
| 759 | HPV39_5543 | ACAACAUAUGCAAUAACCAUUCAGG |
| 760 | HPV39_5512 | GUUGCCAUUGGUGCCUUCUGGACCA |
| 761 | HPV39_5487 | UUGCUUUACCAAGUACUACUCCACA |
| 762 | HPV39_5462 | AUGCCUGUAAAUACUGGUCCUGAUA |
| 763 | HPV39_5436 | CUAUUCCUUUUAGUACCUCAUGGAA |
| 764 | HPV39_5409 | CAGCAUCUACUAAAUAUGCCAAUAC |
| 765 | HPV39_5384 | GGCUCACUACCUUCUGUGGCUUCUU |
| 766 | HPV39_5359 | GGAUUCGGGCACUACAUAUAACACA |
| 767 | HPV39_5305 | AUAUGCUGAUGUGGACAAUAACACA |
| 768 | HPV39_5264 | CACGCUGAGCCCUCUGAUGCUUCAG |
| 769 | HPV39_5239 | AAGCAUUGAAUUACAGCCCCUAGUU |
| 770 | HPV39_5209 | CCAUGACAUUAGUAGUAUUGCUCCU |
| 771 | HPV39_5178 | GCACACAAAUUGGAGCGCAAGUACA |
| 772 | HPV39_5121 | AAGGAACAGUAAGGUUUAGUAGGCU |
| 773 | HPV39_5018 | GAGCCUGUUGAUACUACAUUAACAU |
| 774 | HPV39_4928 | UAUAGUAGAGCACAUCAGCAGGUUC |
| 775 | HPV39_4889 | CCUACACCUGGAAUCAGUCGUGUGG |
| 776 | HPV39_4778 | UCGGGUAAUAUAUUUGUCAGUACCC |
| 777 | HPV39_4736 | ACGGAUCCUUCCUUAAUUGAGGUUC |
| 778 | HPV39_4706 | ACCUCUACUAGUUAUACUAACCCUG |
| 779 | HPV39_4621 | CACCUCUGGAUUUGAAAUUACUUCU |
| 780 | HPV39_4596 | GAACACCAGUACCAACAUUUACAGG |
| 781 | HPV39_4571 | GAGGACUCAAGUGUUAUAACCUCUG |
| 782 | HPV39_4546 | UGAGCCAUCUAUUGUGCAAUUGGUG |
| 783 | HPV39_4487 | ACUGUUGUAGAUGUGUCUCCUGCAC |
| 784 | HPV39_4358 | GGUACUACACUUGCUGACAAAAUUU |
| 785 | HPV39_4333 | ACCAGACGUUGUUGAUAAAGUUGAG |
| 786 | HPV39_4297 | CCUAUAUAGAACCUGUAAACAAUCG |
| 787 | HPV39_4239 | UACUAAUAAACAUGGUUUCCCACCG |
| 788 | HPV39_4195 | AUUGUGCAUAACUACUGUACAUAGC |
| 789 | HPV39_4158 | GGCAAUGGAUAUGAUAUAGUACUGU |
| 790 | HPV39_4133 | UGCCCAUGUGGUUGUUGCAUAGACU |
| 791 | HPV39_4046 | CGUAUGUGUGGAUAAUUGUGUUUGU |
| 792 | HPV39_3888 | CAUUGGGUUACAUGACAUUGUAAAG |
| 793 | HPV39_3854 | GACACUGUUAAAAUACCUUCUAGUG |
| 794 | HPV39_3818 | ACAUAUGCCACAGAGUCACAACGCC |
| 795 | HPV39_3641 | AGACGGUACCUCAGUUGUGGUAACA |
| 796 | HPV39_3616 | CAGUAACAGUACAGGCCACAACACA |
| 797 | HPV39_3591 | UGGACCAUCUUAACAACCCACUCCA |
| 798 | HPV39_3556 | AGUCACAGAGCCCACUGAGCCCGAC |
| 799 | HPV39_3458 | GAAUUAUCAAACACCACCGCGACCC |
| 800 | HPV39_3426 | ACGGAUCGGUACCCACUACUGAACU |
| 801 | HPV39_3328 | UAUUCAAGAUGCGGAAAGGUAUGGG |
| 802 | HPV39_3301 | GCACCUAAAAGUAUACUAUGAAGUG |
| 803 | HPV39_3199 | GAACUAUGUAUUAUGGGGUGCUAUA |
| 804 | HPV39_3174 | AUGAUGGGGACAAAUGUAAUGCUAU |
| 805 | HPV39_3067 | UGAAUACAAUACAGAGGAGUGGACA |
| 806 | HPV39_2986 | GGUGCCAACCAUAAACAUUUCAAAA |
| 807 | HPV39_2636 | ACGAUAGGUGGCCAUAUUUACGUAG |
| 808 | HPV39_2542 | GGGUAUGCAAUAAGUUUAGAUAGGA |
| 809 | HPV39_2479 | UUAGAUGAUGCAACCGGUACCUGCU |
| 810 | HPV39_2412 | UAUUUCAUAUGUAAACUCCACCAGC |
| 811 | HPV39_2338 | GUUAUAUAUGGACCUGCGAAUACAG |
| 812 | HPV39_2235 | GAGACCCAUAGUACAAUUCUUAAGA |
| 813 | HPV39_2205 | GUGUAGUAAAUGUGAUGAAGGCGGG |
| 814 | HPV39_2056 | GCAAUGUUAGCAGAUUGUAACAGUA |
| 815 | HPV39_1974 | UAGUGUAUUUGACCUAUCGGACAUG |
| 816 | HPV39_1906 | AGUGUGGUAACAGGGGAUACGCCAG |
| 817 | HPV39_1881 | GUAUCGCACAGGUAUAUCCAAUAUU |
| 818 | HPV39_1835 | UUCUGGAGCCUCCUAAACUGCGCAG |
| 819 | HPV39_1789 | GGAAAGGGAUUAAGUACAUUGUUAC |
| 820 | HPV39_1716 | CUUAGACACAAAACAAGGAGUACUA |
| 821 | HPV39_1645 | GUACAUCCAACUAUUGCAGAAGGAU |
| 822 | HPV39_1568 | UAUCCUUUACUGACCUGGUACGUAC |
| 823 | HPV39_1531 | GCUGCAAUGCUAACACAAUUUAAAG |
| 824 | HPV39_1478 | CCAAAUCUCCAACUGCACAAAUUAA |
| 825 | HPV39_1453 | GCUAUAGAUAGUGAAAACCAGGAUC |
| 826 | HPV39_1390 | AAUGGGGAUGCUGAAGGGGAACAUG |
| 827 | HPV39_1283 | GCAGUACGCAGGCAACACAAACGGU |
| 828 | HPV39_1251 | GGGAACACUACAGGAAAUUUCAUUA |
| 829 | HPV39_1189 | AAGUAUACAGACAGCAGUGGCGACA |
| 830 | HPV39_1083 | UGAUUCCACAGAUAUUUGUGUACAG |
| 831 | HPV39_876 | CUCACUAGGAUUUGUGUGUCCGUGG |
| 832 | HPV39_839 | GGGAUACUCUGCGACAACUACAGCA |
| 833 | HPV39_803 | GUAACAACACACUGCAGCUGGUAGU |
| 834 | HPV39_595 | CGUGGACCAAAGCCCACCUUGCAGG |
| 835 | HPV39_567 | GAGAAACCCAAGUAUAACAUCAGAU |
| 836 | HPV39_464 | CACCUAAAUAGCAAACGAAGAUUUC |
| 837 | HPV39_336 | AGCUACGAUAUUACUCGGACUCGGU |
| 838 | HPV39_284 | GAACCACUAGCUGCAUGCCAAUCAU |
| 839 | HPV39_259 | UUUAUAUGUAGUAUAUAGGGACGGG |
| 840 | HPV39_212 | AGACGACCACUACAGCAAACCGAGG |
| 841 | HPV39_7808 | GUUGGGCAUACAUACCUAUACUUUU |

In one embodiment, the present invention provides an isolated polynucleotide for specific hybridization to HPV 51 consisting essentially of a sequence or a complement thereof selected from the group consisting of SEQ ID NOs 975-1120: (See Table 8). In some embodiments, the present invention provides a set of polynucleotides for specific hybridization to HPV 51, wherein the set comprises at least one polynucleotide consisting essentially of a sequence or a complement thereof selected from the group consisting of: SEQ ID NOs: 975-1120. In certain embodiments, the methods of the present invention utilize a set of polynucleotide probes for specific hybridization to HPV 51 comprising SEQ ID NOs: 975-1120.

TABLE 8

Polyribonucleotide probes for
determining HPV 51 nucleic acid.

| | | |
|---|---|---|
| SEQ ID NO: | Name | Sequence |

| | | |
|---|---|---|
| 975 | HPV51_7766 | UUGUGUUCUGCCUAUGCUUGCAACA |
| 976 | HPV51_7716 | CCAUCUUACUCAUAUGCAGGUGUGC |
| 977 | HPV51_7689 | GUGCCAAGUUUCUAUCCUACUUAUA |
| 978 | HPV51_7593 | CCGCCCUAUAAUAAUUUAACUGCUU |
| 979 | HPV51_7566 | CUUUAACAAUUGUUGGCACACUGUU |
| 980 | HPV51_7536 | GCUAGUCAUACAACCUAUUAGUCAU |
| 981 | HPV51_7510 | CCUUGUACUUGGCGCGCCUUACCGG |
| 982 | HPV51_7485 | UAGUGCAUACAUCCGCCCGCCCACG |
| 983 | HPV51_7427 | AAGUUUUAAACCACAACUGCCAGUU |
| 984 | HPV51_7394 | GAUUUCGGUUCGUGUACUUUUAGUA |
| 985 | HPV51_7368 | CAGCUGCAGCCAUUUUGAGUGCAAC |
| 986 | HPV51_7265 | AGGGUGGUGUUUCGGUGGCGUCCCU |
| 987 | HPV51_7236 | UGUGGGUAUUACAUUAUCCCCGUAG |
| 988 | HPV51_7211 | CAUUUGUAUGACAUGUACGGGUGUA |
| 989 | HPV51_7131 | GUUGUUCCUGUAUGUAUGAGUUAUG |
| 990 | HPV51_7071 | GUAUGCCUGUAUGUAUAUGUUUGUG |
| 991 | HPV51_6979 | UCAUCGGCAUCCUCUUCCUCUUCCU |
| 992 | HPV51_6939 | CGUACAACGCAAGCCCAGACCAGGC |
| 993 | HPV51_6738 | AACAUUACCUCCGUCUGCUAGUUUG |
| 994 | HPV51_6707 | CUACCAUUCUUGAACAGUGGAAUUU |
| 995 | HPV51_6671 | CUACAGAGGUAAUGGCUUAUUUACA |
| 996 | HPV51_6597 | CUUUAAGCAAUAUAUUAGGCAUGGG |
| 997 | HPV51_6572 | UUUCCCCAACAUUUACUCCAAGUAA |
| 998 | HPV51_6543 | UUUAACUAUUAGCACUGCCACUGCU |
| 999 | HPV51_6514 | ACCUGUGUUGAUACUACCAGAAGUA |
| 1000 | HPV51_6385 | UAUAUAUACUCUGCUACUCCCAGUG |
| 1001 | HPV51_6360 | UAAUGGCCGUGACCCUAUAGAAAGU |
| 1002 | HPV51_6307 | CUUGUAGGUGUUGGGGAAGACAUUC |
| 1003 | HPV51_6160 | GCCACCAAAUCAGACGUCCCUUUGG |
| 1004 | HPV51_6084 | ACUUGUAUCCUCUGUCAUUCAGGAU |
| 1005 | HPV51_5962 | GUUGACAACAAACAGACUCAGUUAU |
| 1006 | HPV51_5922 | AAAUGGCAAUGCACAACAAGAUGUU |
| 1007 | HPV51_5897 | AUGACACAGAAAAUUCACGCAUAGC |
| 1008 | HPV51_5773 | CCGGAUCCAAAUUUAUAUAAUCCAG |
| 1009 | HPV51_5707 | AAAGUAUCUGCAUUUCAAUACAGGG |
| 1010 | HPV51_5682 | AACCUCAACGCGUGCUGCUAUUCCU |
| 1011 | HPV51_5641 | AGACUAAUAACAUUAGGACAUCCCU |
| 1012 | HPV51_5590 | ACAGAAGAAUAUAUCACACGCACCG |
| 1013 | HPV51_5565 | UGCACCUGUGUCUCGAAUUGUGAAU |
| 1014 | HPV51_5469 | UAUACACAUUUACUACGCAAACGCC |
| 1015 | HPV51_5444 | AGGUGGGGAUUACUAUUUGUGGCCC |
| 1016 | HPV51_5418 | GACACCAAGCAUUCUAUUGUUAUAC |
| 1017 | HPV51_5393 | GCCUUAUGUUCCCCACACUUCCAUU |
| 1018 | HPV51_5368 | UAUUGCCCACAUCUCCUACAGUAUG |
| 1019 | HPV51_5343 | CCUAUUCAUACAGGGCCUGAUGUGG |
| 1020 | HPV51_5281 | CUUCAUCUAUGUCUUCAUCUUAUGC |
| 1021 | HPV51_5247 | CACUCCUCUUUGUCUAGGCAGUUGC |
| 1022 | HPV51_5189 | UGAUUUAGAUGAAGCUGAAACAGGU |
| 1023 | HPV51_5142 | CAGCCUUUACUUUCACCUUCUAAUA |
| 1024 | HPV51_5117 | UGCACCAGCUGAUGAACUUGAAAUG |
| 1025 | HPV51_4967 | UCUGGAUAUUAUUACACUGCACCGC |
| 1026 | HPV51_4926 | ACUUUUGAGGAACCUGAUGCUGUUG |
| 1027 | HPV51_4901 | UUUUGAGCCUAUUGACACAUCCAUA |
| 1028 | HPV51_4825 | CCUACACACAGGUUAAAGUUACAAA |
| 1029 | HPV51_4800 | GCUGCUCCCCGCUUGUAUAGUAAGU |
| 1030 | HPV51_4762 | CUAUUAGCAGCACACCUACUCCAGG |
| 1031 | HPV51_4733 | UGCAUCCAAUGUCAGUACUGGUACU |
| 1032 | HPV51_4676 | UUUACUAGUACACUACUCUGGUACU |
| 1033 | HPV51_4633 | CAUCCAUUGAGGCUCCACAAUCUGG |
| 1034 | HPV51_4578 | GGUACUGUACAUGUUUCUAGUACUA |
| 1035 | HPV51_4526 | UACUUCAUCUUCCACAACAACCCCU |
| 1036 | HPV51_4483 | GGUCUCCUAUACCUACCUUUACUGG |
| 1037 | HPV51_4458 | GAGGACUCUAGUAUUAUUCAGUCUG |
| 1038 | HPV51_4425 | CACCAUACUGAACCUUCUAUAGUAA |
| 1039 | HPV51_4400 | GCCACCUAUUAUAAUUGACCUAUGG |
| 1040 | HPV51_4373 | AGGCGUGGUGGAUAUUGCUCCUGCA |
| 1041 | HPV51_4337 | UACUGGAUAUAUCCCUUUAGGUGGU |
| 1042 | HPV51_4253 | GGCCGAUAAAAUAUUACAGUGGAGU |
| 1043 | HPV51_4223 | UGUUGUGAAUAAGGUUGAAGGUACU |
| 1044 | HPV51_4131 | AAUAUGGUGGCUACACGUGCACGGC |
| 1045 | HPV51_4009 | UGUUGCAACAUCCCAAUUAACUACA |
| 1046 | HPV51_3964 | CGUGUUUGCAGCUGCCUUAUUAUUA |
| 1047 | HPV51_3939 | UGUUGCCGCUACUGCUGUCCCAAUA |
| 1048 | HPV51_3861 | GACAUAUUGUAACCAUUGCAGUGUU |
| 1049 | HPV51_3816 | GUACAUAUAUACUGUCACAAGCCAA |
| 1050 | HPV51_3778 | GGGAAUUAUGACACUGUAACUAGUG |
| 1051 | HPV51_3714 | GUGCACAUCAACGGGAAACAUUUAU |
| 1052 | HPV51_3689 | GGCAUUGUUACCAUUGUGUUUGACA |
| 1053 | HPV51_3552 | CAACUCAGACUGCGUUUAUAGUGCA |
| 1054 | HPV51_3495 | CAAACAACCAAAUACACUGUGGAAG |
| 1055 | HPV51_3463 | CUCCACAAUCUCCCCACUGUCCGUG |
| 1056 | HPV51_3438 | GACAGCGACUUACUGAGCCCGACUC |
| 1057 | HPV51_3413 | GAAGCCCAGACACAACAGCGAAAAC |
| 1058 | HPV51_3379 | GACCAAUCCCCUUACCACCUGCGUG |
| 1059 | HPV51_3354 | UUGAACAACUAUCAAACACCCCAAC |
| 1060 | HPV51_3329 | GACGCGUUAUCCACUACUACAACUG |
| 1061 | HPV51_3284 | GGUACUGUAAUAACAUGUCCUGAAU |
| 1062 | HPV51_3259 | ACAACAGUGGGAGGUCUAUAUGUAU |
| 1063 | HPV51_3234 | AAGAUGAAGCCAAAAUAUAUGGGGC |
| 1064 | HPV51_3176 | GACUAUACGGGUAUAUAUUACACUG |
| 1065 | HPV51_3151 | GUGGGUAAAGACAAAUGGAAAUGUG |
| 1066 | HPV51_3102 | CAAUGGACUAUACAAGCUGGAAAUU |
| 1067 | HPV51_3017 | GAACUAUGGUGUGUGGCUCCCAAGC |
| 1068 | HPV51_2992 | AUGGACAAUGCGGGAGACAUGUUAU |
| 1069 | HPV51_2967 | ACAAAUCAGACUAUAACAUGGAACC |
| 1070 | HPV51_2942 | AUGCACAUGGCCUUACAAUCGCUUA |
| 1071 | HPV51_2914 | AAAACAAAAGGCCUGUCAAGCAAUU |
| 1072 | HPV51_2889 | AGGUAGUACCAGCAACAACAGUAUC |
| 1073 | HPV51_2864 | AGAAACUUACGAACAAUCAAUCACC |
| 1074 | HPV51_2829 | GAUAUGAAGCUGCUAUGUUUUAUGC |
| 1075 | HPV51_2623 | GGGAAUGCUGUGUAUACAUUGAAUG |
| 1076 | HPV51_2545 | GAGGAUGCAAACCUAAUGUAUUUAC |
| 1077 | HPV51_2363 | AGCCACUAGAGGAUGCUAAAAUAGC |
| 1078 | HPV51_2307 | GUUUAUGCAAGGGUCCAUUAUUUCA |
| 1079 | HPV51_2280 | GUCAUUAUUUGCAAUGAGCCUAAUG |
| 1080 | HPV51_2243 | AUUGCAUAGUCAUAUAUGGCCCACC |
| 1081 | HPV51_2121 | UGAUAGAGCAAAGGAUGGAGGCAAC |
| 1082 | HPV51_2089 | UUAUCUAUGUCAGCCUGGAUAAGGU |
| 1083 | HPV51_2061 | GCAUUACAAACGAGCACAAAGAAAA |
| 1084 | HPV51_2036 | UAAAAGAUUGUGGGACCAUGGCACG |
| 1085 | HPV51_1927 | GACCAUGAAGUAUUAGAUGAUAGUG |
| 1086 | HPV51_1854 | ACGACAAACGCAACUACAACAUAGU |
| 1087 | HPV51_1819 | AGCAAUACAUAUGGAGAGACACCUG |
| 1088 | HPV51_1600 | CCAUUUUGCAUGUACUACCAUAUAC |
| 1089 | HPV51_1559 | UUUCCCCAAUGGUAGCAGAAAAUUU |
| 1090 | HPV51_1534 | GAUUGGGUUUGUGCAUUGUUUGGCG |
| 1091 | HPV51_1489 | AAUGAGUUGGUACGGGUGUUUAAAA |
| 1092 | HPV51_1438 | GCAAAAGCAACGUUAAUGGCAAAAU |
| 1093 | HPV51_1386 | CUGUGCAAAUGUAGAACUAAACAGU |
| 1094 | HPV51_1317 | UGGCGGUUCACAGAACAGUGUGUGU |
| 1095 | HPV51_1228 | AGGAGAUUACUGGACAGUUAUCCGG |
| 1096 | HPV51_1203 | UCAGGCAAACGAGUCACAAGUUAAA |
| 1097 | HPV51_1178 | AUCAAAACAACACACACAGCCAUAG |
| 1098 | HPV51_1130 | GAAAGUUUCUAGUCAGCCCGCGAAG |
| 1099 | HPV51_1101 | AAACAAAGAGGCUGUGCAUCAGUUA |
| 1100 | HPV51_1076 | UGUUUCAGGCCCAAGAAUUACAGGC |
| 1101 | HPV51_1047 | UCAGGCGGAACAGGAGACAGCACGG |
| 1102 | HPV51_982 | GAAAAUGCAGAUGAUACAGGAUCUG |
| 1103 | HPV51_957 | AGAUAAUGUUUCGGAUGAUGAGGAU |
| 1104 | HPV51_862 | CUAGCAACGGCGAUGGACUGUGAAG |
| 1105 | HPV51_832 | AAGCCUGGUUUGCCCGUGUUGUGCG |
| 1106 | HPV51_800 | CGCGUUGUACAGCAGAUGUUAAUGG |
| 1107 | HPV51_770 | CUGGCAGUGGAAAGCAGUGGAGACA |
| 1108 | HPV51_745 | UUGCAGGUGUUCAAGUGUAGUACAA |
| 1109 | HPV51_720 | CGUGUUACAGAAUUGAAGCUCCGUG |
| 1110 | HPV51_686 | GACCAGCUACCAGAAAGACGGGCUG |
| 1111 | HPV51_661 | GGAGGAUGAAGUAGAUAAUAUGCGU |
| 1112 | HPV51_552 | AUAAAGCCAUGCGUGGUAAUGUACC |
| 1113 | HPV51_503 | GCGCUAAUUGCUGGCAACGUACACG |
| 1114 | HPV51_418 | AGACCACUUGGGCCUGAAGAAAAGC |
| 1115 | HPV51_348 | UGGUACUACAUUAGAGGCAAUUACU |
| 1116 | HPV51_323 | AUAGACGUUAUAGCAGGUCUGUGUA |
| 1117 | HPV51_209 | GUAGAGCAGAUGUAUAUAAUGUAGC |
| 1118 | HPV51_160 | UCUAUGCACAAUAUACAGGUAGUGU |
| 1119 | HPV51_103 | GAAGACAAGAGGGAAAGACCACGAA |
| 1120 | HPV51_75 | GGUAAAAGUAUAGAAGAACACCAUG |

In one embodiment, the present invention provides an isolated polynucleotide for specific hybridization to HPV 52 consisting essentially of a sequence or a complement thereof selected from the group consisting of SEQ ID NOs: 1121-1252 (See Table 9). In some embodiments, the present invention provides a set of polynucleotides for specific hybridization to HPV 52, wherein the set comprises at least one polynucleotide consisting essentially of a sequence or a complement thereof selected from the group consisting of: SEQ ID NOs: 1121-1252. In certain embodiments, the methods of the present invention utilize a set of polynucleotide probes for specific hybridization to HPV 52 comprising SEQ ID NOs: 1121-1252.

TABLE 9

Polyribonucleotide probes for
determining HPV 52 nucleic acid.

| | | |
|---|---|---|
| SEQ ID NO: | Name | Sequence |

| | | |
|---|---|---|
| 1121 | HPV52_7871 | UGUUACUCACCAGGUGUGCACUACA |
| 1122 | HPV52_7837 | CGCCAAAUAUGUCUUGUAAAACAUG |
| 1123 | HPV52_7812 | UGUUGGCUUACACAAGUACAUCCUA |
| 1124 | HPV52_7732 | CAAUACAUUGCCUAACAUUGCAUGU |
| 1125 | HPV52_7701 | GCUGACUCACAGGUCCUGCAGUGCA |
| 1126 | HPV52_7676 | GUUGUCCCGCCUAAACUGACUUCUU |
| 1127 | HPV52_7651 | UCCUGCAGUCCACUGGUCUACACUU |
| 1128 | HPV52_7540 | CCAUUUUAAAUCCUAACCGAAUUCG |
| 1129 | HPV52_7509 | CUCUCCAUUUUGUACCAUUUUGUAC |
| 1130 | HPV52_7473 | GUGUCCUACUUUGUUACACUACUAA |
| 1131 | HPV52_7448 | UACCCUGUGUCCCCUGCCCUACCCU |
| 1132 | HPV52_7421 | GCUCCUAAUCUAUUGCAUCUCCUGC |
| 1133 | HPV52_7396 | CACCCACAUGAGUAACAAUACAGUU |
| 1134 | HPV52_7307 | CAGUUCCUGUAUGUAUGUUUUGUGU |
| 1135 | HPV52_7266 | UUUGCAUGUUAUGUAUGUGUGUGCA |
| 1136 | HPV52_7241 | AUUGUUUUGUGUGUGUACUGUGUUG |
| 1137 | HPV52_7200 | UGUCAAACACAGGUUAAAAGGUAAC |
| 1138 | HPV52_7168 | GGUAAUUGUCUGUGUCAUGUAUGUG |
| 1139 | HPV52_7143 | GUUAAAAGGUAACCAUUGUCUGUUG |
| 1140 | HPV52_7112 | GGCCCCACGUACCUCCACAAAGAAG |
| 1141 | HPV52_7087 | CCAAACUAAAACGCCCUGCAUCAUC |
| 1142 | HPV52_7062 | UUACAGGCAGGGCUACAGGCUAGGC |
| 1143 | HPV52_6977 | AAAGGACUAUAUGUUUUGGGAGGUG |
| 1144 | HPV52_6949 | CACCACCUAAAGGAAAGGAAGAUCC |
| 1145 | HPV52_6915 | GUCACUUCUACUGCUAUAACUUGUC |
| 1146 | HPV52_6880 | CACCGUCUGCAUCUUUGGAGGACAC |
| 1147 | HPV52_6828 | CAUAAGAUGGAUGCCACUAUUUUAG |
| 1148 | HPV52_6484 | AAGGGUCUAACUCUGGCAAUACUGC |
| 1149 | HPV52_6459 | CCUGUGCCAGGUGAUUUAUAUAUAC |
| 1150 | HPV52_6368 | CGAGCCAUAUGGUGACAGUUUGUUC |
| 1151 | HPV52_6326 | UAGCAGUGUAUGUAAGUAUCCAGAU |
| 1152 | HPV52_6275 | GGAUUUUAAUACCUUGCAAGCUAGU |
| 1153 | HPV52_6216 | CAGCUCAUUAACAGUGUAAUACAGG |
| 1154 | HPV52_6058 | CUGGUAAACCUGGUAUAGAUAAUAG |
| 1155 | HPV52_6026 | GUUUGAUGAUACUGAAACCAGUAAC |
| 1156 | HPV52_5540 | GCUCCAUCUACAUCUAUUAUUGUUG |
| 1157 | HPV52_5515 | UCCUUUUGUUCCUAUAGCCCCUACA |
| 1158 | HPV52_5490 | CAUUACCUUCGUUACCCACACAUAC |
| 1159 | HPV52_5460 | CUAUGUCCAUUGAGUCAGGUCCUGA |
| 1160 | HPV52_5435 | GGUAUUGACUUUGUAUAUCAACCCA |
| 1161 | HPV52_5385 | CUUCCACACUUUCUACCCAUAAUAA |
| 1162 | HPV52_5360 | UUGCAGCAACCCACGUUUCACUUAC |
| 1163 | HPV52_5314 | CCCUUACACUAUUAAUGAUGGUUUG |
| 1164 | HPV52_5289 | AACCUUUAUUACCACAGUCUGUGUC |
| 1165 | HPV52_5264 | GAAGUUCAGGAAGACAUAGAAUUGC |
| 1166 | HPV52_5239 | UGAUAUUAGUCCUAUCCAGCCUGCU |
| 1167 | HPV52_5076 | AACUUUUACCUGCACCGGAUCCUGA |
| 1168 | HPV52_5036 | GGCGUUGAUACAGAUGAAACUAUAA |
| 1169 | HPV52_4990 | GUCAUCACCACAGAAAUUAGUAACA |
| 1170 | HPV52_4933 | CCUUGGUUUAUAUAGCCGUGCCACA |
| 1171 | HPV52_4884 | GCAGUGUAACAAGUAGUACACCUAU |
| 1172 | HPV52_4859 | ACAUUUGUUACCUCUACUGACAGCA |
| 1173 | HPV52_4821 | CUAUUAGUACACACACCUAUGAAGA |
| 1174 | HPV52_4796 | GGUCAUGUAUUGUUUUCUAGUCCAA |
| 1175 | HPV52_4742 | CCUACAUUCACUGAACCAUCUAUAA |
| 1176 | HPV52_4710 | CAUCUGUACAAUCAGUUUCUACACA |
| 1177 | HPV52_4655 | ACAACAUCUGCAAAUAAUACUCCUG |
| 1178 | HPV52_4628 | AUUCCAUCAGCAACAGGGUUUGAUG |
| 1179 | HPV52_4593 | CAACAUUUAUUGAGUCUGGCGCACC |
| 1180 | HPV52_4556 | CCCUUAGAACCAUCUAUAGUUUCUA |
| 1181 | HPV52_4504 | UAGUAUUACCACGUCCACCAUUCGU |
| 1182 | HPV52_4479 | CAUUGUCCACUCGUCCUCCCACUAG |
| 1183 | HPV52_4452 | GCUCUGGUGGUAGGGCAGGCUAUGU |
| 1184 | HPV52_4424 | GGAGGUUUGGGUAUAGGUACAGGUG |
| 1185 | HPV52_4392 | UUUUAAAAUAUGGCAGCCUAGGGGU |
| 1186 | HPV52_4250 | UAGCUUGUCGCAAUGAGAUACAGAC |
| 1187 | HPV52_4157 | AUAACUGUACAUGUAGAUUGGCUAC |
| 1188 | HPV52_4114 | UGUUUUGUAUUCACUGUCAUGCACA |
| 1189 | HPV52_4055 | AUCUAUUGGGUCACCAUUUAAAGUG |
| 1190 | HPV52_4017 | UAUGCGCAGGUGUUGGUGCUGGUGC |
| 1191 | HPV52_3982 | CAGUGCUUAGGCCGCUCUUGCUAUC |
| 1192 | HPV52_3887 | AACACCCAACACAAGCCAAUAUUGC |
| 1193 | HPV52_3832 | GGUGUCAUGUCAUUGUGAUAUUUGU |
| 1194 | HPV52_3762 | CAGUGAUGAAACACAACGUCAACAA |
| 1195 | HPV52_3681 | GUAUGUUCAAAUUUCAUCUACCUGG |
| 1196 | HPV52_3593 | CAACUUGUACUGCACCUAUAAUACA |
| 1197 | HPV52_3541 | CGGGGACUCGUCACUGCAACUGAGU |
| 1198 | HPV52_3509 | UGCGGGGACAACAAUCCGUGGACAG |
| 1199 | HPV52_3484 | AACACCAAGUACCCCAACAACCUUU |
| 1200 | HPV52_3437 | UACAACCACCACAGAAACGACGACG |
| 1201 | HPV52_3406 | GCAGUGUCCGUGGGUGCCAAAGACA |
| 1202 | HPV52_3381 | AUGCACCGAAACCUCCAAGACCUCC |
| 1203 | HPV52_3208 | GGGUUAUAUUAUUGGUGUGAUGGAG |
| 1204 | HPV52_3176 | GUACAAUUGUAGAAGGACAAGUAGA |
| 1205 | HPV52_3125 | CUAUGGAUUAUACAAACUGGAAGGA |
| 1206 | HPV52_3081 | UGGGUAUACAAUAACAGUGCAAUAC |
| 1207 | HPV52_3036 | UCUAGAAAUGUGGCGUGCAGAACCA |
| 1208 | HPV52_3009 | AGAUGGAUGGACAUUACAACAAACA |
| 1209 | HPV52_2975 | CAUUGGAGGCAUUAAACAAAACACA |
| 1210 | HPV52_2887 | CUGGGAAUAACUCAUAUAGGCCACC |
| 1211 | HPV52_2847 | GACUCGAAUGGAAUGUGUUUUGUUU |
| 1212 | HPV52_2815 | GACCUAAACGCACAAAUUGAACAUU |
| 1213 | HPV52_2788 | CUAGAUCUAUACGAAGCUGAUAGUA |
| 1214 | HPV52_2578 | GGCCAUAUUUACAUAGUAGAUUGGU |
| 1215 | HPV52_2548 | CAAAUACAAAUGCAGGAACAGAUCC |
| 1216 | HPV52_2403 | GUGGGUAUGAUAGAUGAUGUAACAC |
| 1217 | HPV52_2327 | GUUCUUAAGUGGAUGUGUAAUAUCC |
| 1218 | HPV52_2143 | AUAGAAUAGAUGAUGGUGGAGAUUG |
| 1219 | HPV52_1909 | GCAUAUUCGAUUUUGGAGAAAUGGU |
| 1220 | HPV52_1822 | CAGGUUUGUCUAAUAUUAGUGAGGU |
| 1221 | HPV52_1789 | GAAGUGCUACCUGUGCAUUAUAUUG |
| 1222 | HPV52_1753 | CAGAAACACAUAUGGUAAUAGAACC |
| 1223 | HPV52_1723 | CCAAACUAAUGUCACAGCUGUUAAA |
| 1224 | HPV52_1670 | GCUUAUACUGCUGCUAAUUAGGUUU |
| 1225 | HPV52_1585 | CAUCAGUUGCAGAAGGAUUAAAAGU |
| 1226 | HPV52_1560 | UGUAUUAUAGGAAUGGGAGUAACAC |
| 1227 | HPV52_1387 | GUAUAGAGGACAAUGAGGAAAAUAG |
| 1228 | HPV52_1330 | GUAACAGUAGUCAAUCAAGUGGGGU |
| 1229 | HPV52_1237 | CAUGUCACGUAGAAGACAGCGGCUA |
| 1230 | HPV52_1207 | AUACAGAGUGUGUUUUACCAAAACG |
| 1231 | HPV52_1143 | GAAAGUGCUGGGCAAGAUGGUGUAG |
| 1232 | HPV52_1099 | UACAUGCUGUGUCUGCAGUAAAACG |
| 1233 | HPV52_1035 | AAUGAACAGGCAGAACAUGAGGCAG |
| 1234 | HPV52_981 | GCAUAUGAUAGUGGAACAGAUCUAA |
| 1235 | HPV52_899 | GGGAUGUACAGGCUGGUUUGAAGUA |
| 1236 | HPV52_853 | ACAACCCUGCAAUGGAGGACCCUGA |
| 1237 | HPV52_781 | GACCUUCGUACUCUACAGCAAAUGC |
| 1238 | HPV52_746 | GCACACUACGGCUAUGCAUUCAUAG |
| 1239 | HPV52_590 | UAGAUCUGCAACCUGAAACAACUGA |
| 1240 | HPV52_557 | GUGGAGACAAAGCAACUAUAAAAGA |
| 1241 | HPV52_532 | CUGUGACCCAAGUGUAACGUCAUGC |
| 1242 | HPV52_483 | AUUAUGGGUCGUUGGACAGGGCGCU |
| 1243 | HPV52_453 | CAUGUUAAUGCAAACAAGCGAUUUC |
| 1244 | HPV52_417 | UGUCAAACGCCAUUAUGUCCUGAAG |
| 1245 | HPV52_352 | AUGGGAAAACAUUAGAAGAGAGGGU |
| 1246 | HPV52_280 | AUGGCGUGUGUAUUAUGUGCCUACG |
| 1247 | HPV52_216 | CGAAGAGAGGUAUACAAGUUUCUAU |
| 1248 | HPV52_170 | GCAUGAAAUAAGGCUGCAGUGUGUG |
| 1249 | HPV52_145 | UGUGUGAGGUGCUGGAAGAAUCGGU |
| 1250 | HPV52_120 | ACACGACCCCGGACCCUGCACGAAU |
| 1251 | HPV52_95 | CACGGCCAUGUUUGAGGAUCCAGCA |
| 1252 | HPV52_70 | UAUAUAGAACACAGUGUAGCUAACG |

In one embodiment, the present invention provides an isolated polynucleotide for specific hybridization to HPV 56 consisting essentially of a sequence or a complement thereof selected from the group consisting of SEQ ID NOs: 1253-1367 (See Table 10). In some embodiments, the present invention provides a set of polynucleotides for specific hybridization to HPV 56, wherein the set comprises at least one polynucleotide consisting essentially of a sequence or a complement thereof selected from the group consisting of: SEQ ID NOs: 1253-1367. In certain embodiments, the methods of the present invention utilize a set of polynucleotide probes for specific hybridization to HPV 56 comprising SEQ ID NOs: 1253-1367.

TABLE 10

Polyribonucleotide probes for
determining HPV 56 nucleic acid.

| | | |
|---|---|---|
| SEQ ID NO: | Name | Sequence |

| | | |
|---|---|---|
| 1253 | HPV56_7754 | GUCAGUAUCUGUUUUGCAAACAUGU |
| 1254 | HPV56_7729 | AAUACACUAUGUAGGCCAAGUAUCU |
| 1255 | HPV56_7697 | UGUGUCUGCAACUUUGGUGUUUUGG |
| 1256 | HPV56_7605 | GUACCGCACCCUGUAUUACUCACAG |
| 1257 | HPV56_7532 | GGCCCUUUUCAGCAGAACAGUUAAU |
| 1258 | HPV56_7506 | GCCUAGUGCCAUUAUUUAAACCAAA |
| 1259 | HPV56_7431 | CAUUUUGUACAUGCAACCGAAUUCG |
| 1260 | HPV56_7366 | GUGUACUAUGUGUAUUGUGCAUACA |
| 1261 | HPV56_7322 | GUGUGUCAUUAUUGUGGCUUUUGUU |
| 1262 | HPV56_7271 | GUCUGUAAUAAACAUGAAUGAGUGC |
| 1263 | HPV56_7111 | UUUGUGUAACUGUGUUUGUGUGUUG |
| 1264 | HPV56_7083 | GUAAAAGGCGGUAGUGUGUUGUUGU |
| 1265 | HPV56_7058 | ACCUCCACCUCUACACCAGCAAAAC |
| 1266 | HPV56_7015 | GUCAAAGCCUGCUGUAGCUACCUCU |
| 1267 | HPV56_6872 | UGUCAACGGGAACAGCCACCAACAG |
| 1268 | HPV56_6823 | CACCAGCCUAGAAGAUAAAUAUAGA |
| 1269 | HPV56_6767 | AAUAUGAAUGCUAACCUACUGGAGG |
| 1270 | HPV56_6727 | CAAAAUUACUUUGUCUGCAGAGGUU |
| 1271 | HPV56_6612 | CUAACAUGACUAUUAGUACUGCUAC |
| 1272 | HPV56_6489 | UGAUUACGUCUGAGGCACAGUUAUU |
| 1273 | HPV56_6421 | UUUAAAGGGUAGCAAUGGUAGAGAA |
| 1274 | HPV56_6393 | UUGGGGAAACAAUACCUGCAGAGUU |
| 1275 | HPV56_6253 | ACCUUUAGACAUUGUACAAUCCACC |
| 1276 | HPV56_6212 | GCUAUGGACUUUAAGGUGUUGCAGG |
| 1277 | HPV56_6151 | GCCUCUUGCAUUAAUUAAUACACCU |
| 1278 | HPV56_6119 | AAGUCCACACAAGUUACCACAGGGG |
| 1279 | HPV56_6094 | ACAUUGGACUAAAGGUGCUGUGUGU |
| 1280 | HPV56_6031 | UAUAUCAGUUGAUGGCAAGCAAACA |
| 1281 | HPV56_5860 | UAUUUAUAAUCCGGACCAGGAACGG |
| 1282 | HPV56_5776 | CAUUCCCAAAGUUAGUGCAUAUCAA |
| 1283 | HPV56_5750 | GUGACUAAGGACAAUACCAAAACAA |
| 1284 | HPV56_5524 | UCCUCCUUUGCAUUAUGGCCUGUGU |
| 1285 | HPV56_5471 | CCUUUGUUCCUCAGUCUCCUUAUGA |
| 1286 | HPV56_5419 | CCAUUUUAUUCAGGUCCUGACAUAG |
| 1287 | HPV56_5394 | CCCUUUAGGUAAUGUGUGGGAAACA |
| 1288 | HPV56_5369 | CUAGUAACACCACUAAUGUAACUGC |
| 1289 | HPV56_5334 | ACACUUACCUAUAAAGCCUUCCACA |
| 1290 | HPV56_5306 | CUAGCCAGUCAGUUGCUACACCUUC |
| 1291 | HPV56_5131 | ACUAUACAAACACGUAGAGGCACAC |
| 1292 | HPV56_4953 | ACCUGCAACAUUAGUAUCUGCUGAU |
| 1293 | HPV56_4885 | GCAGCUCCUAGAUUAUAUAGAAAAG |
| 1294 | HPV56_4818 | AUUUGCUGUUCACGGUUCUGGUACA |
| 1295 | HPV56_4754 | GCAAUAUUUUAAUUAGCACACCCAC |
| 1296 | HPV56_4682 | GUACCCAUAUAACCAAUCCGUUAUU |
| 1297 | HPV56_4657 | ACCUCUAGUACUGUACAUGUCAGUA |
| 1298 | HPV56_4572 | AGGGAUUCCUAAUUUUACUGGGUCU |
| 1299 | HPV56_4546 | GAGUCCAGUGUUAUAGAAUCUGGUG |
| 1300 | HPV56_4474 | ACUCCGGCGCGACCACCUAUUGUUG |
| 1301 | HPV56_4429 | GGCUAUGUUCCAUUGGGGUCUAGGC |
| 1302 | HPV56_4206 | UAGUACUGUUACUACUAUGGUUGCC |
| 1303 | HPV56_4150 | CUGUGCUGUGUAUAUAUUUACAUGC |
| 1304 | HPV56_4082 | GUUUUGGUUUGUUAUAGCCACAUCC |
| 1305 | HPV56_4045 | CCUCUGUGUUUUCCAGUUGUAUAUU |
| 1306 | HPV56_4018 | GUCAUGUUGUCCCGCUUUUGCUAUC |
| 1307 | HPV56_3993 | UGCUUUUGUGUUUGUUUGCUUGUGU |
| 1308 | HPV56_3937 | UGCUACGCAUAUAUAUUGCAACCAU |
| 1309 | HPV56_3912 | GUGAAGUGUACCUGCCAUACAUUGC |
| 1310 | HPV56_3844 | CAAAUGAGUUUUCCAUAAAGUGCUG |
| 1311 | HPV56_3819 | CAGUAGUGUACAGGUUAGUUUGGGA |
| 1312 | HPV56_3717 | CAUAUCAUUGGACAAGUACAGACAA |
| 1313 | HPV56_3571 | CAGUAGAAGUAGAAGUAUCAACAAC |
| 1314 | HPV56_3546 | ACAUCAGCGACACAGACAAUACCGA |
| 1315 | HPV56_3488 | GAAUCAGAAUUUGACUCCUCCAGAG |
| 1316 | HPV56_3463 | ACCAGGAAAACGACCCAGACUACGG |
| 1317 | HPV56_3438 | ACCAAGACGCCGCAGUAUCCCACAG |
| 1318 | HPV56_3390 | AAUACAACACCCACAAGACCACCAC |
| 1319 | HPV56_3247 | CUACACAGACUUUGAACAAGAGGCC |
| 1320 | HPV56_3197 | GGGGUAGACUAUAGAGGUAUAUAUU |
| 1321 | HPV56_3129 | GUAUGCAAUAUGUAGCCUGGAAAUA |
| 1322 | HPV56_3024 | CAUUAAGAGACACAUGCGAGGAACU |
| 1323 | HPV56_2978 | GCACUGGAAUCAUUAAGUACAACAA |
| 1324 | HPV56_2896 | CAUUACUGUACUAAACCACCAGAUG |
| 1325 | HPV56_2738 | AGAAAACAAUGGAGACGCUUUCCCA |
| 1326 | HPV56_2683 | AAUGUUUCUUUACAAGGACGUGGUC |
| 1327 | HPV56_2562 | CCUAUGCUAGAUGCUAAAUUACGAU |
| 1328 | HPV56_2530 | GUCCACCAUUACUAAUUACAACCAA |
| 1329 | HPV56_2398 | AUGCUAAACUUGGGUUGUUGGAUGA |
| 1330 | HPV56_2269 | GUUUGGUACUUUGUGGACCGCCAAA |
| 1331 | HPV56_2124 | CAGUGGAUAAAGCACAUAUGUAGUA |
| 1332 | HPV56_1957 | AAGUAACAGAUGAUAGCCAAAUUGC |
| 1333 | HPV56_1896 | CACAGUUUACAGGAUAGUCAAUUUG |
| 1334 | HPV56_1837 | AUAUUAGUGAUGUGUAUGGAGACAC |
| 1335 | HPV56_1756 | CACAGGAGCAAAUGUUAAUUCAACC |
| 1336 | HPV56_1436 | GCAGGACUUGUUUAAAAGUAGCAAU |
| 1337 | HPV56_1411 | ACAAUGAAACGCCAACACAACAAUU |
| 1338 | HPV56_1377 | GAGGACUCUGUAAUACAUAUGGAUA |
| 1339 | HPV56_1346 | CUCACAAAACAGUACCUAUAGUAAC |
| 1340 | HPV56_1321 | GGUGCGGGAAUACACAAAAUGGAGG |
| 1341 | HPV56_1296 | GUAGAUGAAGAGGUACAGGGACGUG |
| 1342 | HPV56_1265 | UACAUUGGAAACUCUGGAAACACCA |
| 1343 | HPV56_1231 | UUUUAUCAGACCUACAAGACAGCGG |
| 1344 | HPV56_1170 | CCAUUAAGGGAUAUUAGUAAUCAGC |
| 1345 | HPV56_1108 | UACAAACAGCACAUGCAGAUAAACA |
| 1346 | HPV56_1078 | GACGCAGAAACAGUCAACAAUUGUU |
| 1347 | HPV56_993 | AGAUGAUGAAAGUGACGAGGAGGAU |
| 1348 | HPV56_943 | UGGUUUGAAGUAGAGGCAAUUGUAG |
| 1349 | HPV56_874 | CGCAUCAAGUAACUAACUGCAAUGG |
| 1350 | HPV56_807 | CCAAAGAGGACCUGCGUGUUGUACA |
| 1351 | HPV56_778 | GUUUGUGGUGCAGUUGGACAUUCAG |
| 1352 | HPV56_751 | AAUACACGUACCUUGUUGUGAGUGU |
| 1353 | HPV56_722 | AGACAAGCUAAACAACAUACGUGUU |
| 1354 | HPV56_619 | ACCUCAAACAGAAAUUGACCUACAG |
| 1355 | HPV56_594 | UGCAAGACGUUGUAUUAGAACUAAC |
| 1356 | HPV56_529 | GGAGACAAACAUCUAGAGAACCUAG |
| 1357 | HPV56_504 | UGGACCGGGUCAUGUUUGGGGUGCU |
| 1358 | HPV56_479 | ACGAUUUCAUCUAAUAGCACAUGGU |
| 1359 | HPV56_423 | AGAUGUCAAAGUCCGUUAACUCCGG |
| 1360 | HPV56_362 | UGGAGCUACACUAGAAAGUAUAACU |
| 1361 | HPV56_292 | CAGUGUGCAGAGUAUGUUUAUUGUU |
| 1362 | HPV56_267 | GUGUAUAGGGAUGAUUUUCCUUAUG |
| 1363 | HPV56_222 | ACACGUGCUGAGGUAUAUAAUUUUG |
| 1364 | HPV56_150 | CACUUGAGUGAGGUAUUAGAAAUAC |
| 1365 | HPV56_115 | UCAACAAUCCACAGGAACGUCCACG |
| 1366 | HPV56_77 | CAGCUUAUUCUGUGUGGACAUAUCC |
| 1367 | HPV56_15 | UACUUUUAUAUAUUGGGAGUGACCG |

In one embodiment, the present invention provides an isolated polynucleotide for specific hybridization to HPV 58 consisting essentially of a sequence or a complement thereof selected from the group consisting of SEQ ID NOs: 1368-1497 (See Table 11). In some embodiments, the present invention provides a set of polynucleotides for specific hybridization to HPV 58, wherein the set comprises at least one polynucleotide consisting essentially of a sequence or a complement thereof selected from the group consisting of: SEQ ID NOs: 1368-1497. In certain embodiments, the methods of the present invention utilize a set of polynucleotide probes for specific hybridization to HPV 58 comprising SEQ ID NOs: 1368-1497.

TABLE 11

Polyribonucleotide probes for
determining HPV 58 nucleic acid.

| | | |
|---|---|---|
| SEQ ID NO: | Name | Sequence |

| | | |
|---|---|---|
| 1368 | HPV58_7715 | GUUUGUUAUGCCAAACUAUGUCUUG |
| 1369 | HPV58_7678 | CUUUCAAUGCUUAAGUGCAGUUUUG |
| 1370 | HPV58_7596 | UCAUAUAUACAUGCAGUGCAGUUGC |
| 1371 | HPV58_7571 | UUUUGCCUAUACUUGCAUAUGUGAC |
| 1372 | HPV58_7546 | UUAAUCCUUUCCCUUCCUGCACUGC |
| 1373 | HPV58_7472 | CAUUUUGUGCAUGUAACCGAUUUCG |
| 1374 | HPV58_7444 | CAGUACUGCCUCCAUUUUACUUUAC |
| 1375 | HPV58_7384 | CUGCCUAUUAUGCAUACCUAUGUAA |
| 1376 | HPV58_7359 | UGUCCCUAAAUUGCCCUACCCUGCC |
| 1377 | HPV58_7334 | UUGGGUGUAUCUAUGAGUAAGGUGC |
| 1378 | HPV58_7266 | CUUGUCAGUUUCCUGUUUCUGUAUA |
| 1379 | HPV58_7232 | GUUAUGUGUCAUGUUUGUGUACAUG |
| 1380 | HPV58_7097 | UACCCGUGCACCAUCCACCAAACGC |
| 1381 | HPV58_7070 | GCCCAGACUAAAACGUUCGGCCCCU |
| 1382 | HPV58_6784 | CACUAACUGCAGAGAUAAUGACAUA |
| 1383 | HPV58_6722 | GGAAUAUGUACGUCAUGUUGAAGAA |
| 1384 | HPV58_6676 | GCACUGAAGUAACUAAGGAAGGUAC |
| 1385 | HPV58_6625 | AGUUAUUUGUUACCGUGGUUGAUAC |
| 1386 | HPV58_6533 | CUCUAUAGUUACCUCAGAAUCACAA |
| 1387 | HPV58_6488 | UACUGCAGUUAUCCAAAGUAGUGCA |
| 1388 | HPV58_6453 | GUCCCGGAUGACCUUUAUAUUAAAG |
| 1389 | HPV58_6428 | UAGGGCUGGAAAACUUGGCGAGGCU |
| 1390 | HPV58_6395 | ACGUGAGCAGAUGUUUGUUAGACAC |
| 1391 | HPV58_6177 | AAUGCAGCUGCUACUGAUUGUCCUC |
| 1392 | HPV58_6055 | CACAGCCAGGGUCUGAUAACAGGGA |
| 1393 | HPV58_6030 | ACUGAAACCAGUAACAGAUAUCCCG |
| 1394 | HPV58_5840 | AUCAGGCUUACAGUAUAGGGUCUUU |
| 1395 | HPV58_5590 | UAGCUAUUUUAUUUUGCGUCGCAGA |
| 1396 | HPV58_5562 | UGGAUGGUGCUGAUUUUAUGUUGCA |
| 1397 | HPV58_5532 | CUCCACUAACUCCUUUUAAUACCAU |
| 1398 | HPV58_5502 | CAUCUAUGUCUAGUCCAUUUAUUCC |
| 1399 | HPV58_5477 | GGUCCAGACAUUGCAUCUUCUGUAA |
| 1400 | HPV58_5452 | CACUCCUCUUGUGUCAUUGGAACCU |
| 1401 | HPV58_5423 | GUGUCCAUACCAUUAAAUACUGGAU |
| 1402 | HPV58_5398 | CUUUGCCACCACACGUACCAGUAAU |
| 1403 | HPV58_5373 | AGAGUCCUCUGCACUCACAUACGUC |
| 1404 | HPV58_5345 | GACGAUGCUGAUACUAUACAUGAUU |
| 1405 | HPV58_5304 | CUCCCUAUAGUAUUAAUGAUGGACU |
| 1406 | HPV58_5258 | CAACAGCAGCAACAAUUUGAAUUAC |
| 1407 | HPV58_5225 | UUAAGUCCCAUACAGCCUGUCCAGG |
| 1408 | HPV58_5200 | GGCUAAAGUACAUUACUACCAAGAC |
| 1409 | HPV58_5161 | AAAGGCUACACUUCGUACUCGCAGU |
| 1410 | HPV58_5050 | ACAUAGUGACAUAUCGCCUGCUCCU |
| 1411 | HPV58_5025 | ACCCUGAGGACACAUUGCAGUUUCA |
| 1412 | HPV58_4977 | CUCCUCAUAGACUUGUAACAUAUGA |
| 1413 | HPV58_4929 | GUCGCAACACCCAACAAGUUAAGGU |
| 1414 | HPV58_4867 | CAAUGUCACGUCUAGCACACCCAUU |
| 1415 | HPV58_4842 | CCUUUGUUAUUUCUACUGACAGUGG |
| 1416 | HPV58_4809 | GCACACAUAGUUAUGAAAACAUACC |
| 1417 | HPV58_4776 | CUGGACAUUUAAUAUUUUCCUCUCC |
| 1418 | HPV58_4741 | AUCCGUACUCCGCCCUCCUGCACCU |
| 1419 | HPV58_4716 | AUUUAAAUCCCUCCUUUACUGAGCC |
| 1420 | HPV58_4658 | CCUGCAAUACUUAAUGUUUCCUCUA |
| 1421 | HPV58_4633 | UAUUACCACCUCUGCAGAUACUACA |
| 1422 | HPV58_4608 | CAAUUCCCACUCCAUCUGGUUUUGA |
| 1423 | HPV58_4583 | AUAGACGCCGGUGCACCAGCCCCAU |
| 1424 | HPV58_4470 | GUACCCCACCGUCUGAGGCUAUACC |
| 1425 | HPV58_4375 | AUUACGAUAUGGUAGCUUAGGGGUG |
| 1426 | HPV58_4278 | CAUCUGCUACACAACUUUACCAAAC |
| 1427 | HPV58_4139 | CACAUGGUGGUAUGGUAUUGUAAAU |
| 1428 | HPV58_4114 | CAAGACUAACUGUAUACUGGUUCUG |
| 1429 | HPV58_4015 | GUGUCUGUGGGGUCGGCUCUACGAA |
| 1430 | HPV58_3990 | GCUGGUGUUGGUGUUGCUGCUUUGG |
| 1431 | HPV58_3954 | GCCAUUGGUGCUAUCUAUUUCUAUA |
| 1432 | HPV58_3845 | ACUGUAUGUAAACCACAAGCCAAUA |
| 1433 | HPV58_3799 | GCAAAUAAGUACUGGUGUUAUGUCA |
| 1434 | HPV58_3737 | ACAUACACAACGGAAACACAACGAC |
| 1435 | HPV58_3711 | GUGACAAAGUAGGAAUUGUUACUGU |
| 1436 | HPV58_3579 | CUAAAGUUUCACCUAUCGUGCAUUU |
| 1437 | HPV58_3544 | UAACUGUACAUACAAAGGGCGGAAC |
| 1438 | HPV58_3487 | GUAUACAGACUGCGCCGUGGACAGU |
| 1439 | HPV58_3462 | GAGACAACACCCAGUACUCCACAAA |
| 1440 | HPV58_3437 | CGACGACUCGAUUUACCAGACUCCA |
| 1441 | HPV58_3412 | CGAAAGUACACAGGGGACAAAGCGA |
| 1442 | HPV58_3350 | CCUAGUGAUCAAAUAUCCACUACUG |
| 1443 | HPV58_3288 | CUAAAACACAAUUAUGGGAGGUACA |
| 1444 | HPV58_3209 | GACUAUGUGGGGUUGUAUUAUAUAC |
| 1445 | HPV58_3184 | AUGUACUUUGGUAGCAGGAGAAGUU |
| 1446 | HPV58_3116 | GACAAUGAUAAAGCAAACACAAUGG |
| 1447 | HPV58_3046 | CUUAGAAGUGUGGUUAUCAGAGCCA |
| 1448 | HPV58_2985 | CAUUAGAGACAUUAAAUGCAUCACC |
| 1449 | HPV58_2943 | CAUCAAAGACUAAAGCGUUUCAAGU |
| 1450 | HPV58_2898 | UGGGAAUAUCACAUUUGUGCCACCA |
| 1451 | HPV58_2873 | GCUAUAAUGUAUACAGCCAGACAAA |
| 1452 | HPV58_2842 | UGAACAUUGGAAACUAAUACGCAUG |
| 1453 | HPV58_2794 | AAUCCUAGACAUAUACGAAGCUGAU |
| 1454 | HPV58_2717 | AAUUAGGCUUAAUAGAGGAAGAGGA |
| 1455 | HPV58_2598 | GOACAGUAGACUAACAGUAUUUGAA |
| 1456 | HPV58_2573 | GCAAAGAUUCACGAUGGCCAUAUUU |
| 1457 | HPV58_2516 | GGGCAUUAGUACAAUUAAAAUGUCC |
| 1458 | HPV58_2482 | GAUGGUAACGACAUUUCAAUAGAUG |
| 1459 | HPV58_2404 | GAUGCUAAACUAGGUAUGAUAGAUG |
| 1460 | HPV58_2278 | AUGUUACUGUGUGGCCCAGCAAAUA |
| 1461 | HPV58_2109 | AAAGCGUGGUAUGACAAUGGGACAA |
| 1462 | HPV58_1885 | AGAUUAACAGUGUUACAGCAUAGCU |
| 1463 | HPV58_1852 | GAUGUGCAAGGGACAACACCAGAAU |
| 1464 | HPV58_1800 | AAGUCAAGCAUGUGCCUUAUAUUGG |
| 1465 | HPV58_1770 | AUGUAUGAUUAUCGAGCCACCAAAA |
| 1466 | HPV58_1643 | AUACACACCUACAAUGUUUAACGUG |
| 1467 | HPV58_1590 | AAGUCCCUCCGUAGCAGAAAGUUUA |
| 1468 | HPV58_1565 | AUUGGUGUAUAACAGGGUAUGGAAU |
| 1469 | HPV58_1498 | GAAGCUUAUGGAGUAAGUUUUAUGG |
| 1470 | HPV58_1456 | CAUAACAGUAAUACUAAAGCAACGC |
| 1471 | HPV58_1402 | ACGGAUGUAGACAGUUGUAAUACUG |
| 1472 | HPV58_1349 | UAAAUGACUCGGAGUCUAGUGGGGU |
| 1473 | HPV58_1313 | CACACCAGGUAGAAAGCCAAAAUGG |
| 1474 | HPV58_1196 | CAAAUGUGUGUGUAUCGUGGAAAUA |
| 1475 | HPV58_1108 | GUGGACGAUAUAAAUGCUGUGUGUG |
| 1476 | HPV58_1083 | AGCGUUGUUUAAUGUACAGGAAGGG |
| 1477 | HPV58_1005 | CGAUAGUGGUACAGAUUUAAUAGAG |
| 1478 | HPV58_958 | CGAAGAACAGGAGAUAAUAUUUCAG |
| 1479 | HPV58_933 | GUUUGAGGUAGAAGCGGUAAUAGAA |
| 1480 | HPV58_837 | UACCAUUGUGUGCCCUAGCUGUGCA |
| 1481 | HPV58_799 | GACGUACGAACCCUACAGCAGCUGC |
| 1482 | HPV58_774 | UUUGUGUAUCAACAGUACAACAACC |
| 1483 | HPV58_749 | GUUACACUUGUGGCACCACGGUUCG |
| 1484 | HPV58_719 | CCACAGCUAAUUACUACAUUGUAAC |
| 1485 | HPV58_667 | UCAGACGAGGAUGAAAUAGGCUUGG |
| 1486 | HPV58_620 | AUCCUGAACCAACUGACCUAUUCUG |
| 1487 | HPV58_585 | CAACCCAACGCUAAGAGAAUAUAUU |
| 1488 | HPV58_560 | CCUGUAACAACGCCAUGAGAGGAAA |
| 1489 | HPV58_533 | CCCCGACGUAGACAAACACAAGUGU |
| 1490 | HPV58_481 | GUUUCAUAAUAUUUCGGGUCGUUGG |
| 1491 | HPV58_360 | AUGGAGACACAUUAGAACAAACACU |
| 1492 | HPV58_302 | GUGUGCUUACGAUUGCUAUCUAAAA |
| 1493 | HPV58_261 | GAAUAGUGUAUAGAGAUGGAAAUCC |
| 1494 | HPV58_184 | AAUCGAAUUGAAAUGCGUUGAAUGC |
| 1495 | HPV58_159 | AGGCGUUGGAGACAUCUGUGCAUGA |
| 1496 | HPV58_134 | CCACGGACAUUGCAUGAUUUGUGUC |
| 1497 | HPV58_104 | AGGACUAUGUUCCAGGACGCAGAGG |

In one embodiment, the present invention provides an isolated polynucleotide for specific hybridization to HPV 59 consisting essentially of a sequence or a complement thereof selected from the group consisting of SEQ ID NOs: 1498-1646 (See Table 12). In some embodiments, the present invention provides a set of polynucleotides for specific hybridization to HPV 59, wherein the set comprises at least one polynucleotide consisting essentially of a sequence or a complement thereof selected from the group consisting of: SEQ ID NOs: 1498-1646. In certain embodiments, the methods of the present invention utilize a set of polynucleotide probes for specific hybridization to HPV 59 comprising SEQ ID NOs: 1498-1646.

TABLE 12

Polyribonucleotide probes for
determining HPV 59 nucleic acid.

| | | |
|---|---|---|
| SEQ ID NO: | Name | Sequence |

| | | |
|---|---|---|
| 1498 | HPV597826 | CAAGUACAUGCACACUUUCUACUUA |
| 1499 | HPV59_7735 | ACUACUGUGCAAUCCAAGAAUGUGU |
| 1500 | HPV59_7657 | CGCCCUUGUUAAUAAAACAGCUUUU |
| 1501 | HPV59_7632 | AACAAUACUUGCAUAACUUUGGUGG |
| 1502 | HPV59_7592 | ACGCCAAAUAGUUAGUCAUCAUCCU |
| 1503 | HPV59_7567 | CCUAGACUACUAACACAACUUACAA |
| 1504 | HPV59_7542 | UCCCCAUCUUGUUUCCUCCUACACG |
| 1505 | HPV59_7474 | UCGGUUACCUUGGUUUAACCUUACC |
| 1506 | HPV59_7429 | GUCCAUUUUAUCCUUUAAAUCCUCC |
| 1507 | HPV59_7392 | CCUGAAUGUCCAGUUUUGCAUUUGC |
| 1508 | HPV59_7367 | AGGUGUGUUUGUUCCUUCAUUUUGU |
| 1509 | HPV59_7340 | CAUUAUUACACAUUGCCCUACUUAC |
| 1510 | HPV59_7309 | GUCCCUUUAUUGUUUCUUUGUCCUU |
| 1511 | HPV59_7218 | GUUUGUCUGCUGUAUGUGUGUAUUU |
| 1512 | HPV59_7152 | GUAUGUGUGCAUGUUGUAUGUUUUG |
| 1513 | HPV59_7117 | GUCUUCCAGAAAAUAGUGUUGUUUG |
| 1514 | HPV59_7086 | CCCCAUCACCAAAACGUGUUAAGCG |
| 1515 | HPV59_7027 | AGCUAGACCUAAGCCCACUAUAGGC |
| 1516 | HPV59_6965 | GAAAGGUUUUCUGCAGAUCUUGAUC |
| 1517 | HPV59_6940 | AAAGUUUUGGCCUGUAGAUCUUAAG |
| 1518 | HPV59_6915 | UUAAACAGGACCCUUAUGACAAACU |
| 1519 | HPV59_6877 | UGCUGCUGUAACUUGUCAAAAGGAC |
| 1520 | HPV59_6852 | UUGACACAUACCGUUUUGUUCAAUC |
| 1521 | HPV59_6688 | UAAAGAAUAUGCCAGACAUGUGGAG |
| 1522 | HPV59_6663 | CUAAUGUAUACACACCUACCAGUUU |
| 1523 | HPV59_6638 | UGUGCUUCUACUACUUCUUCUAUUC |
| 1524 | HPV59_6463 | AGGCAGUUAUUUAUAUUCCCCUUCC |
| 1525 | HPV59_6408 | GUGAUCAACUUCCUGAAUCACUAUA |
| 1526 | HPV59_6248 | GAUAACAAAAGUGAAGUACCAUUGG |
| 1527 | HPV59_6223 | GGCUAUGGACUUUAAAUUGUUGCAG |
| 1528 | HPV59_6136 | UACUACUGUGGUUCAGGGCGAUUGU |
| 1529 | HPV59_6020 | GAUACCAAAGAUACACGUGAUAAUG |
| 1530 | HPV59_5991 | CUGAAAACUCUCAUGUAGCAUCUGC |
| 1531 | HPV59_5912 | GUAGGUGUUGAAAUCGGUCGGGGCC |
| 1532 | HPV59_5882 | CCUAACUCUCAACGCUUGGUCUGGG |
| 1533 | HPV59_5857 | CCUUCCAGAUAACACAGUAUAUGAU |
| 1534 | HPV59_5798 | GUGUCUGCAUAUCAAUACAGAGUAU |
| 1535 | HPV59_5765 | AAAGGUGGUAAUGGUAGACAGGAUG |
| 1536 | HPV59_5701 | UAUUUUCUACCACGCAGGCAGUUCC |
| 1537 | HPV59_5676 | CUGAUGAGUAUGUCACCCGUACCAG |
| 1538 | HPV59_5642 | CUACCUCCACCUUCGGUAGCUAAGG |
| 1539 | HPV59_5498 | CCUUUACCACCAUACAGUCUAUUAA |
| 1540 | HPV59_5473 | GUUGAACCCACUUAUUCUACUACAC |
| 1541 | HPV59_5441 | GACCCGAUAUAGUUUUACCUAAUAC |
| 1542 | HPV59_5416 | GCCUGGGAUGUUCCUGUAAAUACAG |
| 1543 | HPV59_5382 | CACCUUUUCAAAUGUAACUGUUCCU |
| 1544 | HPV59_5357 | UGUCAUUAACACGGUCGGCAUCUAG |
| 1545 | HPV59_5315 | CCAACACUGCAUUUACAAUUCCUAA |
| 1546 | HPV59_5290 | ACAGAUGAAGCACCUACUAGUACUG |
| 1547 | HPV59_5250 | GGCUGCUACUGAUGAUAUAUAUGAU |
| 1548 | HPV59_5225 | AAUUGCAACCUCUUGUUUCUUCCCA |
| 1549 | HPV59_5200 | CCUAUACCACAUGCUGAAGAUAUUG |
| 1550 | HPV59_5088 | AACAUCCAGACGCAGCACUGUAAGG |
| 1551 | HPV59_5046 | CCCGGACUUUAUGGAUAUAGUUCGU |
| 1552 | HPV59_5013 | AUUAACUUUUGACCCCUCAUCAGAG |
| 1553 | HPV59_4988 | CUGCUUAUGAUCCAAUUGAUACUAC |
| 1554 | HPV59_4958 | GUCCAUCCACAUUUGUUACAUAUGA |
| 1555 | HPV59_4923 | ACAAGUUCGGGUGUCUAACGCUGAC |
| 1556 | HPV59_4896 | ACCUAGAUUGUACAGUAGGGCUAAU |
| 1557 | HPV59_4871 | AUCCAACAGUACGUCGUGUGGCUGG |
| 1558 | HPV59_4740 | CCAAACAGGUGAAAUUUCUGGUAAU |
| 1559 | HPV59_4685 | GUAGCUCUAGUUUUAUAAAUCCUGC |
| 1560 | HPV59_4660 | ACCCCAACCUCUUCUGUUCAAAUUA |
| 1561 | HPV59_4607 | CAGGAUUUGAAAUAUCUACCUCUAG |
| 1562 | HPV59_4555 | GAUUCUAGUGUUAUAACAUCUGGAG |
| 1563 | HPV59_4522 | CCUACAGAUCCAUCUAUAGUUACAU |
| 1564 | HPV59_4495 | CCACCAGUAGUUAUUGAACCUGUUG |
| 1565 | HPV59_4470 | UAUAGUAGAUGUAUCGCCUGCUAAA |
| 1566 | HPV59_4362 | AUUGCAGUGGACCAGCCUAGGAAUA |
| 1567 | HPV59_4238 | CCCAUCGUGCUGCUCGUCGUAAACG |
| 1568 | HPV59_4109 | GCAAUACUGUCCAUACAAUAAUUGC |
| 1569 | HPV59_4084 | UCCACUGUUACUACUAUAUGCCCAU |
| 1570 | HPV59_4029 | UGGUUAUCACCUCCUCAUAUGAGUG |
| 1571 | HPV59_3991 | GUGUGCAUAUACAUGGUUACUAGUA |
| 1572 | HPV59_3966 | UCCCGCUUCUGCAAUCUGUCUAUAU |
| 1573 | HPV59_3913 | AACCCUUGUAUUUGUGUGUUGUGUU |
| 1574 | HPV59_3858 | UGCAAAUGUAACACAAGCCAAUACU |
| 1575 | HPV59_3832 | GGUAUAUGAGUGUGUAAUGGUUGUU |
| 1576 | HPV59_3754 | UAACAUAUACAAGCGAAACACAACG |
| 1577 | HPV59_3718 | GAAACAGAGGAUCAGCCAAAACAGG |
| 1578 | HPV59_3686 | UGAAAAUAUUUCCUCUACCUGGCAU |
| 1579 | HPV59_3589 | UCCCUUGCAGUAACACUACGCCUAU |
| 1580 | HPV59_3562 | AUCCAGGCAACAACCCGCGACGGCA |
| 1581 | HPV59_3537 | UGUGACAACCCAGUCGUCCGUUUGC |
| 1582 | HPV59_3512 | GUCUACCAGCGUGUCAGUGGACUAC |
| 1583 | HPV59_3474 | AAGCGACCAAGACAGUGUGGAUACA |
| 1584 | HPV59_3392 | GCAACUAUCAUACCCCUCCGCAACG |
| 1585 | HPV59_3354 | ACCAGUGACGAGCAAGUAUCCACUG |
| 1586 | HPV59_3319 | GCAAGGUUAUUGAUUGUUAUGACUC |
| 1587 | HPV59_3291 | ACAGACAAGUGGGAAGUGCAUUAUA |
| 1588 | HPV59_3237 | GAGGAACAGGUGUACUAUGUAAAAU |
| 1589 | HPV59_3204 | GUGGACUUUUGGGGACUAUAUUAUA |
| 1590 | HPV59_3170 | UGAUGUAGGACAGUGGUGUAAAACC |
| 1591 | HPV59_3134 | GCAUUACACAAGCUGGACAUUUAUA |
| 1592 | HPV59_3109 | CCAUCUGCAGCAAGGAAAACACAAU |
| 1593 | HPV59_3050 | UGUUUCUUGCAUUGUCCAUUGCUCA |
| 1594 | HPV59_3023 | AGGUGCUGUUUGCCAUAGUUCUUGG |
| 1595 | HPV59_2980 | ACCGUACUUCCACUGUAAUGCCCUG |
| 1596 | HPV59_2948 | CAAGGCAUGUGAAGCUAUUGAACUG |
| 1597 | HPV59_2881 | CAGCAAGAGAGAACAAUAUACAUAC |
| 1598 | HPV59_2757 | CUUUCGCAGCGUUUAAGUGUGUUAC |
| 1599 | HPV59_2732 | GAAGAUGCAGACAGUGAUGGACACC |
| 1600 | HPV59_2706 | GCAGAUUAGAUUUGAACGAGGAAGA |
| 1601 | HPV59_2577 | GGUGGCCAUAUUUAAAUAGCAGAUU |
| 1602 | HPV59_2508 | GGCACCUAGUACAAAUUAAAUGUCC |
| 1603 | HPV59_2450 | GAUACAUAUAUGCGAAAUGCUUUGG |
| 1604 | HPV59_2396 | GAUCGUAAAUUAGCUAUGCUAGACG |
| 1605 | HPV59_2371 | UCACUUUUGGCUAGAACCUUUAACA |
| 1606 | HPV59_2264 | AAUUGCAUUGUGCUGUGUGGGCCAG |
| 1607 | HPV59_2123 | CAGUGGAUAAAAUGGAGAUGUGAUA |
| 1608 | HPV59_2002 | AGAUAGUAAUAGUAACGCCGCUGCA |
| 1609 | HPV59_1909 | UAGCGUGUUUGACCUGUCAGAAAUG |
| 1610 | HPV59_1838 | AUUAGUGAAGUUAUAGGGGAAACGC |
| 1611 | HPV59_1754 | CCAGAUACGUGCAUGUUAAUUGAAC |
| 1612 | HPV59_1729 | AGGACUUAGCACAUUACUACAUGUA |
| 1613 | HPV59_1662 | CAUGGGGAGUAGUAAUAUUAGCAUU |
| 1614 | HPV59_1614 | UAAUACAACCCUAUGUGCUAUAUGC |
| 1615 | HPV59_1585 | UCCAACUGUAGCAGAAGGAUUUAAA |
| 1616 | HPV59_1374 | GUAGCGACAGCAGUAACAUGGAUGU |
| 1617 | HPV59_1348 | UGUUUGUAGCGACAGUCAAAUAGAC |
| 1618 | HPV59_1323 | CUGGAAAUGGGGAUAGCAAUGGCAG |
| 1619 | HPV59_1298 | GAGACUCAGGUAACCGUGGAGAAUA |
| 1620 | HPV59_1242 | GAAGGUUAAUAACAGUGCCAGACAG |
| 1621 | HPV59_1212 | CAGUAAAUGUUAACCACCCAAAAGU |
| 1622 | HPV59_1155 | ACAGUAGUGAGAAAGCGGCGGCAGG |
| 1623 | HPV59_1130 | CGAAAGUUUGGGUGCAGUAUAGAAA |
| 1624 | HPV59_1105 | UGCACGGGAAAUGCAUGUUUUAAAA |
| 1625 | HPV59_1073 | GCCUUGUUUAAUGUGCAGGAAGCCC |
| 1626 | HPV59_954 | CAGGUGACAAAAUUUCAGAUGACGA |
| 1627 | HPV59_814 | GUUUAUGGACACACUAUCCUUUGUG |
| 1628 | HPV59_773 | GUAGAAACCUCGCAAGACGGAUUGC |
| 1629 | HPV59_684 | AUCCUUUGCUACUAGCUAGACGAGC |
| 1630 | HPV59_632 | UUACCUGACUCCGACUCCGAGAAUG |
| 1631 | HPV59_605 | GAAGUUGACCUUGUGUGCUACGAGC |
| 1632 | HPV59_569 | GACAUUGUUUUAGAUUUGGAACCAC |
| 1633 | HPV59_541 | AAUGCAUGGACCAAAAGCAACACUU |
| 1634 | HPV59_499 | AGACAGCAACGACAAGCGCGUAGUG |
| 1635 | HPV59_459 | AGGACAGUGUCGUGGGUGUCGGACC |
| 1636 | HPV59_379 | CUAAAACCUCUAUGUCCAACAGAUA |
| 1637 | HPV59_354 | GCUGCUGAUACGCUGUUAUAGAUGC |
| 1638 | HPV59_329 | CUGAAACCAAGACACCGUUACAUGA |
| 1639 | HPV59_304 | UCCGUGUAUGGAGAAACAUUAGAGG |
| 1640 | HPV59_228 | CUGUACACCGUAUGCAGCGUGUCUG |
| 1641 | HPV59_169 | CUGCAAGAAAGAGAGGUAUUUGAAU |
| 1642 | HPV59_130 | CAUGAUAUUCGCAUCAAUUGUGUGU |
| 1643 | HPV59_105 | GAGCACAACAUUGAAUAUUCCUCUG |
| 1644 | HPV59_74 | CUACACAACGACCAUACAAACUGCC |
| 1645 | HPV59_49 | AACGGCAUGGCACGCUUUGAGGAUC |
| 1646 | HPV59_24 | UAAAGGUAGUUGAAAAGAAAAGGGC |

In one embodiment, the present invention provides an isolated polynucleotide for specific hybridization to HPV 66 consisting essentially of a sequence or a complement thereof selected from the group consisting of SEQ ID NOs: 1647-1767 (See Table 13). In some embodiments, the present invention provides a set of polynucleotides for specific hybridization to HPV 66, wherein the set comprises at least one polynucleotide consisting essentially of a sequence or a complement thereof selected from the group consisting of: SEQ ID NOs: 1647-1767. In certain embodiments, the methods of the present invention utilize a set of polynucleotide probes for specific hybridization to HPV 66 comprising SEQ ID NOs: 1647-1767.

TABLE 13

Polyribonucleotide probes for
determining HPV 66 nucleic acid.

| | | |
|---|---|---|
| SEQ ID NO: | Name | Sequence |

| | | |
|---|---|---|
| 1647 | HPV66_7794 | GUCGUGCUAAAACAGGUUUCUUUUA |
| 1648 | HPV66_7737 | GUAUCUGUCUUGCAAAUAUGUAACC |
| 1649 | HPV66_7712 | GUGUAGCCCUUAUUGUAUAAGCCAA |
| 1650 | HPV66_7687 | GGUGUUUGCAAUAUAUUUUGUUGGC |
| 1651 | HPV66_7611 | UUACUCACCUGUAUUUCUGUGCCAA |
| 1652 | HPV66_7586 | GGUAUGUACACUGCCUUACCCUGUA |
| 1653 | HPV66_7521 | CAAAACGACUUUUCAGCAAAACAGU |
| 1654 | HPV66_7496 | CUAGCCUUUUGUCCUUAUUUAAACC |
| 1655 | HPV66_7466 | CAUUUUAUGCAUGCAACCGAAUUCG |
| 1656 | HPV66_7441 | CAAACUCCAUUUUAGUGCUGUACGC |
| 1657 | HPV66_7416 | GUUUGUAUGCACUAUAGUAACACAC |
| 1658 | HPV66_7377 | GUGGUGUUCCUUACUGUUUAAUGUU |
| 1659 | HPV66_7352 | CCUUGGGCAGUGUGUGUCAGGUUAG |
| 1660 | HPV66_7299 | AACAUGCAUGGUUACUUUUACGCGU |
| 1661 | HPV66_7246 | GCUAUGUGUAUGUAUGACUGUAUGU |
| 1662 | HPV66_7183 | UGUAUGGUUGUGCUUGUACUGUAUG |
| 1663 | HPV66_7122 | UUCCUCUUCUUCACCAGCUAAACGU |
| 1664 | HPV66_7097 | CUAAAAGGCGGGCGGCUCCUACCUC |
| 1665 | HPV66_7071 | UAGACCCAAGGCUAGUGUAUCUGCC |
| 1666 | HPV66_7000 | AGCUUUUCUGCAGACCUGGAUCAGU |
| 1667 | HPV66_6956 | AUCCCCUGGCUAAAUAUAAGUUUUG |
| 1668 | HPV66_6858 | AUCCCCACCAGUUGCAACUAGCUUA |
| 1669 | HPV66_6720 | CAAUCAAUACCUUCGCCAUGUGGAG |
| 1670 | HPV66_6692 | CAUUAACUAAAUAUGAUGCCCGUGA |
| 1671 | HPV66_6666 | CAUGACUAUUAAUGCAGCUAAAAGC |
| 1672 | HPV66_6540 | GAUUACCUCUGAGGCCCAAUUAUUU |
| 1673 | HPV66_6498 | UCCUCCCAGUUCUGUAUAUGUUGCU |
| 1674 | HPV66_6466 | UUGUAUUGGAAGGGUGGCAAUGGCA |
| 1675 | HPV66_6433 | GCAGGUAAUGUUGGGGAAGCCAUUC |
| 1676 | HPV66_6274 | AAGCUAUUACAGGAAUCAAAGGCUG |
| 1677 | HPV66_6220 | ACCCCGAUAGAGGACGGUGACAUGG |
| 1678 | HPV66_6195 | UUGUCCACCUCUUGCAUUAGUUAAU |
| 1679 | HPV66_6170 | AGUCUACACCAGGUAAUACAGGGGA |
| 1680 | HPV66_6145 | CAUUGGACUAAGGGCGCGGUGUGUA |
| 1681 | HPV66_6061 | AUAGAAGAUAGCCGGGACAAUAUAU |
| 1682 | HPV66_6031 | GAGGUCUCUAAUUUAGCAGGUAAUA |
| 1683 | HPV66_5999 | GUCAUCCAUUAUUUAAUAGGCUGGA |
| 1684 | HPV66_5904 | UCCAUCUUUCUAUAAUCCUGACCAG |
| 1685 | HPV66_5836 | GUUAGUGCAUAUCAGUAUAGAGUGU |
| 1686 | HPV66_5811 | UGGUACCAAAACAAACAUCCCUAAA |
| 1687 | HPV66_5783 | GCCAUCCUUAUUACUCUGUUUCCAA |
| 1688 | HPV66_5718 | GGAUACAUAUGUAAAACGUACCAGU |
| 1689 | HPV66_5565 | AUACAGGGAGCUACAUUUGCACUAU |
| 1690 | HPV66_5520 | CCCUUCGUACCUCAGUCUCCUUCUG |
| 1691 | HPV66_5469 | CCAUUUUAUUCAGGUCCUGAUAUAG |
| 1692 | HPV66_5427 | ACAGCUAAUGUUACUGCCCCUUUGG |
| 1693 | HPV66_5400 | CCUUCUACAUUAUCCUUUGCUAGUA |
| 1694 | HPV66_5374 | CACCUUCUGCACAAUUACCUAUUAA |
| 1695 | HPV66_5187 | CAAACACGUAGGGGUACGCAAAUAG |
| 1696 | HPV66_5128 | CAUUUACUACACGUAGAACAGGUGU |
| 1697 | HPV66_5003 | CCCCACAACAUUAAUAUCUGCUGAU |
| 1698 | HPV66_4943 | CAGGUUAUAUAGUAGGGCUUUUCAG |
| 1699 | HPV66_4918 | CAGGUUUUAGACGCCUUGCUGCUCC |
| 1700 | HPV66_4873 | CUAUACACGGUACUGGCAACGAACC |
| 1701 | HPV66_4831 | CUGGAAUACAUAGCUAUGAGGAAAU |
| 1702 | HPV66_4801 | CUGGUAAUAUUUUGAUUAGCACUCC |
| 1703 | HPV66_4760 | UGAUCCUCCAGUAAUUGAGGCUCCA |
| 1704 | HPV66_4729 | GUAGUACUACUAUAACAAACCCACU |
| 1705 | HPV66_4704 | CCCACAUCUAGUACUGUACAUGUAA |
| 1706 | HPV66_4617 | GGGGCUGGUGUUCCCAAUUUUACUG |
| 1707 | HPV66_4544 | UGUGGUGGAGUCAGUUGGGCCUACA |
| 1708 | HPV66_4509 | ACUAUAGUUGAUGUCACUCCUGCAC |
| 1709 | HPV66_4209 | GUGUAUAUAUUGCCAUGCUUUGUGG |
| 1710 | HPV66_4038 | UGCGCUUUGCUUUUGUGUUUGUCUG |
| 1711 | HPV66_3990 | GUAAUCGCCAUAUAUUGCAACCAUU |
| 1712 | HPV66_3965 | AUUGUAACACUGGGAAAGGUAACGU |
| 1713 | HPV66_3915 | GCUAAGCAUAUAUAUUGCACCCAUU |
| 1714 | HPV66_3890 | UGAAGUGUAAUUGCCAUACAUUGCU |
| 1715 | HPV66_3821 | CAAAUGAGUUGUCCAUAAAGUGUUG |
| 1716 | HPV66_3796 | ACCUAGUGUACAGGUUAUUUUGGGA |
| 1717 | HPV66_3702 | GGACAAGUACAGAUAAUAAAGACAG |
| 1718 | HPV66_3586 | UGAUAAAACUACGCCUGUAAUCCAU |
| 1719 | HPV66_3536 | AACAACGCCAACAGUAGAAGUCCAC |
| 1720 | HPV66_3470 | GAAUCAGAACCUGACUCCUCCAGAG |
| 1721 | HPV66_3445 | ACCAGGAAAACGACCCAGAGCAAGU |
| 1722 | HPV66_3296 | ACCGAGAGUAUUUACUGUCCUGACU |
| 1723 | HPV66_3228 | AUUACACAGACUUUGAACAGGAGGC |
| 1724 | HPV66_3181 | GGUGGAUUACAGAGGCAUAUAUUAU |
| 1725 | HPV66_3144 | AUAAUGGAGAGUGUGGGUGGUGUAA |
| 1726 | HPV66_3109 | UUGUAUGGAAUAUGUGGUGUGGAAA |
| 1727 | HPV66_3017 | ACAUGUGAUGAACUGUGGCGCACGG |
| 1728 | HPV66_2961 | CACUGGAAGCAAUAAGUAACACAAU |
| 1729 | HPV66_2878 | CAUUAAUGUACUAAACCACCAGAUG |
| 1730 | HPV66_2614 | CCAUUAGAUAACAAUGGUAAUCCUG |
| 1731 | HPV66_2411 | CAGAUACGUGUUGGAGAUACAUAGA |
| 1732 | HPV66_2374 | CUAGACAAUGCCAAAUUAGGUUUGC |
| 1733 | HPV66_2254 | UUGGUACUGUGUGGACCACCAAAUA |
| 1734 | HPV66_2104 | UGCCAGUGGAUAAAGCAUAUAUGUA |
| 1735 | HPV66_1941 | AGUAACAGAUGAUAGCCAAAUUGCC |
| 1736 | HPV66_1875 | GCAACACAGUUUACAAGACAAUCAA |
| 1737 | HPV66_1739 | CACAAGAGCAAAUGUUAAUUCAACC |
| 1738 | HPV66_1649 | GGGGAGUAAUUGUAAUGAUGCUAAU |
| 1739 | HPV66_1612 | UGUGUGUACUAUCAUAUGCAAUGCU |
| 1740 | HPV66_1532 | GUUGUAACGAUUGGAUAUGUGCAAU |
| 1741 | HPV66_1484 | GAGUGCCAUAUACAGAGUUGGUGCG |
| 1742 | HPV66_1436 | GUAGUAACGUACAAGGAAGAUUACA |
| 1743 | HPV66_1403 | CACCAACACACCAAUUGCAGGAACU |
| 1744 | HPV66_1363 | CACUCGGUAUCAAAUAUGGAUAUAG |
| 1745 | HPV66_1332 | UGGAGGCUCGCAAAACAGUAAUUGU |
| 1746 | HPV66_1298 | ACGAAAAGGGAAAUGGGUGCGGGAG |
| 1747 | HPV66_1273 | UUGGAAACAUCACAACAGGUAGAAU |
| 1748 | HPV66_1226 | GGCUAAUAUUAUCAGAAGACAGCGG |
| 1749 | HPV66_1165 | GGUAGUCCCUUAAGUGAUAUUAGUA |
| 1750 | HPV66_1106 | AAGUACAAACAGCACAUGCAGAUGC |
| 1751 | HPV66_939 | UGGAUGGUUUCAGGUAGAAGCAAUU |
| 1752 | HPV66_874 | CGCAUCAUCUAAAUAACUGCAAUGG |
| 1753 | HPV66_819 | UACGUGUGGUACAACAGCUGCUUAU |
| 1754 | HPV66_791 | UUGGACAUUCAGAGUACCAAAGAGG |
| 1755 | HPV66_759 | UACCUUGUUGUAAGUGUGAGUUGGU |
| 1756 | HPV66_604 | UAUAUUAGAACUUGCACCGCAAACG |
| 1757 | HPV66_579 | GUAAAGUACCAACGUUGCAAGAGGU |
| 1758 | HPV66_554 | AGAAUCUACAGUAUAACCAUGCAUG |
| 1759 | HPV66_529 | GGAGACAUACGAGUAGACAAGCUAC |
| 1760 | HPV66_504 | UGGACCGGGUCAUGUUUGCAGUGUU |
| 1761 | HPV66_462 | CACUGUGAACAUAAAAGACGAUUUC |
| 1762 | HPV66_346 | AUAAAUAUUCAGUGUAUGGGGCAAC |
| 1763 | HPV66_291 | GCAGUAUGUAGGGUAUGUUUAUUGU |
| 1764 | HPV66_150 | CAUCUGAGCGAGGUAUUACAAAUAC |
| 1765 | HPV66_115 | UCAGCAAUACACAGGAACGUCCACG |
| 1766 | HPV66_88 | GCCUGUAGAUAUCCAUGGAUUCCAU |
| 1767 | HPV66_63 | GUACAUAUAAAAGGCAGCCUGUUGU |

In one embodiment, the present invention provides an isolated polynucleotide for specific hybridization to HPV 68 consisting essentially of a sequence or a complement thereof selected from the group consisting of SEQ ID NOs: 1768-1875 (See Table 14). In some embodiments, the present invention provides a set of polynucleotides for specific hybridization to HPV68, wherein the set comprises at least one polynucleotide consisting essentially of a sequence or a complement thereof selected from the group consisting of: SEQ ID NOs: 1768-1875. In certain embodiments, the methods of the present invention utilize a set of polynucleotide probes for specific hybridization to HPV 68 comprising SEQ ID NOs: 1768-1875.

TABLE 14

Polyribonucleotide probes for
determining HPV 68 nucleic acid.

| | | |
|---|---|---|
| SEQ ID NO: | Name | Sequence |

| | | |
|---|---|---|
| 1768 | HPV68_7798 | CUGAACACAGCAGUUCUCUAUACUA |
| 1769 | HPV68_7696 | GGCACACAUACCAAUACUUUUACUU |
| 1770 | HPV68_7661 | CUACAUCCAUAAAUUUGUGCAACCG |
| 1771 | HPV68_7628 | UGUCUGGUAGUGUAAGUUAUACAGU |
| 1772 | HPV68_7597 | GCCAGUAUAACUACUUUUGCAUUCA |
| 1773 | HPV68_7527 | CCUCCCUUGUAAUAAAACUGCUUUU |
| 1774 | HPV68_7502 | CAAUAGUUUGGCAACCAACGUAUCU |
| 1775 | HPV68_7452 | UCGUACUGGCGCACCUUAGUUAGUC |
| 1776 | HPV68_7427 | CCCACAUAGUUGGCACCAGUAACAG |
| 1777 | HPV68_7352 | GUCGUUGGUACUAUUUGCUUUUAGA |
| 1778 | HPV68_7325 | UGGCCGGGUUGUGUGCGACCGCUUU |
| 1779 | HPV68_7300 | AACUAUACCGUGUGGCCAUUUUGUA |
| 1780 | HPV68_7258 | CCUAAGGUGUGUUACAUUAUAUGCA |
| 1781 | HPV68_7186 | CUGUGACUAACAUAUGUCCUUGUUU |
| 1782 | HPV68_7159 | UAUGUCCGUGUCCUUUGUGGUUGCA |
| 1783 | HPV68_7108 | GUGUAUGUUUGCAAGUAUGUGUGUA |
| 1784 | HPV68_7078 | GUGUAUGUGCAUGUAUGUGUAUGUG |
| 1785 | HPV68_7053 | UGUGUCAUGUUGGUGUUGGUAUGUU |
| 1786 | HPV68_7028 | UGUUGUUUGUCUGUGUGGUUGUAUA |
| 1787 | HPV68_6961 | ACCACAUCUACCUCUAAACACAAAC |
| 1788 | HPV68_6898 | UUACAGGCAGGUGUUCGCAGACGGC |
| 1789 | HPV68_6873 | AUUCCCAUUAGGACGCAAAUUUCUG |
| 1790 | HPV68_6848 | AAAAGUUUAGUUCUGAACUGGACCA |
| 1791 | HPV68_6809 | CCUAUGAUGGUCUUAACUUUUGGAA |
| 1792 | HPV68_6749 | ACCUACAAUCAGCAGCAAUUACAUG |
| 1793 | HPV68_6719 | CAUCUGCUAGUCUUGUAGAUACAUA |
| 1794 | HPV68_6541 | GUACCAGCUGUGUAUGAUUCUAAUA |
| 1795 | HPV68_6516 | AUUGUCCACUACUACAGACUCUACU |
| 1796 | HPV68_6337 | GAAACUCCUAGUAGUUAUGUGUAUG |
| 1797 | HPV68_6312 | GUAUAUUAAGGGCACUGACAUUCGU |
| 1798 | HPV68_6145 | GUACCUUUGGAUAUAUGUCAAUCUG |
| 1799 | HPV68_6118 | GGUACAUUACAAGAAACGAAAAGCG |
| 1800 | HPV68_6052 | GAAUUGGUAAAUACUCCUAUUGAGG |
| 1801 | HPV68_6016 | CCUACCAAUGUACAACAAGGGGACU |
| 1802 | HPV68_5924 | AUGUUGCAGUGGACUGUAAACAAAC |
| 1803 | HPV68_5874 | UGAAAAUUCCCCGUUUUCCUCUAAU |
| 1804 | HPV68_5743 | CCUGAGUCUACAUUAUAUAAUCCAG |
| 1805 | HPV68_5625 | CCAUCCAUAUUUUAAGGUUCCUAUG |
| 1806 | HPV68_5600 | GUACAUCUAGGUUAUUAACUGUAGG |
| 1807 | HPV68_5380 | CAAUUGAUACAACCUUUGCCAUAAC |
| 1808 | HPV68_5355 | CAGUUGCCUUUAACACCCUCUACUC |
| 1809 | HPV68_5326 | CUGAUGUUGUAUUACCAUCUACAAC |
| 1810 | HPV68_5301 | UGGAACACGCCUGUAAAUACUGGUC |
| 1811 | HPV68_5270 | UACUAAUACUACCAUUCCUCUUGGU |
| 1812 | HPV68_5245 | UGGCUUCUGCUGCAUCCACUACAUA |
| 1813 | HPV68_5220 | CGUUCCCACAUAUCAGUUCCUUCAU |
| 1814 | HPV68_5158 | CACCUGAUACUGACAAUACUACAGU |
| 1815 | HPV68_5126 | GGACCCUAUGGAUAACUUAUAUGAU |
| 1816 | HPV68_5101 | AACCAUUGGUUGCCCCUGAGCAGGC |
| 1817 | HPV68_5066 | UAGUAACAUUACCCCUGCUGACAGC |
| 1818 | HPV68_5006 | GACCAUGUUUACACGCCGAGGUACA |
| 1819 | HPV68_4973 | AACAGUACGUUUUAGCAGAGUAGGC |
| 1820 | HPV68_4877 | UACUACUCUUACAUAUGAACCUGCU |
| 1821 | HPV68_4823 | AACGCACCCUUCAUCAUUUGUAACA |
| 1822 | HPV68_4692 | GUAUUUGCAACACAUGGCACUGGUA |
| 1823 | HPV68_4636 | UGUUUGUAAGUACCCCUACAUCAGG |
| 1824 | HPV68_4595 | UAUAAUAGAAGUGCCACAAACAGGU |
| 1825 | HPV68_4570 | CUAACCCUGCAUUUACAGACCCGAC |
| 1826 | HPV68_4498 | CUACCACUACACCGGCAGUUUUAGA |
| 1827 | HPV68_4452 | GUACCAACAUUUACAGGCACCUCUG |
| 1828 | HPV68_4427 | CAGUGUUAUUACAUCUGGGACACCA |
| 1829 | HPV68_4395 | GAACCCUCCAUUGUGCAAUUGGUGG |
| 1830 | HPV68_4323 | GGAAAACCUAAUACUGUUGUGGAUG |
| 1831 | HPV68_4206 | GGUACUACACUUGCAGACAAAAUAU |
| 1832 | HPV68_4046 | CAGUAACUGUUAUAGUGUGCAUUUG |
| 1833 | HPV68_4012 | GUGGUUAUUACACAGUCUUACUCUU |
| 1834 | HPV68_3966 | CAUUUGAGGUGUUUGCUGUAUACCU |
| 1835 | HPV68_3867 | GCAUGUAUAUAUGUUGCACUGUCCC |
| 1836 | HPV68_3796 | CCCACACUGUACACUAUAUGUAUAU |
| 1837 | HPV68_3766 | GGGGUAUAUGACAUUAUAAGUGUGU |
| 1838 | HPV68_3728 | GAAACUGUUAAACUACCAUCUAGUG |
| 1839 | HPV68_3697 | UGUUUCAGAAGCACAACGUGACAAG |
| 1840 | HPV68_3514 | AAGACGGAGCCUUUGUUGUGGUGAC |
| 1841 | HPV68_3489 | UCAGUAGAAGUGCAGGCCAAAACAA |
| 1842 | HPV68_3438 | AGCCCUCUGAGCCCGACAACGUGUC |
| 1843 | HPV68_3316 | UACUGAAUCUGUUGCCGACCUACAG |
| 1844 | HPV68_3291 | GUACCACUGACGGAAAAGUAUCCAC |
| 1845 | HPV68_3188 | UAUUACGAAAGGUUUAUGCAGGAUG |
| 1846 | HPV68_3129 | AAACCCAAGGGCGUGUGGAUUACUG |
| 1847 | HPV68_3079 | UGUAGUGUGGGGUACAAUUUACUUU |
| 1848 | HPV68_3054 | GGGACAAGAGUAACUCAAUGCAUUA |
| 1849 | HPV68_2978 | AGUAAUGAACUAUGGCAUACAAAGC |
| 1850 | HPV68_2927 | AGCCUUGCUAAAACUGCAUAUAGUG |
| 1851 | HPV68_2776 | UAACUAUUGGAAUUGUGUGCGACUG |
| 1852 | HPV68_2523 | GUAUUUACAUAGUAGACUAACCGUG |
| 1853 | HPV68_2496 | UAACCCUGUAGAAGACAAUAGGUGG |
| 1854 | HPV68_2429 | GUUUAGAUAGAAAACACAGACACCU |
| 1855 | HPV68_2301 | UUCAGCAAGUCACUUUUGGUUAGAG |
| 1856 | HPV68_2186 | AAGGCACGCCAAAACGAAAUUGUAU |
| 1857 | HPV68_1684 | UUGCAUGUUCCAGACAGCUGUAUGC |
| 1858 | HPV68_1358 | CACCUACUACCCAACUUAAAGUAUU |
| 1859 | HPV68_1333 | GAUAGUGAAAACCAGGAUCCUAAAU |
| 1860 | HPV68_1166 | CAAGACAACCGGCGUAUACAGUGCC |
| 1861 | HPV68_1141 | UCACUAAAUGUAAGCAGUACACAGG |
| 1862 | HPV68_1116 | AGCAAAGUCGCCAUUACAGGAAUUA |
| 1863 | HPV68_1091 | CAGACAGUAUAGAAAGCAGUCCUUU |
| 1864 | HPV68_897 | UAAACAAACAGGUGACACAGUCUCA |
| 1865 | HPV68_772 | UCACUAAAUUUUGUGUGUCCGUGGU |
| 1866 | HPV68_745 | CGGACACUACAACAGCUGUUUAUGG |
| 1867 | HPV68_685 | CUGUGUUGUAAGUGUAACAAGGCAC |
| 1868 | HPV68_518 | UGUUAGAGCUAUGUCCAUACAAUGA |
| 1869 | HPV68_487 | CAUGGACCAAAGCCCACCGUGCAGG |
| 1870 | HPV68_358 | CACCUAACAACAAAACGAAGAUUAC |
| 1871 | HPV68_253 | GUGUAUGCAACUACAUUAGAAACCA |
| 1872 | HPV68_228 | GGAACUACGAUAUUACUCGGAAUCG |
| 1873 | HPV68_150 | UGACCUAUGUGUAGUGUAUAGAGAC |
| 1874 | HPV68_117 | ACAACGGACAGAGGUAUAUGAAUUU |
| 1875 | HPV68_3 | GGCGCUAUUUCACAACCCUGAGGAA |

In one embodiment, the present invention provides an isolated polynucleotide for specific hybridization to HPV 82 consisting essentially of a sequence or a complement thereof selected from the group consisting of SEQ ID NOs: 1876-2026 (See Table 15). In some embodiments, the present invention provides a set of polynucleotides for specific hybridization to HPV 82, wherein the set comprises at least one polynucleotide consisting essentially of a sequence or a complement thereof selected from the group consisting of: SEQ ID NOs: 1876-2026. In certain embodiments, the methods of the present invention utilize a set of polynucleotide probes for specific hybridization to HPV 82 comprising SEQ ID NOs: 1876-2026.

TABLE 15

Polyribonucleotide probes for
determining HPV 82 nucleic acid.

| | | |
|---|---|---|
| SEQ ID NO: | Name | Sequence |

| | | |
|---|---|---|
| 1876 | HPV82_7835 | UUGUGUUUUGCCUAUGCUUGCAACA |
| 1877 | HPV82_7785 | AUGUAUUACUCAUCUGCAGGUGUGC |
| 1878 | HPV82_7760 | GCCAAGUUUCUAUCCUACCUAUAAA |
| 1879 | HPV82_7735 | GGCAGGUCAUGAACUAAAUGUCUCU |
| 1880 | HPV82_7662 | CCGCCCUGUAAUAAUUUAUAUGCUU |
| 1881 | HPV82_7612 | CACACCACAUUACUCAUUUGUACUU |
| 1882 | HPV82_7550 | UGGUAUGUACAUCCCGCCCGCCCAC |
| 1883 | HPV82_7525 | GGCAUAACCCUUAAUUCUUUUGGCA |
| 1884 | HPV82_7494 | CAACUUUUGAACCACACUACCUAUG |
| 1885 | HPV82_7408 | GCAUGUACCACAGGAUUCCAUUUUG |
| 1886 | HPV82_7373 | GCAGCACACUUGUAUAUAUAUGUUC |
| 1887 | HPV82_7348 | AUUGCCCUACCCAUAUUUGUGGCUU |
| 1888 | HPV82_7319 | GUUAAGGGUGGUGUUUAGGUGGCGU |
| 1889 | HPV82_7191 | GUAUGGUUUCUGUGUGGUUUACUAA |
| 1890 | HPV82_7130 | GUGUGCGUGUUGUGUGUAUUUGUGU |
| 1891 | HPV82_7086 | CGCCCUGCCUAUGUAUGUGUUUGUG |
| 1892 | HPV82_7030 | CCCCAUCCUCUUCCGCUUCCUCGUC |
| 1893 | HPV82_7001 | CAAACCCAGACCAGGCCUUAAAAGG |
| 1894 | HPV82_6939 | UCUUUGGAUUUGGAUCAGUUUGCAU |
| 1895 | HPV82_6880 | CUAAAGAAGACCCUUUGGCAAAAUA |
| 1896 | HPV82_6755 | GGAUUCUACAAUUUUAGAACAGUGG |
| 1897 | HPV82_6651 | UUUAAGCAGUACAUUAGGCAUGGGG |
| 1898 | HPV82_6626 | UGCACAAACAUUUACUCCAGCAAAC |
| 1899 | HPV82_6589 | CCAAUUUAACCAUUAGCACUGCUGU |
| 1900 | HPV82_6459 | GGUUCUAUGAUAACCUCUGAUUCUC |
| 1901 | HPV82_6433 | GUUAUAUUUAUUCAGCUACUCCCAG |
| 1902 | HPV82_6408 | GGUGCUGGCCGCGACCCUAUUAGUA |
| 1903 | HPV82_6383 | AGACAAGGCUUAUAUUAAGGGUACU |
| 1904 | HPV82_6358 | CUGGUGUGGUUGGUGAUGCCAUUCC |
| 1905 | HPV82_6281 | AGCAGAUACAUAUGGCAAUUCUAUG |
| 1906 | HPV82_6247 | CUGUGUGUAAAUACCCUGAUUACUU |
| 1907 | HPV82_6210 | GCUACUAAAUCAGAUGUUCCAUUGG |
| 1908 | HPV82_6137 | UGUGUCUACUGUCAUUGAGGAUGGC |
| 1909 | HPV82_6039 | AUUAUAGGCUGCGCUCCUCCUAUUG |
| 1910 | HPV82_6012 | GUGGACAACAAACAAACUCAGUUAU |
| 1911 | HPV82_5840 | UAAUCCAGACACAGAUCGUUUGGUG |
| 1912 | HPV82_5815 | UUGGUCUUCCUGAUCCUAAUUUGUU |
| 1913 | HPV82_5738 | UACACGUGCUGAAAUACCUAAGGUA |
| 1914 | HPV82_5654 | AACCCGCACCGGCAUAUAUUAUUAU |
| 1915 | HPV82_5628 | CGCAUUGUCAACACAGAAGAAUAUA |
| 1916 | HPV82_5603 | GUAUUUACCACCUGCACCAGUGUCA |
| 1917 | HPV82_5519 | UAUACAUAUUUGUUACGCAAACGCC |
| 1918 | HPV82_5494 | GGUGGGGAUUACUACUUUGUGGCCG |
| 1919 | HPV82_5467 | GACACACAACAUGCUAUUGUUAUAC |
| 1920 | HPV82_5442 | GCCCUUUAUUCCACACACAUCUAUU |
| 1921 | HPV82_5417 | UGUUACCUACUUCACCCACUGUGUG |
| 1922 | HPV82_5392 | CCUAUUCAUACGGGUCCUGAUGUUG |
| 1923 | HPV82_5345 | CAUCUUAUGCUAAUGUUACUAUCCC |
| 1924 | HPV82_5320 | CCUUCAUUGUCUUCCUCUGUUUCUU |
| 1925 | HPV82_5294 | CAUUUUCUCCUUUGUCUACACAACU |
| 1926 | HPV82_5269 | CAAACCACACCUAUGCUUCGCUCUC |
| 1927 | HPV82_5244 | UGAAACAGGUUUUAUGCAGCCUACA |
| 1928 | HPV82_5182 | CCUUUACUUUCCCCUUCUACUAAUA |
| 1929 | HPV82_5143 | AUAAGUAGUAUUGCACCUGCUGAGG |
| 1930 | HPV82_5009 | AUAUUAUUAAACUGCACCGCCCUGC |
| 1931 | HPV82_4976 | CUACUGAUGUUGCACCAGAUCCUGA |
| 1932 | HPV82_4951 | GAUACAUCAUUGUCCUUUGAGGAAC |
| 1933 | HPV82_4898 | UUAGUAAGCCCUCUACAUUUGUUAC |
| 1934 | HPV82_4872 | GGUUAAGGUUACUAAUCCAGACUUU |
| 1935 | HPV82_4847 | GUUUAUAUAGCAGGGCAUUUUCACA |
| 1936 | HPV82_4790 | GUAAGGAACCCAUUAGCAGUACACC |
| 1937 | HPV82_4765 | GUAUUUGCCUCCAAUGUUACUACUG |
| 1938 | HPV82_4706 | AUAUAUUUACCAGUACCCCUACGUC |
| 1939 | HPV82_4667 | CAUUUAUUGAGGCACCACAAUCAGG |
| 1940 | HPV82_4627 | ACAAGCACUAACAUUGAAAAUCCCU |
| 1941 | HPV82_4558 | AUUACUUCCUCUUCUACAACAACUC |
| 1942 | HPV82_4511 | AUUCAGGCUCUACUAUACCUACCUU |
| 1943 | HPV82_4425 | UCCGGCCAGGCCUCCAAUUAUUAUU |
| 1944 | HPV82_4400 | GACGGCCUGGUGUUGUAGAUAUUGC |
| 1945 | HPV82_4259 | UUAUUCCUAAGGUAAAGGGCACUAC |
| 1946 | HPV82_4214 | AAUUAUAUUCCACAUGCAAAGCUGC |
| 1947 | HPV82_4165 | ACAAUGGUGGCUGCACGUGCACGGC |
| 1948 | HPV82_4036 | CCACAUCACCUUUAACUACAUUUAC |
| 1949 | HPV82_3976 | AAUCCCAAUAUGUGUUUGCAGCAGC |
| 1950 | HPV82_3876 | UGUAUAUAGUUACUCGCAACCAUUG |
| 1951 | HPV82_3801 | GUCAUUGGGUAUUAUGACAGUGUAA |
| 1952 | HPV82_3776 | UUAAAGUACCAUCAAGUGUGACAGU |
| 1953 | HPV82_3746 | CACACCAACGUCAAAAGUUUAUUGA |
| 1954 | HPV82_3704 | GUAAUACAAAAGCAGGCAUUGUUAC |
| 1955 | HPV82_3668 | UGUUUAAAGAAGUGUCAUCUACCUG |
| 1956 | HPV82_3580 | GCAACUAAAACUGCGUUUAUAGUUC |
| 1957 | HPV82_3544 | GGAACUGCAGGCCCAAACACCGGAG |
| 1958 | HPV82_3519 | CACCUGCGACCACCAAAUACACUGU |
| 1959 | HPV82_3487 | GACUCCUCCACAGUCACCCCGCUGU |
| 1960 | HPV82_3449 | CACCACAACAACGAAAACGACAGCG |
| 1961 | HPV82_3404 | CGACCAAUACCUAUUCCGCCUCCGC |
| 1962 | HPV82_3362 | CACCCUCUACUACAACUGUUGAACA |
| 1963 | HPV82_3337 | GUAUCUAGUACCUACAGCACCCCGU |
| 1964 | HPV82_3295 | GAGGUAUAUAUGUGUGGCAAUGUAA |
| 1965 | HPV82_3198 | CGUGGACUAUACAGGUAUUUAUUAC |
| 1966 | HPV82_3131 | UGGACUAUACAUGUUGGACAUAUGU |
| 1967 | HPV82_3105 | GUUUGAUGGGAAUAAGGACAAUACA |
| 1968 | HPV82_3036 | AUGCUAUGAACUAUGGGGCGAGGCC |
| 1969 | HPV82_2977 | GCAUUAGAAUCGCUAAACAAAUCUG |
| 1970 | HPV82_2937 | AUCAAAACAAAAGGCCUGCCAAGCC |
| 1971 | HPV82_2912 | AUCAAGUAGUACCAGCAUCGGCAGU |
| 1972 | HPV82_2887 | GAAAGAAACAUGCAAACCCUUAACC |
| 1973 | HPV82_2751 | GACCCUAUGUCAUCGUUUAAAUGUG |
| 1974 | HPV82_2650 | GGAAUCCUGUAUAUGCACUAAAUGA |
| 1975 | HPV82_2519 | GCUGCAAAUUGUAUGCCCACCAUUG |
| 1976 | HPV82_2454 | GACCAGUACCUAAGAAAUUUCCUAA |
| 1977 | HPV82_2196 | CGAUACCAGGGUAUUAACUUUAUGU |
| 1978 | HPV82_2138 | GUAUAGAUGUGACAAAGUGCAAGAC |
| 1979 | HPV82_2113 | CACUAACAAUGUCAGCAUGGAUUAG |
| 1980 | HPV82_2088 | CACUACAAACGAGCACAAAGAAAAU |
| 1981 | HPV82_1999 | AAUUGGCUGAUACAGAUAGCAAUGC |
| 1982 | HPV82_1951 | UUGACCAUGAUGUAGUAGACGAUAG |
| 1983 | HPV82_1914 | AGCACGUUUGAACUAUCGCAAAUGG |
| 1984 | HPV82_1889 | ACAACUACAGCACAGUUUUGAUGAU |
| 1985 | HPV82_1841 | CAUUAGUAGCACAUAUGGCGAAACA |
| 1986 | HPV82_1774 | UUAUAGAACCACCUAAGCUACGUAG |
| 1987 | HPV82_1723 | CCAUUGCCAAAUGUUUAGGUACAUU |
| 1988 | HPV82_1685 | ACUGUUAGCUAGAUUUACAUGUGCC |
| 1989 | HPV82_1660 | CAUGUGAUUGGGGUACUAUUGUGCU |
| 1990 | HPV82_1633 | GUAUGUACUACCAUAUACAAUGCCU |
| 1991 | HPV82_1571 | UGCCUUAUUUGGGGUAUCGCCAAUG |
| 1992 | HPV82_1546 | AAACAUGCUGCACGGACUGGGUAUG |
| 1993 | HPV82_1518 | GAGUUGGUAAGGGUAUUUAAAAGUG |
| 1994 | HPV82_1460 | CAAUGCAAAAGCAAUGUUUAUGGCA |
| 1995 | HPV82_1417 | CCAAUGUAGGACUAAACAGUAUAUG |
| 1996 | HPV82_1392 | GACCUGGAAACAAACGAAAAUGCUA |
| 1997 | HPV82_1320 | GAUGGGCAAAAUGACGGGUCACAAC |
| 1998 | HPV82_1294 | AGACUGUGGAAGGACCCUUACAGGU |
| 1999 | HPV82_1242 | AGGAGAUUACUGGACAGUUAUCCGG |
| 2000 | HPV82_1203 | CAGCAACAACCAAAACAGGCAAACC |
| 2001 | HPV82_1156 | GCAGCCCAUUAAAAGACAUUACAAA |
| 2002 | HPV82_1093 | AAACACAGGCACACAAAGAGGCUGU |
| 2003 | HPV82_1068 | GCACAGGCGUUGUUGCAGGUCCAAG |
| 2004 | HPV82_1035 | AAUAGUAUUUGUAGUCAGGCGGAAC |
| 2005 | HPV82_987 | GAUACAAAUGAUACAGGGUCUGAUA |
| 2006 | HPV82_955 | CGGGAGAUAAUAUAUCAGACGAUGA |
| 2007 | HPV82_867 | ACAUCGGCAAUGGACAGUGAAGGUA |
| 2008 | HPV82_833 | UAAGCCUGGUGUGCCCGUGGUGUGC |
| 2009 | HPV82_807 | AUUUCAGCAAAUGUUACUGGGCGAC |
| 2010 | HPV82_782 | AAAGCAGUGGAGACAGCCUUCGCAU |
| 2011 | HPV82_748 | UGCAGGUGUUCGAGUGUUGUACAGC |
| 2012 | HPV82_723 | GUGUUACAGAAUUAAAGUGCACUGU |
| 2013 | HPV82_608 | UAACACCACAACCUGAAAUUGACUU |
| 2014 | HPV82_583 | CAAUUAAAGGACAUAGUGUUGGAGU |
| 2015 | HPV82_539 | UAGUGAAACCCAGGUGUAAUAACGC |
| 2016 | HPV82_514 | AUUGCAGAAAACCACCAAGACAACG |
| 2017 | HPV82_440 | AGAAAAGCAAAAGGUGGUGGACGAC |
| 2018 | HPV82_415 | GAUGUCAGAGACCACUUGGGCCUGA |
| 2019 | HPV82_361 | CAUUAGAGGCCAUUACUAACAAAAG |
| 2020 | HPV82_332 | AAGGUAUAGUAGGUCUGUGUAUGGU |
| 2021 | HPV82_265 | GGGACAAUACGCCAUAUGCAGCAUG |
| 2022 | HPV82_234 | GUAGCAUUUACAGAACUUAGGAUUG |
| 2023 | HPV82_209 | GUUGUGUAGAGCAGAUGUGUAUAAU |
| 2024 | HPV82_164 | GUCUAUGCACAAUAUUCAGGUAUUG |
| 2025 | HPV82_139 | ACGAAUUAUGUGAAGCCUGCAAUAC |
| 2026 | HPV82_105 | UUUGAAGACAUAAGAGAAAGACCAC |

### Hybridization

The methods of the present invention comprises contacting the one or more polynucleotide probes with the sample under a hybridization condition sufficient for the one or more polynucleotide probes to hybridize to the target nucleic acid in the sample to form double-stranded nucleic acid hybrids. Preferably, the one or more polynucleotide probes is diluted in a probe diluent that also can act as a neutralizing hybridization buffer. The diluent can be used to dissolve and dilute the probe and also help restore the sample to about a neutral pH, e.g., about pH 6 to about pH 9, to provide a more favorable environment for hybridization. Sufficient volume of probe diluent, preferably one-half volume, can be used to neutralize one and one-half volume of base-treated sample. Preferably, the probe diluent is a 2-[bis(2-Hydroxyethyl) amino] ethane sulfonic acid (BES, Sigma, St. Louis, Mo.)/sodium acetate buffer. Most preferably, the probe diluent is a mixture of 2 M BES, 1 M sodium acetate, 0.05% of the antimicrobial agent NaN₃, 5 mM of the metal chelating agent EDTA, 0.4% of the detergent Tween™-20 and 20% of the hybridization accelerator dextran sulfate. The pH of the probe diluent can be about 5 to about 5.5.

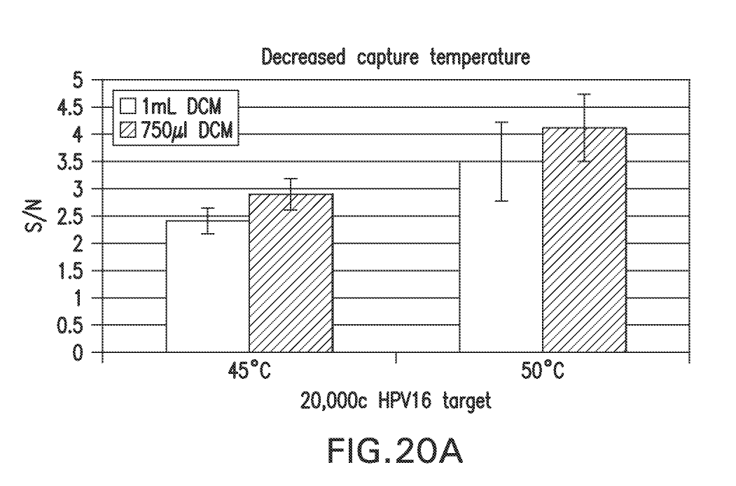

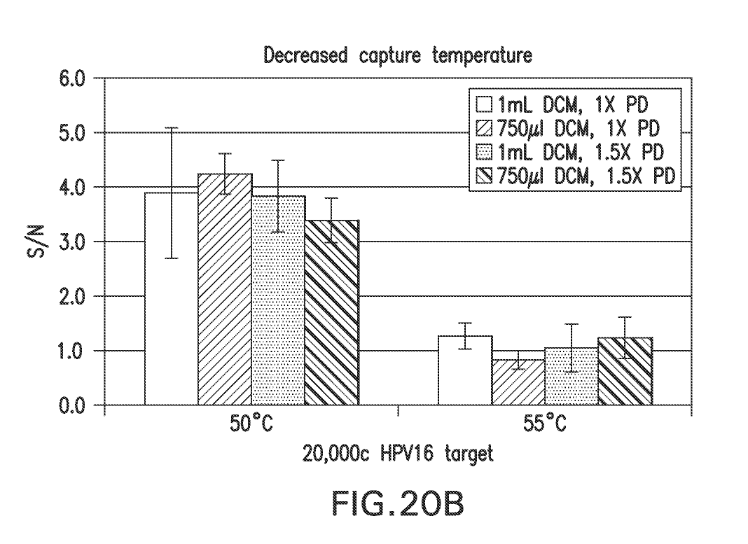

Thus, for example, after treatment with base, an aliquot of sample can be removed from the sample tube and combined with a sufficient amount of probe to allow hybridization to occur under a hybridization condition. The hybridization condition is sufficient to allow the one or more polynucleotide probes to anneal to a corresponding complementary nucleic acid sequence, if present, in the sample to form double-stranded nucleic acid hybrids. The probes and sample nucleic acids can be incubated for a hybridization time, preferably at least about 5 minutes, to allow the one or more polynucleotide probes to anneal to a corresponding complementary nucleic acid sequence. The hybridization condition can comprise a hybridization temperature of at least about 20° C., preferably about 50 to about 80° C. In certain embodiments, the hybridization is performed at a temperature of less than 55° C. In other embodiments when synRNA probes are used and when the sample containing the target nucleic acid contains a large volume of collection medium (i.e. >1 ml), the hybridization temperature is between 45° C. and 55° C. and preferably is about 50° C. (see FIGS. 20A and 20B). Lowering the hybridization temperature provides the ability to detect 20,000 copies of HPV target nucleic acid in an assay. For any given target to be determined and the one or more polynucleotides employed, one of ordinary skill in the art can readily determine the desired hybridization condition by routine experimentation.

The present invention also allows for hybridization of probes to targets in the presence of anti-hybrid antibody that is immunospecific to double-stranded nucleic acid hybrids (i.e. the anti-hybrid antibody can be added at the same time or before the probes are added to the sample containing the target nucleic acid). This allows for reduction in the time to perform an assay.

### Anti-Hybrid Antibodies

The double-stranded nucleic acid hybrids formed in accordance with the present invention can be detected using an antibody that is immunospecific to double-stranded nucleic acid hybrids. The antibody is immunospecific to double-stranded hybrids, such as but not limited to RNA/DNA; DNA/DNA; RNA/RNA; and mimics thereof, where “mimics” as defined herein, refers to molecules that behave similarly to RNA/DNA, DNA/DNA, or RNA/RNA hybrids. The anti-double-stranded nucleic acid hybrid antibody (i.e., “anti-hybrid” antibody) that is utilized will depend on the type of double-stranded nucleic acid hybrid formed. In one embodiment, the antibody is immunospecific to RNA/DNA hybrids.

It will be understood by those skilled in the art that either polyclonal or monoclonal anti-hybrid antibodies can be used and/or immobilized on a solid support or phase in the present assay as described below. Monoclonal antibody prepared using standard techniques can be used in place of the polyclonal antibodies. Also included are immunofragments or derivatives of antibodies specific for double-stranded hybrids, where such fragments or derivatives contain binding regions of the antibody.

For example, a polyclonal RNA:DNA hybrid antibody derived from goats immunized with an RNA:DNA hybrid can be used. Hybrid-specific antibody can be purified from the goat serum by affinity purification against RNA:DNA hybrid immobilized on a solid support, for example as described in Kitawaga et al., Mol. Immunology, 19:413 (1982); and U.S. Pat. No. 4,732,847, each of which is incorporated herein by reference.

Other suitable methods of producing or isolating antibodies, including human or artificial antibodies, can be used, including, for example, methods which select recombinant antibody (e.g. single chain Fv or Fab, or other fragments thereof) from a library, or which rely upon immunization of transgenic animals (e.g., mice) capable of producing a repertoire of human antibodies (see, e.g. Jakobovits et al. Proc. Natl. Acad. Sci. USA, 90:2551 (1993); Jakobovits et al., Nature, 362: 255 (1993); and U.S. Pat. Nos. 5,545,806 and 5,545,807).

In one embodiment, the target nucleic acid to be determined is DNA (e.g., HPV 18 genomic DNA) or RNA (e.g., mRNA, ribosomal RNA, nucleolar RNA, transfer RNA, viral RNA, heterogeneous nuclear RNA), wherein the one or more polynucleotide probes are polyribonucleotides or polydeoxyribonucleotides, respectively. According to this embodiment, the double-stranded nucleic acid hybrids (i.e. DN/RNA hybrids) formed can be detected using an antibody that is immunospecific to RNA:DNA hybrids.

In a preferred embodiment of the present invention, a polyclonal anti-RNA/DNA hybrid antibody is derived from goats immunized with an RNA/DNA hybrid. Hybrid-specific antibody is purified from the goat serum by affinity purification against RNA/DNA hybrid immobilized on a solid support. Monoclonal antibody prepared using standard techniques can be used in place of the polyclonal antibodies.

While any vertebrate may be used for the preparation of anti-RNA/DNA hybrid monoclonal antibodies, goats or rabbits are preferred. Preferably, a goat or rabbit is immunized with a synthetic poly(A)-poly(dT) hybrid by injecting the hybrid into the animal in accordance with conventional injection procedures. Polyclonal antibodies may be collected and purified from the blood of the animal with antibodies specific for the species of the immunized animal in accordance with well-known antibody isolation techniques. For the production of monoclonal antibodies, the spleen can be removed from the animal after a sufficient amount of time, and splenocytes can be fused with the appropriate myeloma cells to produce hybridomas. Hybridomas can then be screened for the ability to secrete the anti-hybrid antibody. Selected hybridomas may then be used for injection into the peritoneal cavity of a second animal for production of ascites fluid, which may be extracted and used as an enriched source of the desired monoclonal antibodies incorporated herein by reference.

In some embodiments, the step of detecting comprises contacting the double-stranded nucleic acid hybrids with a first anti-hybrid antibody to capture the double-stranded nucleic acid hybrids, wherein the first anti-hybrid antibody is immunospecific to double-stranded nucleic acid hybrids. In one embodiment, the first anti-hybrid antibody is immobilized onto a solid support such as a test tube surface. It will be understood by those skilled in the art that a solid support includes polystyrene, polyethylene, polypropylene, polycarbonate or any solid plastic material in the shape of test tubes, beads, microparticles, dip-sticks or the like. Examples of a solid support also includes, without limitation, glass beads, silica beads, glass test tubes, and any other appropriate shape made of glass. A functionalized solid support such as plastic, silica, or glass that has been modified so that the surface contains carboxyl, amino, hydrazide or aldehyde groups can also be used. Immobilization of the antibody can be direct or indirect. Preferably, test tubes are directly coated with anti-hybrid antibody in accordance with methods known to those skilled in the art or briefly described below. The antibody can also be biotinylated and subsequently immobilized on, for example streptavidin coated tubes or silica, or modified by other methods to covalently bind to the solid phase. Solubilized biotinylated antibody can be immobilized on the streptavidin coated tubes before capture of the hybridized samples as described below or in conjunction with the addition of the hybridized samples to simultaneously immobilize the biotinylated antibody and capture the hybrids.

In another embodiment, the first anti-hybrid antibody is attached to the solid phase in accordance with the method of Fleminger et al., Appl. Biochem. Biotech. 23:123 (1990), by oxidizing the carbohydrate portion of the antibody with periodate to yield reactive aldehyde groups. The aldehyde groups are then reacted with a hydrazide-modified solid phase such as MicroBind-HZ™ microtiter plates available from Dynatech Laboratories (Chantilly, Va.). Passive coating of the antibody according to the well known method of Esser, P., Nunc Bulletin No. 6 (November 1988) (Nunc, Roskilde, Denmark) can also be employed.

In other embodiments, Ventrex Star™ tubes (Ventrex Laboratories Inc., Portland, Me.) are coated with streptavidin by the method of Haun et al., Anal. Biochem. 191:337-342 (1990). After binding of streptavidin, a biotinylated goat polyclonal antibody as described above, or otherwise produced by methods known to those skilled in the art, is bound to the immobilized streptavidin. Following antibody binding, tubes can be post-coated with a detergent such as Tween™-20 and sucrose to block unbound sites on the tube and stabilize the bound proteins as described by Esser, Nunc Bulletin No. 8, pp. 1-5 (December 1990) and Nunc Bulletin No. 9, pp. 1-4 (June 1991) (Nunc, Roskilde, Denmark) and Ansari, et al., J. Immunol. Methods, 84:117 (1985). Preferably, each tube is coated with between 10 ng and 100 μg biotinylated antibody. Most preferably each tube is coated with approximately 250 ng of biotinylated antibody.

As discussed above, the solid phase can be coated with functional antibody fragments or derivatized functional fragments of the anti-hybrid antibody.

In some embodiments, hybridized samples are incubated in tubes coated with the first anti-hybrid antibody for a sufficient amount of time to allow capture of the double-stranded nucleic acid hybrids by the immobilized capture antibodies. The hybrids can be bound to the immobilized antibodies by incubation, for example incubation for about 5 minutes to about 24 hours at about 15 to about 65° C. In some embodiments, the incubation time is about 30 to about 120 minutes at about 20 to about 40° C., with shaking at about 300 to about 1200 rpm. In another embodiment, capture occurs with incubation at about one hour at about room temperature with vigorous shaking on a rotary platform. It will be understood by those skilled in the art that the incubation time, temperature, and/or shaking can be varied to achieve alternative capture kinetics as desired.

In other embodiments, the first anti-hybrid antibody is coupled to a magnetic bead (e.g., COOH-beads) to capture double-stranded nucleic acid hybrids. Magnetic bead-based technology is well known in the art. In some embodiments, magnetic silica beads having derivatized surfaces for reacting with antibody can be employed.

In one embodiment, the step of detecting further comprises providing a second anti-hybrid antibody that is immunospecific to double-stranded nucleic acid hybrids, wherein the second anti-hybrid antibody is detectably labeled either directly or indirectly.

For example, in some embodiments, an anti-hybrid antibody as described above can be conjugated to a detectable label to provide the second anti-hybrid antibody for detection of the double-stranded nucleic acid hybrids. Conjugation methods for labeling are well known in the art. Preferably, an antibody, such as the mouse monoclonal antibody deposited with the American Type Culture Collection as ATCC Accession number HB-8730, is conjugated to a detectable label such as alkaline phosphatase. It will be understood by those skilled in the art that any detectable label such as an enzyme, a fluorescent molecule, or a biotin-avidin conjugate can be used.

The antibody conjugate can be produced by well known methods such as direct reduction of the monoclonal antibody with dithiothreitol (DTT) to yield monovalent antibody fragments. The reduced antibody can then be directly conjugated to maleimated alkaline phosphatase by the methods of Ishikawa et al., J. Immunoassay 4:209-237 (1983) and Means et al., Chem. 1: 2-12 (1990), and the resulting conjugate can be purified by HPLC.

In another embodiment, the double-stranded nucleic acid hybrids can be detected indirectly, for example using an unlabelled anti-hybrid antibody for which a labeled antibody is specific. For example, the second anti-hybrid antibody can be a mouse immunoglobulin that is detected by a labeled goat anti-mouse antibody.

The double-stranded nucleic acid hybrids can be contacted with the second anti-hybrid antibody under a binding condition that is sufficient to provide for specific antibody-antigen binding (i.e., antibody/double-stranded nucleic acid hybrid binding), while minimizing non-specific binding. The binding condition preferably comprises a binding buffer comprising 0.1 M Tris-HCl, pH 7.5, 0.6 M NaCl to reduce cross reaction of antibody with other nucleic acid species, ZnCl₂ and MgCl₂ for stabilizing alkaline phosphatase, normal goat serum to block non-specific interaction of conjugate with the capture surface, 0.25% of the detergent Tween™-20 to block non-specific binding of conjugate, and sodium azide as a preservative. Reactions can then be washed with a wash buffer (e.g. 0.1 M Tris-HCl, pH 7.5, 0.6 M NaCl, 0.25% Tween™-20, and sodium azide) to remove as much of the unbound or non-specifically bound second anti-hybrid antibody as possible. The second anti-hybrid antibody that is bound to the double-stranded nucleic acid hybrids can subsequently be detected, for example by colorimetry or chemiluminescence methods as described by e.g. Coutlee, et al., J. Clin. Microbiol. 27:1002-1007 (1989). For example, bound alkaline phosphatase conjugate can be detected by chemiluminescence with a reagent such as a Lumi-Phos™ 530 reagent (Lumigen, Detroit, Mich.) using a detector such as an E/Lumina™ luminometer (Source Scientific Systems, Inc., Garden Grove, Calif.), an Optocomp I™ Luminometer (MGM Instruments, Hamden, Conn.), or the like.

In some embodiments, the one or more polynucleotides can be conjugated to a label, such as an enzyme, or to a hapten such as biotin, that is then detected with a labeled anti-hapten antibody.

Thus, target-specific oligoribonucleotides or oligodeoxynucleotides can be designed using commercially available bioinformatics software. For example, for the detection of dsDNA targets, DNA can be denatured, hybridized to the RNA probes, and captured via anti-RNA:DNA hybrid antibodies on a solid support. Detection can be performed by various methods, including anti-RNA:DNA hybrid antibodies conjugated with alkaline phosphatase for chemiluminescent detection. Alternatively, other detection methods can be employed, for example using anti-RNA:DNA hybrid antibodies conjugated with phycoerythrin, suitable for detection by fluorescence.

In other embodiments, the methods of the present invention, optionally, further comprise a step of amplification of the target nucleic acid. Amplification techniques are known in the art and may be utilized. For example, Whole Genome Amplification (WGA) may be employed. WGA is an isothermal process that uses non-specific primers to generate amplicons using the target nucleic acid sequence as a template. For example, Phi 29 DNA polymerase can be used in combination with non-specific primers to amplify target nucleic acid sequences. The polymerase can move along the target nucleic acid sequence displacing the complementary strand. The displaced strand becomes a template for replication allowing high yields of high-molecular weight DNA to be generated. For example, helicase-dependent amplification may be employed.

### Kits

In other aspects, the present invention provides a kit comprising the necessary components and reagents for performing the methods of the present invention. The kit can comprise at least one of the following: an inert sample collection device, such as a dacron swab for exfoliated cell sample collection; a sample transport medium for stabilization of the sample during transport to the laboratory for analysis; a base, or a hydrolysis reagent; one or more polynucleotide probes specific for the target nucleic acid to be determined; neutralizing probe diluent; anti-hybrid antibody coated test tubes; and any necessary controls.

Preferably, the sample transport medium is Specimen Transport Medium; the base is 0.415 M NaOH; the neutralizing probe diluent is a BES/sodium acetate buffer; the test tubes are Ventrex Star™ tubes coated with a polyclonal anti-hybrid antibody; and the conjugated anti-hybrid antibody is a mouse monoclonal antibody conjugated to alkaline phosphatase. Preferably, the kit also contains a substrate for the chemiluminescent detection of alkaline phosphatase, such as a CDP-Star® with Emerald II (Applied Biosystems, Bedford, Mass.).

The present invention will be illustrated in more detail by way of Examples, but it is to be noted that the invention is not limited to the Examples.

### EXAMPLES

### Example 1

### Polynucleotide Probes for Determining HPV 18 or HPV 16 DNA

Oligoarray 2.0 was chosen as the tool with which to identify RNA probes specific for HPV 18 or HPV 16 DNA. A database of sequences to be checked against, in this case, HPV high risk and low risk types: 1, 2, 3, 4, 5, 6, 8, 11, 13, 16, 18, 26, 30, 31, 33, 34, 35, 39, 40, 42, 43, 44, 51, 52, 53, 54, 56, 58, 59, 61, 62, 66, 67, 68, 69, 70, 71, 72, 73, 74, 81, 82, 83, 84, and 89 was provided and the sequence of interest, i.e., HPV16 or HPV 18 was then BLASTed against the database to search for any regions of identity, and the similarities were stored. Tm and % GC were then computed for ribonucleotides of a specified length and compared to the parameters, after which secondary structure was examined. Cross hybridization was checked with the Mfold package, using the similarity determined by BLAST.

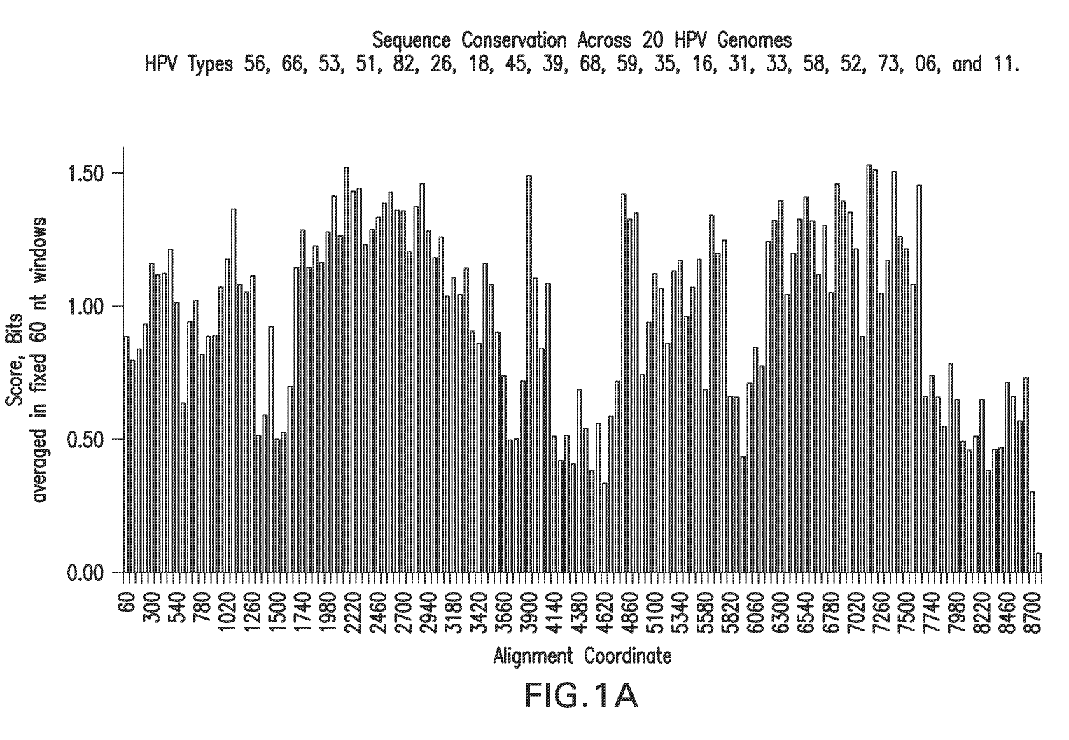

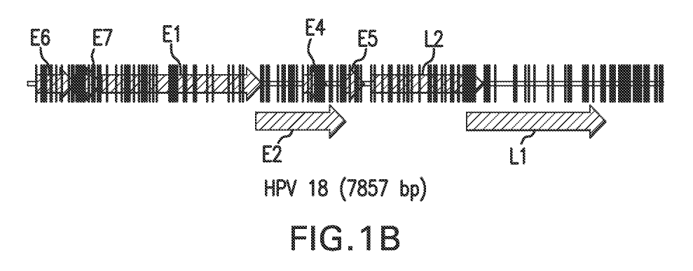

The parameters of the Oligoarray 2.0 program were set to look for ribonucleotides of 25 nt length, Tm range of 55-95° C., a GC range of 35-65%, and no secondary structure or cross-hybridization at 55° C. or below. Using these parameters to determine ribonucleotide probes for HPV18 (with a modified BLAST database that did not include HPV45, as we are not interested in specificity against that type) resulted in 145 ribonucleotides (for HPV 18) and 127 ribonucleotides (for HPV 16) covering a total of about 3.7 kb of the target (i.e., HPV 18 or HPV 16 viral DNA). The sequences of the ribonucleotide probes that were selected are shown Tables 1 and 2 above. Sequence conservation across 20 HPV genomes is shown in FIG. 1a. As schematically shown in FIG. 1b for HPV 18, all regions of the HPV 18 genome were represented in the respective probes.

RNA oligos were ordered from IDT technologies, at the 250 nM scale, with standard desalting. Oligos were stored in Ambion's RNA Storage Solution (1 mM Sodium Citrate, pH 6.4). The synthetic ribonucleotide probes are hereinafter referred to as “synRNA.”

### Example 2

### Protocol for Detecting HPV 18 DNA Using HPV 18 SynRNA

The hybridization and detection protocol was performed essentially as described in Table 16.

TABLE 16
Protocol

| | | |
|---|---|---|
| Denature | 1 | Sample nucleic acid was denatured with alkali and heat. |
| Hybridize/ | 2 | Synthetic RNA probes were added to sample, hybridized, |
| Capture | | and neutralized |
| | 3 | Synthetic RNA probe/target DNA hybrids were captured |
| | | with anti-hybrid antibody immobilized on a substrate |
| Conjugate | 4 | Alkaline phosphatase-conjugated anti-hybrid antibodies |
| | | were added |
| Wash | 5 | Samples were washed |
| Detection | 6 | Alkaline-phosphatase-activated chemiluminescent |
| | | substrate was added |
| Read | 7 | Samples were read using a luminometer |

### Example 3

### Results

To remove as much variability as possible, data was analyzed as (S−N)/N, expressed as (S/N)−1. When signal=noise, data value=0.0.

A. Specificity Demonstrated with HPV 18 synRNA

As shown in Table 17, the synthetic RNA probes (synRNAs) designed for HPV 18 showed no cross-reactivity with either HPV 6 or HPV 16 at up to 10⁹ copies/assay (200 ng/ml). synRNA=3.7 kb coverage of HPV 18 DNA; 25 mers@1.34 nM final in hybridization.

TABLE 17
Specificity of HPV18 synRNA

| | Input Copies | Avg RLU | S − N | (S/N) − 1 |
|---|---|---|---|---|
| HPV 18 | 0 | 55 | 0 | 0.0 |
| | 5000 | 167 | 113 | 2.1 |
| | 10\hat{ }4 | 238 | 183 | 3.4 |
| | 10\hat{ }5 | 2044 | 1989 | 36.5 |
| HPV 16 | 0 | 53 | 0 | 0.0 |
| | 10\hat{ }7 | 79 | 26 | 0.5 |
| | 10\hat{ }8 | 59 | 6 | 0.1 |
| | 10\hat{ }9 | 84 | 32 | 0.6 |
| HPV 6 | 0 | 51 | 0 | 0.0 |
| | 10\hat{ }7 | 51 | 0 | 0.0 |
| | 10\hat{ }8 | 54 | 3 | 0.1 |
| | 10\hat{ }9 | 60 | 9 | 0.2 |

B. Cross-Reactivity of HPV18 SynRNA with HPV45

HPV 18 synRNA was not designed to be specific against HPV45 because HPV45 was not part of the specificity design. Accordingly, as shown in Table 18, synRNA for HPV 18 showed cross-reactivity against HPV 45 plasmid only starting at between 10⁶ and 10⁷ copies of plasmid. synRNA=3.7 kb coverage of HPV 18 DNA; 25 mers@1.34 nM final in hybridization.

TABLE 18
Limited Cross-reactivity of HPV18 synRNA with HPV45

| | Input Copies | | Avg RLU | S − N | (S/N) − 1 |
|---|---|---|---|---|---|
| HPV18 | 0 | c | 44 | 0 | 0.0 |
| 3.7 kb RNA | | | | | |
| | 2500 | c | 105 | 61 | 1.4 |
| | 5000 | c | 111 | 67 | 1.5 |
| | 10\hat{ }4 | c | 184 | 140 | 3.2 |
| HPV45 | 0 | c | 39 | 0 | 0.0 |
| 3.7 kb RNA | | | | | |
| | 10\hat{ }5 | c | 51 | 12 | 0.3 |
| | 10\hat{ }6 | c | 70 | 31 | 0.8 |
| | 10\hat{ }7 | c | 334 | 296 | 7.7 |

C. Determining Specificity with HPV16 synRNA

As shown in Table 19, HPV16 synRNA is unable to detect HPVs 6, 18, or 45 at up to 10⁹ copies/assay (200 ng/ml). synRNA=3.175 kb coverage of HPV 16 DNA; 25 mers@1.34 nM final in hybridization.

TABLE 19
Specificity of HPV16 synRNA

| | Input Copies | | Avg RLU | (S/N) − 1 | % CV |
|---|---|---|---|---|---|
| HPV 16 | 0 | c | 24 | 0.0 | 5% |
| | 5000 | c | 85 | 2.5 | 3% |
| | 10\hat{ }4 | c | 157 | 5.5 | 3% |
| | 10\hat{ }5 | c | 1270 | 51.4 | 2% |
| HPV 18 | 0 | c | 24 | 0.0 | 0% |
| | 10\hat{ }7 | c | 25 | 0.0 | 7% |
| | 10\hat{ }8 | c | 24 | 0.0 | 2% |
| | 10\hat{ }9 | c | 25 | 0.0 | 5% |
| HPV 45 | 0 | c | 25 | 0.0 | 6% |
| | 10\hat{ }7 | c | 26 | 0.0 | 5% |
| | 10\hat{ }8 | c | 28 | 0.1 | 17% |
| | 10\hat{ }9 | c | 38 | 0.5 | 3% |
| HPV 6 | 0 | c | 29 | 0.0 | 33% |
| | 10\hat{ }7 | c | 24 | −0.2 | 2% |
| | 10\hat{ }8 | c | 26 | −0.1 | 2% |
| | 10\hat{ }9 | c | 24 | −0.2 | 5% |

D. Deterring Different HPV Types

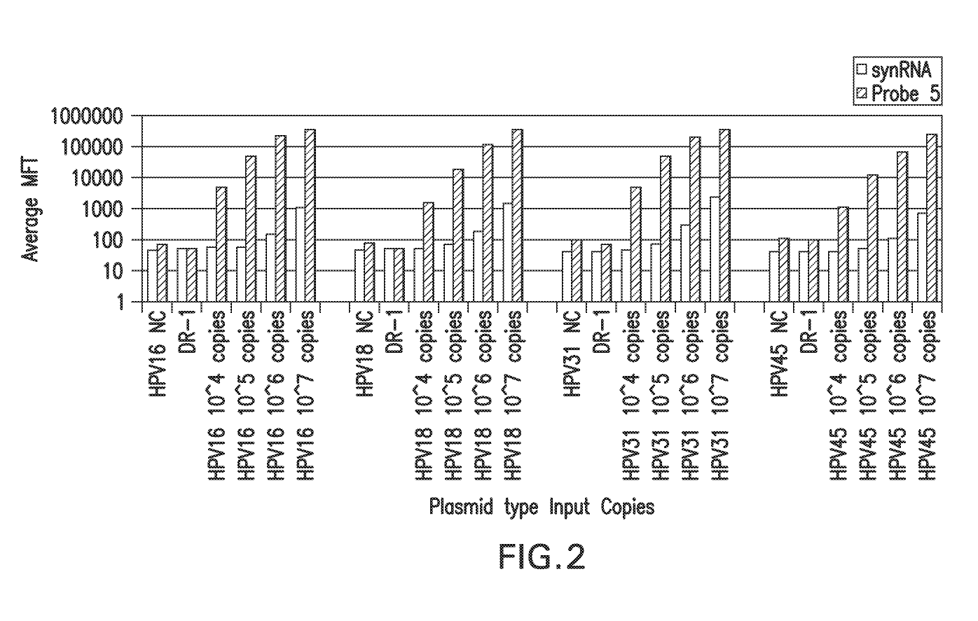

About 0.5 kb coverage of specific 25mer probes was provided for HPVs 16, 18, 31, and 45. As shown in FIG. 2, each HPV type was detected at 10⁶ copies. synRNA probes should be equally applicable to detection of whichever HPV types are desired.

E. Effect of SynRNA Coverage on Sensitivity of Detection

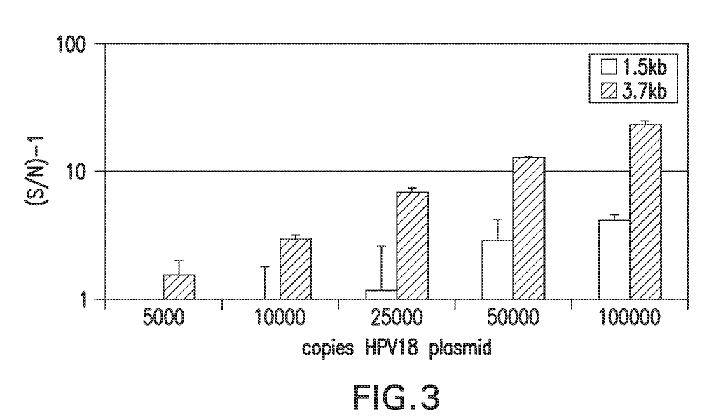

Total coverage of synRNA probe affected signal in the assay. Increasing coverage improved signal in a non-linear fashion, probably due to base-stacking effects and loosening of secondary structure on the single-stranded DNA target as more synRNA probes are hybridized. As shown in FIG. 3, at 3.7 kb of coverage, the sensitivity of detection was at 5,000 copies/assay.

F. Effect of SynRNA Concentration on Sensitivity of Detection

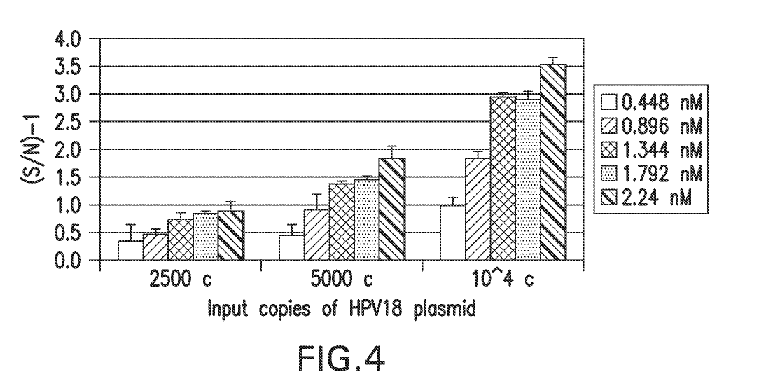

As shown in FIG. 4, increasing the concentration of synRNA increased sensitivity of detection. 25mer synRNA oligos had Tms about 45 to about 60° C. Increasing probe concentration raised that Tm, resulting in more efficient hybridization. synRNA=3.7 kb coverage; 25mers@concentrations shown in FIG. 4.

G. Effect of SynRNA Size on Sensitivity of Detection

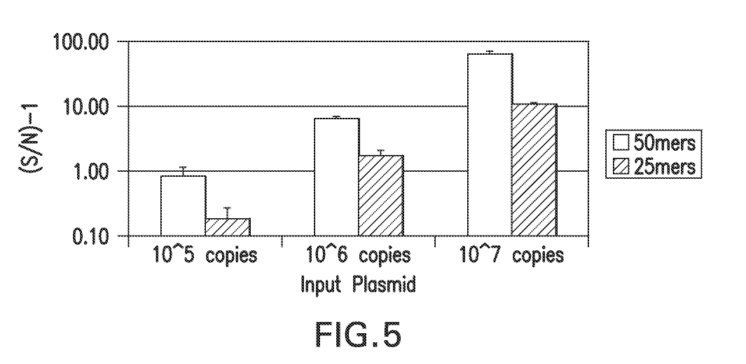

As shown in FIG. 5, given equivalent coverage, longer synRNA provided increased sensitivity.

H. Effect of SynRNA Contiguity on Sensitivity of Detection

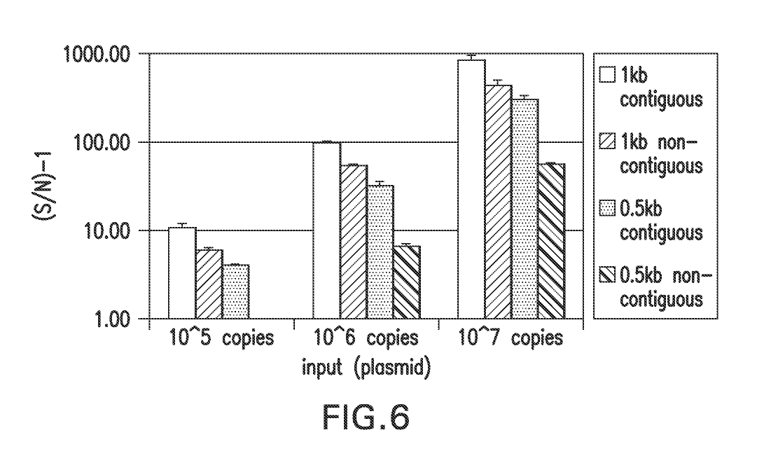

As shown in FIG. 6, sensitivity increased as synRNA probes targeted adjacent regions. Without being held to a particular theory, it is believed that hybridization efficiency improved as the binding of one probe relaxed secondary structure on the target strand, providing a more accessible template for hybridization of the adjacent synRNA.

I. HPV 16 and HPV 18 are Detected at Equivalent Levels

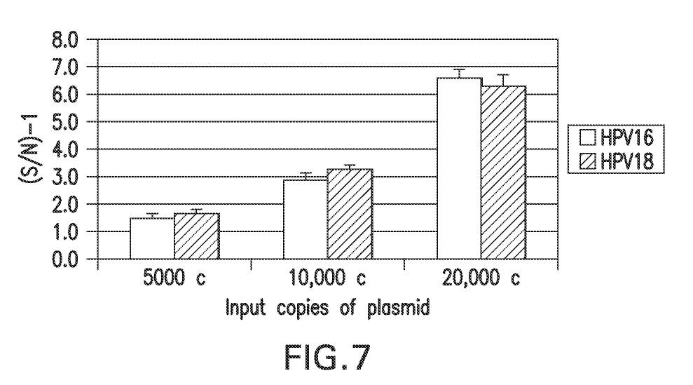

As shown in FIG. 7, HPV16 synRNA, with about 3.175 kb coverage, and HPV18, with about 3.7 kb coverage, gave about similar results. Both synRNAs were able to detect their respective targets at a concentration of 5,000 copies.

J. Comparison of Different SynRNA Synthesis Chemistries

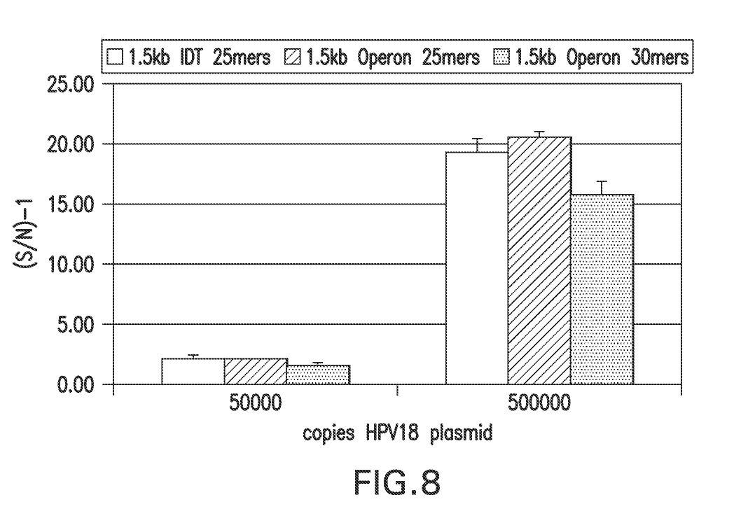

SynRNAs were prepared by TOM amidite chemistry (Operon Biotechnologies, Inc., Huntsville, Ala.) or by tBDMS chemistry (Integrated DNA Technologies (IDT)). As shown in FIG. 8, 25mers of comparable quality can be provided using different chemical synthesis methods.

K. Detection at Different Temperatures

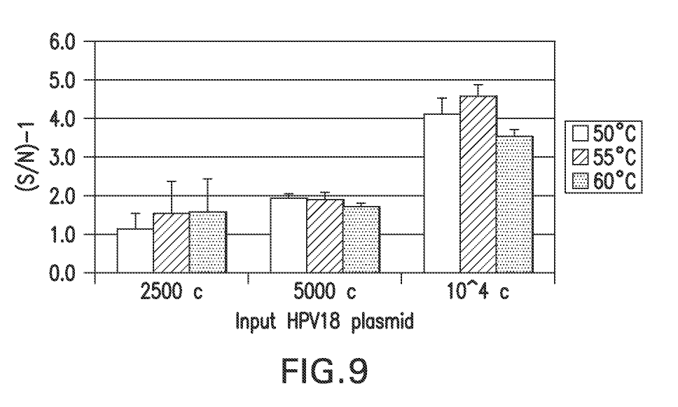

With no RNA-dependant background occurring from synRNA, the hybridization temperature can be reduced, if desired, to provide a more tolerable condition for antibody/antigen interactions (FIG. 9).

L. Exogenous Rnase is Unnecessary for Detection

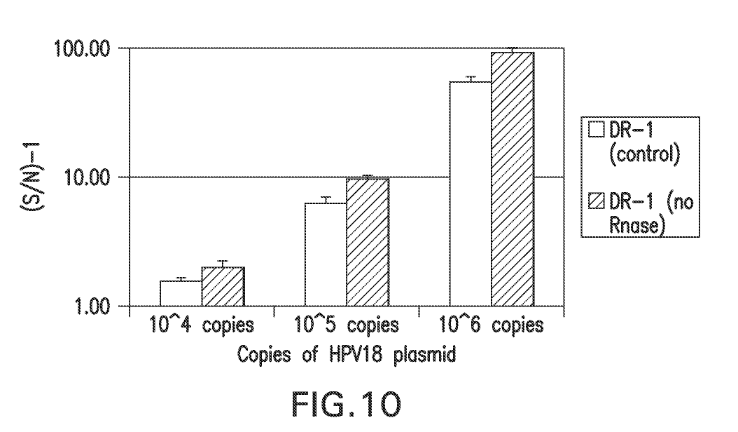

synRNAs are largely devoid of secondary structure. This eliminates non-specific RNA-based background arising from anti-RNA:DNA hybrid antibodies recognizing long RNA secondary structures. With RNA not bound to DNA no longer contributing to background signal, the use of RNase A in the assay becomes unnecessary (FIG. 10).

M. Discussion

The method provided specificity and decreased background, and does not require RNase and is compatible with various media including SurePath, PC, STM and DCM.

The method provided a LOD with a 0.5 kb target coverage is of 5 pg/mL for HPV18 with an S/N=3, whereas 2.5 kb target coverage could allow target detection to 1 pg/mL.

### Example 4

### Target Capture and Amplification

The inclusion of a target amplification component provided enhanced sensitivity. The method detected as low as 10 copies of HPV plasmids or 10 SiHa cells comprising HPV nucleic acid target. The method also provided robust specificity, the ability to distinguish HPV 16 or HPV18 plasmid from all other high- and low-risk HPV types.

Target amplification can involve e.g. generating short amplicons with sequence-specific primers (e.g. Polymerase Chain Reaction) or large amplicons with multiple random primers (e.g. Whole Genome Amplification). Amplified targets can be captured and detected on a variety of different detection platforms.

Hybrid-specific antibodies were coupled to magnetic beads and employed in combination with short type-specific RNA probes for target capture. The sample processing procedure involved capture of targets pre-target amplification and the detection procedure involves capture of targets post-target amplification. To enhance assay sensitivity the isothermal WGA technology was utilized to produce non-specific amplification of any captured targets.

The nucleic acid target of interest was immobilized on a solid support with the use of type-specific RNA probes to form nucleic acid hybrids and anti-RNA:DNA hybrid-specific antibodies to capture, concentrate and purify. The sample preparation process produced single-stranded DNA targets free of amplification inhibitors and non-specific targets and allowed for multiple targets to be captured simultaneously. This was demonstrated by coupling hybrid capture antibodies to magnetic beads and using HPV sequence-specific RNA probes for detection.

Magnetic beads coupled with anti-hybrid antibodies were used to specifically capture amplicons generated by WGA. Short RNA probes were used for specific detection. In addition, anti-RNA:DNA hybrid antibodies coupled with alkaline phosphatase was used for detection.

Table 20 shows a flowchart representing a method steps in accordance with one embodiment. Detection reagent 1 is preferably the detection reagent 1 provided in the digene Hybrid Capture Kit and detection reagent 2 is preferably the detection reagent 2 provided in the digene Hybrid Capture Kit. Detection Reagent 1 comprises alkaline phosphatase-conjugated antibodies to RNA:DNA hybrids and Detection Reagent 2 comprises CDP-Star® with Emerald II (chemiluminescent substrate).

TABLE 20
Protocol.

Assay Flow Chart
Target Denaturation
RNA probe hybridization and capture with anti-hybrid antibody
Wash
Isothermal Amplification
Amplicon Denaturation
RNA probe hybridization and capture with anti-hybrid antibody
Detection Reagent 1
Wash
Detection Reagent 2

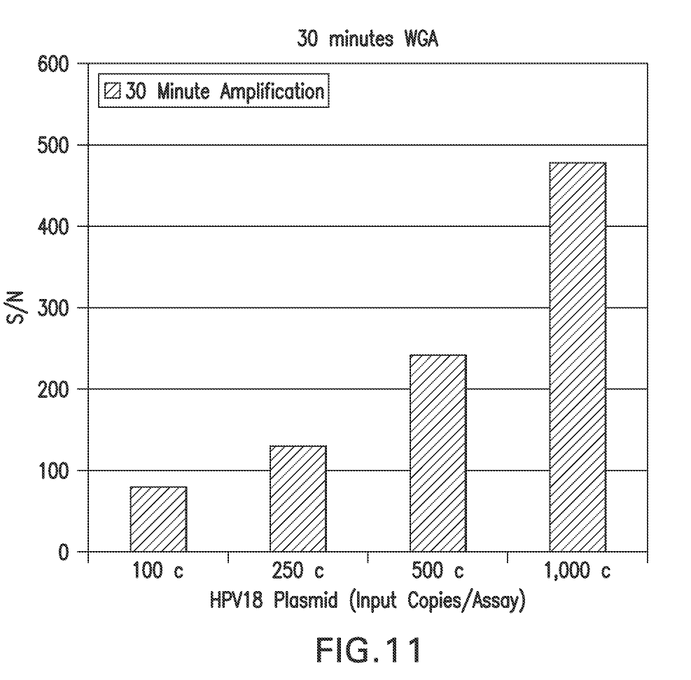

One hundred (100) copies of HPV18 plasmid are obtained after 30 minutes of WGA (FIG. 11)

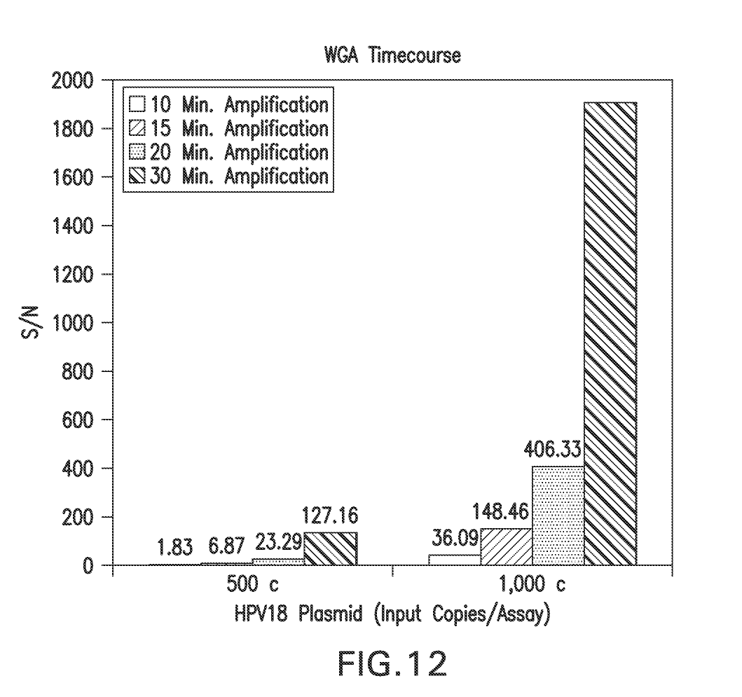

Five hundred (500) copies of HPV18 plasmid are detected after 15 minutes of WGA; and detection of 1000 copies of HPV18 plasmid are obtained after only 10 minutes of WGA (FIG. 12).

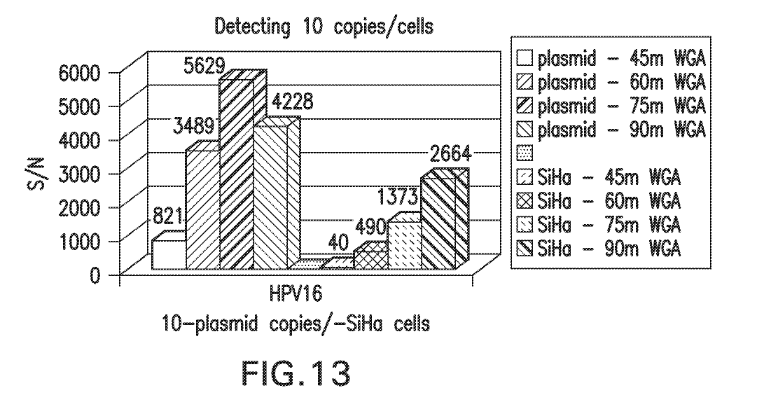

Ten (10) copies of plasmid or 10 SiHa cells comprising HPV nucleic acid are detected with longer amplification times of 45 minutes or greater (FIG. 13).

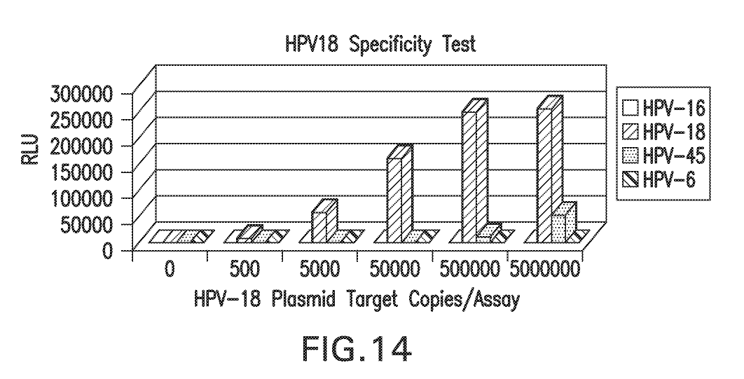

FIG. 14 shows specificity for HPV18.

The results demonstrated that after 45 minutes of amplification, as little as 10 copies of plasmid or 10 SiHa cells can be detected; and about 1000 copies of plasmid can be detected after only 10 minutes of amplification.

### Example 5

### Synthetic Type-Specific Biotinylated DNA Probes DNA Probes

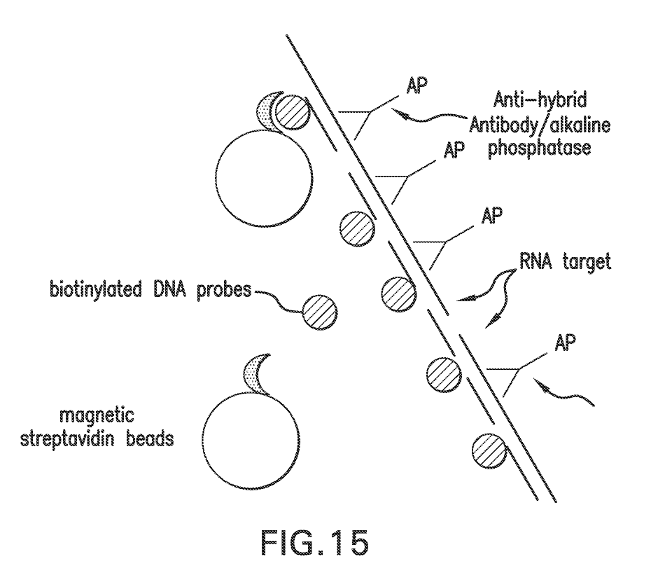

In another embodiment, synthetic type-specific biotinylated DNA probes are used to form double-stranded hybrids with target mRNA (FIG. 15). Hybrids are captured on magnetic streptavidin beads. Signal amplification and detection is performed with anti-hybrid antibody/alkaline phosphatase and the resulting chemiluminescent signal is detected.

### Example 6

### Sample Assay Flow

Predenatured samples are transferred to a multiwell plate. Probes in neutralizing solution are added to the denatured sample and incubated with shaking at room temperature for about 1 minute to neutralize the sample. The neutralized samples are transferred to a plate containing immobilized anti-RNA:DNA hybrid antibodies so that target DNA is allowed to hybridize to the synthetic RNA probes and also to be captured by the immobilized antibodies. The incubation is at about 55° C. for about 120 min. Anti-RNA:DNA hybrid antibodies conjugated with alkaline phosphatase are added at room temp and incubated for about 30 min. After the conjugated antibody step, the plate is washed for about 12 min. A dioxetane substrate is added and incubated for 15 minutes. The plate is then read with a luminometer.

Hybridization and hybrid capture by anti-RNA:DNA hybrid antibodies are performed in the same step at about 55° C. and may include shaking.

1. A method for determining the presence of a target nucleic acid in a sample, the method comprising:
a) contacting one or more polynucleotide probes with the sample under a hybridization condition sufficient for the one or more polynucleotide probes to hybridize to the target nucleic acid in the sample to form double-stranded nucleic acid hybrids, wherein the one or more polynucleotide probes does not hybridize to a variant of the target nucleic acid; and
b) detecting the double-stranded nucleic acid hybrids, wherein detecting comprises contacting the double-stranded nucleic acid hybrids with a first anti-hybrid antibody that is immunospecific to double-stranded nucleic acid hybrids, whereby detection of the double-stranded nucleic acid hybrids determines the target nucleic acid in the sample.

2. The method of claim 1 wherein the detecting further comprises providing a second anti-hybrid antibody that is immunospecific to double-stranded nucleic acid hybrids, wherein the second anti-hybrid antibody is detectably labeled.

3. The method of claim 1 wherein the at least one probe and the anti-hybrid antibody are added in the same step.

4. The method of claim 1, wherein the target nucleic acid is an HPV nucleic acid.

5. The method of claim 4, wherein the HPV nucleic acid is HPV DNA of a high risk HPV type.

6. The method of claim 5, wherein the HPV type is HPV 16, wherein the variant is a nucleic acid of a type selected from the group consisting of: HPV 1, 2, 3, 4, 5, 6, 8, 11, 13, 18, 26, 30, 31, 33, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

7. The method of claim 5, wherein the HPV type is HPV 18, wherein the variant is nucleic acid of a type selected from the group consisting of: HPV 1, 2, 3, 4, 5, 6, 8, 11, 13, 16, 26, 30, 31, 33, 34, 35, 39, 40, 42, 43, 44, 45, 51, 52, 53, 54, 56, 58, 59, 61, 62, 66, 67, 68, 69, 70, 71, 72, 73, 74, 81, 82, 83, 84, and 89.

8. The method of claim 5, wherein the HPV type is HPV 45, wherein the variant is nucleic acid of a type selected from the group consisting of: HPV 1, 2, 3, 4, 5, 6, 8, 11, 13, 16, 18, 26, 30, 31, 33, 34, 35, 39, 40, 42, 43, 44, 51, 52, 53, 54, 56, 58, 59, 61, 62, 66, 67, 68, 69, 70, 71, 72, 73, 74, 81, 82, 83, 84, and 89.

9. The method of claim 5 wherein the HPV type is a hrHPV type and wherein the variant is a nucleic acid of low risk HPV type.

10. The method of claim 5, wherein the one or more polynucleotide probes consist essentially of a sequence or a complement thereof selected from the group consisting of SEQ ID NOs: 1-2026.

11. A method for determining the presence of HPV 18 DNA in a sample, the method comprising:
a) contacting one or more polynucleotide probes or a complement thereof with the sample under a hybridization condition sufficient to allow the one or polynucleotides to anneal to a corresponding complementary nucleic acid sequence in the sample to form double-stranded nucleic acid hybrids, wherein the one or more polynucleotide probes is a set of nucleic acid probes comprising at least one nucleic acid sequence chosen from the group consisting of: SEQ ID NOs:163-309; and
b) detecting double-stranded nucleic acid hybrids, whereby detection of the double-stranded nucleic acid hybrids indicates the presence of HPV 18 DNA in the sample.

12. A method for determining the presence of HPV 16 DNA in a sample, the method comprising:
a) contacting one or more polynucleotide probes or a complement thereof with the sample under a hybridization condition sufficient to allow the one or polynucleotides to anneal to a corresponding complementary nucleic acid sequence in the sample to form double-stranded nucleic acid hybrids, wherein the one or more polynucleotide probes is a set of nucleic acid probes comprising at least one nucleic acid sequence chosen from the group consisting of: SEQ ID NOs:1-162; and
b) detecting double-stranded nucleic acid hybrids, whereby detection of the double-stranded nucleic acid hybrids indicates the presence of HPV 16 DNA in the sample.

13. A method for determining the presence of HPV 45 DNA in a sample, the method comprising:
a) contacting one or more polynucleotide probes or a complement thereof with the sample under a hybridization condition sufficient to allow the one or polynucleotides to anneal to a corresponding complementary nucleic acid sequence in the sample to form double-stranded nucleic acid hybrids, wherein the one or more polynucleotide probes is a set of nucleic acid probes comprising at least one nucleic acid sequence chosen from the group consisting of: SEQ ID NOs:842-974; and
b) detecting double-stranded nucleic acid hybrids, whereby detection of the double-stranded nucleic acid hybrids indicates the presence of HPV 45 DNA in the sample.

14. A probe set selected from the group consisting of SEQ ID NO; 1-162 (HPV 16); 163-309(HPV 18); 842-974(HPV 45); 310-454(HPV 31); 455-579(HPV 33); 580-722(HPV 35); 723-841(HPV 39); 975-1120(HPV 51); 1121-1252(HPV 52); 1253-1367(HPV 56); 1368-1497(HPV 58); 1498-1646(HPV 59); 1647-1767(HPV 66); 1768-1875(HPV 68); and 1876-2026(HPV 82).

15. The method of claim 1 wherein the one or more polynucleotide probes is a mixture of probe sets comprising the probes set forth in SEQ ID NO: 1-2026.

16. The method of claim 1 wherein the hybridization is performed at about 45 to about 55° C.

17. A kit comprising the probe set of claim 14.


SEQUENCE LISTING (2026 sequences)

SEQ ID NO: 1 - RNA, 25 nt, Artificial Sequence
  Description of Artificial Sequence Synthetic probe
ggguuacacauuuacaagcaacuua

SEQ ID NO: 2 - RNA, 25 nt, Artificial Sequence
  Description of Artificial Sequence Synthetic probe
acaugggugugugcaaaccgauuuu

SEQ ID NO: 3 - RNA, 25 nt, Artificial Sequence
  Description of Artificial Sequence Synthetic probe
cuguguaaagguuagucauacauug

SEQ ID NO: 4 - RNA, 25 nt, Artificial Sequence
  Description of Artificial Sequence Synthetic probe
aaugucacccuaguucauacaugaa

SEQ ID NO: 5 - RNA, 25 nt, Artificial Sequence
  Description of Artificial Sequence Synthetic probe
agguuuaaacuucuaaggccaacua

SEQ ID NO: 6 - RNA, 25 nt, Artificial Sequence
  Description of Artificial Sequence Synthetic probe
ggcuuguuuuaacuaaccuaauugc

SEQ ID NO: 7 - RNA, 25 nt, Artificial Sequence
  Description of Artificial Sequence Synthetic probe
caacgccuuacauaccgcuguuagg

SEQ ID NO: 8 - RNA, 25 nt, Artificial Sequence
  Description of Artificial Sequence Synthetic probe
cugaaucacuauguacauuguguca

SEQ ID NO: 9 - RNA, 25 nt, Artificial Sequence
  Description of Artificial Sequence Synthetic probe
gcacugcuugccaaccauuccauug

SEQ ID NO: 10 - RNA, 25 nt, Artificial Sequence
  Description of Artificial Sequence Synthetic probe
ugccaaaucccuguuuuccugaccu

SEQ ID NO: 11 - RNA, 25 nt, Artificial Sequence
  Description of Artificial Sequence Synthetic probe
uuguacguuuccugcuugccaugcg

SEQ ID NO: 12 - RNA, 25 nt, Artificial Sequence
  Description of Artificial Sequence Synthetic probe
cuaugucagcaacuaugguuuaaac

SEQ ID NO: 13 - RNA, 25 nt, Artificial Sequence
  Description of Artificial Sequence Synthetic probe
cccauuuuguagcuucaaccgaauu

SEQ ID NO: 14 - RNA, 25 nt, Artificial Sequence
  Description of Artificial Sequence Synthetic probe
auauacuauauuuuguagcgccagg

SEQ ID NO: 15 - RNA, 25 nt, Artificial Sequence
  Description of Artificial Sequence Synthetic probe
uauaaacuauauuugcuacauccug

SEQ ID NO: 16 - RNA, 25 nt, Artificial Sequence
  Description of Artificial Sequence Synthetic probe
ccuacuaauuguguugugguuauuc

SEQ ID NO: 17 - RNA, 25 nt, Artificial Sequence
  Description of Artificial Sequence Synthetic probe
guguaacuauugugucaugcaacau

SEQ ID NO: 18 - RNA, 25 nt, Artificial Sequence
  Description of Artificial Sequence Synthetic probe
uguaugguauaauaaacacgugugu

SEQ ID NO: 19 - RNA, 25 nt, Artificial Sequence
  Description of Artificial Sequence Synthetic probe
auauuaaguuguauguguguuugua

SEQ ID NO: 20 - RNA, 25 nt, Artificial Sequence
  Description of Artificial Sequence Synthetic probe
guaugugcuuguaugugcuuguaaa

SEQ ID NO: 21 - RNA, 25 nt, Artificial Sequence
  Description of Artificial Sequence Synthetic probe
uaguguuguuuguuguguauauguu

SEQ ID NO: 22 - RNA, 25 nt, Artificial Sequence
  Description of Artificial Sequence Synthetic probe
uguaaguauuguauguauguugaau

SEQ ID NO: 23 - RNA, 25 nt, Artificial Sequence
  Description of Artificial Sequence Synthetic probe
aucuaccucuacaacugcuaaacgc

SEQ ID NO: 24 - RNA, 25 nt, Artificial Sequence
  Description of Artificial Sequence Synthetic probe
aacgaaaagcuacacccaccaccuc

SEQ ID NO: 25 - RNA, 25 nt, Artificial Sequence
  Description of Artificial Sequence Synthetic probe
ggccaaaccaaaauuuacauuagga

SEQ ID NO: 26 - RNA, 25 nt, Artificial Sequence
  Description of Artificial Sequence Synthetic probe
agcaccuaaagaagaugauccccuu

SEQ ID NO: 27 - RNA, 25 nt, Artificial Sequence
  Description of Artificial Sequence Synthetic probe
uuuguaacccaggcaauugcuuguc

SEQ ID NO: 28 - RNA, 25 nt, Artificial Sequence
  Description of Artificial Sequence Synthetic probe
aggcacacuagaagauacuuauagg

SEQ ID NO: 29 - RNA, 25 nt, Artificial Sequence
  Description of Artificial Sequence Synthetic probe
cagacguuaugacauacauacauuc

SEQ ID NO: 30 - RNA, 25 nt, Artificial Sequence
  Description of Artificial Sequence Synthetic probe
gccauaucuacuucagaaacuacau

SEQ ID NO: 31 - RNA, 25 nt, Artificial Sequence
  Description of Artificial Sequence Synthetic probe
cugaugcccaaauauucaauaaacc

SEQ ID NO: 32 - RNA, 25 nt, Artificial Sequence
  Description of Artificial Sequence Synthetic probe
ccaguucaaauuauuuuccuacacc

SEQ ID NO: 33 - RNA, 25 nt, Artificial Sequence
  Description of Artificial Sequence Synthetic probe
ggcucugggucuacugcaaauuuag

SEQ ID NO: 34 - RNA, 25 nt, Artificial Sequence
  Description of Artificial Sequence Synthetic probe
ggugaaaauguaccagacgauuuau

SEQ ID NO: 35 - RNA, 25 nt, Artificial Sequence
  Description of Artificial Sequence Synthetic probe
gucagaaccauauggcgacagcuua

SEQ ID NO: 36 - RNA, 25 nt, Artificial Sequence
  Description of Artificial Sequence Synthetic probe
guuccacuggauauuuguacaucua

SEQ ID NO: 37 - RNA, 25 nt, Artificial Sequence
  Description of Artificial Sequence Synthetic probe
ccaccauuagaguuaauaaacacag

SEQ ID NO: 38 - RNA, 25 nt, Artificial Sequence
  Description of Artificial Sequence Synthetic probe
aauguugcaguaaauccaggugauu

SEQ ID NO: 39 - RNA, 25 nt, Artificial Sequence
  Description of Artificial Sequence Synthetic probe
cagguguggauaauagagaauguau

SEQ ID NO: 40 - RNA, 25 nt, Artificial Sequence
  Description of Artificial Sequence Synthetic probe
cagaaaaugcuagugcuuaugcagc

SEQ ID NO: 41 - RNA, 25 nt, Artificial Sequence
  Description of Artificial Sequence Synthetic probe
uauuuagaauacauuuaccugaccc

SEQ ID NO: 42 - RNA, 25 nt, Artificial Sequence
  Description of Artificial Sequence Synthetic probe
uaaaguaucaggauuacaauacagg

SEQ ID NO: 43 - RNA, 25 nt, Artificial Sequence
  Description of Artificial Sequence Synthetic probe
cuaacaauaacaaaauauuaguucc

SEQ ID NO: 44 - RNA, 25 nt, Artificial Sequence
  Description of Artificial Sequence Synthetic probe
gcaggaacauccagacuacuugcag

SEQ ID NO: 45 - RNA, 25 nt, Artificial Sequence
  Description of Artificial Sequence Synthetic probe
guuauuacauguuacgaaaacgacg

SEQ ID NO: 46 - RNA, 25 nt, Artificial Sequence
  Description of Artificial Sequence Synthetic probe
acaauuauugcugaugcaggugacu

SEQ ID NO: 47 - RNA, 25 nt, Artificial Sequence
  Description of Artificial Sequence Synthetic probe
uauaguuccagggucuccacaauau

SEQ ID NO: 48 - RNA, 25 nt, Artificial Sequence
  Description of Artificial Sequence Synthetic probe
cugaccaagcuccuucauuaauucc

SEQ ID NO: 49 - RNA, 25 nt, Artificial Sequence
  Description of Artificial Sequence Synthetic probe
cagguccugauauacccauuaauau

SEQ ID NO: 50 - RNA, 25 nt, Artificial Sequence
  Description of Artificial Sequence Synthetic probe
guggugcauacaauauuccuuuagu

SEQ ID NO: 51 - RNA, 25 nt, Artificial Sequence
  Description of Artificial Sequence Synthetic probe
cagguuauauuccugcaaauacaac

SEQ ID NO: 52 - RNA, 25 nt, Artificial Sequence
  Description of Artificial Sequence Synthetic probe
ccaucuguacccucuacaucuuuau

SEQ ID NO: 53 - RNA, 25 nt, Artificial Sequence
  Description of Artificial Sequence Synthetic probe
uacagauacuucuacaaccccggua

SEQ ID NO: 54 - RNA, 25 nt, Artificial Sequence
  Description of Artificial Sequence Synthetic probe
auuuaugcagaugacuuuauuacag

SEQ ID NO: 55 - RNA, 25 nt, Artificial Sequence
  Description of Artificial Sequence Synthetic probe
ccucaccuacuucuauuaauaaugg

SEQ ID NO: 56 - RNA, 25 nt, Artificial Sequence
  Description of Artificial Sequence Synthetic probe
acauauacuaccacuucacaugcag

SEQ ID NO: 57 - RNA, 25 nt, Artificial Sequence
  Description of Artificial Sequence Synthetic probe
acuauugauccugcagaagaaauag

SEQ ID NO: 58 - RNA, 25 nt, Artificial Sequence
  Description of Artificial Sequence Synthetic probe
uggaaaaucuauaggugcuaaggua

SEQ ID NO: 59 - RNA, 25 nt, Artificial Sequence
  Description of Artificial Sequence Synthetic probe
gguaauaaacaaacacuacguacuc

SEQ ID NO: 60 - RNA, 25 nt, Artificial Sequence
  Description of Artificial Sequence Synthetic probe
uaggcguacuggcauuagguacagu

SEQ ID NO: 61 - RNA, 25 nt, Artificial Sequence
  Description of Artificial Sequence Synthetic probe
aauaguauuaauauagcuccagauc

SEQ ID NO: 62 - RNA, 25 nt, Artificial Sequence
  Description of Artificial Sequence Synthetic probe
gcauaugaagguauagauguggaua

SEQ ID NO: 63 - RNA, 25 nt, Artificial Sequence
  Description of Artificial Sequence Synthetic probe
ccacucccacuaaacuuauuacaua

SEQ ID NO: 64 - RNA, 25 nt, Artificial Sequence
  Description of Artificial Sequence Synthetic probe
ggauuauauagucgcacaacacaac

SEQ ID NO: 65 - RNA, 25 nt, Artificial Sequence
  Description of Artificial Sequence Synthetic probe
cuaacacaguaacuaguagcacacc

SEQ ID NO: 66 - RNA, 25 nt, Artificial Sequence
  Description of Artificial Sequence Synthetic probe
gauacauuuauuguuagcacaaacc

SEQ ID NO: 67 - RNA, 25 nt, Artificial Sequence
  Description of Artificial Sequence Synthetic probe
gcauuuuacacuuucaucauccacu

SEQ ID NO: 68 - RNA, 25 nt, Artificial Sequence
  Description of Artificial Sequence Synthetic probe
cauaauaaucccacuuucacugacc

SEQ ID NO: 69 - RNA, 25 nt, Artificial Sequence
  Description of Artificial Sequence Synthetic probe
uaauacuguuacuacuguuacuaca

SEQ ID NO: 70 - RNA, 25 nt, Artificial Sequence
  Description of Artificial Sequence Synthetic probe
acuacuucaacugauaccacaccug

SEQ ID NO: 71 - RNA, 25 nt, Artificial Sequence
  Description of Artificial Sequence Synthetic probe
ugcaccaacaucuguaccuuccauu

SEQ ID NO: 72 - RNA, 25 nt, Artificial Sequence
  Description of Artificial Sequence Synthetic probe
gaagaaacuaguuuuauugaugcug

SEQ ID NO: 73 - RNA, 25 nt, Artificial Sequence
  Description of Artificial Sequence Synthetic probe
uacagauacacuugcuccuguaaga

SEQ ID NO: 74 - RNA, 25 nt, Artificial Sequence
  Description of Artificial Sequence Synthetic probe
cggacgcacuggguauauuccauug

SEQ ID NO: 75 - RNA, 25 nt, Artificial Sequence
  Description of Artificial Sequence Synthetic probe
auuacaauauggaaguaugggugua

SEQ ID NO: 76 - RNA, 25 nt, Artificial Sequence
  Description of Artificial Sequence Synthetic probe
cggcuacccaacuuuauaaaacaug

SEQ ID NO: 77 - RNA, 25 nt, Artificial Sequence
  Description of Artificial Sequence Synthetic probe
acaaugcgacacaaacguucugcaa

SEQ ID NO: 78 - RNA, 25 nt, Artificial Sequence
  Description of Artificial Sequence Synthetic probe
aauuguuguauaccauaacuuacua

SEQ ID NO: 79 - RNA, 25 nt, Artificial Sequence
  Description of Artificial Sequence Synthetic probe
auauguacauaauguaauuguuaca

SEQ ID NO: 80 - RNA, 25 nt, Artificial Sequence
  Description of Artificial Sequence Synthetic probe
cucugcguuuagguguuuuauugua

SEQ ID NO: 81 - RNA, 25 nt, Artificial Sequence
  Description of Artificial Sequence Synthetic probe
uauuacuauuguggauaacagcagc

SEQ ID NO: 82 - RNA, 25 nt, Artificial Sequence
  Description of Artificial Sequence Synthetic probe
ugcuuuugucugugucuacauacac

SEQ ID NO: 83 - RNA, 25 nt, Artificial Sequence
  Description of Artificial Sequence Synthetic probe
ugcauccacaacauuacuggcgugc

SEQ ID NO: 84 - RNA, 25 nt, Artificial Sequence
  Description of Artificial Sequence Synthetic probe
cagugucuacuggauuuaugucuau

SEQ ID NO: 85 - RNA, 25 nt, Artificial Sequence
  Description of Artificial Sequence Synthetic probe
ugauagugaauggcaacgugaccaa

SEQ ID NO: 86 - RNA, 25 nt, Artificial Sequence
  Description of Artificial Sequence Synthetic probe
cauuggacaggacauaauguaaaac

SEQ ID NO: 87 - RNA, 25 nt, Artificial Sequence
  Description of Artificial Sequence Synthetic probe
uguauacugcagugucgucuacaug

SEQ ID NO: 88 - RNA, 25 nt, Artificial Sequence
  Description of Artificial Sequence Synthetic probe
cuaauacuuuaaaauguuuaagaua

SEQ ID NO: 89 - RNA, 25 nt, Artificial Sequence
  Description of Artificial Sequence Synthetic probe
guaacacuacacccauaguacauuu

SEQ ID NO: 90 - RNA, 25 nt, Artificial Sequence
  Description of Artificial Sequence Synthetic probe
cacaaaggacggauuaacuguaaua

SEQ ID NO: 91 - RNA, 25 nt, Artificial Sequence
  Description of Artificial Sequence Synthetic probe
aauccucacugcauuuaacagcuca

SEQ ID NO: 92 - RNA, 25 nt, Artificial Sequence
  Description of Artificial Sequence Synthetic probe
uuguugcacagagacucaguggaca

SEQ ID NO: 93 - RNA, 25 nt, Artificial Sequence
  Description of Artificial Sequence Synthetic probe
cggaaaccccugccacaccacuaag

SEQ ID NO: 94 - RNA, 25 nt, Artificial Sequence
  Description of Artificial Sequence Synthetic probe
acgacuauccagcgaccaagaucag

SEQ ID NO: 95 - RNA, 25 nt, Artificial Sequence
  Description of Artificial Sequence Synthetic probe
gacccauaccaaagccgucgccuug

SEQ ID NO: 96 - RNA, 25 nt, Artificial Sequence
  Description of Artificial Sequence Synthetic probe
ugaaauuauuaggcagcacuuggcc

SEQ ID NO: 97 - RNA, 25 nt, Artificial Sequence
  Description of Artificial Sequence Synthetic probe
gucagguaauauuauguccuacauc

SEQ ID NO: 98 - RNA, 25 nt, Artificial Sequence
  Description of Artificial Sequence Synthetic probe
ggaauacgaacauauuuugugcagu

SEQ ID NO: 99 - RNA, 25 nt, Artificial Sequence
  Description of Artificial Sequence Synthetic probe
gggucaaguugacuauuaugguuua

SEQ ID NO: 100 - RNA, 25 nt, Artificial Sequence
  Description of Artificial Sequence Synthetic probe
aagaagcaucaguaacugugguaga

SEQ ID NO: 101 - RNA, 25 nt, Artificial Sequence
  Description of Artificial Sequence Synthetic probe
uauacaaacuggacacauauauaua

SEQ ID NO: 102 - RNA, 25 nt, Artificial Sequence
  Description of Artificial Sequence Synthetic probe
guggaagugcaguuugauggagaca

SEQ ID NO: 103 - RNA, 25 nt, Artificial Sequence
  Description of Artificial Sequence Synthetic probe
guuagccuugaaguguauuuaacug

SEQ ID NO: 104 - RNA, 25 nt, Artificial Sequence
  Description of Artificial Sequence Synthetic probe
uaaugaaaaguggacauuacaagac

SEQ ID NO: 105 - RNA, 25 nt, Artificial Sequence
  Description of Artificial Sequence Synthetic probe
gaacugcaacuaacguuagaaacaa

SEQ ID NO: 106 - RNA, 25 nt, Artificial Sequence
  Description of Artificial Sequence Synthetic probe
cuggcuguaucaaagaauaaagcau

SEQ ID NO: 107 - RNA, 25 nt, Artificial Sequence
  Description of Artificial Sequence Synthetic probe
gccagagaaaugggauuuaaacaua

SEQ ID NO: 108 - RNA, 25 nt, Artificial Sequence
  Description of Artificial Sequence Synthetic probe
cgccuagaaugugcuauuuauuaca

SEQ ID NO: 109 - RNA, 25 nt, Artificial Sequence
  Description of Artificial Sequence Synthetic probe
accuacgugaccauauagacuauug

SEQ ID NO: 110 - RNA, 25 nt, Artificial Sequence
  Description of Artificial Sequence Synthetic probe
aaaauacuaacacauuaugaaaaug

SEQ ID NO: 111 - RNA, 25 nt, Artificial Sequence
  Description of Artificial Sequence Synthetic probe
uaaugaguuuccauuugacgaaaac

SEQ ID NO: 112 - RNA, 25 nt, Artificial Sequence
  Description of Artificial Sequence Synthetic probe
auaauagauuggugguguuuacauu

SEQ ID NO: 113 - RNA, 25 nt, Artificial Sequence
  Description of Artificial Sequence Synthetic probe
uacaucuaacauuaaugcugguaca

SEQ ID NO: 114 - RNA, 25 nt, Artificial Sequence
  Description of Artificial Sequence Synthetic probe
uauggauguaaagcauagaccauug

SEQ ID NO: 115 - RNA, 25 nt, Artificial Sequence
  Description of Artificial Sequence Synthetic probe
cuguuggaacuacauagaugacaau

SEQ ID NO: 116 - RNA, 25 nt, Artificial Sequence
  Description of Artificial Sequence Synthetic probe
gcaagggucuguaauauguuuugua

SEQ ID NO: 117 - RNA, 25 nt, Artificial Sequence
  Description of Artificial Sequence Synthetic probe
uaugaguuuaaugaaauuucugcaa

SEQ ID NO: 118 - RNA, 25 nt, Artificial Sequence
  Description of Artificial Sequence Synthetic probe
auuacuauauggugcagcuaacaca

SEQ ID NO: 119 - RNA, 25 nt, Artificial Sequence
  Description of Artificial Sequence Synthetic probe
aggugauuggaagcaaauuguuaug

SEQ ID NO: 120 - RNA, 25 nt, Artificial Sequence
  Description of Artificial Sequence Synthetic probe
auaaaauauagaugugauaggguag

SEQ ID NO: 121 - RNA, 25 nt, Artificial Sequence
  Description of Artificial Sequence Synthetic probe
acgauaaugacauaguagacgauag

SEQ ID NO: 122 - RNA, 25 nt, Artificial Sequence
  Description of Artificial Sequence Synthetic probe
aaugauuguacauuugaauuaucac

SEQ ID NO: 123 - RNA, 25 nt, Artificial Sequence
  Description of Artificial Sequence Synthetic probe
uauaaaacagguauaucaaauauua

SEQ ID NO: 124 - RNA, 25 nt, Artificial Sequence
  Description of Artificial Sequence Synthetic probe
uaugaugauagagccuccaaaauug

SEQ ID NO: 125 - RNA, 25 nt, Artificial Sequence
  Description of Artificial Sequence Synthetic probe
aacuauuaugugugucuccaaugug

SEQ ID NO: 126 - RNA, 25 nt, Artificial Sequence
  Description of Artificial Sequence Synthetic probe
gggaaugguuguguuacuauuagua

SEQ ID NO: 127 - RNA, 25 nt, Artificial Sequence
  Description of Artificial Sequence Synthetic probe
uuuggacuuacacccaguauagcug

SEQ ID NO: 128 - RNA, 25 nt, Artificial Sequence
  Description of Artificial Sequence Synthetic probe
guguugcgauugguguauugcugca

SEQ ID NO: 129 - RNA, 25 nt, Artificial Sequence
  Description of Artificial Sequence Synthetic probe
gaccauuuaaaaguaauaaaucaac

SEQ ID NO: 130 - RNA, 25 nt, Artificial Sequence
  Description of Artificial Sequence Synthetic probe
aauuuaaagaguuauacggggugag

SEQ ID NO: 131 - RNA, 25 nt, Artificial Sequence
  Description of Artificial Sequence Synthetic probe
cuauaugccaaacaccacuuacaaa

SEQ ID NO: 132 - RNA, 25 nt, Artificial Sequence
  Description of Artificial Sequence Synthetic probe
uugcagucaguacaguaguggaagu

SEQ ID NO: 133 - RNA, 25 nt, Artificial Sequence
  Description of Artificial Sequence Synthetic probe
auguagucaguauagugguggaagu

SEQ ID NO: 134 - RNA, 25 nt, Artificial Sequence
  Description of Artificial Sequence Synthetic probe
aagggcgccaugagacugaaacacc

SEQ ID NO: 135 - RNA, 25 nt, Artificial Sequence
  Description of Artificial Sequence Synthetic probe
auuauuugaaagcgaagacagcggg

SEQ ID NO: 136 - RNA, 25 nt, Artificial Sequence
  Description of Artificial Sequence Synthetic probe
ccuagauuaaaagcuauauguauag

SEQ ID NO: 137 - RNA, 25 nt, Artificial Sequence
  Description of Artificial Sequence Synthetic probe
gugauauuaguggauguguagacaa

SEQ ID NO: 138 - RNA, 25 nt, Artificial Sequence
  Description of Artificial Sequence Synthetic probe
uagagaugcaguacagguucuaaaa

SEQ ID NO: 139 - RNA, 25 nt, Artificial Sequence
  Description of Artificial Sequence Synthetic probe
uuacugcacaggaagcaaaacaaca

SEQ ID NO: 140 - RNA, 25 nt, Artificial Sequence
  Description of Artificial Sequence Synthetic probe
uaaugauuauuuaacacaggcagaa

SEQ ID NO: 141 - RNA, 25 nt, Artificial Sequence
  Description of Artificial Sequence Synthetic probe
auuugguagauuuuauaguaaauga

SEQ ID NO: 142 - RNA, 25 nt, Artificial Sequence
  Description of Artificial Sequence Synthetic probe
ugacagugauacaggugaagauuug

SEQ ID NO: 143 - RNA, 25 nt, Artificial Sequence
  Description of Artificial Sequence Synthetic probe
agaaaccauaaucuaccauggcuga

SEQ ID NO: 144 - RNA, 25 nt, Artificial Sequence
  Description of Artificial Sequence Synthetic probe
cguacuuuggaagaccuguuaaugg

SEQ ID NO: 145 - RNA, 25 nt, Artificial Sequence
  Description of Artificial Sequence Synthetic probe
uuguugcaagugugacucuacgcuu

SEQ ID NO: 146 - RNA, 25 nt, Artificial Sequence
  Description of Artificial Sequence Synthetic probe
ggacagagcccauuacaauauugua

SEQ ID NO: 147 - RNA, 25 nt, Artificial Sequence
  Description of Artificial Sequence Synthetic probe
gagauacaccuacauugcaugaaua

SEQ ID NO: 148 - RNA, 25 nt, Artificial Sequence
  Description of Artificial Sequence Synthetic probe
agaucaucaagaacacguagagaaa

SEQ ID NO: 149 - RNA, 25 nt, Artificial Sequence
  Description of Artificial Sequence Synthetic probe
uccauaauauaaggggucgguggac

SEQ ID NO: 150 - RNA, 25 nt, Artificial Sequence
  Description of Artificial Sequence Synthetic probe
uauuaacugucaaaagccacugugu

SEQ ID NO: 151 - RNA, 25 nt, Artificial Sequence
  Description of Artificial Sequence Synthetic probe
uagaacagcaauacaacaaaccguu

SEQ ID NO: 152 - RNA, 25 nt, Artificial Sequence
  Description of Artificial Sequence Synthetic probe
acauuauuguuauaguuuguaugga

SEQ ID NO: 153 - RNA, 25 nt, Artificial Sequence
  Description of Artificial Sequence Synthetic probe
aguuuuauucuaaaauuagugagua

SEQ ID NO: 154 - RNA, 25 nt, Artificial Sequence
  Description of Artificial Sequence Synthetic probe
uaugcuguaugugauaaauguuuaa

SEQ ID NO: 155 - RNA, 25 nt, Artificial Sequence
  Description of Artificial Sequence Synthetic probe
cgggauuuaugcauaguauauagag

SEQ ID NO: 156 - RNA, 25 nt, Artificial Sequence
  Description of Artificial Sequence Synthetic probe
caguuacugcgacgugagguauaug

SEQ ID NO: 157 - RNA, 25 nt, Artificial Sequence
  Description of Artificial Sequence Synthetic probe
gagcugcaaacaacuauacaugaua

SEQ ID NO: 158 - RNA, 25 nt, Artificial Sequence
  Description of Artificial Sequence Synthetic probe
cagaaaguuaccacaguuaugcaca

SEQ ID NO: 159 - RNA, 25 nt, Artificial Sequence
  Description of Artificial Sequence Synthetic probe
aagagaacugcaauguuucaggacc

SEQ ID NO: 160 - RNA, 25 nt, Artificial Sequence
  Description of Artificial Sequence Synthetic probe
ccgguuaguauaaaagcagacauuu

SEQ ID NO: 161 - RNA, 25 nt, Artificial Sequence
  Description of Artificial Sequence Synthetic probe
auaaaacuaagggcguaaccgaaau

SEQ ID NO: 162 - RNA, 25 nt, Artificial Sequence
  Description of Artificial Sequence Synthetic probe
uguaugugcuuguaugugcuuguaa

SEQ ID NO: 163 - RNA, 25 nt, Artificial Sequence
  Description of Artificial Sequence Synthetic probe
uugggcagcacauacuauacuuuuc

SEQ ID NO: 164 - RNA, 25 nt, Artificial Sequence
  Description of Artificial Sequence Synthetic probe
uaagcugugcauacauaguuuaugc

SEQ ID NO: 165 - RNA, 25 nt, Artificial Sequence
  Description of Artificial Sequence Synthetic probe
cugucuacccuuaacaugaacuaua

SEQ ID NO: 166 - RNA, 25 nt, Artificial Sequence
  Description of Artificial Sequence Synthetic probe
guacaacuacuuucauguccaacau

SEQ ID NO: 167 - RNA, 25 nt, Artificial Sequence
  Description of Artificial Sequence Synthetic probe
auccacucccuaaguaauaaaacug

SEQ ID NO: 168 - RNA, 25 nt, Artificial Sequence
  Description of Artificial Sequence Synthetic probe
gcuacaacaauugcuugcauaacua

SEQ ID NO: 169 - RNA, 25 nt, Artificial Sequence
  Description of Artificial Sequence Synthetic probe
uugaacaauuggcgcgccucuuugg

SEQ ID NO: 170 - RNA, 25 nt, Artificial Sequence
  Description of Artificial Sequence Synthetic probe
cuuuugggcacugcuccuacauauu

SEQ ID NO: 171 - RNA, 25 nt, Artificial Sequence
  Description of Artificial Sequence Synthetic probe
caauacaguacgcuggcacuauugc

SEQ ID NO: 172 - RNA, 25 nt, Artificial Sequence
  Description of Artificial Sequence Synthetic probe
uggcuuaugucugugguuuucugca

SEQ ID NO: 173 - RNA, 25 nt, Artificial Sequence
  Description of Artificial Sequence Synthetic probe
ccauuuuauccuacaauccuccauu

SEQ ID NO: 174 - RNA, 25 nt, Artificial Sequence
  Description of Artificial Sequence Synthetic probe
uauaaaacugcacaccuuacagcau

SEQ ID NO: 175 - RNA, 25 nt, Artificial Sequence
  Description of Artificial Sequence Synthetic probe
gggcuauauauuguccuguauuuca

SEQ ID NO: 176 - RNA, 25 nt, Artificial Sequence
  Description of Artificial Sequence Synthetic probe
guuugugguauggguguugcuuguu

SEQ ID NO: 177 - RNA, 25 nt, Artificial Sequence
  Description of Artificial Sequence Synthetic probe
ccuagugaguaacaacuguauuugu

SEQ ID NO: 178 - RNA, 25 nt, Artificial Sequence
  Description of Artificial Sequence Synthetic probe
uugugguucuguguguuaugugguu

SEQ ID NO: 179 - RNA, 25 nt, Artificial Sequence
  Description of Artificial Sequence Synthetic probe
guuacuauauuuguugguauguggc

SEQ ID NO: 180 - RNA, 25 nt, Artificial Sequence
  Description of Artificial Sequence Synthetic probe
cauuguaugguauguaugguuguug

SEQ ID NO: 181 - RNA, 25 nt, Artificial Sequence
  Description of Artificial Sequence Synthetic probe
ccuguguuuguguuuguuguaugau

SEQ ID NO: 182 - RNA, 25 nt, Artificial Sequence
  Description of Artificial Sequence Synthetic probe
gugccaggaaguaauaugugugugu

SEQ ID NO: 183 - RNA, 25 nt, Artificial Sequence
  Description of Artificial Sequence Synthetic probe
aaaccugccaagcgugugcguguac

SEQ ID NO: 184 - RNA, 25 nt, Artificial Sequence
  Description of Artificial Sequence Synthetic probe
ugcuccaucugccacuacgucuucu

SEQ ID NO: 185 - RNA, 25 nt, Artificial Sequence
  Description of Artificial Sequence Synthetic probe
cuuuagacuuagaucaauauccccu

SEQ ID NO: 186 - RNA, 25 nt, Artificial Sequence
  Description of Artificial Sequence Synthetic probe
ugcaccggcugaaaauaaggauccc

SEQ ID NO: 187 - RNA, 25 nt, Artificial Sequence
  Description of Artificial Sequence Synthetic probe
guacaaucuguugcuauuaccuguc

SEQ ID NO: 188 - RNA, 25 nt, Artificial Sequence
  Description of Artificial Sequence Synthetic probe
gcaguauagcagacauguugaggaa

SEQ ID NO: 189 - RNA, 25 nt, Artificial Sequence
  Description of Artificial Sequence Synthetic probe
gggcaauaugaugcuaccaaauuua

SEQ ID NO: 190 - RNA, 25 nt, Artificial Sequence
  Description of Artificial Sequence Synthetic probe
ccaguaccaauuuaacaauaugugc

SEQ ID NO: 191 - RNA, 25 nt, Artificial Sequence
  Description of Artificial Sequence Synthetic probe
guauucucccucuccaaguggcucu

SEQ ID NO: 192 - RNA, 25 nt, Artificial Sequence
  Description of Artificial Sequence Synthetic probe
gccucaauccuuauauauuaaaggc

SEQ ID NO: 193 - RNA, 25 nt, Artificial Sequence
  Description of Artificial Sequence Synthetic probe
agauacuaaaugugagguaccauug

SEQ ID NO: 194 - RNA, 25 nt, Artificial Sequence
  Description of Artificial Sequence Synthetic probe
cacaguuuuggaagauggugauaug

SEQ ID NO: 195 - RNA, 25 nt, Artificial Sequence
  Description of Artificial Sequence Synthetic probe
uaaaucgcguccuuuaucacagggc

SEQ ID NO: 196 - RNA, 25 nt, Artificial Sequence
  Description of Artificial Sequence Synthetic probe
uucugaggacguuagggacaaugug

SEQ ID NO: 197 - RNA, 25 nt, Artificial Sequence
  Description of Artificial Sequence Synthetic probe
guucccaugccgccacgucuaaugu

SEQ ID NO: 198 - RNA, 25 nt, Artificial Sequence
  Description of Artificial Sequence Synthetic probe
guuccugcaggugguggcaauaagc

SEQ ID NO: 199 - RNA, 25 nt, Artificial Sequence
  Description of Artificial Sequence Synthetic probe
gcaagaguuguaaauaccgaugauu

SEQ ID NO: 200 - RNA, 25 nt, Artificial Sequence
  Description of Artificial Sequence Synthetic probe
cguauaucuuccaccuccuucugug

SEQ ID NO: 201 - RNA, 25 nt, Artificial Sequence
  Description of Artificial Sequence Synthetic probe
caguauauugguauacaugguacac

SEQ ID NO: 202 - RNA, 25 nt, Artificial Sequence
  Description of Artificial Sequence Synthetic probe
ccauuguaucacccacggccccugc

SEQ ID NO: 203 - RNA, 25 nt, Artificial Sequence
  Description of Artificial Sequence Synthetic probe
uuaccaucuacuaccucuguauggc

SEQ ID NO: 204 - RNA, 25 nt, Artificial Sequence
  Description of Artificial Sequence Synthetic probe
uguauacacggguccugauauuaca

SEQ ID NO: 205 - RNA, 25 nt, Artificial Sequence
  Description of Artificial Sequence Synthetic probe
ucccuuuaaccuccucuugggaugu

SEQ ID NO: 206 - RNA, 25 nt, Artificial Sequence
  Description of Artificial Sequence Synthetic probe
gccucuuccuauaguaauguaacgg

SEQ ID NO: 207 - RNA, 25 nt, Artificial Sequence
  Description of Artificial Sequence Synthetic probe
aucgcguucuacuaccuccuuugca

SEQ ID NO: 208 - RNA, 25 nt, Artificial Sequence
  Description of Artificial Sequence Synthetic probe
acauggacccugcagugccuguacc

SEQ ID NO: 209 - RNA, 25 nt, Artificial Sequence
  Description of Artificial Sequence Synthetic probe
cagccuuuaguaucugccacggagg

SEQ ID NO: 210 - RNA, 25 nt, Artificial Sequence
  Description of Artificial Sequence Synthetic probe
accuuccccagaauauauugaacug

SEQ ID NO: 211 - RNA, 25 nt, Artificial Sequence
  Description of Artificial Sequence Synthetic probe
uuacccgcagcgguacacaaauagg

SEQ ID NO: 212 - RNA, 25 nt, Artificial Sequence
  Description of Artificial Sequence Synthetic probe
ggacuguucgcuuuaguagauuagg

SEQ ID NO: 213 - RNA, 25 nt, Artificial Sequence
  Description of Artificial Sequence Synthetic probe
gacacuacauuaacauuugauccuc

SEQ ID NO: 214 - RNA, 25 nt, Artificial Sequence
  Description of Artificial Sequence Synthetic probe
cacguccauccucuuuaauuacaua

SEQ ID NO: 215 - RNA, 25 nt, Artificial Sequence
  Description of Artificial Sequence Synthetic probe
ucaguggcuaacccugaguuucuua

SEQ ID NO: 216 - RNA, 25 nt, Artificial Sequence
  Description of Artificial Sequence Synthetic probe
uacaaacauuugcuucuucugguac

SEQ ID NO: 217 - RNA, 25 nt, Artificial Sequence
  Description of Artificial Sequence Synthetic probe
cguccauuauugaaguuccacaaac

SEQ ID NO: 218 - RNA, 25 nt, Artificial Sequence
  Description of Artificial Sequence Synthetic probe
ccacaaccaauuuuaccaauccugc

SEQ ID NO: 219 - RNA, 25 nt, Artificial Sequence
  Description of Artificial Sequence Synthetic probe
ccuucgucuaccucugugucuauuu

SEQ ID NO: 220 - RNA, 25 nt, Artificial Sequence
  Description of Artificial Sequence Synthetic probe
acaucugcggguacaacuacaccug

SEQ ID NO: 221 - RNA, 25 nt, Artificial Sequence
  Description of Artificial Sequence Synthetic probe
ugcaccuaggccuacguuuacuggc

SEQ ID NO: 222 - RNA, 25 nt, Artificial Sequence
  Description of Artificial Sequence Synthetic probe
aggacuccagugugguuacaucagg

SEQ ID NO: 223 - RNA, 25 nt, Artificial Sequence
  Description of Artificial Sequence Synthetic probe
agugguggauguugguccuacacgu

SEQ ID NO: 224 - RNA, 25 nt, Artificial Sequence
  Description of Artificial Sequence Synthetic probe
acauuccauugggugggcguuccaa

SEQ ID NO: 225 - RNA, 25 nt, Artificial Sequence
  Description of Artificial Sequence Synthetic probe
auugcaauggucaagccuugguaua

SEQ ID NO: 226 - RNA, 25 nt, Artificial Sequence
  Description of Artificial Sequence Synthetic probe
ggcuucgguaacugacuuauauaaa

SEQ ID NO: 227 - RNA, 25 nt, Artificial Sequence
  Description of Artificial Sequence Synthetic probe
uaauaaaaguaugguaucccaccgu

SEQ ID NO: 228 - RNA, 25 nt, Artificial Sequence
  Description of Artificial Sequence Synthetic probe
cccauguuacuauugcauauacaug

SEQ ID NO: 229 - RNA, 25 nt, Artificial Sequence
  Description of Artificial Sequence Synthetic probe
cugccacagcauucacaguauaugu

SEQ ID NO: 230 - RNA, 25 nt, Artificial Sequence
  Description of Artificial Sequence Synthetic probe
guguauauugugguaauaacguccc

SEQ ID NO: 231 - RNA, 25 nt, Artificial Sequence
  Description of Artificial Sequence Synthetic probe
augcauguaugugugcugccauguc

SEQ ID NO: 232 - RNA, 25 nt, Artificial Sequence
  Description of Artificial Sequence Synthetic probe
gcuguaguaccaauauguuaucacu

SEQ ID NO: 233 - RNA, 25 nt, Artificial Sequence
  Description of Artificial Sequence Synthetic probe
auauuggugggauacaugacaaugu

SEQ ID NO: 234 - RNA, 25 nt, Artificial Sequence
  Description of Artificial Sequence Synthetic probe
uguugcaauuccagauaguguacaa

SEQ ID NO: 235 - RNA, 25 nt, Artificial Sequence
  Description of Artificial Sequence Synthetic probe
cauaccauagugaaacacaaagaac

SEQ ID NO: 236 - RNA, 25 nt, Artificial Sequence
  Description of Artificial Sequence Synthetic probe
cuauagagauauaucauccaccugg

SEQ ID NO: 237 - RNA, 25 nt, Artificial Sequence
  Description of Artificial Sequence Synthetic probe
acagauugcgaaaacauagcgacca

SEQ ID NO: 238 - RNA, 25 nt, Artificial Sequence
  Description of Artificial Sequence Synthetic probe
aagacggaaacucuguagugguaac

SEQ ID NO: 239 - RNA, 25 nt, Artificial Sequence
  Description of Artificial Sequence Synthetic probe
cagcuacaccuacaggcaacaacaa

SEQ ID NO: 240 - RNA, 25 nt, Artificial Sequence
  Description of Artificial Sequence Synthetic probe
ggaccugucaacccacuucucggug

SEQ ID NO: 241 - RNA, 25 nt, Artificial Sequence
  Description of Artificial Sequence Synthetic probe
uggacucgcggagaagcagcauugu

SEQ ID NO: 242 - RNA, 25 nt, Artificial Sequence
  Description of Artificial Sequence Synthetic probe
cggcugcuacacgaccuggacacug

SEQ ID NO: 243 - RNA, 25 nt, Artificial Sequence
  Description of Artificial Sequence Synthetic probe
auuccagcaccguguccgugggcac

SEQ ID NO: 244 - RNA, 25 nt, Artificial Sequence
  Description of Artificial Sequence Synthetic probe
ccgcuacucagcuuguuaaacagcu

SEQ ID NO: 245 - RNA, 25 nt, Artificial Sequence
  Description of Artificial Sequence Synthetic probe
gggaaguacauuuugggaauaaugu

SEQ ID NO: 246 - RNA, 25 nt, Artificial Sequence
  Description of Artificial Sequence Synthetic probe
gaaggguacaacacguuuuauauag

SEQ ID NO: 247 - RNA, 25 nt, Artificial Sequence
  Description of Artificial Sequence Synthetic probe
caaaaccgcuaccuguguaagucac

SEQ ID NO: 248 - RNA, 25 nt, Artificial Sequence
  Description of Artificial Sequence Synthetic probe
auaugacugaugcaggaacauggga

SEQ ID NO: 249 - RNA, 25 nt, Artificial Sequence
  Description of Artificial Sequence Synthetic probe
uauguagcaugggacaguguguauu

SEQ ID NO: 250 - RNA, 25 nt, Artificial Sequence
  Description of Artificial Sequence Synthetic probe
ggccaaacaguacaaguauauuuug

SEQ ID NO: 251 - RNA, 25 nt, Artificial Sequence
  Description of Artificial Sequence Synthetic probe
gaauacagaaccuacucacugcuuu

SEQ ID NO: 252 - RNA, 25 nt, Artificial Sequence
  Description of Artificial Sequence Synthetic probe
aagucgauacaaaaccgaggauugg

SEQ ID NO: 253 - RNA, 25 nt, Artificial Sequence
  Description of Artificial Sequence Synthetic probe
acauggcauacagacauuaaaccac

SEQ ID NO: 254 - RNA, 25 nt, Artificial Sequence
  Description of Artificial Sequence Synthetic probe
guugggaaaaugcaauauucuuugc

SEQ ID NO: 255 - RNA, 25 nt, Artificial Sequence
  Description of Artificial Sequence Synthetic probe
cauagacagccaaauacaguauugg

SEQ ID NO: 256 - RNA, 25 nt, Artificial Sequence
  Description of Artificial Sequence Synthetic probe
gcaaaggauaauagauggccauauu

SEQ ID NO: 257 - RNA, 25 nt, Artificial Sequence
  Description of Artificial Sequence Synthetic probe
ccuccaauacuacuaaccacaaaua

SEQ ID NO: 258 - RNA, 25 nt, Artificial Sequence
  Description of Artificial Sequence Synthetic probe
cuuugauaccuauaugagaaaugcg

SEQ ID NO: 259 - RNA, 25 nt, Artificial Sequence
  Description of Artificial Sequence Synthetic probe
cagauacuaagguggccauguuaga

SEQ ID NO: 260 - RNA, 25 nt, Artificial Sequence
  Description of Artificial Sequence Synthetic probe
cugcgauaccaacaaauagaguuua

SEQ ID NO: 261 - RNA, 25 nt, Artificial Sequence
  Description of Artificial Sequence Synthetic probe
cacaguggauacgauuuagauguuc

SEQ ID NO: 262 - RNA, 25 nt, Artificial Sequence
  Description of Artificial Sequence Synthetic probe
ugaauaugccuuauuagcagacagc

SEQ ID NO: 263 - RNA, 25 nt, Artificial Sequence
  Description of Artificial Sequence Synthetic probe
gagcugacagaugaaagcgauaugg

SEQ ID NO: 264 - RNA, 25 nt, Artificial Sequence
  Description of Artificial Sequence Synthetic probe
cugaguggauacaaagacuuacuau

SEQ ID NO: 265 - RNA, 25 nt, Artificial Sequence
  Description of Artificial Sequence Synthetic probe
uauuagugaaguaaugggagacaca

SEQ ID NO: 266 - RNA, 25 nt, Artificial Sequence
  Description of Artificial Sequence Synthetic probe
cacguaccugaaacuuguauguuaa

SEQ ID NO: 267 - RNA, 25 nt, Artificial Sequence
  Description of Artificial Sequence Synthetic probe
guugcuaaagguuuaaguacguugu

SEQ ID NO: 268 - RNA, 25 nt, Artificial Sequence
  Description of Artificial Sequence Synthetic probe
caaaugugguaagaguagacuaaca

SEQ ID NO: 269 - RNA, 25 nt, Artificial Sequence
  Description of Artificial Sequence Synthetic probe
guauuaauauuagcccuguugcguu

SEQ ID NO: 270 - RNA, 25 nt, Artificial Sequence
  Description of Artificial Sequence Synthetic probe
ucaaugucuagacuguaaaugggga

SEQ ID NO: 271 - RNA, 25 nt, Artificial Sequence
  Description of Artificial Sequence Synthetic probe
acacauaugggcuaucauuuacaga

SEQ ID NO: 272 - RNA, 25 nt, Artificial Sequence
  Description of Artificial Sequence Synthetic probe
acaauaaacaaggagcuauguuagc

SEQ ID NO: 273 - RNA, 25 nt, Artificial Sequence
  Description of Artificial Sequence Synthetic probe
ccacaauguaccauagcacaauuaa

SEQ ID NO: 274 - RNA, 25 nt, Artificial Sequence
  Description of Artificial Sequence Synthetic probe
acgguacaagugacaauagcaauau

SEQ ID NO: 275 - RNA, 25 nt, Artificial Sequence
  Description of Artificial Sequence Synthetic probe
cacagagggcaacaacagcagugua

SEQ ID NO: 276 - RNA, 25 nt, Artificial Sequence
  Description of Artificial Sequence Synthetic probe
cggcaguacggaggcuauagacaac

SEQ ID NO: 277 - RNA, 25 nt, Artificial Sequence
  Description of Artificial Sequence Synthetic probe
aacuacaaauggcgaacauggcggc

SEQ ID NO: 278 - RNA, 25 nt, Artificial Sequence
  Description of Artificial Sequence Synthetic probe
gcggcuggagguggauacagaguua

SEQ ID NO: 279 - RNA, 25 nt, Artificial Sequence
  Description of Artificial Sequence Synthetic probe
cacaaguguugcauguuuuaaaacg

SEQ ID NO: 280 - RNA, 25 nt, Artificial Sequence
  Description of Artificial Sequence Synthetic probe
acaaggaacauuuugugaacaggca

SEQ ID NO: 281 - RNA, 25 nt, Artificial Sequence
  Description of Artificial Sequence Synthetic probe
ggcugguuuuauguacaagcuauug

SEQ ID NO: 282 - RNA, 25 nt, Artificial Sequence
  Description of Artificial Sequence Synthetic probe
cguggugugcaucccagcaguaagc

SEQ ID NO: 283 - RNA, 25 nt, Artificial Sequence
  Description of Artificial Sequence Synthetic probe
uuucugaacacccuguccuuugugu

SEQ ID NO: 284 - RNA, 25 nt, Artificial Sequence
  Description of Artificial Sequence Synthetic probe
uagaaagcucagcagacgaccuucg

SEQ ID NO: 285 - RNA, 25 nt, Artificial Sequence
  Description of Artificial Sequence Synthetic probe
ugugaagccagaauugagcuaguag

SEQ ID NO: 286 - RNA, 25 nt, Artificial Sequence
  Description of Artificial Sequence Synthetic probe
gaagaaaacgaugaaauagauggag

SEQ ID NO: 287 - RNA, 25 nt, Artificial Sequence
  Description of Artificial Sequence Synthetic probe
ucacgagcaauuaagcgacucagag

SEQ ID NO: 288 - RNA, 25 nt, Artificial Sequence
  Description of Artificial Sequence Synthetic probe
augaaauuccgguugaccuucuaug

SEQ ID NO: 289 - RNA, 25 nt, Artificial Sequence
  Description of Artificial Sequence Synthetic probe
auuguauugcauuuagagccccaaa

SEQ ID NO: 290 - RNA, 25 nt, Artificial Sequence
  Description of Artificial Sequence Synthetic probe
uaugcauggaccuaaggcaacauug

SEQ ID NO: 291 - RNA, 25 nt, Artificial Sequence
  Description of Artificial Sequence Synthetic probe
ccaacgacgcagagaaacacaagua

SEQ ID NO: 292 - RNA, 25 nt, Artificial Sequence
  Description of Artificial Sequence Synthetic probe
gcaaccgagcacgacaggaacgacu

SEQ ID NO: 293 - RNA, 25 nt, Artificial Sequence
  Description of Artificial Sequence Synthetic probe
aacauagcugggcacuauagaggcc

SEQ ID NO: 294 - RNA, 25 nt, Artificial Sequence
  Description of Artificial Sequence Synthetic probe
uuauucagacucuguguauggagac

SEQ ID NO: 295 - RNA, 25 nt, Artificial Sequence
  Description of Artificial Sequence Synthetic probe
gugguguauagagacaguauacccc

SEQ ID NO: 296 - RNA, 25 nt, Artificial Sequence
  Description of Artificial Sequence Synthetic probe
guauuggaacuuacagagguauuug

SEQ ID NO: 297 - RNA, 25 nt, Artificial Sequence
  Description of Artificial Sequence Synthetic probe
gcaagacauagaaauaaccugugua

SEQ ID NO: 298 - RNA, 25 nt, Artificial Sequence
  Description of Artificial Sequence Synthetic probe
ugugcacggaacugaacacuucacu

SEQ ID NO: 299 - RNA, 25 nt, Artificial Sequence
  Description of Artificial Sequence Synthetic probe
acaccacaauacuauggcgcgcuuu

SEQ ID NO: 300 - RNA, 25 nt, Artificial Sequence
  Description of Artificial Sequence Synthetic probe
ccugguauuagucauuuuccugucc

SEQ ID NO: 301 - RNA, 25 nt, Artificial Sequence
  Description of Artificial Sequence Synthetic probe
cuaguuugguggauacauaucguuu

SEQ ID NO: 302 - RNA, 25 nt, Artificial Sequence
  Description of Artificial Sequence Synthetic probe
acucccacaagcauauuuuaucaug

SEQ ID NO: 303 - RNA, 25 nt, Artificial Sequence
  Description of Artificial Sequence Synthetic probe
guagugauguuccugauucagauuu

SEQ ID NO: 304 - RNA, 25 nt, Artificial Sequence
  Description of Artificial Sequence Synthetic probe
gaccacuaugaaaaugacaguaaag

SEQ ID NO: 305 - RNA, 25 nt, Artificial Sequence
  Description of Artificial Sequence Synthetic probe
cuguuuacaauaucagauaguggcu

SEQ ID NO: 306 - RNA, 25 nt, Artificial Sequence
  Description of Artificial Sequence Synthetic probe
aguccacgguuacaagaaauaucuu

SEQ ID NO: 307 - RNA, 25 nt, Artificial Sequence
  Description of Artificial Sequence Synthetic probe
agcccgacgagccgaaccacaacgu

SEQ ID NO: 308 - RNA, 25 nt, Artificial Sequence
  Description of Artificial Sequence Synthetic probe
uuauuaauaaggugccugcggugcc

SEQ ID NO: 309 - RNA, 25 nt, Artificial Sequence
  Description of Artificial Sequence Synthetic probe
augcugcaugccauaaauguauaga

SEQ ID NO: 310 - RNA, 25 nt, Artificial Sequence
  Description of Artificial Sequence Synthetic probe
guuuucgguuacaguuuuacaagca

SEQ ID NO: 311 - RNA, 25 nt, Artificial Sequence
  Description of Artificial Sequence Synthetic probe
ccaagguugugucaugcauuauaaa

SEQ ID NO: 312 - RNA, 25 nt, Artificial Sequence
  Description of Artificial Sequence Synthetic probe
ccuugauugcagugcuggcuuuugc

SEQ ID NO: 313 - RNA, 25 nt, Artificial Sequence
  Description of Artificial Sequence Synthetic probe
ccuacacaccuuaaacugcuuuuag

SEQ ID NO: 314 - RNA, 25 nt, Artificial Sequence
  Description of Artificial Sequence Synthetic probe
uguaguucaacuaugugucaugcac

SEQ ID NO: 315 - RNA, 25 nt, Artificial Sequence
  Description of Artificial Sequence Synthetic probe
ccaguccaacuuugcaauuauacua

SEQ ID NO: 316 - RNA, 25 nt, Artificial Sequence
  Description of Artificial Sequence Synthetic probe
cuaacacaccuugccaacauauaau

SEQ ID NO: 317 - RNA, 25 nt, Artificial Sequence
  Description of Artificial Sequence Synthetic probe
aacauucuggcuuguaguuuccugc

SEQ ID NO: 318 - RNA, 25 nt, Artificial Sequence
  Description of Artificial Sequence Synthetic probe
caugcuaguacaacuaugcugaugc

SEQ ID NO: 319 - RNA, 25 nt, Artificial Sequence
  Description of Artificial Sequence Synthetic probe
cauuuuaaaucccuaaccguuuucg

SEQ ID NO: 320 - RNA, 25 nt, Artificial Sequence
  Description of Artificial Sequence Synthetic probe
cuacuccauuuugauuuuaugcagc

SEQ ID NO: 321 - RNA, 25 nt, Artificial Sequence
  Description of Artificial Sequence Synthetic probe
uaguaaaaguuguacacccgguccg

SEQ ID NO: 322 - RNA, 25 nt, Artificial Sequence
  Description of Artificial Sequence Synthetic probe
caauagucauguacuuauuucugcc

SEQ ID NO: 323 - RNA, 25 nt, Artificial Sequence
  Description of Artificial Sequence Synthetic probe
uguuccuacuuguuccugcuccucc

SEQ ID NO: 324 - RNA, 25 nt, Artificial Sequence
  Description of Artificial Sequence Synthetic probe
guuguccuuauauacacccuauuag

SEQ ID NO: 325 - RNA, 25 nt, Artificial Sequence
  Description of Artificial Sequence Synthetic probe
auauguguauaccuguguguguugu

SEQ ID NO: 326 - RNA, 25 nt, Artificial Sequence
  Description of Artificial Sequence Synthetic probe
gcuguauuguauauguguguguuug

SEQ ID NO: 327 - RNA, 25 nt, Artificial Sequence
  Description of Artificial Sequence Synthetic probe
ugugucuguauguguaugugcuugu

SEQ ID NO: 328 - RNA, 25 nt, Artificial Sequence
  Description of Artificial Sequence Synthetic probe
aucuaccacuacaccagcaaaacgu

SEQ ID NO: 329 - RNA, 25 nt, Artificial Sequence
  Description of Artificial Sequence Synthetic probe
guccuaaauuuaaagcagguaaacg

SEQ ID NO: 330 - RNA, 25 nt, Artificial Sequence
  Description of Artificial Sequence Synthetic probe
cccaaggaagauccauuuaaagauu

SEQ ID NO: 331 - RNA, 25 nt, Artificial Sequence
  Description of Artificial Sequence Synthetic probe
cagguucuuuggaggauaccuauag

SEQ ID NO: 332 - RNA, 25 nt, Artificial Sequence
  Description of Artificial Sequence Synthetic probe
gcaauugcaaacagugauacuacau

SEQ ID NO: 333 - RNA, 25 nt, Artificial Sequence
  Description of Artificial Sequence Synthetic probe
guaguaccaauaugucuguuugugc

SEQ ID NO: 334 - RNA, 25 nt, Artificial Sequence
  Description of Artificial Sequence Synthetic probe
auacuuuccuacaccuagcggcucc

SEQ ID NO: 335 - RNA, 25 nt, Artificial Sequence
  Description of Artificial Sequence Synthetic probe
gcuccgguucaacagcuacuuuagc

SEQ ID NO: 336 - RNA, 25 nt, Artificial Sequence
  Description of Artificial Sequence Synthetic probe
ugaaucggucccuacugacuuauau

SEQ ID NO: 337 - RNA, 25 nt, Artificial Sequence
  Description of Artificial Sequence Synthetic probe
gacacuaaaaguaauguuccuuugg

SEQ ID NO: 338 - RNA, 25 nt, Artificial Sequence
  Description of Artificial Sequence Synthetic probe
gcuauuaccccuggugauuguccuc

SEQ ID NO: 339 - RNA, 25 nt, Artificial Sequence
  Description of Artificial Sequence Synthetic probe
caacuguguuuacuugguugcaaac

SEQ ID NO: 340 - RNA, 25 nt, Artificial Sequence
  Description of Artificial Sequence Synthetic probe
cggugguccuggcacugauaauagg

SEQ ID NO: 341 - RNA, 25 nt, Artificial Sequence
  Description of Artificial Sequence Synthetic probe
uuccauaccuaaaucugacaauccu

SEQ ID NO: 342 - RNA, 25 nt, Artificial Sequence
  Description of Artificial Sequence Synthetic probe
agugcuaggcugcuuacaguaggcc

SEQ ID NO: 343 - RNA, 25 nt, Artificial Sequence
  Description of Artificial Sequence Synthetic probe
gaaccaacauauauuaucacgcagg

SEQ ID NO: 344 - RNA, 25 nt, Artificial Sequence
  Description of Artificial Sequence Synthetic probe
ugucccagugucuaaaguuguaagc

SEQ ID NO: 345 - RNA, 25 nt, Artificial Sequence
  Description of Artificial Sequence Synthetic probe
gcgaggcuacugucuacuuaccacc

SEQ ID NO: 346 - RNA, 25 nt, Artificial Sequence
  Description of Artificial Sequence Synthetic probe
gccccuacaacgccacaagugucua

SEQ ID NO: 347 - RNA, 25 nt, Artificial Sequence
  Description of Artificial Sequence Synthetic probe
uacacagguuuucccauuuccuuug

SEQ ID NO: 348 - RNA, 25 nt, Artificial Sequence
  Description of Artificial Sequence Synthetic probe
cugauguaccuauagagcaugcacc

SEQ ID NO: 349 - RNA, 25 nt, Artificial Sequence
  Description of Artificial Sequence Synthetic probe
uuuugacauucccauauuuucuggg

SEQ ID NO: 350 - RNA, 25 nt, Artificial Sequence
  Description of Artificial Sequence Synthetic probe
aaauaccacugugccacuaaguaca

SEQ ID NO: 351 - RNA, 25 nt, Artificial Sequence
  Description of Artificial Sequence Synthetic probe
cugcuguacaguccacaucugcugu

SEQ ID NO: 352 - RNA, 25 nt, Artificial Sequence
  Description of Artificial Sequence Synthetic probe
uggauacaccugccacacauaaugu

SEQ ID NO: 353 - RNA, 25 nt, Artificial Sequence
  Description of Artificial Sequence Synthetic probe
augcaaccuuuaggggcgucugcaa

SEQ ID NO: 354 - RNA, 25 nt, Artificial Sequence
  Description of Artificial Sequence Synthetic probe
uaauccugcaggugaaaguauugaa

SEQ ID NO: 355 - RNA, 25 nt, Artificial Sequence
  Description of Artificial Sequence Synthetic probe
uggugcuacuauuggugcaagggug

SEQ ID NO: 356 - RNA, 25 nt, Artificial Sequence
  Description of Artificial Sequence Synthetic probe
auaaacaaacuuugcgcacucguag

SEQ ID NO: 357 - RNA, 25 nt, Artificial Sequence
  Description of Artificial Sequence Synthetic probe
cacuguuagauauaguagacuaggu

SEQ ID NO: 358 - RNA, 25 nt, Artificial Sequence
  Description of Artificial Sequence Synthetic probe
cccgacuuucuagauauuauagcau

SEQ ID NO: 359 - RNA, 25 nt, Artificial Sequence
  Description of Artificial Sequence Synthetic probe
uacaucgcauaauauagccccugau

SEQ ID NO: 360 - RNA, 25 nt, Artificial Sequence
  Description of Artificial Sequence Synthetic probe
ccuaugaaacuguaaaugcugaaga

SEQ ID NO: 361 - RNA, 25 nt, Artificial Sequence
  Description of Artificial Sequence Synthetic probe
gcuccaaaacagcuaauuacauaug

SEQ ID NO: 362 - RNA, 25 nt, Artificial Sequence
  Description of Artificial Sequence Synthetic probe
guaaggcuacacaacaaguaaaagu

SEQ ID NO: 363 - RNA, 25 nt, Artificial Sequence
  Description of Artificial Sequence Synthetic probe
cauaacaaguagcacacccauucca

SEQ ID NO: 364 - RNA, 25 nt, Artificial Sequence
  Description of Artificial Sequence Synthetic probe
caggucauuuacuacuuucaucauc

SEQ ID NO: 365 - RNA, 25 nt, Artificial Sequence
  Description of Artificial Sequence Synthetic probe
cagccuccuacaccugcagaaacau

SEQ ID NO: 366 - RNA, 25 nt, Artificial Sequence
  Description of Artificial Sequence Synthetic probe
gcacacaugaaaauccuacuuuuac

SEQ ID NO: 367 - RNA, 25 nt, Artificial Sequence
  Description of Artificial Sequence Synthetic probe
cagacacaacaccugcaauuuuaga

SEQ ID NO: 368 - RNA, 25 nt, Artificial Sequence
  Description of Artificial Sequence Synthetic probe
ucuggguuugacauugcuacaacug

SEQ ID NO: 369 - RNA, 25 nt, Artificial Sequence
  Description of Artificial Sequence Synthetic probe
uccuauaccacacccuccuacaaca

SEQ ID NO: 370 - RNA, 25 nt, Artificial Sequence
  Description of Artificial Sequence Synthetic probe
gaauuguugauguuggugccccugc

SEQ ID NO: 371 - RNA, 25 nt, Artificial Sequence
  Description of Artificial Sequence Synthetic probe
ccucuauaguaagucuuguugaaga

SEQ ID NO: 372 - RNA, 25 nt, Artificial Sequence
  Description of Artificial Sequence Synthetic probe
caccaguuagcauugacccuguagg

SEQ ID NO: 373 - RNA, 25 nt, Artificial Sequence
  Description of Artificial Sequence Synthetic probe
ucugaggcaaguauaccuauuagac

SEQ ID NO: 374 - RNA, 25 nt, Artificial Sequence
  Description of Artificial Sequence Synthetic probe
ucuuaguacacguccuucuacagua

SEQ ID NO: 375 - RNA, 25 nt, Artificial Sequence
  Description of Artificial Sequence Synthetic probe
auauuaagguaugguaguaugggug

SEQ ID NO: 376 - RNA, 25 nt, Artificial Sequence
  Description of Artificial Sequence Synthetic probe
ccaucagacguuauaccuaaaauag

SEQ ID NO: 377 - RNA, 25 nt, Artificial Sequence
  Description of Artificial Sequence Synthetic probe
acgcucuacaaaacgcacuaaacgu

SEQ ID NO: 378 - RNA, 25 nt, Artificial Sequence
  Description of Artificial Sequence Synthetic probe
uuauugcaaccucuccauuacguug

SEQ ID NO: 379 - RNA, 25 nt, Artificial Sequence
  Description of Artificial Sequence Synthetic probe
gucgguauaugcaacacuacuauua

SEQ ID NO: 380 - RNA, 25 nt, Artificial Sequence
  Description of Artificial Sequence Synthetic probe
ucauacguccacuugugcugucugu

SEQ ID NO: 381 - RNA, 25 nt, Artificial Sequence
  Description of Artificial Sequence Synthetic probe
ugugugcuacuauuugugugucuug

SEQ ID NO: 382 - RNA, 25 nt, Artificial Sequence
  Description of Artificial Sequence Synthetic probe
caacaggauauaugacuauuuagcc

SEQ ID NO: 383 - RNA, 25 nt, Artificial Sequence
  Description of Artificial Sequence Synthetic probe
uuggacauguacagauggaaaacau

SEQ ID NO: 384 - RNA, 25 nt, Artificial Sequence
  Description of Artificial Sequence Synthetic probe
uguaugaacaagugucaucuacaug

SEQ ID NO: 385 - RNA, 25 nt, Artificial Sequence
  Description of Artificial Sequence Synthetic probe
cugcaacuacaccuauaauacacuu

SEQ ID NO: 386 - RNA, 25 nt, Artificial Sequence
  Description of Artificial Sequence Synthetic probe
aaccaaacaagggcugucaguuguc

SEQ ID NO: 387 - RNA, 25 nt, Artificial Sequence
  Description of Artificial Sequence Synthetic probe
ugugggguuaucagugcagcugcau

SEQ ID NO: 388 - RNA, 25 nt, Artificial Sequence
  Description of Artificial Sequence Synthetic probe
ccaagaacagagccagagcacagaa

SEQ ID NO: 389 - RNA, 25 nt, Artificial Sequence
  Description of Artificial Sequence Synthetic probe
gaauuccaaaaccugcgccuugggc

SEQ ID NO: 390 - RNA, 25 nt, Artificial Sequence
  Description of Artificial Sequence Synthetic probe
uccuuugcugggauuguuacaaagc

SEQ ID NO: 391 - RNA, 25 nt, Artificial Sequence
  Description of Artificial Sequence Synthetic probe
gaaucuguauuuagcagugacgaaa

SEQ ID NO: 392 - RNA, 25 nt, Artificial Sequence
  Description of Artificial Sequence Synthetic probe
ggcauuuauuauguacaugaaggac

SEQ ID NO: 393 - RNA, 25 nt, Artificial Sequence
  Description of Artificial Sequence Synthetic probe
uguggaagggcaaguuaauuguaag

SEQ ID NO: 394 - RNA, 25 nt, Artificial Sequence
  Description of Artificial Sequence Synthetic probe
uauguauagauggccaauguacugu

SEQ ID NO: 395 - RNA, 25 nt, Artificial Sequence
  Description of Artificial Sequence Synthetic probe
caccaugcauuauacuaacuggaaa

SEQ ID NO: 396 - RNA, 25 nt, Artificial Sequence
  Description of Artificial Sequence Synthetic probe
ggugcaauuugauggugauguacac

SEQ ID NO: 397 - RNA, 25 nt, Artificial Sequence
  Description of Artificial Sequence Synthetic probe
uugaacuguauuuaacugcaccuac

SEQ ID NO: 398 - RNA, 25 nt, Artificial Sequence
  Description of Artificial Sequence Synthetic probe
gacuggacaaugcagcaaacaaguc

SEQ ID NO: 399 - RNA, 25 nt, Artificial Sequence
  Description of Artificial Sequence Synthetic probe
gccuuacaagcuauugaacuacaaa

SEQ ID NO: 400 - RNA, 25 nt, Artificial Sequence
  Description of Artificial Sequence Synthetic probe
ccagcguugucaguaucaaaggcca

SEQ ID NO: 401 - RNA, 25 nt, Artificial Sequence
  Description of Artificial Sequence Synthetic probe
gggaauacacaguauuaaccaccag

SEQ ID NO: 402 - RNA, 25 nt, Artificial Sequence
  Description of Artificial Sequence Synthetic probe
gacuauuggaaacauauucgacuug

SEQ ID NO: 403 - RNA, 25 nt, Artificial Sequence
  Description of Artificial Sequence Synthetic probe
gacucuuucucaacguuuaaaugug

SEQ ID NO: 404 - RNA, 25 nt, Artificial Sequence
  Description of Artificial Sequence Synthetic probe
uaaauuugcacgaggaagaggacaa

SEQ ID NO: 405 - RNA, 25 nt, Artificial Sequence
  Description of Artificial Sequence Synthetic probe
ugacagauggccauaccuacauagc

SEQ ID NO: 406 - RNA, 25 nt, Artificial Sequence
  Description of Artificial Sequence Synthetic probe
cccuguaucuauagauguaaagcau

SEQ ID NO: 407 - RNA, 25 nt, Artificial Sequence
  Description of Artificial Sequence Synthetic probe
auuaccuacgaaaugcacuagaugg

SEQ ID NO: 408 - RNA, 25 nt, Artificial Sequence
  Description of Artificial Sequence Synthetic probe
uaauacauggugcaccuaauacagg

SEQ ID NO: 409 - RNA, 25 nt, Artificial Sequence
  Description of Artificial Sequence Synthetic probe
aggugacuggagggacauaguaaag

SEQ ID NO: 410 - RNA, 25 nt, Artificial Sequence
  Description of Artificial Sequence Synthetic probe
guagaugugacaaaguuagugacga

SEQ ID NO: 411 - RNA, 25 nt, Artificial Sequence
  Description of Artificial Sequence Synthetic probe
cugacagugauaguaaugcaugugc

SEQ ID NO: 412 - RNA, 25 nt, Artificial Sequence
  Description of Artificial Sequence Synthetic probe
gacacaacauuugauuugucccaaa

SEQ ID NO: 413 - RNA, 25 nt, Artificial Sequence
  Description of Artificial Sequence Synthetic probe
guauguuaauucagccacccaaauu

SEQ ID NO: 414 - RNA, 25 nt, Artificial Sequence
  Description of Artificial Sequence Synthetic probe
uuacaaaguuuagcauguuccuggg

SEQ ID NO: 415 - RNA, 25 nt, Artificial Sequence
  Description of Artificial Sequence Synthetic probe
gcaaccauauuguuuguauugccau

SEQ ID NO: 416 - RNA, 25 nt, Artificial Sequence
  Description of Artificial Sequence Synthetic probe
guugcagaaggauuuaaaacccuau

SEQ ID NO: 417 - RNA, 25 nt, Artificial Sequence
  Description of Artificial Sequence Synthetic probe
agcugcguuuggaguuacagguaca

SEQ ID NO: 418 - RNA, 25 nt, Artificial Sequence
  Description of Artificial Sequence Synthetic probe
aaagcacauguacugauuggugugu

SEQ ID NO: 419 - RNA, 25 nt, Artificial Sequence
  Description of Artificial Sequence Synthetic probe
gaacuaauuaggccauuucaaagca

SEQ ID NO: 420 - RNA, 25 nt, Artificial Sequence
  Description of Artificial Sequence Synthetic probe
gguaaagcugcuauguuagguaaau

SEQ ID NO: 421 - RNA, 25 nt, Artificial Sequence
  Description of Artificial Sequence Synthetic probe
ccaacacguaauauauugcaagugu

SEQ ID NO: 422 - RNA, 25 nt, Artificial Sequence
  Description of Artificial Sequence Synthetic probe
acauagugaacgagagaaugaaacu

SEQ ID NO: 423 - RNA, 25 nt, Artificial Sequence
  Description of Artificial Sequence Synthetic probe
uaaguuguaaugguagugacgggac

SEQ ID NO: 424 - RNA, 25 nt, Artificial Sequence
  Description of Artificial Sequence Synthetic probe
cagguagaggagcaacaaacaacau

SEQ ID NO: 425 - RNA, 25 nt, Artificial Sequence
  Description of Artificial Sequence Synthetic probe
ugaaguggaaacgcagcagauggua

SEQ ID NO: 426 - RNA, 25 nt, Artificial Sequence
  Description of Artificial Sequence Synthetic probe
acucuuugaacuuccagacagcggg

SEQ ID NO: 427 - RNA, 25 nt, Artificial Sequence
  Description of Artificial Sequence Synthetic probe
cacgguuaaaagcuauaugcauaga

SEQ ID NO: 428 - RNA, 25 nt, Artificial Sequence
  Description of Artificial Sequence Synthetic probe
gcggaggaacaugcagaggcugugc

SEQ ID NO: 429 - RNA, 25 nt, Artificial Sequence
  Description of Artificial Sequence Synthetic probe
gaggauaugguugacuuuauugaca

SEQ ID NO: 430 - RNA, 25 nt, Artificial Sequence
  Description of Artificial Sequence Synthetic probe
acgaaaaugaagacaguagugauac

SEQ ID NO: 431 - RNA, 25 nt, Artificial Sequence
  Description of Artificial Sequence Synthetic probe
cagacaggggacaacauuucagagg

SEQ ID NO: 432 - RNA, 25 nt, Artificial Sequence
  Description of Artificial Sequence Synthetic probe
gguugguuuuauguagaagcaguaa

SEQ ID NO: 433 - RNA, 25 nt, Artificial Sequence
  Description of Artificial Sequence Synthetic probe
agacuguaacuacaauggcugaucc

SEQ ID NO: 434 - RNA, 25 nt, Artificial Sequence
  Description of Artificial Sequence Synthetic probe
cucauuuggaaucgugugccccaac

SEQ ID NO: 435 - RNA, 25 nt, Artificial Sequence
  Description of Artificial Sequence Synthetic probe
gcauauugcaagagcuguuaauggg

SEQ ID NO: 436 - RNA, 25 nt, Artificial Sequence
  Description of Artificial Sequence Synthetic probe
guacagagcacacaaguagauauuc

SEQ ID NO: 437 - RNA, 25 nt, Artificial Sequence
  Description of Artificial Sequence Synthetic probe
cuuuuguugucaguguaagucuaca

SEQ ID NO: 438 - RNA, 25 nt, Artificial Sequence
  Description of Artificial Sequence Synthetic probe
ggacacauccaauuacaauaucguu

SEQ ID NO: 439 - RNA, 25 nt, Artificial Sequence
  Description of Artificial Sequence Synthetic probe
gaggaugucauagacaguccagcug

SEQ ID NO: 440 - RNA, 25 nt, Artificial Sequence
  Description of Artificial Sequence Synthetic probe
uguuaugagcaauuacccgacagcu

SEQ ID NO: 441 - RNA, 25 nt, Artificial Sequence
  Description of Artificial Sequence Synthetic probe
uguuagauuugcaaccugaggcaac

SEQ ID NO: 442 - RNA, 25 nt, Artificial Sequence
  Description of Artificial Sequence Synthetic probe
gaaacaccuacguugcaagacuaug

SEQ ID NO: 443 - RNA, 25 nt, Artificial Sequence
  Description of Artificial Sequence Synthetic probe
cucguacugaaacccaaguguaaac

SEQ ID NO: 444 - RNA, 25 nt, Artificial Sequence
  Description of Artificial Sequence Synthetic probe
cguugcauagcauguuggagaagac

SEQ ID NO: 445 - RNA, 25 nt, Artificial Sequence
  Description of Artificial Sequence Synthetic probe
gauuccacaacauaggaggaaggug

SEQ ID NO: 446 - RNA, 25 nt, Artificial Sequence
  Description of Artificial Sequence Synthetic probe
gguauagauauaguguguauggaac

SEQ ID NO: 447 - RNA, 25 nt, Artificial Sequence
  Description of Artificial Sequence Synthetic probe
cggaguguguacaaaauguuuaaga

SEQ ID NO: 448 - RNA, 25 nt, Artificial Sequence
  Description of Artificial Sequence Synthetic probe
uaguauauagggacgacacaccaca

SEQ ID NO: 449 - RNA, 25 nt, Artificial Sequence
  Description of Artificial Sequence Synthetic probe
cagaaacagagguauuagauuuugc

SEQ ID NO: 450 - RNA, 25 nt, Artificial Sequence
  Description of Artificial Sequence Synthetic probe
agauugaauugugucuacugcaaag

SEQ ID NO: 451 - RNA, 25 nt, Artificial Sequence
  Description of Artificial Sequence Synthetic probe
auuggaaauacccuacgaugaacua

SEQ ID NO: 452 - RNA, 25 nt, Artificial Sequence
  Description of Artificial Sequence Synthetic probe
ggaaauugcaugaacuaagcucggc

SEQ ID NO: 453 - RNA, 25 nt, Artificial Sequence
  Description of Artificial Sequence Synthetic probe
gugcaaaccuacagacgccauguuc

SEQ ID NO: 454 - RNA, 25 nt, Artificial Sequence
  Description of Artificial Sequence Synthetic probe
cgguugguauauaaagcacauagua

SEQ ID NO: 455 - RNA, 25 nt, Artificial Sequence
  Description of Artificial Sequence Synthetic probe
ccguuuuaggucauauuggucauuu

SEQ ID NO: 456 - RNA, 25 nt, Artificial Sequence
  Description of Artificial Sequence Synthetic probe
ugagucacuaccuguuuauuaccag

SEQ ID NO: 457 - RNA, 25 nt, Artificial Sequence
  Description of Artificial Sequence Synthetic probe
guaugccaaacuaugccuuguaaaa

SEQ ID NO: 458 - RNA, 25 nt, Artificial Sequence
  Description of Artificial Sequence Synthetic probe
caguuuuggcuuacacaauugcuuu

SEQ ID NO: 459 - RNA, 25 nt, Artificial Sequence
  Description of Artificial Sequence Synthetic probe
ucauauauacaugcagugcaauugc

SEQ ID NO: 460 - RNA, 25 nt, Artificial Sequence
  Description of Artificial Sequence Synthetic probe
guuugucuguacuugcugcauugac

SEQ ID NO: 461 - RNA, 25 nt, Artificial Sequence
  Description of Artificial Sequence Synthetic probe
uuaauccuuuucuuuccugcacugu

SEQ ID NO: 462 - RNA, 25 nt, Artificial Sequence
  Description of Artificial Sequence Synthetic probe
auacccuaugacauuggcagaacag

SEQ ID NO: 463 - RNA, 25 nt, Artificial Sequence
  Description of Artificial Sequence Synthetic probe
guuugucuguacuugcugcauuggc

SEQ ID NO: 464 - RNA, 25 nt, Artificial Sequence
  Description of Artificial Sequence Synthetic probe
uuaauccuuuucuuuccugcacugu

SEQ ID NO: 465 - RNA, 25 nt, Artificial Sequence
  Description of Artificial Sequence Synthetic probe
auacccuaugacauuggcagaacag

SEQ ID NO: 466 - RNA, 25 nt, Artificial Sequence
  Description of Artificial Sequence Synthetic probe
guccauauuguacaauuuccuccau

SEQ ID NO: 467 - RNA, 25 nt, Artificial Sequence
  Description of Artificial Sequence Synthetic probe
ccuacauguuuaguauugcuuuacc

SEQ ID NO: 468 - RNA, 25 nt, Artificial Sequence
  Description of Artificial Sequence Synthetic probe
caauguaccuaccuuuauuucccua

SEQ ID NO: 469 - RNA, 25 nt, Artificial Sequence
  Description of Artificial Sequence Synthetic probe
guauugcuugcccuacccugcauug

SEQ ID NO: 470 - RNA, 25 nt, Artificial Sequence
  Description of Artificial Sequence Synthetic probe
gguguaccuauaugaguaaggaguu

SEQ ID NO: 471 - RNA, 25 nt, Artificial Sequence
  Description of Artificial Sequence Synthetic probe
uguacuuguuugugugcauguucua

SEQ ID NO: 472 - RNA, 25 nt, Artificial Sequence
  Description of Artificial Sequence Synthetic probe
cugucuauguacuuuguguuguugu

SEQ ID NO: 473 - RNA, 25 nt, Artificial Sequence
  Description of Artificial Sequence Synthetic probe
cacccgcacaucgucugcaaaacgc

SEQ ID NO: 474 - RNA, 25 nt, Artificial Sequence
  Description of Artificial Sequence Synthetic probe
gcaaaaccuaaacuuaaacgugcag

SEQ ID NO: 475 - RNA, 25 nt, Artificial Sequence
  Description of Artificial Sequence Synthetic probe
gguaaauauacauuuugggaagugg

SEQ ID NO: 476 - RNA, 25 nt, Artificial Sequence
  Description of Artificial Sequence Synthetic probe
guuuaacaccuccuccaucugcuag

SEQ ID NO: 477 - RNA, 25 nt, Artificial Sequence
  Description of Artificial Sequence Synthetic probe
cacaaguaacuagugacaguacaua

SEQ ID NO: 478 - RNA, 25 nt, Artificial Sequence
  Description of Artificial Sequence Synthetic probe
gguuacuuccgaaucucaguuauuu

SEQ ID NO: 479 - RNA, 25 nt, Artificial Sequence
  Description of Artificial Sequence Synthetic probe
ggaacuacugccucuauucaaagca

SEQ ID NO: 480 - RNA, 25 nt, Artificial Sequence
  Description of Artificial Sequence Synthetic probe
uucccgaugaccuguacauuaaagg

SEQ ID NO: 481 - RNA, 25 nt, Artificial Sequence
  Description of Artificial Sequence Synthetic probe
agggcugguacauuaggagaggcug

SEQ ID NO: 482 - RNA, 25 nt, Artificial Sequence
  Description of Artificial Sequence Synthetic probe
cugccaaugauuguccaccuuuaga

SEQ ID NO: 483 - RNA, 25 nt, Artificial Sequence
  Description of Artificial Sequence Synthetic probe
agguguugcuuguacuaaugcagca

SEQ ID NO: 484 - RNA, 25 nt, Artificial Sequence
  Description of Artificial Sequence Synthetic probe
uauguuuacuuggauguaagccucc

SEQ ID NO: 485 - RNA, 25 nt, Artificial Sequence
  Description of Artificial Sequence Synthetic probe
uggacaaccgggugcugauaauagg

SEQ ID NO: 486 - RNA, 25 nt, Artificial Sequence
  Description of Artificial Sequence Synthetic probe
acacugaaaccgguaacaaguaucc

SEQ ID NO: 487 - RNA, 25 nt, Artificial Sequence
  Description of Artificial Sequence Synthetic probe
uguaggccuugaaauagguagaggg

SEQ ID NO: 488 - RNA, 25 nt, Artificial Sequence
  Description of Artificial Sequence Synthetic probe
uaaauuuggauuuccugacaccucc

SEQ ID NO: 489 - RNA, 25 nt, Artificial Sequence
  Description of Artificial Sequence Synthetic probe
cccaaaguaucaggcuugcaauaua

SEQ ID NO: 490 - RNA, 25 nt, Artificial Sequence
  Description of Artificial Sequence Synthetic probe
gcugacuuuguuuuacauccuaguu

SEQ ID NO: 491 - RNA, 25 nt, Artificial Sequence
  Description of Artificial Sequence Synthetic probe
uuuugacaccauuguuguagacggu

SEQ ID NO: 492 - RNA, 25 nt, Artificial Sequence
  Description of Artificial Sequence Synthetic probe
cuagcccauuuguuccuauuucgcc

SEQ ID NO: 493 - RNA, 25 nt, Artificial Sequence
  Description of Artificial Sequence Synthetic probe
uacuccuguuaugucuggcccugau

SEQ ID NO: 494 - RNA, 25 nt, Artificial Sequence
  Description of Artificial Sequence Synthetic probe
ccagcaaugugucuauaccuuuaaa

SEQ ID NO: 495 - RNA, 25 nt, Artificial Sequence
  Description of Artificial Sequence Synthetic probe
auacaguacguuugcaacaacacgu

SEQ ID NO: 496 - RNA, 25 nt, Artificial Sequence
  Description of Artificial Sequence Synthetic probe
auguacacaccccaaugcaacacuc

SEQ ID NO: 497 - RNA, 25 nt, Artificial Sequence
  Description of Artificial Sequence Synthetic probe
gauguuuaugcugacgauguggaua

SEQ ID NO: 498 - RNA, 25 nt, Artificial Sequence
  Description of Artificial Sequence Synthetic probe
cuuuacaugauacuucuacaucguc

SEQ ID NO: 499 - RNA, 25 nt, Artificial Sequence
  Description of Artificial Sequence Synthetic probe
ccgugccaaaugaacaauaugaauu

SEQ ID NO: 500 - RNA, 25 nt, Artificial Sequence
  Description of Artificial Sequence Synthetic probe
aguccuauugugccuuuagaccaca

SEQ ID NO: 501 - RNA, 25 nt, Artificial Sequence
  Description of Artificial Sequence Synthetic probe
ggagcuagaauacauuauuaucagg

SEQ ID NO: 502 - RNA, 25 nt, Artificial Sequence
  Description of Artificial Sequence Synthetic probe
cguagacauacugugcguuuuagua

SEQ ID NO: 503 - RNA, 25 nt, Artificial Sequence
  Description of Artificial Sequence Synthetic probe
ccugaagacacauuacaauuucaac

SEQ ID NO: 504 - RNA, 25 nt, Artificial Sequence
  Description of Artificial Sequence Synthetic probe
uuauauagucgcaauacccaacagg

SEQ ID NO: 505 - RNA, 25 nt, Artificial Sequence
  Description of Artificial Sequence Synthetic probe
uguaacaucaagcacgcccauucca

SEQ ID NO: 506 - RNA, 25 nt, Artificial Sequence
  Description of Artificial Sequence Synthetic probe
uuguuguuuccacagacaguaguaa

SEQ ID NO: 507 - RNA, 25 nt, Artificial Sequence
  Description of Artificial Sequence Synthetic probe
gcacacaaaguuaugaaaacauacc

SEQ ID NO: 508 - RNA, 25 nt, Artificial Sequence
  Description of Artificial Sequence Synthetic probe
cuggacauuuuauauuuucuucccc

SEQ ID NO: 509 - RNA, 25 nt, Artificial Sequence
  Description of Artificial Sequence Synthetic probe
uacacccuccagcgccugcagaagc

SEQ ID NO: 510 - RNA, 25 nt, Artificial Sequence
  Description of Artificial Sequence Synthetic probe
gggagucaucuauucaaacuauuuc

SEQ ID NO: 511 - RNA, 25 nt, Artificial Sequence
  Description of Artificial Sequence Synthetic probe
acuacaucugcagauacuacaccug

SEQ ID NO: 512 - RNA, 25 nt, Artificial Sequence
  Description of Artificial Sequence Synthetic probe
ccccaucuauuccuacaccaucagg

SEQ ID NO: 513 - RNA, 25 nt, Artificial Sequence
  Description of Artificial Sequence Synthetic probe
gacucgucuauagugucauuaauag

SEQ ID NO: 514 - RNA, 25 nt, Artificial Sequence
  Description of Artificial Sequence Synthetic probe
uacuguagacacuguuggaccuuua

SEQ ID NO: 515 - RNA, 25 nt, Artificial Sequence
  Description of Artificial Sequence Synthetic probe
ccuugcagccuauacguccuccggu

SEQ ID NO: 516 - RNA, 25 nt, Artificial Sequence
  Description of Artificial Sequence Synthetic probe
acugacccaccuacagcugcaaucc

SEQ ID NO: 517 - RNA, 25 nt, Artificial Sequence
  Description of Artificial Sequence Synthetic probe
aggaaguaccauagcagaucaaauu

SEQ ID NO: 518 - RNA, 25 nt, Artificial Sequence
  Description of Artificial Sequence Synthetic probe
cauggugguguuuuaacauuguugu

SEQ ID NO: 519 - RNA, 25 nt, Artificial Sequence
  Description of Artificial Sequence Synthetic probe
gcauaugacacaacaagaguaaugu

SEQ ID NO: 520 - RNA, 25 nt, Artificial Sequence
  Description of Artificial Sequence Synthetic probe
uuuggguguuugugggaucuccuuu

SEQ ID NO: 521 - RNA, 25 nt, Artificial Sequence
  Description of Artificial Sequence Synthetic probe
ugguugcugguguugguauugcugc

SEQ ID NO: 522 - RNA, 25 nt, Artificial Sequence
  Description of Artificial Sequence Synthetic probe
cuacugugcaaauaaguacuggauu

SEQ ID NO: 523 - RNA, 25 nt, Artificial Sequence
  Description of Artificial Sequence Synthetic probe
cauuuguaacugaacagcaacaaca

SEQ ID NO: 524 - RNA, 25 nt, Artificial Sequence
  Description of Artificial Sequence Synthetic probe
uauaguucuaugucauccaccuggc

SEQ ID NO: 525 - RNA, 25 nt, Artificial Sequence
  Description of Artificial Sequence Synthetic probe
uaguucuaacguugcaccuauagug

SEQ ID NO: 526 - RNA, 25 nt, Artificial Sequence
  Description of Artificial Sequence Synthetic probe
gcacaaacaagcagcggacugugug

SEQ ID NO: 527 - RNA, 25 nt, Artificial Sequence
  Description of Artificial Sequence Synthetic probe
uggacaauagaacagcacguacugc

SEQ ID NO: 528 - RNA, 25 nt, Artificial Sequence
  Description of Artificial Sequence Synthetic probe
ccccuuacaaagcuguucugugcag

SEQ ID NO: 529 - RNA, 25 nt, Artificial Sequence
  Description of Artificial Sequence Synthetic probe
accacaagcagcggccaaacgacga

SEQ ID NO: 530 - RNA, 25 nt, Artificial Sequence
  Description of Artificial Sequence Synthetic probe
acauacagacagacaacgauaaccg

SEQ ID NO: 531 - RNA, 25 nt, Artificial Sequence
  Description of Artificial Sequence Synthetic probe
cgucuauaucuagcaaccaaauauc

SEQ ID NO: 532 - RNA, 25 nt, Artificial Sequence
  Description of Artificial Sequence Synthetic probe
cuaugguuacagggaaaguagauua

SEQ ID NO: 533 - RNA, 25 nt, Artificial Sequence
  Description of Artificial Sequence Synthetic probe
ggauuauacaaacuggggugaaaua

SEQ ID NO: 534 - RNA, 25 nt, Artificial Sequence
  Description of Artificial Sequence Synthetic probe
aguaacugugcaauaugacaaugac

SEQ ID NO: 535 - RNA, 25 nt, Artificial Sequence
  Description of Artificial Sequence Synthetic probe
auaguacaagccaauggacauugca

SEQ ID NO: 536 - RNA, 25 nt, Artificial Sequence
  Description of Artificial Sequence Synthetic probe
caucaaagaccaaagcauuucaagu

SEQ ID NO: 537 - RNA, 25 nt, Artificial Sequence
  Description of Artificial Sequence Synthetic probe
gggauuuucacauuuaugccaccag

SEQ ID NO: 538 - RNA, 25 nt, Artificial Sequence
  Description of Artificial Sequence Synthetic probe
gugcuuuauuguauacagccaaaca

SEQ ID NO: 539 - RNA, 25 nt, Artificial Sequence
  Description of Artificial Sequence Synthetic probe
gcugauaaaacugauuuaccaucac

SEQ ID NO: 540 - RNA, 25 nt, Artificial Sequence
  Description of Artificial Sequence Synthetic probe
cccaguguaugcaauaaaugaugaa

SEQ ID NO: 541 - RNA, 25 nt, Artificial Sequence
  Description of Artificial Sequence Synthetic probe
cucuagauggccauauuuacauagu

SEQ ID NO: 542 - RNA, 25 nt, Artificial Sequence
  Description of Artificial Sequence Synthetic probe
uuaaaauguccaccacugcuucuua

SEQ ID NO: 543 - RNA, 25 nt, Artificial Sequence
  Description of Artificial Sequence Synthetic probe
gaugauuacaugagaaaugcguuag

SEQ ID NO: 544 - RNA, 25 nt, Artificial Sequence
  Description of Artificial Sequence Synthetic probe
uagaugauguaacgccaauaaguug

SEQ ID NO: 545 - RNA, 25 nt, Artificial Sequence
  Description of Artificial Sequence Synthetic probe
gcuguaugcuaauuuguggaccagc

SEQ ID NO: 546 - RNA, 25 nt, Artificial Sequence
  Description of Artificial Sequence Synthetic probe
gagaccaauaguacaguuguuaaga

SEQ ID NO: 547 - RNA, 25 nt, Artificial Sequence
  Description of Artificial Sequence Synthetic probe
gcagauucaaauaguaaugcugcug

SEQ ID NO: 548 - RNA, 25 nt, Artificial Sequence
  Description of Artificial Sequence Synthetic probe
augauaacgaguuaacggacgauag

SEQ ID NO: 549 - RNA, 25 nt, Artificial Sequence
  Description of Artificial Sequence Synthetic probe
ggagccaaacaugugcauuguauug

SEQ ID NO: 550 - RNA, 25 nt, Artificial Sequence
  Description of Artificial Sequence Synthetic probe
aacauguaugguuauagagccacca

SEQ ID NO: 551 - RNA, 25 nt, Artificial Sequence
  Description of Artificial Sequence Synthetic probe
cagguuaacaguagcaaaacuaaug

SEQ ID NO: 552 - RNA, 25 nt, Artificial Sequence
  Description of Artificial Sequence Synthetic probe
guauaacaggauauggaauuagucc

SEQ ID NO: 553 - RNA, 25 nt, Artificial Sequence
  Description of Artificial Sequence Synthetic probe
ggccuauggaauaaguuuuauggaa

SEQ ID NO: 554 - RNA, 25 nt, Artificial Sequence
  Description of Artificial Sequence Synthetic probe
cguugcaggaaauuaguaauguucu

SEQ ID NO: 555 - RNA, 25 nt, Artificial Sequence
  Description of Artificial Sequence Synthetic probe
gagacaaauguagauagcugugaaa

SEQ ID NO: 556 - RNA, 25 nt, Artificial Sequence
  Description of Artificial Sequence Synthetic probe
uaaaugacuuagaaucuaguggggu

SEQ ID NO: 557 - RNA, 25 nt, Artificial Sequence
  Description of Artificial Sequence Synthetic probe
gaaagucaaaauggcgacacaaacu

SEQ ID NO: 558 - RNA, 25 nt, Artificial Sequence
  Description of Artificial Sequence Synthetic probe
aacucagcagaugguacaacaggua

SEQ ID NO: 559 - RNA, 25 nt, Artificial Sequence
  Description of Artificial Sequence Synthetic probe
aucgugcugcaaacccguguagaac

SEQ ID NO: 560 - RNA, 25 nt, Artificial Sequence
  Description of Artificial Sequence Synthetic probe
uucacaaagugcugcggaggacguu

SEQ ID NO: 561 - RNA, 25 nt, Artificial Sequence
  Description of Artificial Sequence Synthetic probe
gcacggauuuacuagaguuuauaga

SEQ ID NO: 562 - RNA, 25 nt, Artificial Sequence
  Description of Artificial Sequence Synthetic probe
gaggaugaaacagcagaugacagug

SEQ ID NO: 563 - RNA, 25 nt, Artificial Sequence
  Description of Artificial Sequence Synthetic probe
ucaucuacaauggccgauccugaag

SEQ ID NO: 564 - RNA, 25 nt, Artificial Sequence
  Description of Artificial Sequence Synthetic probe
agugaauauugugugcccuaccugu

SEQ ID NO: 565 - RNA, 25 nt, Artificial Sequence
  Description of Artificial Sequence Synthetic probe
ccauacagcaacuacuuaugggcac

SEQ ID NO: 566 - RNA, 25 nt, Artificial Sequence
  Description of Artificial Sequence Synthetic probe
aacaguacagcaagugaccuacgaa

SEQ ID NO: 567 - RNA, 25 nt, Artificial Sequence
  Description of Artificial Sequence Synthetic probe
guugucacacuuguaacaccacagu

SEQ ID NO: 568 - RNA, 25 nt, Artificial Sequence
  Description of Artificial Sequence Synthetic probe
acagcugauuacuacauuguaaccu

SEQ ID NO: 569 - RNA, 25 nt, Artificial Sequence
  Description of Artificial Sequence Synthetic probe
auauccugaaccaacugaccuauac

SEQ ID NO: 570 - RNA, 25 nt, Artificial Sequence
  Description of Artificial Sequence Synthetic probe
gagaggacacaagccaacguuaaag

SEQ ID NO: 571 - RNA, 25 nt, Artificial Sequence
  Description of Artificial Sequence Synthetic probe
guagagaaacugcacugugacgugu

SEQ ID NO: 572 - RNA, 25 nt, Artificial Sequence
  Description of Artificial Sequence Synthetic probe
auuucgggucguugggcagggcgcu

SEQ ID NO: 573 - RNA, 25 nt, Artificial Sequence
  Description of Artificial Sequence Synthetic probe
cgacauguggauuuaaacaaacgau

SEQ ID NO: 574 - RNA, 25 nt, Artificial Sequence
  Description of Artificial Sequence Synthetic probe
ugucaaagaccuuuguguccucaag

SEQ ID NO: 575 - RNA, 25 nt, Artificial Sequence
  Description of Artificial Sequence Synthetic probe
cuguguuugcgguucuuaucuaaaa

SEQ ID NO: 576 - RNA, 25 nt, Artificial Sequence
  Description of Artificial Sequence Synthetic probe
gagggaaauccauuuggaauaugua

SEQ ID NO: 577 - RNA, 25 nt, Artificial Sequence
  Description of Artificial Sequence Synthetic probe
ccuuugcaacgaucugagguauaug

SEQ ID NO: 578 - RNA, 25 nt, Artificial Sequence
  Description of Artificial Sequence Synthetic probe
cauugaacuacagugcguggaaugc

SEQ ID NO: 579 - RNA, 25 nt, Artificial Sequence
  Description of Artificial Sequence Synthetic probe
acgacuauguuucaagacacugagg

SEQ ID NO: 580 - RNA, 25 nt, Artificial Sequence
  Description of Artificial Sequence Synthetic probe
cacauuguuauaugcacacaggugu

SEQ ID NO: 581 - RNA, 25 nt, Artificial Sequence
  Description of Artificial Sequence Synthetic probe
caugcauguaaaacauuacucacug

SEQ ID NO: 582 - RNA, 25 nt, Artificial Sequence
  Description of Artificial Sequence Synthetic probe
cacauccugccaacuuuaaguuaaa

SEQ ID NO: 583 - RNA, 25 nt, Artificial Sequence
  Description of Artificial Sequence Synthetic probe
cuaaagggcuuuaauugcacaccuu

SEQ ID NO: 584 - RNA, 25 nt, Artificial Sequence
  Description of Artificial Sequence Synthetic probe
aacacuuguaacagugcuuuuaggc

SEQ ID NO: 585 - RNA, 25 nt, Artificial Sequence
  Description of Artificial Sequence Synthetic probe
cuugauucaucuugcaguauuaguc

SEQ ID NO: 586 - RNA, 25 nt, Artificial Sequence
  Description of Artificial Sequence Synthetic probe
guguuccugauauauauuguuugcc

SEQ ID NO: 587 - RNA, 25 nt, Artificial Sequence
  Description of Artificial Sequence Synthetic probe
gguugcuguugguaagcuuuauaug

SEQ ID NO: 588 - RNA, 25 nt, Artificial Sequence
  Description of Artificial Sequence Synthetic probe
guguccuuuacauuaccuuucaacc

SEQ ID NO: 589 - RNA, 25 nt, Artificial Sequence
  Description of Artificial Sequence Synthetic probe
gagcuuacauaauuacaugacagcu

SEQ ID NO: 590 - RNA, 25 nt, Artificial Sequence
  Description of Artificial Sequence Synthetic probe
uuguaugacuauggugcaccgauau

SEQ ID NO: 591 - RNA, 25 nt, Artificial Sequence
  Description of Artificial Sequence Synthetic probe
gccauaaagugauguguguguuuau

SEQ ID NO: 592 - RNA, 25 nt, Artificial Sequence
  Description of Artificial Sequence Synthetic probe
guacuuaguguguaguaguucagua

SEQ ID NO: 593 - RNA, 25 nt, Artificial Sequence
  Description of Artificial Sequence Synthetic probe
uugugcaauguguuguacgugggug

SEQ ID NO: 594 - RNA, 25 nt, Artificial Sequence
  Description of Artificial Sequence Synthetic probe
cauggcguguaaauguguguauaau

SEQ ID NO: 595 - RNA, 25 nt, Artificial Sequence
  Description of Artificial Sequence Synthetic probe
uguuguggugccuguuuguguugua

SEQ ID NO: 596 - RNA, 25 nt, Artificial Sequence
  Description of Artificial Sequence Synthetic probe
cauguauacuguguguuauguguug

SEQ ID NO: 597 - RNA, 25 nt, Artificial Sequence
  Description of Artificial Sequence Synthetic probe
gcgugcagcuccagcaucuacaucu

SEQ ID NO: 598 - RNA, 25 nt, Artificial Sequence
  Description of Artificial Sequence Synthetic probe
caccaaaaccuaaagaugauccauu

SEQ ID NO: 599 - RNA, 25 nt, Artificial Sequence
  Description of Artificial Sequence Synthetic probe
acauaucgcuauguaacaucacagg

SEQ ID NO: 600 - RNA, 25 nt, Artificial Sequence
  Description of Artificial Sequence Synthetic probe
aacccguccauuuuagaggauugga

SEQ ID NO: 601 - RNA, 25 nt, Artificial Sequence
  Description of Artificial Sequence Synthetic probe
guacaaauaugucuguguguucugc

SEQ ID NO: 602 - RNA, 25 nt, Artificial Sequence
  Description of Artificial Sequence Synthetic probe
guaaccuccgaugcacaaauauuua

SEQ ID NO: 603 - RNA, 25 nt, Artificial Sequence
  Description of Artificial Sequence Synthetic probe
guacuaguuauuuuccuacuccuag

SEQ ID NO: 604 - RNA, 25 nt, Artificial Sequence
  Description of Artificial Sequence Synthetic probe
aguaccugcagaccuauauauuaag

SEQ ID NO: 605 - RNA, 25 nt, Artificial Sequence
  Description of Artificial Sequence Synthetic probe
uucugagccauauggagauauguua

SEQ ID NO: 606 - RNA, 25 nt, Artificial Sequence
  Description of Artificial Sequence Synthetic probe
ccuagauauaugcaguuccauuugc

SEQ ID NO: 607 - RNA, 25 nt, Artificial Sequence
  Description of Artificial Sequence Synthetic probe
ccuuuggaguuacuaaacacuguac

SEQ ID NO: 608 - RNA, 25 nt, Artificial Sequence
  Description of Artificial Sequence Synthetic probe
accagguaaaagcaggagaaugucc

SEQ ID NO: 609 - RNA, 25 nt, Artificial Sequence
  Description of Artificial Sequence Synthetic probe
caauuguguuuaauagguuguaggc

SEQ ID NO: 610 - RNA, 25 nt, Artificial Sequence
  Description of Artificial Sequence Synthetic probe
gauaacagggaaugcauuucuaugg

SEQ ID NO: 611 - RNA, 25 nt, Artificial Sequence
  Description of Artificial Sequence Synthetic probe
ugguaacucugguaacucugguaca

SEQ ID NO: 612 - RNA, 25 nt, Artificial Sequence
  Description of Artificial Sequence Synthetic probe
uguacaggaguugaaguaggucgug

SEQ ID NO: 613 - RNA, 25 nt, Artificial Sequence
  Description of Artificial Sequence Synthetic probe
ucccugccuccagcguuugguuugg

SEQ ID NO: 614 - RNA, 25 nt, Artificial Sequence
  Description of Artificial Sequence Synthetic probe
aaaauagcaguacccaagguaucug

SEQ ID NO: 615 - RNA, 25 nt, Artificial Sequence
  Description of Artificial Sequence Synthetic probe
gcaggcaguucuaggcuauuagcug

SEQ ID NO: 616 - RNA, 25 nt, Artificial Sequence
  Description of Artificial Sequence Synthetic probe
ucuaacgaagccacugucuaccugc

SEQ ID NO: 617 - RNA, 25 nt, Artificial Sequence
  Description of Artificial Sequence Synthetic probe
cccacagguccuauauauucuauua

SEQ ID NO: 618 - RNA, 25 nt, Artificial Sequence
  Description of Artificial Sequence Synthetic probe
uauuacuaacucuguacuaccggua

SEQ ID NO: 619 - RNA, 25 nt, Artificial Sequence
  Description of Artificial Sequence Synthetic probe
ggccagacauuguauuuaacucuaa

SEQ ID NO: 620 - RNA, 25 nt, Artificial Sequence
  Description of Artificial Sequence Synthetic probe
ggcuaugauauuccuauaacagcag

SEQ ID NO: 621 - RNA, 25 nt, Artificial Sequence
  Description of Artificial Sequence Synthetic probe
guuccuagcaauacuacuauaccau

SEQ ID NO: 622 - RNA, 25 nt, Artificial Sequence
  Description of Artificial Sequence Synthetic probe
cuccuauagauacugaggaagauau

SEQ ID NO: 623 - RNA, 25 nt, Artificial Sequence
  Description of Artificial Sequence Synthetic probe
cacauaccacuguuucaacaucauu

SEQ ID NO: 624 - RNA, 25 nt, Artificial Sequence
  Description of Artificial Sequence Synthetic probe
uuacaacauguaccauccucuuuac

SEQ ID NO: 625 - RNA, 25 nt, Artificial Sequence
  Description of Artificial Sequence Synthetic probe
aguggaaaagcuauaggggcacggg

SEQ ID NO: 626 - RNA, 25 nt, Artificial Sequence
  Description of Artificial Sequence Synthetic probe
guaauaaacguacuaugcauacacg

SEQ ID NO: 627 - RNA, 25 nt, Artificial Sequence
  Description of Artificial Sequence Synthetic probe
ugcacuaacaucuaggaaaggcacu

SEQ ID NO: 628 - RNA, 25 nt, Artificial Sequence
  Description of Artificial Sequence Synthetic probe
auggacauuauagcuuuacauaggc

SEQ ID NO: 629 - RNA, 25 nt, Artificial Sequence
  Description of Artificial Sequence Synthetic probe
ggauauuagcuuagcuccggauccu

SEQ ID NO: 630 - RNA, 25 nt, Artificial Sequence
  Description of Artificial Sequence Synthetic probe
gauacaaccuuacaauuugagcaug

SEQ ID NO: 631 - RNA, 25 nt, Artificial Sequence
  Description of Artificial Sequence Synthetic probe
gacuucuccugcaaaacuuauuaca

SEQ ID NO: 632 - RNA, 25 nt, Artificial Sequence
  Description of Artificial Sequence Synthetic probe
gauuauauaguaaagguacccagca

SEQ ID NO: 633 - RNA, 25 nt, Artificial Sequence
  Description of Artificial Sequence Synthetic probe
gcaauaauauaacuaauagcacgcc

SEQ ID NO: 634 - RNA, 25 nt, Artificial Sequence
  Description of Artificial Sequence Synthetic probe
caggucauuuuguacuuucaucauc

SEQ ID NO: 635 - RNA, 25 nt, Artificial Sequence
  Description of Artificial Sequence Synthetic probe
cacccacccacgccugcagaaacuu

SEQ ID NO: 636 - RNA, 25 nt, Artificial Sequence
  Description of Artificial Sequence Synthetic probe
gugacauccauaaguacacaugaua

SEQ ID NO: 637 - RNA, 25 nt, Artificial Sequence
  Description of Artificial Sequence Synthetic probe
cuacagauaccacaccugcuauuuu

SEQ ID NO: 638 - RNA, 25 nt, Artificial Sequence
  Description of Artificial Sequence Synthetic probe
cuacaacagguuuuacaauaaccac

SEQ ID NO: 639 - RNA, 25 nt, Artificial Sequence
  Description of Artificial Sequence Synthetic probe
ccuguuguuacaccaagggucccac

SEQ ID NO: 640 - RNA, 25 nt, Artificial Sequence
  Description of Artificial Sequence Synthetic probe
cuauagugucauuaguagaggaaac

SEQ ID NO: 641 - RNA, 25 nt, Artificial Sequence
  Description of Artificial Sequence Synthetic probe
gacacaauuggcccuuuagauucuu

SEQ ID NO: 642 - RNA, 25 nt, Artificial Sequence
  Description of Artificial Sequence Synthetic probe
ggcugccacaaacauuccuauacga

SEQ ID NO: 643 - RNA, 25 nt, Artificial Sequence
  Description of Artificial Sequence Synthetic probe
uuccacuggguacaacaccuccaac

SEQ ID NO: 644 - RNA, 25 nt, Artificial Sequence
  Description of Artificial Sequence Synthetic probe
ggcacagguggaagaucuggauaug

SEQ ID NO: 645 - RNA, 25 nt, Artificial Sequence
  Description of Artificial Sequence Synthetic probe
aacuauaucguacuugcaaagcugc

SEQ ID NO: 646 - RNA, 25 nt, Artificial Sequence
  Description of Artificial Sequence Synthetic probe
cacaaaaggucuacaaaacguguua

SEQ ID NO: 647 - RNA, 25 nt, Artificial Sequence
  Description of Artificial Sequence Synthetic probe
guaacauguguguauggugguuuua

SEQ ID NO: 648 - RNA, 25 nt, Artificial Sequence
  Description of Artificial Sequence Synthetic probe
ggcaguacaguaauuguauacaaac

SEQ ID NO: 649 - RNA, 25 nt, Artificial Sequence
  Description of Artificial Sequence Synthetic probe
gaugauuaacgcucaugcacaauau

SEQ ID NO: 650 - RNA, 25 nt, Artificial Sequence
  Description of Artificial Sequence Synthetic probe
cuacuugcuuuuguuguuucuugcu

SEQ ID NO: 651 - RNA, 25 nt, Artificial Sequence
  Description of Artificial Sequence Synthetic probe
acuguggguuacuguagcaacacca

SEQ ID NO: 652 - RNA, 25 nt, Artificial Sequence
  Description of Artificial Sequence Synthetic probe
cuaucugugucauuauacucagcau

SEQ ID NO: 653 - RNA, 25 nt, Artificial Sequence
  Description of Artificial Sequence Synthetic probe
gugucugcuuguacguucgcuauug

SEQ ID NO: 654 - RNA, 25 nt, Artificial Sequence
  Description of Artificial Sequence Synthetic probe
ugugcuuuugugugcuuuugugcuu

SEQ ID NO: 655 - RNA, 25 nt, Artificial Sequence
  Description of Artificial Sequence Synthetic probe
agcuuccaguacuguguugcugugc

SEQ ID NO: 656 - RNA, 25 nt, Artificial Sequence
  Description of Artificial Sequence Synthetic probe
cacaguuacagugucuaaaggauau

SEQ ID NO: 657 - RNA, 25 nt, Artificial Sequence
  Description of Artificial Sequence Synthetic probe
cuuacacaacagaauaucaaaggga

SEQ ID NO: 658 - RNA, 25 nt, Artificial Sequence
  Description of Artificial Sequence Synthetic probe
auggagauggacauguacaaacgau

SEQ ID NO: 659 - RNA, 25 nt, Artificial Sequence
  Description of Artificial Sequence Synthetic probe
acugcacaaacaaagaccggugugg

SEQ ID NO: 660 - RNA, 25 nt, Artificial Sequence
  Description of Artificial Sequence Synthetic probe
caguguugacagaggggucuacucu

SEQ ID NO: 661 - RNA, 25 nt, Artificial Sequence
  Description of Artificial Sequence Synthetic probe
agcgagugcgacucagugccgugga

SEQ ID NO: 662 - RNA, 25 nt, Artificial Sequence
  Description of Artificial Sequence Synthetic probe
accgagcuccccuacaaccccacca

SEQ ID NO: 663 - RNA, 25 nt, Artificial Sequence
  Description of Artificial Sequence Synthetic probe
agaagacaaaucacaaacgacuucg

SEQ ID NO: 664 - RNA, 25 nt, Artificial Sequence
  Description of Artificial Sequence Synthetic probe
cccauaccaaagccugcuccguggg

SEQ ID NO: 665 - RNA, 25 nt, Artificial Sequence
  Description of Artificial Sequence Synthetic probe
uuuagcagcacagaacuauccacug

SEQ ID NO: 666 - RNA, 25 nt, Artificial Sequence
  Description of Artificial Sequence Synthetic probe
uuauguuacuuuuagggaagaggcu

SEQ ID NO: 667 - RNA, 25 nt, Artificial Sequence
  Description of Artificial Sequence Synthetic probe
augugcaucaggguguagaaacaua

SEQ ID NO: 668 - RNA, 25 nt, Artificial Sequence
  Description of Artificial Sequence Synthetic probe
guauauguacuguuguaaagggacu

SEQ ID NO: 669 - RNA, 25 nt, Artificial Sequence
  Description of Artificial Sequence Synthetic probe
gaagcacaauuugauggugauaaac

SEQ ID NO: 670 - RNA, 25 nt, Artificial Sequence
  Description of Artificial Sequence Synthetic probe
caacugaguauagcacagaggacug

SEQ ID NO: 671 - RNA, 25 nt, Artificial Sequence
  Description of Artificial Sequence Synthetic probe
aaaagccaaagcaaugcaagcaauu

SEQ ID NO: 672 - RNA, 25 nt, Artificial Sequence
  Description of Artificial Sequence Synthetic probe
aagugguuccaacgcaggccauuuc

SEQ ID NO: 673 - RNA, 25 nt, Artificial Sequence
  Description of Artificial Sequence Synthetic probe
augggaauuaaaacucuuaaccacc

SEQ ID NO: 674 - RNA, 25 nt, Artificial Sequence
  Description of Artificial Sequence Synthetic probe
guauuggaaacugauucgucuugaa

SEQ ID NO: 675 - RNA, 25 nt, Artificial Sequence
  Description of Artificial Sequence Synthetic probe
gcacauguuugucugaucacauaca

SEQ ID NO: 676 - RNA, 25 nt, Artificial Sequence
  Description of Artificial Sequence Synthetic probe
agaggucaaagaaaaugauggagac

SEQ ID NO: 677 - RNA, 25 nt, Artificial Sequence
  Description of Artificial Sequence Synthetic probe
ggacguggugcagauuaaauuugca

SEQ ID NO: 678 - RNA, 25 nt, Artificial Sequence
  Description of Artificial Sequence Synthetic probe
guaguggucuuuacauuucacaaug

SEQ ID NO: 679 - RNA, 25 nt, Artificial Sequence
  Description of Artificial Sequence Synthetic probe
cagguggccauacuuacauagcagg

SEQ ID NO: 680 - RNA, 25 nt, Artificial Sequence
  Description of Artificial Sequence Synthetic probe
ccauguggcauauauagaccaauau

SEQ ID NO: 681 - RNA, 25 nt, Artificial Sequence
  Description of Artificial Sequence Synthetic probe
cagccauuauaugaugccaaaauag

SEQ ID NO: 682 - RNA, 25 nt, Artificial Sequence
  Description of Artificial Sequence Synthetic probe
cuaaugcauuucuuacaaggagcua

SEQ ID NO: 683 - RNA, 25 nt, Artificial Sequence
  Description of Artificial Sequence Synthetic probe
uugcauacuaauauauggagcacca

SEQ ID NO: 684 - RNA, 25 nt, Artificial Sequence
  Description of Artificial Sequence Synthetic probe
gauaucaacaaguagauuuuguggc

SEQ ID NO: 685 - RNA, 25 nt, Artificial Sequence
  Description of Artificial Sequence Synthetic probe
cacaguggauuaaaaggcgaugugc

SEQ ID NO: 686 - RNA, 25 nt, Artificial Sequence
  Description of Artificial Sequence Synthetic probe
cagaaacuaauaguaaugcaugugc

SEQ ID NO: 687 - RNA, 25 nt, Artificial Sequence
  Description of Artificial Sequence Synthetic probe
uauuagugagguugauggagaaaca

SEQ ID NO: 688 - RNA, 25 nt, Artificial Sequence
  Description of Artificial Sequence Synthetic probe
cguaguaccccagcugcguuauauu

SEQ ID NO: 689 - RNA, 25 nt, Artificial Sequence
  Description of Artificial Sequence Synthetic probe
gcuauguauuucagcugcaaguaug

SEQ ID NO: 690 - RNA, 25 nt, Artificial Sequence
  Description of Artificial Sequence Synthetic probe
gggcuaugguaauucuagcauuauu

SEQ ID NO: 691 - RNA, 25 nt, Artificial Sequence
  Description of Artificial Sequence Synthetic probe
guguggcgaacuuuaaacauauaac

SEQ ID NO: 692 - RNA, 25 nt, Artificial Sequence
  Description of Artificial Sequence Synthetic probe
guggccgcauuuggaauagccccaa

SEQ ID NO: 693 - RNA, 25 nt, Artificial Sequence
  Description of Artificial Sequence Synthetic probe
caacgcgagacauaauacaaauacu

SEQ ID NO: 694 - RNA, 25 nt, Artificial Sequence
  Description of Artificial Sequence Synthetic probe
agcgaugaaagacaugaugagacuc

SEQ ID NO: 695 - RNA, 25 nt, Artificial Sequence
  Description of Artificial Sequence Synthetic probe
caguggggauaguauaaccucuagu

SEQ ID NO: 696 - RNA, 25 nt, Artificial Sequence
  Description of Artificial Sequence Synthetic probe
auacaguugaacaauguaguauggg

SEQ ID NO: 697 - RNA, 25 nt, Artificial Sequence
  Description of Artificial Sequence Synthetic probe
uacacgagauacaacagguagaggg

SEQ ID NO: 698 - RNA, 25 nt, Artificial Sequence
  Description of Artificial Sequence Synthetic probe
cgauuauuugaacuaccagacagcg

SEQ ID NO: 699 - RNA, 25 nt, Artificial Sequence
  Description of Artificial Sequence Synthetic probe
cuaguaguccacuuagcagcgugag

SEQ ID NO: 700 - RNA, 25 nt, Artificial Sequence
  Description of Artificial Sequence Synthetic probe
caaagaggcuguacagguccuaaaa

SEQ ID NO: 701 - RNA, 25 nt, Artificial Sequence
  Description of Artificial Sequence Synthetic probe
gaaacagagacagcacaagcauuau

SEQ ID NO: 702 - RNA, 25 nt, Artificial Sequence
  Description of Artificial Sequence Synthetic probe
gacgaaaaugaagaugacugugaca

SEQ ID NO: 703 - RNA, 25 nt, Artificial Sequence
  Description of Artificial Sequence Synthetic probe
uagacguacgggauccaguguagag

SEQ ID NO: 704 - RNA, 25 nt, Artificial Sequence
  Description of Artificial Sequence Synthetic probe
auaaucuacaauggcugauccugca

SEQ ID NO: 705 - RNA, 25 nt, Artificial Sequence
  Description of Artificial Sequence Synthetic probe
aauagugugccccggcuguucacag

SEQ ID NO: 706 - RNA, 25 nt, Artificial Sequence
  Description of Artificial Sequence Synthetic probe
cacauugacauacguaaauuggaag

SEQ ID NO: 707 - RNA, 25 nt, Artificial Sequence
  Description of Artificial Sequence Synthetic probe
uguaaaugugaggcgacacuacguc

SEQ ID NO: 708 - RNA, 25 nt, Artificial Sequence
  Description of Artificial Sequence Synthetic probe
ccagacaccuccaauuauaauauug

SEQ ID NO: 709 - RNA, 25 nt, Artificial Sequence
  Description of Artificial Sequence Synthetic probe
agauacuauugacgguccagcugga

SEQ ID NO: 710 - RNA, 25 nt, Artificial Sequence
  Description of Artificial Sequence Synthetic probe
uauguuuuagauuuggaacccgagg

SEQ ID NO: 711 - RNA, 25 nt, Artificial Sequence
  Description of Artificial Sequence Synthetic probe
guguaaucaugcauggagaaauaac

SEQ ID NO: 712 - RNA, 25 nt, Artificial Sequence
  Description of Artificial Sequence Synthetic probe
gaaaccaacacguagagaaaccgag

SEQ ID NO: 713 - RNA, 25 nt, Artificial Sequence
  Description of Artificial Sequence Synthetic probe
ccaguugaaaagcaaagacauuuag

SEQ ID NO: 714 - RNA, 25 nt, Artificial Sequence
  Description of Artificial Sequence Synthetic probe
uauaguguguauggagaaacguuag

SEQ ID NO: 715 - RNA, 25 nt, Artificial Sequence
  Description of Artificial Sequence Synthetic probe
ccauauggaguaugcaugaaauguu

SEQ ID NO: 716 - RNA, 25 nt, Artificial Sequence
  Description of Artificial Sequence Synthetic probe
guguauaguauauagagaaggccag

SEQ ID NO: 717 - RNA, 25 nt, Artificial Sequence
  Description of Artificial Sequence Synthetic probe
gguauaugacuuugcaugcuaugau

SEQ ID NO: 718 - RNA, 25 nt, Artificial Sequence
  Description of Artificial Sequence Synthetic probe
gcaaacaagaauuacagcggaguga

SEQ ID NO: 719 - RNA, 25 nt, Artificial Sequence
  Description of Artificial Sequence Synthetic probe
gguagaagaaagcauccaugaaauu

SEQ ID NO: 720 - RNA, 25 nt, Artificial Sequence
  Description of Artificial Sequence Synthetic probe
cgaccuuacaaacugcaugauuugu

SEQ ID NO: 721 - RNA, 25 nt, Artificial Sequence
  Description of Artificial Sequence Synthetic probe
cgguauguuucaggacccagcugaa

SEQ ID NO: 722 - RNA, 25 nt, Artificial Sequence
  Description of Artificial Sequence Synthetic probe
acgguugccauaaaagcagaagugc

SEQ ID NO: 723 - RNA, 25 nt, Artificial Sequence
  Description of Artificial Sequence Synthetic probe
cacacaauaguuuaugcaaccgaaa

SEQ ID NO: 724 - RNA, 25 nt, Artificial Sequence
  Description of Artificial Sequence Synthetic probe
caggaaugugucuuacaguauaagu

SEQ ID NO: 725 - RNA, 25 nt, Artificial Sequence
  Description of Artificial Sequence Synthetic probe
cuugcuuaauuaaauaguuggccug

SEQ ID NO: 726 - RNA, 25 nt, Artificial Sequence
  Description of Artificial Sequence Synthetic probe
ccacccuauguaauaaaacugcuuu

SEQ ID NO: 727 - RNA, 25 nt, Artificial Sequence
  Description of Artificial Sequence Synthetic probe
caauacuuuggcaacauccauaucu

SEQ ID NO: 728 - RNA, 25 nt, Artificial Sequence
  Description of Artificial Sequence Synthetic probe
ccuuauuacucaucauccuguccag

SEQ ID NO: 729 - RNA, 25 nt, Artificial Sequence
  Description of Artificial Sequence Synthetic probe
uucacccugcauaguuggcacuggu

SEQ ID NO: 730 - RNA, 25 nt, Artificial Sequence
  Description of Artificial Sequence Synthetic probe
cauuuuauacuucgccauuuugugg

SEQ ID NO: 731 - RNA, 25 nt, Artificial Sequence
  Description of Artificial Sequence Synthetic probe
ucauacauaaucuauaugcccuacc

SEQ ID NO: 732 - RNA, 25 nt, Artificial Sequence
  Description of Artificial Sequence Synthetic probe
augacaguuucaugugugauugcac

SEQ ID NO: 733 - RNA, 25 nt, Artificial Sequence
  Description of Artificial Sequence Synthetic probe
ccuuauguguugaguguauaugugu

SEQ ID NO: 734 - RNA, 25 nt, Artificial Sequence
  Description of Artificial Sequence Synthetic probe
ccuuguuauguguguguauguuguu

SEQ ID NO: 735 - RNA, 25 nt, Artificial Sequence
  Description of Artificial Sequence Synthetic probe
cgugugucuaaauaaugcaugugua

SEQ ID NO: 736 - RNA, 25 nt, Artificial Sequence
  Description of Artificial Sequence Synthetic probe
cuuccucguccucagcuacuaaaca

SEQ ID NO: 737 - RNA, 25 nt, Artificial Sequence
  Description of Artificial Sequence Synthetic probe
gcccuacuauagguccccgaaagcg

SEQ ID NO: 738 - RNA, 25 nt, Artificial Sequence
  Description of Artificial Sequence Synthetic probe
uggaacuugaucaauucccuuuggg

SEQ ID NO: 739 - RNA, 25 nt, Artificial Sequence
  Description of Artificial Sequence Synthetic probe
agauccauaugacggucuaaaguuu

SEQ ID NO: 740 - RNA, 25 nt, Artificial Sequence
  Description of Artificial Sequence Synthetic probe
ccuacagucugcagccauuacaugu

SEQ ID NO: 741 - RNA, 25 nt, Artificial Sequence
  Description of Artificial Sequence Synthetic probe
ccaguuugguagacacuuacagaua

SEQ ID NO: 742 - RNA, 25 nt, Artificial Sequence
  Description of Artificial Sequence Synthetic probe
uuuugcuguagcuccuccaccaucu

SEQ ID NO: 743 - RNA, 25 nt, Artificial Sequence
  Description of Artificial Sequence Synthetic probe
gaauuccucuauauuggacaauugg

SEQ ID NO: 744 - RNA, 25 nt, Artificial Sequence
  Description of Artificial Sequence Synthetic probe
ccuucuacauaugauccuucuaagu

SEQ ID NO: 745 - RNA, 25 nt, Artificial Sequence
  Description of Artificial Sequence Synthetic probe
aucuaccucuauagagucuuccaua

SEQ ID NO: 746 - RNA, 25 nt, Artificial Sequence
  Description of Artificial Sequence Synthetic probe
acugccccucucccagcgguuccau

SEQ ID NO: 747 - RNA, 25 nt, Artificial Sequence
  Description of Artificial Sequence Synthetic probe
cgugcaaaccccgguaguucuguau

SEQ ID NO: 748 - RNA, 25 nt, Artificial Sequence
  Description of Artificial Sequence Synthetic probe
ccaauuguauauuaagggcacagau

SEQ ID NO: 749 - RNA, 25 nt, Artificial Sequence
  Description of Artificial Sequence Synthetic probe
acaguauguucuucuguuuacguag

SEQ ID NO: 750 - RNA, 25 nt, Artificial Sequence
  Description of Artificial Sequence Synthetic probe
gaacuaguaaacaccccuauugagg

SEQ ID NO: 751 - RNA, 25 nt, Artificial Sequence
  Description of Artificial Sequence Synthetic probe
caugcaagcccaauaauguaucuac

SEQ ID NO: 752 - RNA, 25 nt, Artificial Sequence
  Description of Artificial Sequence Synthetic probe
ccauuuucaucaaccaccaauaagg

SEQ ID NO: 753 - RNA, 25 nt, Artificial Sequence
  Description of Artificial Sequence Synthetic probe
gacacccauuauauaauagacagga

SEQ ID NO: 754 - RNA, 25 nt, Artificial Sequence
  Description of Artificial Sequence Synthetic probe
ccuuauauaauccagaaacacaacg

SEQ ID NO: 755 - RNA, 25 nt, Artificial Sequence
  Description of Artificial Sequence Synthetic probe
ccgauccuaauaaauucaguauucc

SEQ ID NO: 756 - RNA, 25 nt, Artificial Sequence
  Description of Artificial Sequence Synthetic probe
uauaggguauuucgcgugacauugc

SEQ ID NO: 757 - RNA, 25 nt, Artificial Sequence
  Description of Artificial Sequence Synthetic probe
uaaaguggguaugaaugguggucgc

SEQ ID NO: 758 - RNA, 25 nt, Artificial Sequence
  Description of Artificial Sequence Synthetic probe
gcucuagauuauuaacaguaggaca

SEQ ID NO: 759 - RNA, 25 nt, Artificial Sequence
  Description of Artificial Sequence Synthetic probe
acaacauaugcaauaaccauucagg

SEQ ID NO: 760 - RNA, 25 nt, Artificial Sequence
  Description of Artificial Sequence Synthetic probe
guugccauuggugccuucuggacca

SEQ ID NO: 761 - RNA, 25 nt, Artificial Sequence
  Description of Artificial Sequence Synthetic probe
uugcuuuaccaaguacuacuccaca

SEQ ID NO: 762 - RNA, 25 nt, Artificial Sequence
  Description of Artificial Sequence Synthetic probe
augccuguaaauacugguccugaua

SEQ ID NO: 763 - RNA, 25 nt, Artificial Sequence
  Description of Artificial Sequence Synthetic probe
cuauuccuuuuaguaccucauggaa

SEQ ID NO: 764 - RNA, 25 nt, Artificial Sequence
  Description of Artificial Sequence Synthetic probe
cagcaucuacuaaauaugccaauac

SEQ ID NO: 765 - RNA, 25 nt, Artificial Sequence
  Description of Artificial Sequence Synthetic probe
ggcucacuaccuucuguggcuucuu

SEQ ID NO: 766 - RNA, 25 nt, Artificial Sequence
  Description of Artificial Sequence Synthetic probe
ggauucgggcacuacauauaacaca

SEQ ID NO: 767 - RNA, 25 nt, Artificial Sequence
  Description of Artificial Sequence Synthetic probe
auaugcugauguggacaauaacaca

SEQ ID NO: 768 - RNA, 25 nt, Artificial Sequence
  Description of Artificial Sequence Synthetic probe
cacgcugagcccucugaugcuucag

SEQ ID NO: 769 - RNA, 25 nt, Artificial Sequence
  Description of Artificial Sequence Synthetic probe
aagcauugaauuacagccccuaguu

SEQ ID NO: 770 - RNA, 25 nt, Artificial Sequence
  Description of Artificial Sequence Synthetic probe
ccaugacauuaguaguauugcuccu

SEQ ID NO: 771 - RNA, 25 nt, Artificial Sequence
  Description of Artificial Sequence Synthetic probe
gcacacaaauuggagcgcaaguaca

SEQ ID NO: 772 - RNA, 25 nt, Artificial Sequence
  Description of Artificial Sequence Synthetic probe
aaggaacaguaagguuuaguaggcu

SEQ ID NO: 773 - RNA, 25 nt, Artificial Sequence
  Description of Artificial Sequence Synthetic probe
gagccuguugauacuacauuaacau

SEQ ID NO: 774 - RNA, 25 nt, Artificial Sequence
  Description of Artificial Sequence Synthetic probe
uauaguagagcacaucagcagguuc

SEQ ID NO: 775 - RNA, 25 nt, Artificial Sequence
  Description of Artificial Sequence Synthetic probe
ccuacaccuggaaucagucgugugg

SEQ ID NO: 776 - RNA, 25 nt, Artificial Sequence
  Description of Artificial Sequence Synthetic probe
ucggguaauauauuugucaguaccc

SEQ ID NO: 777 - RNA, 25 nt, Artificial Sequence
  Description of Artificial Sequence Synthetic probe
acggauccuuccuuaauugagguuc

SEQ ID NO: 778 - RNA, 25 nt, Artificial Sequence
  Description of Artificial Sequence Synthetic probe
accucuacuaguuauacuaacccug

SEQ ID NO: 779 - RNA, 25 nt, Artificial Sequence
  Description of Artificial Sequence Synthetic probe
caccucuggauuugaaauuacuucu

SEQ ID NO: 780 - RNA, 25 nt, Artificial Sequence
  Description of Artificial Sequence Synthetic probe
gaacaccaguaccaacauuuacagg

SEQ ID NO: 781 - RNA, 25 nt, Artificial Sequence
  Description of Artificial Sequence Synthetic probe
gaggacucaaguguuauaaccucug

SEQ ID NO: 782 - RNA, 25 nt, Artificial Sequence
  Description of Artificial Sequence Synthetic probe
ugagccaucuauugugcaauuggug

SEQ ID NO: 783 - RNA, 25 nt, Artificial Sequence
  Description of Artificial Sequence Synthetic probe
acuguuguagaugugucuccugcac

SEQ ID NO: 784 - RNA, 25 nt, Artificial Sequence
  Description of Artificial Sequence Synthetic probe
gguacuacacuugcugacaaaauuu

SEQ ID NO: 785 - RNA, 25 nt, Artificial Sequence
  Description of Artificial Sequence Synthetic probe
accagacguuguugauaaaguugag

SEQ ID NO: 786 - RNA, 25 nt, Artificial Sequence
  Description of Artificial Sequence Synthetic probe
ccuauauagaaccuguaaacaaucg

SEQ ID NO: 787 - RNA, 25 nt, Artificial Sequence
  Description of Artificial Sequence Synthetic probe
uacuaauaaacaugguuucccaccg

SEQ ID NO: 788 - RNA, 25 nt, Artificial Sequence
  Description of Artificial Sequence Synthetic probe
auugugcauaacuacuguacauagc

SEQ ID NO: 789 - RNA, 25 nt, Artificial Sequence
  Description of Artificial Sequence Synthetic probe
ggcaauggauaugauauaguacugu

SEQ ID NO: 790 - RNA, 25 nt, Artificial Sequence
  Description of Artificial Sequence Synthetic probe
ugcccaugugguuguugcauagacu

SEQ ID NO: 791 - RNA, 25 nt, Artificial Sequence
  Description of Artificial Sequence Synthetic probe
cguauguguggauaauuguguuugu

SEQ ID NO: 792 - RNA, 25 nt, Artificial Sequence
  Description of Artificial Sequence Synthetic probe
cauuggguuacaugacauuguaaag

SEQ ID NO: 793 - RNA, 25 nt, Artificial Sequence
  Description of Artificial Sequence Synthetic probe
gacacuguuaaaauaccuucuagug

SEQ ID NO: 794 - RNA, 25 nt, Artificial Sequence
  Description of Artificial Sequence Synthetic probe
acauaugccacagagucacaacgcc

SEQ ID NO: 795 - RNA, 25 nt, Artificial Sequence
  Description of Artificial Sequence Synthetic probe
agacgguaccucaguugugguaaca

SEQ ID NO: 796 - RNA, 25 nt, Artificial Sequence
  Description of Artificial Sequence Synthetic probe
caguaacaguacaggccacaacaca

SEQ ID NO: 797 - RNA, 25 nt, Artificial Sequence
  Description of Artificial Sequence Synthetic probe
uggaccaucuuaacaacccacucca

SEQ ID NO: 798 - RNA, 25 nt, Artificial Sequence
  Description of Artificial Sequence Synthetic probe
agucacagagcccacugagcccgac

SEQ ID NO: 799 - RNA, 25 nt, Artificial Sequence
  Description of Artificial Sequence Synthetic probe
gaauuaucaaacaccaccgcgaccc

SEQ ID NO: 800 - RNA, 25 nt, Artificial Sequence
  Description of Artificial Sequence Synthetic probe
acggaucgguacccacuacugaacu

SEQ ID NO: 801 - RNA, 25 nt, Artificial Sequence
  Description of Artificial Sequence Synthetic probe
uauucaagaugcggaaagguauggg

SEQ ID NO: 802 - RNA, 25 nt, Artificial Sequence
  Description of Artificial Sequence Synthetic probe
gcaccuaaaaguauacuaugaagug

SEQ ID NO: 803 - RNA, 25 nt, Artificial Sequence
  Description of Artificial Sequence Synthetic probe
gaacuauguauuauggggugcuaua

SEQ ID NO: 804 - RNA, 25 nt, Artificial Sequence
  Description of Artificial Sequence Synthetic probe
augauggggacaaauguaaugcuau

SEQ ID NO: 805 - RNA, 25 nt, Artificial Sequence
  Description of Artificial Sequence Synthetic probe
ugaauacaauacagaggaguggaca

SEQ ID NO: 806 - RNA, 25 nt, Artificial Sequence
  Description of Artificial Sequence Synthetic probe
ggugccaaccauaaacauuucaaaa

SEQ ID NO: 807 - RNA, 25 nt, Artificial Sequence
  Description of Artificial Sequence Synthetic probe
acgauagguggccauauuuacguag

SEQ ID NO: 808 - RNA, 25 nt, Artificial Sequence
  Description of Artificial Sequence Synthetic probe
ggguaugcaauaaguuuagauagga

SEQ ID NO: 809 - RNA, 25 nt, Artificial Sequence
  Description of Artificial Sequence Synthetic probe
uuagaugaugcaaccgguaccugcu

SEQ ID NO: 810 - RNA, 25 nt, Artificial Sequence
  Description of Artificial Sequence Synthetic probe
uauuucauauguaaacuccaccagc

SEQ ID NO: 811 - RNA, 25 nt, Artificial Sequence
  Description of Artificial Sequence Synthetic probe
guuauauauggaccugcgaauacag

SEQ ID NO: 812 - RNA, 25 nt, Artificial Sequence
  Description of Artificial Sequence Synthetic probe
gagacccauaguacaauucuuaaga

SEQ ID NO: 813 - RNA, 25 nt, Artificial Sequence
  Description of Artificial Sequence Synthetic probe
guguaguaaaugugaugaaggcggg

SEQ ID NO: 814 - RNA, 25 nt, Artificial Sequence
  Description of Artificial Sequence Synthetic probe
gcaauguuagcagauuguaacagua

SEQ ID NO: 815 - RNA, 25 nt, Artificial Sequence
  Description of Artificial Sequence Synthetic probe
uaguguauuugaccuaucggacaug

SEQ ID NO: 816 - RNA, 25 nt, Artificial Sequence
  Description of Artificial Sequence Synthetic probe
agugugguaacaggggauacgccag

SEQ ID NO: 817 - RNA, 25 nt, Artificial Sequence
  Description of Artificial Sequence Synthetic probe
guaucgcacagguauauccaauauu

SEQ ID NO: 818 - RNA, 25 nt, Artificial Sequence
  Description of Artificial Sequence Synthetic probe
uucuggagccuccuaaacugcgcag

SEQ ID NO: 819 - RNA, 25 nt, Artificial Sequence
  Description of Artificial Sequence Synthetic probe
ggaaagggauuaaguacauuguuac

SEQ ID NO: 820 - RNA, 25 nt, Artificial Sequence
  Description of Artificial Sequence Synthetic probe
cuuagacacaaaacaaggaguacua

SEQ ID NO: 821 - RNA, 25 nt, Artificial Sequence
  Description of Artificial Sequence Synthetic probe
guacauccaacuauugcagaaggau

SEQ ID NO: 822 - RNA, 25 nt, Artificial Sequence
  Description of Artificial Sequence Synthetic probe
uauccuuuacugaccugguacguac

SEQ ID NO: 823 - RNA, 25 nt, Artificial Sequence
  Description of Artificial Sequence Synthetic probe
gcugcaaugcuaacacaauuuaaag

SEQ ID NO: 824 - RNA, 25 nt, Artificial Sequence
  Description of Artificial Sequence Synthetic probe
ccaaaucuccaacugcacaaauuaa

SEQ ID NO: 825 - RNA, 25 nt, Artificial Sequence
  Description of Artificial Sequence Synthetic probe
gcuauagauagugaaaaccaggauc

SEQ ID NO: 826 - RNA, 25 nt, Artificial Sequence
  Description of Artificial Sequence Synthetic probe
aauggggaugcugaaggggaacaug

SEQ ID NO: 827 - RNA, 25 nt, Artificial Sequence
  Description of Artificial Sequence Synthetic probe
gcaguacgcaggcaacacaaacggu

SEQ ID NO: 828 - RNA, 25 nt, Artificial Sequence
  Description of Artificial Sequence Synthetic probe
gggaacacuacaggaaauuucauua

SEQ ID NO: 829 - RNA, 25 nt, Artificial Sequence
  Description of Artificial Sequence Synthetic probe
aaguauacagacagcaguggcgaca

SEQ ID NO: 830 - RNA, 25 nt, Artificial Sequence
  Description of Artificial Sequence Synthetic probe
ugauuccacagauauuuguguacag

SEQ ID NO: 831 - RNA, 25 nt, Artificial Sequence
  Description of Artificial Sequence Synthetic probe
cucacuaggauuuguguguccgugg

SEQ ID NO: 832 - RNA, 25 nt, Artificial Sequence
  Description of Artificial Sequence Synthetic probe
gggauacucugcgacaacuacagca

SEQ ID NO: 833 - RNA, 25 nt, Artificial Sequence
  Description of Artificial Sequence Synthetic probe
guaacaacacacugcagcugguagu

SEQ ID NO: 834 - RNA, 25 nt, Artificial Sequence
  Description of Artificial Sequence Synthetic probe
cguggaccaaagcccaccuugcagg

SEQ ID NO: 835 - RNA, 25 nt, Artificial Sequence
  Description of Artificial Sequence Synthetic probe
gagaaacccaaguauaacaucagau

SEQ ID NO: 836 - RNA, 25 nt, Artificial Sequence
  Description of Artificial Sequence Synthetic probe
caccuaaauagcaaacgaagauuuc

SEQ ID NO: 837 - RNA, 25 nt, Artificial Sequence
  Description of Artificial Sequence Synthetic probe
agcuacgauauuacucggacucggu

SEQ ID NO: 838 - RNA, 25 nt, Artificial Sequence
  Description of Artificial Sequence Synthetic probe
gaaccacuagcugcaugccaaucau

SEQ ID NO: 839 - RNA, 25 nt, Artificial Sequence
  Description of Artificial Sequence Synthetic probe
uuuauauguaguauauagggacggg

SEQ ID NO: 840 - RNA, 25 nt, Artificial Sequence
  Description of Artificial Sequence Synthetic probe
agacgaccacuacagcaaaccgagg

SEQ ID NO: 841 - RNA, 25 nt, Artificial Sequence
  Description of Artificial Sequence Synthetic probe
guugggcauacauaccuauacuuuu

SEQ ID NO: 842 - RNA, 25 nt, Artificial Sequence
  Description of Artificial Sequence Synthetic probe
ggcccuauaacacauaccuuuucuu

SEQ ID NO: 843 - RNA, 25 nt, Artificial Sequence
  Description of Artificial Sequence Synthetic probe
ccaacaaucugucuacuuguuacau

SEQ ID NO: 844 - RNA, 25 nt, Artificial Sequence
  Description of Artificial Sequence Synthetic probe
uaauuggcguguagaaccacuuucu

SEQ ID NO: 845 - RNA, 25 nt, Artificial Sequence
  Description of Artificial Sequence Synthetic probe
gcacaacuguauccacacccuaugu

SEQ ID NO: 846 - RNA, 25 nt, Artificial Sequence
  Description of Artificial Sequence Synthetic probe
acauaguuuaaccuacuggcgcgcc

SEQ ID NO: 847 - RNA, 25 nt, Artificial Sequence
  Description of Artificial Sequence Synthetic probe
cuaaacuggcacauuuacaaccccu

SEQ ID NO: 848 - RNA, 25 nt, Artificial Sequence
  Description of Artificial Sequence Synthetic probe
guggcuuauaugugaccuuuuaaac

SEQ ID NO: 849 - RNA, 25 nt, Artificial Sequence
  Description of Artificial Sequence Synthetic probe
gcauccauuuuacuuauaauccucc

SEQ ID NO: 850 - RNA, 25 nt, Artificial Sequence
  Description of Artificial Sequence Synthetic probe
cuuuguacccuauauucuuuccugu

SEQ ID NO: 851 - RNA, 25 nt, Artificial Sequence
  Description of Artificial Sequence Synthetic probe
uaauaguguuguguaggguugcacc

SEQ ID NO: 852 - RNA, 25 nt, Artificial Sequence
  Description of Artificial Sequence Synthetic probe
gguguuacuguacauaauuguggua

SEQ ID NO: 853 - RNA, 25 nt, Artificial Sequence
  Description of Artificial Sequence Synthetic probe
guguauguaugaaugugccuugugg

SEQ ID NO: 854 - RNA, 25 nt, Artificial Sequence
  Description of Artificial Sequence Synthetic probe
uacuguauuuuguuuguuugcgugc

SEQ ID NO: 855 - RNA, 25 nt, Artificial Sequence
  Description of Artificial Sequence Synthetic probe
caucuaggccugccaaacguguacg

SEQ ID NO: 856 - RNA, 25 nt, Artificial Sequence
  Description of Artificial Sequence Synthetic probe
gcuuccacgucuacugcaucuacug

SEQ ID NO: 857 - RNA, 25 nt, Artificial Sequence
  Description of Artificial Sequence Synthetic probe
cuaccauaggaccucguaagcgucc

SEQ ID NO: 858 - RNA, 25 nt, Artificial Sequence
  Description of Artificial Sequence Synthetic probe
guucaggcuggguuacgucguaggc

SEQ ID NO: 859 - RNA, 25 nt, Artificial Sequence
  Description of Artificial Sequence Synthetic probe
auacuacaccuccagaaaagcagga

SEQ ID NO: 860 - RNA, 25 nt, Artificial Sequence
  Description of Artificial Sequence Synthetic probe
aucaguugcuguuaccugucaaaag

SEQ ID NO: 861 - RNA, 25 nt, Artificial Sequence
  Description of Artificial Sequence Synthetic probe
uuuaagcaguauaguagacaugugg

SEQ ID NO: 862 - RNA, 25 nt, Artificial Sequence
  Description of Artificial Sequence Synthetic probe
gccaaguacauaugacccuacuaag

SEQ ID NO: 863 - RNA, 25 nt, Artificial Sequence
  Description of Artificial Sequence Synthetic probe
ggcucuauuauuacuucugauucuc

SEQ ID NO: 864 - RNA, 25 nt, Artificial Sequence
  Description of Artificial Sequence Synthetic probe
guuguguguauuccccuucucccag

SEQ ID NO: 865 - RNA, 25 nt, Artificial Sequence
  Description of Artificial Sequence Synthetic probe
gcuaauaugcgugaaaccccuggca

SEQ ID NO: 866 - RNA, 25 nt, Artificial Sequence
  Description of Artificial Sequence Synthetic probe
uacggaccuauauauuaaaggcacu

SEQ ID NO: 867 - RNA, 25 nt, Artificial Sequence
  Description of Artificial Sequence Synthetic probe
cauuagacauuugucaauccaucug

SEQ ID NO: 868 - RNA, 25 nt, Artificial Sequence
  Description of Artificial Sequence Synthetic probe
uugcaggauacaaagugcgagguuc

SEQ ID NO: 869 - RNA, 25 nt, Artificial Sequence
  Description of Artificial Sequence Synthetic probe
gcacaauugcaaccuggugacuguc

SEQ ID NO: 870 - RNA, 25 nt, Artificial Sequence
  Description of Artificial Sequence Synthetic probe
agcuguuauuacgcaggauguuagg

SEQ ID NO: 871 - RNA, 25 nt, Artificial Sequence
  Description of Artificial Sequence Synthetic probe
guagcuuuacccgauccuaauaaau

SEQ ID NO: 872 - RNA, 25 nt, Artificial Sequence
  Description of Artificial Sequence Synthetic probe
gcuguuccuaagguauccgcauauc

SEQ ID NO: 873 - RNA, 25 nt, Artificial Sequence
  Description of Artificial Sequence Synthetic probe
accuaauggugcagguaauaaacag

SEQ ID NO: 874 - RNA, 25 nt, Artificial Sequence
  Description of Artificial Sequence Synthetic probe
uaggcaauccauauuuuaggguugu

SEQ ID NO: 875 - RNA, 25 nt, Artificial Sequence
  Description of Artificial Sequence Synthetic probe
cuucuguggccagaguugucagcac

SEQ ID NO: 876 - RNA, 25 nt, Artificial Sequence
  Description of Artificial Sequence Synthetic probe
cacacaauauuauuuauggccaugg

SEQ ID NO: 877 - RNA, 25 nt, Artificial Sequence
  Description of Artificial Sequence Synthetic probe
ucuccuaccaaugcuuccaccacca

SEQ ID NO: 878 - RNA, 25 nt, Artificial Sequence
  Description of Artificial Sequence Synthetic probe
ccauacuccuauguggccuaguaca

SEQ ID NO: 879 - RNA, 25 nt, Artificial Sequence
  Description of Artificial Sequence Synthetic probe
auacuggcccggacauuauauugcc

SEQ ID NO: 880 - RNA, 25 nt, Artificial Sequence
  Description of Artificial Sequence Synthetic probe
aguaccauuaacaucugcaugggau

SEQ ID NO: 881 - RNA, 25 nt, Artificial Sequence
  Description of Artificial Sequence Synthetic probe
uacugcugcauccucuuacaguaau

SEQ ID NO: 882 - RNA, 25 nt, Artificial Sequence
  Description of Artificial Sequence Synthetic probe
caaaguauuccuugaccaugccuuc

SEQ ID NO: 883 - RNA, 25 nt, Artificial Sequence
  Description of Artificial Sequence Synthetic probe
caccuagcacuauacacaaaucauu

SEQ ID NO: 884 - RNA, 25 nt, Artificial Sequence
  Description of Artificial Sequence Synthetic probe
gacuucccaccuccugcguccacua

SEQ ID NO: 885 - RNA, 25 nt, Artificial Sequence
  Description of Artificial Sequence Synthetic probe
cuacaaaugauagugaccuguuuga

SEQ ID NO: 886 - RNA, 25 nt, Artificial Sequence
  Description of Artificial Sequence Synthetic probe
ccauugcugcuacagaggaaauuga

SEQ ID NO: 887 - RNA, 25 nt, Artificial Sequence
  Description of Artificial Sequence Synthetic probe
cacuguuagauuuaguagauugggu

SEQ ID NO: 888 - RNA, 25 nt, Artificial Sequence
  Description of Artificial Sequence Synthetic probe
ccaguaauguuccugauuccgauuu

SEQ ID NO: 889 - RNA, 25 nt, Artificial Sequence
  Description of Artificial Sequence Synthetic probe
gacaccacacuauccuuugagccua

SEQ ID NO: 890 - RNA, 25 nt, Artificial Sequence
  Description of Artificial Sequence Synthetic probe
ucguugguuacauuugauaauccag

SEQ ID NO: 891 - RNA, 25 nt, Artificial Sequence
  Description of Artificial Sequence Synthetic probe
aaucaacagguccguguguccaccu

SEQ ID NO: 892 - RNA, 25 nt, Artificial Sequence
  Description of Artificial Sequence Synthetic probe
caucuucugggucagguacggaacc

SEQ ID NO: 893 - RNA, 25 nt, Artificial Sequence
  Description of Artificial Sequence Synthetic probe
ugguacaccaacaucgggcagccau

SEQ ID NO: 894 - RNA, 25 nt, Artificial Sequence
  Description of Artificial Sequence Synthetic probe
gcauuuucugaucccucuauuauug

SEQ ID NO: 895 - RNA, 25 nt, Artificial Sequence
  Description of Artificial Sequence Synthetic probe
cucuguuucuauuucgucaacuagu

SEQ ID NO: 896 - RNA, 25 nt, Artificial Sequence
  Description of Artificial Sequence Synthetic probe
uguuggacaucacaccuaccgugga

SEQ ID NO: 897 - RNA, 25 nt, Artificial Sequence
  Description of Artificial Sequence Synthetic probe
uugccucuggugcuccgguucccac

SEQ ID NO: 898 - RNA, 25 nt, Artificial Sequence
  Description of Artificial Sequence Synthetic probe
caggucuaauacuguuguggauguu

SEQ ID NO: 899 - RNA, 25 nt, Artificial Sequence
  Description of Artificial Sequence Synthetic probe
uuuacaguggucuagccuugggaua

SEQ ID NO: 900 - RNA, 25 nt, Artificial Sequence
  Description of Artificial Sequence Synthetic probe
guuuaauaaaccaugguaucccacc

SEQ ID NO: 901 - RNA, 25 nt, Artificial Sequence
  Description of Artificial Sequence Synthetic probe
auaccugugaugugcauguuguugu

SEQ ID NO: 902 - RNA, 25 nt, Artificial Sequence
  Description of Artificial Sequence Synthetic probe
gcaugcuuuacacaccauacaauaa

SEQ ID NO: 903 - RNA, 25 nt, Artificial Sequence
  Description of Artificial Sequence Synthetic probe
gcauuugcuguauacauuuguugcu

SEQ ID NO: 904 - RNA, 25 nt, Artificial Sequence
  Description of Artificial Sequence Synthetic probe
ugugugugcuuuugcuugguuguug

SEQ ID NO: 905 - RNA, 25 nt, Artificial Sequence
  Description of Artificial Sequence Synthetic probe
gugccuuuaugugugcugcaauguc

SEQ ID NO: 906 - RNA, 25 nt, Artificial Sequence
  Description of Artificial Sequence Synthetic probe
gggauacaugacuauaugaaucugu

SEQ ID NO: 907 - RNA, 25 nt, Artificial Sequence
  Description of Artificial Sequence Synthetic probe
uuccuaacaguguacaaaucucggu

SEQ ID NO: 908 - RNA, 25 nt, Artificial Sequence
  Description of Artificial Sequence Synthetic probe
uacucagaaauauccuccaccuggc

SEQ ID NO: 909 - RNA, 25 nt, Artificial Sequence
  Description of Artificial Sequence Synthetic probe
uaagauauaggcuacgcaaauaugc

SEQ ID NO: 910 - RNA, 25 nt, Artificial Sequence
  Description of Artificial Sequence Synthetic probe
agaaggaaaguguguagugguaaca

SEQ ID NO: 911 - RNA, 25 nt, Artificial Sequence
  Description of Artificial Sequence Synthetic probe
cuguguucaaguacaaguaacaaca

SEQ ID NO: 912 - RNA, 25 nt, Artificial Sequence
  Description of Artificial Sequence Synthetic probe
ucacagagcagcaccacggacgugu

SEQ ID NO: 913 - RNA, 25 nt, Artificial Sequence
  Description of Artificial Sequence Synthetic probe
cacauccagacgccggcuacuaagc

SEQ ID NO: 914 - RNA, 25 nt, Artificial Sequence
  Description of Artificial Sequence Synthetic probe
agacagcuacaacacgccuccacgu

SEQ ID NO: 915 - RNA, 25 nt, Artificial Sequence
  Description of Artificial Sequence Synthetic probe
gaaauaguaauacgugggaaguaca

SEQ ID NO: 916 - RNA, 25 nt, Artificial Sequence
  Description of Artificial Sequence Synthetic probe
guguuagcuauugggguguauauua

SEQ ID NO: 917 - RNA, 25 nt, Artificial Sequence
  Description of Artificial Sequence Synthetic probe
gggauaugggacaaaacagcagcau

SEQ ID NO: 918 - RNA, 25 nt, Artificial Sequence
  Description of Artificial Sequence Synthetic probe
gaacuauguaguaugggacaguaua

SEQ ID NO: 919 - RNA, 25 nt, Artificial Sequence
  Description of Artificial Sequence Synthetic probe
cgugcacguauacuuugauggcaac

SEQ ID NO: 920 - RNA, 25 nt, Artificial Sequence
  Description of Artificial Sequence Synthetic probe
gaauacagaaccgucgcaguguuuu

SEQ ID NO: 921 - RNA, 25 nt, Artificial Sequence
  Description of Artificial Sequence Synthetic probe
agcaaguauaacaaugaggaaugga

SEQ ID NO: 922 - RNA, 25 nt, Artificial Sequence
  Description of Artificial Sequence Synthetic probe
uacagcaagggaacaugguauuacc

SEQ ID NO: 923 - RNA, 25 nt, Artificial Sequence
  Description of Artificial Sequence Synthetic probe
uggcaacuuauacguuuggaaaaug

SEQ ID NO: 924 - RNA, 25 nt, Artificial Sequence
  Description of Artificial Sequence Synthetic probe
gacaguaaagacauaaacagccaaa

SEQ ID NO: 925 - RNA, 25 nt, Artificial Sequence
  Description of Artificial Sequence Synthetic probe
gacgaugaagaugcagacaccgaag

SEQ ID NO: 926 - RNA, 25 nt, Artificial Sequence
  Description of Artificial Sequence Synthetic probe
acgguauuuacauuuccacaugcau

SEQ ID NO: 927 - RNA, 25 nt, Artificial Sequence
  Description of Artificial Sequence Synthetic probe
cauccaauauugauccagcaaaaga

SEQ ID NO: 928 - RNA, 25 nt, Artificial Sequence
  Description of Artificial Sequence Synthetic probe
gcuaaaauguccuccaauccuauua

SEQ ID NO: 929 - RNA, 25 nt, Artificial Sequence
  Description of Artificial Sequence Synthetic probe
agcagauacuaagguagccauguug

SEQ ID NO: 930 - RNA, 25 nt, Artificial Sequence
  Description of Artificial Sequence Synthetic probe
guuuuauacauuuccuacaaggugc

SEQ ID NO: 931 - RNA, 25 nt, Artificial Sequence
  Description of Artificial Sequence Synthetic probe
ggcacuaaaggaauuucuuaaagga

SEQ ID NO: 932 - RNA, 25 nt, Artificial Sequence
  Description of Artificial Sequence Synthetic probe
uuguugcacguaccugaaacaugua

SEQ ID NO: 933 - RNA, 25 nt, Artificial Sequence
  Description of Artificial Sequence Synthetic probe
cuaacuguugcaaaaggcuuaagca

SEQ ID NO: 934 - RNA, 25 nt, Artificial Sequence
  Description of Artificial Sequence Synthetic probe
gcccauauccaauguuuagauugua

SEQ ID NO: 935 - RNA, 25 nt, Artificial Sequence
  Description of Artificial Sequence Synthetic probe
ggguaauggcuauauuuggaguuaa

SEQ ID NO: 936 - RNA, 25 nt, Artificial Sequence
  Description of Artificial Sequence Synthetic probe
cugucauuuacggauuugguuagaa

SEQ ID NO: 937 - RNA, 25 nt, Artificial Sequence
  Description of Artificial Sequence Synthetic probe
ggcaguauuuaaagacauauauggg

SEQ ID NO: 938 - RNA, 25 nt, Artificial Sequence
  Description of Artificial Sequence Synthetic probe
aaaggagcuauuacaagcaaguaac

SEQ ID NO: 939 - RNA, 25 nt, Artificial Sequence
  Description of Artificial Sequence Synthetic probe
auccgcauugcaguauuacagaacu

SEQ ID NO: 940 - RNA, 25 nt, Artificial Sequence
  Description of Artificial Sequence Synthetic probe
aguagugacaaugcagaaaauguag

SEQ ID NO: 941 - RNA, 25 nt, Artificial Sequence
  Description of Artificial Sequence Synthetic probe
uaguacacaaaguagugguggggau

SEQ ID NO: 942 - RNA, 25 nt, Artificial Sequence
  Description of Artificial Sequence Synthetic probe
uaaacacuaaugcggaaaauggcgg

SEQ ID NO: 943 - RNA, 25 nt, Artificial Sequence
  Description of Artificial Sequence Synthetic probe
uggaagcugcagagacucagguaac

SEQ ID NO: 944 - RNA, 25 nt, Artificial Sequence
  Description of Artificial Sequence Synthetic probe
guccacgguuacaagaaauuucauu

SEQ ID NO: 945 - RNA, 25 nt, Artificial Sequence
  Description of Artificial Sequence Synthetic probe
cagcuaaguguggauacggaucuaa

SEQ ID NO: 946 - RNA, 25 nt, Artificial Sequence
  Description of Artificial Sequence Synthetic probe
gguguugcaucuuuuaaaacgaaag

SEQ ID NO: 947 - RNA, 25 nt, Artificial Sequence
  Description of Artificial Sequence Synthetic probe
caugcgcaggaaguucagaaugaug

SEQ ID NO: 948 - RNA, 25 nt, Artificial Sequence
  Description of Artificial Sequence Synthetic probe
acaauuauccauuugugaacaggca

SEQ ID NO: 949 - RNA, 25 nt, Artificial Sequence
  Description of Artificial Sequence Synthetic probe
guaauggcugguucuuuguagaaac

SEQ ID NO: 950 - RNA, 25 nt, Artificial Sequence
  Description of Artificial Sequence Synthetic probe
cuaaccaauaaucuacaauggcgga

SEQ ID NO: 951 - RNA, 25 nt, Artificial Sequence
  Description of Artificial Sequence Synthetic probe
ggaccuuagaacacuacagcagcug

SEQ ID NO: 952 - RNA, 25 nt, Artificial Sequence
  Description of Artificial Sequence Synthetic probe
cagaauugagcuuacaguagagagc

SEQ ID NO: 953 - RNA, 25 nt, Artificial Sequence
  Description of Artificial Sequence Synthetic probe
agauccuguugaccuguuguguuac

SEQ ID NO: 954 - RNA, 25 nt, Artificial Sequence
  Description of Artificial Sequence Synthetic probe
ugcauuuggaaccucagaaugaauu

SEQ ID NO: 955 - RNA, 25 nt, Artificial Sequence
  Description of Artificial Sequence Synthetic probe
ccccgggaaacacugcaagaaauug

SEQ ID NO: 956 - RNA, 25 nt, Artificial Sequence
  Description of Artificial Sequence Synthetic probe
caaguauagcaauaaguaugcaugg

SEQ ID NO: 957 - RNA, 25 nt, Artificial Sequence
  Description of Artificial Sequence Synthetic probe
acggcaagaaagacuucgcagacgu

SEQ ID NO: 958 - RNA, 25 nt, Artificial Sequence
  Description of Artificial Sequence Synthetic probe
aguguaauacauguugugaccaggc

SEQ ID NO: 959 - RNA, 25 nt, Artificial Sequence
  Description of Artificial Sequence Synthetic probe
agcauagcuggacaguaccgagggc

SEQ ID NO: 960 - RNA, 25 nt, Artificial Sequence
  Description of Artificial Sequence Synthetic probe
ccuuaaggacaaacgaagauuucac

SEQ ID NO: 961 - RNA, 25 nt, Artificial Sequence
  Description of Artificial Sequence Synthetic probe
aacucuguauauggagagacacugg

SEQ ID NO: 962 - RNA, 25 nt, Artificial Sequence
  Description of Artificial Sequence Synthetic probe
uguauagagacuguauagcauaugc

SEQ ID NO: 963 - RNA, 25 nt, Artificial Sequence
  Description of Artificial Sequence Synthetic probe
ggaacgcacagagguauaucaauuu

SEQ ID NO: 964 - RNA, 25 nt, Artificial Sequence
  Description of Artificial Sequence Synthetic probe
uauugccuguguauauugcaaagca

SEQ ID NO: 965 - RNA, 25 nt, Artificial Sequence
  Description of Artificial Sequence Synthetic probe
ugaauacaucacuacaagacguauc

SEQ ID NO: 966 - RNA, 25 nt, Artificial Sequence
  Description of Artificial Sequence Synthetic probe
aagcuaccagauuugugcacagaau

SEQ ID NO: 967 - RNA, 25 nt, Artificial Sequence
  Description of Artificial Sequence Synthetic probe
ugacgauccaaagcaacgacccuac

SEQ ID NO: 968 - RNA, 25 nt, Artificial Sequence
  Description of Artificial Sequence Synthetic probe
aaagugcauuacaggauggcgcgcu

SEQ ID NO: 969 - RNA, 25 nt, Artificial Sequence
  Description of Artificial Sequence Synthetic probe
ccugguauuagucauuuuccugucc

SEQ ID NO: 970 - RNA, 25 nt, Artificial Sequence
  Description of Artificial Sequence Synthetic probe
ugguggauacauaucguuuugugca

SEQ ID NO: 971 - RNA, 25 nt, Artificial Sequence
  Description of Artificial Sequence Synthetic probe
auggccauauuuagaaaguagggug

SEQ ID NO: 972 - RNA, 25 nt, Artificial Sequence
  Description of Artificial Sequence Synthetic probe
guuguuuacaauaucagauaguggc

SEQ ID NO: 973 - RNA, 25 nt, Artificial Sequence
  Description of Artificial Sequence Synthetic probe
acuaccagcccgacgagccgaacca

SEQ ID NO: 974 - RNA, 25 nt, Artificial Sequence
  Description of Artificial Sequence Synthetic probe
ugccugcggugccagaaaccauuga

SEQ ID NO: 975 - RNA, 25 nt, Artificial Sequence
  Description of Artificial Sequence Synthetic probe
uuguguucugccuaugcuugcaaca

SEQ ID NO: 976 - RNA, 25 nt, Artificial Sequence
  Description of Artificial Sequence Synthetic probe
ccaucuuacucauaugcaggugugc

SEQ ID NO: 977 - RNA, 25 nt, Artificial Sequence
  Description of Artificial Sequence Synthetic probe
gugccaaguuucuauccuacuuaua

SEQ ID NO: 978 - RNA, 25 nt, Artificial Sequence
  Description of Artificial Sequence Synthetic probe
ccgcccuauaauaauuuaacugcuu

SEQ ID NO: 979 - RNA, 25 nt, Artificial Sequence
  Description of Artificial Sequence Synthetic probe
cuuuaacaauuguuggcacacuguu

SEQ ID NO: 980 - RNA, 25 nt, Artificial Sequence
  Description of Artificial Sequence Synthetic probe
gcuagucauacaaccuauuagucau

SEQ ID NO: 981 - RNA, 25 nt, Artificial Sequence
  Description of Artificial Sequence Synthetic probe
ccuuguacuuggcgcgccuuaccgg

SEQ ID NO: 982 - RNA, 25 nt, Artificial Sequence
  Description of Artificial Sequence Synthetic probe
uagugcauacauccgcccgcccacg

SEQ ID NO: 983 - RNA, 25 nt, Artificial Sequence
  Description of Artificial Sequence Synthetic probe
aaguuuuaaaccacaacugccaguu

SEQ ID NO: 984 - RNA, 25 nt, Artificial Sequence
  Description of Artificial Sequence Synthetic probe
gauuucgguucguguacuuuuagua

SEQ ID NO: 985 - RNA, 25 nt, Artificial Sequence
  Description of Artificial Sequence Synthetic probe
cagcugcagccauuuugagugcaac

SEQ ID NO: 986 - RNA, 25 nt, Artificial Sequence
  Description of Artificial Sequence Synthetic probe
agggugguguuucgguggcgucccu

SEQ ID NO: 987 - RNA, 25 nt, Artificial Sequence
  Description of Artificial Sequence Synthetic probe
uguggguauuacauuauccccguag

SEQ ID NO: 988 - RNA, 25 nt, Artificial Sequence
  Description of Artificial Sequence Synthetic probe
cauuuguaugacauguacgggugua

SEQ ID NO: 989 - RNA, 25 nt, Artificial Sequence
  Description of Artificial Sequence Synthetic probe
guuguuccuguauguaugaguuaug

SEQ ID NO: 990 - RNA, 25 nt, Artificial Sequence
  Description of Artificial Sequence Synthetic probe
guaugccuguauguauauguuugug

SEQ ID NO: 991 - RNA, 25 nt, Artificial Sequence
  Description of Artificial Sequence Synthetic probe
ucaucggcauccucuuccucuuccu

SEQ ID NO: 992 - RNA, 25 nt, Artificial Sequence
  Description of Artificial Sequence Synthetic probe
cguacaacgcaagcccagaccaggc

SEQ ID NO: 993 - RNA, 25 nt, Artificial Sequence
  Description of Artificial Sequence Synthetic probe
aacauuaccuccgucugcuaguuug

SEQ ID NO: 994 - RNA, 25 nt, Artificial Sequence
  Description of Artificial Sequence Synthetic probe
cuaccauucuugaacaguggaauuu

SEQ ID NO: 995 - RNA, 25 nt, Artificial Sequence
  Description of Artificial Sequence Synthetic probe
cuacagagguaauggcuuauuuaca

SEQ ID NO: 996 - RNA, 25 nt, Artificial Sequence
  Description of Artificial Sequence Synthetic probe
cuuuaagcaauauauuaggcauggg

SEQ ID NO: 997 - RNA, 25 nt, Artificial Sequence
  Description of Artificial Sequence Synthetic probe
uuuccccaacauuuacuccaaguaa

SEQ ID NO: 998 - RNA, 25 nt, Artificial Sequence
  Description of Artificial Sequence Synthetic probe
uuuaacuauuagcacugccacugcu

SEQ ID NO: 999 - RNA, 25 nt, Artificial Sequence
  Description of Artificial Sequence Synthetic probe
accuguguugauacuaccagaagua

SEQ ID NO: 1000 - RNA, 25 nt, Artificial Sequence
  Description of Artificial Sequence Synthetic probe
uauauauacucugcuacucccagug

SEQ ID NO: 1001 - RNA, 25 nt, Artificial Sequence
  Description of Artificial Sequence Synthetic probe
uaauggccgugacccuauagaaagu

SEQ ID NO: 1002 - RNA, 25 nt, Artificial Sequence
  Description of Artificial Sequence Synthetic probe
cuuguagguguuggggaagacauuc

SEQ ID NO: 1003 - RNA, 25 nt, Artificial Sequence
  Description of Artificial Sequence Synthetic probe
gccaccaaaucagacgucccuuugg

SEQ ID NO: 1004 - RNA, 25 nt, Artificial Sequence
  Description of Artificial Sequence Synthetic probe
acuuguauccucugucauucaggau

SEQ ID NO: 1005 - RNA, 25 nt, Artificial Sequence
  Description of Artificial Sequence Synthetic probe
guugacaacaaacagacucaguuau

SEQ ID NO: 1006 - RNA, 25 nt, Artificial Sequence
  Description of Artificial Sequence Synthetic probe
aaauggcaaugcacaacaagauguu

SEQ ID NO: 1007 - RNA, 25 nt, Artificial Sequence
  Description of Artificial Sequence Synthetic probe
augacacagaaaauucacgcauagc

SEQ ID NO: 1008 - RNA, 25 nt, Artificial Sequence
  Description of Artificial Sequence Synthetic probe
ccggauccaaauuuauauaauccag

SEQ ID NO: 1009 - RNA, 25 nt, Artificial Sequence
  Description of Artificial Sequence Synthetic probe
aaaguaucugcauuucaauacaggg

SEQ ID NO: 1010 - RNA, 25 nt, Artificial Sequence
  Description of Artificial Sequence Synthetic probe
aaccucaacgcgugcugcuauuccu

SEQ ID NO: 1011 - RNA, 25 nt, Artificial Sequence
  Description of Artificial Sequence Synthetic probe
agacuaauaacauuaggacaucccu

SEQ ID NO: 1012 - RNA, 25 nt, Artificial Sequence
  Description of Artificial Sequence Synthetic probe
acagaagaauauaucacacgcaccg

SEQ ID NO: 1013 - RNA, 25 nt, Artificial Sequence
  Description of Artificial Sequence Synthetic probe
ugcaccugugucucgaauugugaau

SEQ ID NO: 1014 - RNA, 25 nt, Artificial Sequence
  Description of Artificial Sequence Synthetic probe
uauacacauuuacuacgcaaacgcc

SEQ ID NO: 1015 - RNA, 25 nt, Artificial Sequence
  Description of Artificial Sequence Synthetic probe
agguggggauuacuauuuguggccc

SEQ ID NO: 1016 - RNA, 25 nt, Artificial Sequence
  Description of Artificial Sequence Synthetic probe
gacaccaagcauucuauuguuauac

SEQ ID NO: 1017 - RNA, 25 nt, Artificial Sequence
  Description of Artificial Sequence Synthetic probe
gccuuauguuccccacacuuccauu

SEQ ID NO: 1018 - RNA, 25 nt, Artificial Sequence
  Description of Artificial Sequence Synthetic probe
uauugcccacaucuccuacaguaug

SEQ ID NO: 1019 - RNA, 25 nt, Artificial Sequence
  Description of Artificial Sequence Synthetic probe
ccuauucauacagggccugaugugg

SEQ ID NO: 1020 - RNA, 25 nt, Artificial Sequence
  Description of Artificial Sequence Synthetic probe
cuucaucuaugucuucaucuuaugc

SEQ ID NO: 1021 - RNA, 25 nt, Artificial Sequence
  Description of Artificial Sequence Synthetic probe
cacuccucuuugucuaggcaguugc

SEQ ID NO: 1022 - RNA, 25 nt, Artificial Sequence
  Description of Artificial Sequence Synthetic probe
ugauuuagaugaagcugaaacaggu

SEQ ID NO: 1023 - RNA, 25 nt, Artificial Sequence
  Description of Artificial Sequence Synthetic probe
cagccuuuacuuucaccuucuaaua

SEQ ID NO: 1024 - RNA, 25 nt, Artificial Sequence
  Description of Artificial Sequence Synthetic probe
ugcaccagcugaugaacuugaaaug

SEQ ID NO: 1025 - RNA, 25 nt, Artificial Sequence
  Description of Artificial Sequence Synthetic probe
ucuggauauuauuacacugcaccgc

SEQ ID NO: 1026 - RNA, 25 nt, Artificial Sequence
  Description of Artificial Sequence Synthetic probe
acuuuugaggaaccugaugcuguug

SEQ ID NO: 1027 - RNA, 25 nt, Artificial Sequence
  Description of Artificial Sequence Synthetic probe
uuuugagccuauugacacauccaua

SEQ ID NO: 1028 - RNA, 25 nt, Artificial Sequence
  Description of Artificial Sequence Synthetic probe
ccuacacacagguuaaaguuacaaa

SEQ ID NO: 1029 - RNA, 25 nt, Artificial Sequence
  Description of Artificial Sequence Synthetic probe
gcugcuccccgcuuguauaguaagu

SEQ ID NO: 1030 - RNA, 25 nt, Artificial Sequence
  Description of Artificial Sequence Synthetic probe
cuauuagcagcacaccuacuccagg

SEQ ID NO: 1031 - RNA, 25 nt, Artificial Sequence
  Description of Artificial Sequence Synthetic probe
ugcauccaaugucaguacugguacu

SEQ ID NO: 1032 - RNA, 25 nt, Artificial Sequence
  Description of Artificial Sequence Synthetic probe
uuuacuaguacacuacucugguacu

SEQ ID NO: 1033 - RNA, 25 nt, Artificial Sequence
  Description of Artificial Sequence Synthetic probe
cauccauugaggcuccacaaucugg

SEQ ID NO: 1034 - RNA, 25 nt, Artificial Sequence
  Description of Artificial Sequence Synthetic probe
gguacuguacauguuucuaguacua

SEQ ID NO: 1035 - RNA, 25 nt, Artificial Sequence
  Description of Artificial Sequence Synthetic probe
uacuucaucuuccacaacaaccccu

SEQ ID NO: 1036 - RNA, 25 nt, Artificial Sequence
  Description of Artificial Sequence Synthetic probe
ggucuccuauaccuaccuuuacugg

SEQ ID NO: 1037 - RNA, 25 nt, Artificial Sequence
  Description of Artificial Sequence Synthetic probe
gaggacucuaguauuauucagucug

SEQ ID NO: 1038 - RNA, 25 nt, Artificial Sequence
  Description of Artificial Sequence Synthetic probe
caccauacugaaccuucuauaguaa

SEQ ID NO: 1039 - RNA, 25 nt, Artificial Sequence
  Description of Artificial Sequence Synthetic probe
gccaccuauuauaauugaccuaugg

SEQ ID NO: 1040 - RNA, 25 nt, Artificial Sequence
  Description of Artificial Sequence Synthetic probe
aggcgugguggauauugcuccugca

SEQ ID NO: 1041 - RNA, 25 nt, Artificial Sequence
  Description of Artificial Sequence Synthetic probe
uacuggauauaucccuuuagguggu

SEQ ID NO: 1042 - RNA, 25 nt, Artificial Sequence
  Description of Artificial Sequence Synthetic probe
ggccgauaaaauauuacaguggagu

SEQ ID NO: 1043 - RNA, 25 nt, Artificial Sequence
  Description of Artificial Sequence Synthetic probe
uguugugaauaagguugaagguacu

SEQ ID NO: 1044 - RNA, 25 nt, Artificial Sequence
  Description of Artificial Sequence Synthetic probe
aauaugguggcuacacgugcacggc

SEQ ID NO: 1045 - RNA, 25 nt, Artificial Sequence
  Description of Artificial Sequence Synthetic probe
uguugcaacaucccaauuaacuaca

SEQ ID NO: 1046 - RNA, 25 nt, Artificial Sequence
  Description of Artificial Sequence Synthetic probe
cguguuugcagcugccuuauuauua

SEQ ID NO: 1047 - RNA, 25 nt, Artificial Sequence
  Description of Artificial Sequence Synthetic probe
uguugccgcuacugcugucccaaua

SEQ ID NO: 1048 - RNA, 25 nt, Artificial Sequence
  Description of Artificial Sequence Synthetic probe
gacauauuguaaccauugcaguguu

SEQ ID NO: 1049 - RNA, 25 nt, Artificial Sequence
  Description of Artificial Sequence Synthetic probe
guacauauauacugucacaagccaa

SEQ ID NO: 1050 - RNA, 25 nt, Artificial Sequence
  Description of Artificial Sequence Synthetic probe
gggaauuaugacacuguaacuagug

SEQ ID NO: 1051 - RNA, 25 nt, Artificial Sequence
  Description of Artificial Sequence Synthetic probe
gugcacaucaacgggaaacauuuau

SEQ ID NO: 1052 - RNA, 25 nt, Artificial Sequence
  Description of Artificial Sequence Synthetic probe
ggcauuguuaccauuguguuugaca

SEQ ID NO: 1053 - RNA, 25 nt, Artificial Sequence
  Description of Artificial Sequence Synthetic probe
caacucagacugcguuuauagugca

SEQ ID NO: 1054 - RNA, 25 nt, Artificial Sequence
  Description of Artificial Sequence Synthetic probe
caaacaaccaaauacacuguggaag

SEQ ID NO: 1055 - RNA, 25 nt, Artificial Sequence
  Description of Artificial Sequence Synthetic probe
cuccacaaucuccccacuguccgug

SEQ ID NO: 1056 - RNA, 25 nt, Artificial Sequence
  Description of Artificial Sequence Synthetic probe
gacagcgacuuacugagcccgacuc

SEQ ID NO: 1057 - RNA, 25 nt, Artificial Sequence
  Description of Artificial Sequence Synthetic probe
gaagcccagacacaacagcgaaaac

SEQ ID NO: 1058 - RNA, 25 nt, Artificial Sequence
  Description of Artificial Sequence Synthetic probe
gaccaauccccuuaccaccugcgug

SEQ ID NO: 1059 - RNA, 25 nt, Artificial Sequence
  Description of Artificial Sequence Synthetic probe
uugaacaacuaucaaacaccccaac

SEQ ID NO: 1060 - RNA, 25 nt, Artificial Sequence
  Description of Artificial Sequence Synthetic probe
gacgcguuauccacuacuacaacug

SEQ ID NO: 1061 - RNA, 25 nt, Artificial Sequence
  Description of Artificial Sequence Synthetic probe
gguacuguaauaacauguccugaau

SEQ ID NO: 1062 - RNA, 25 nt, Artificial Sequence
  Description of Artificial Sequence Synthetic probe
acaacagugggaggucuauauguau

SEQ ID NO: 1063 - RNA, 25 nt, Artificial Sequence
  Description of Artificial Sequence Synthetic probe
aagaugaagccaaaauauauggggc

SEQ ID NO: 1064 - RNA, 25 nt, Artificial Sequence
  Description of Artificial Sequence Synthetic probe
gacuauacggguauauauuacacug

SEQ ID NO: 1065 - RNA, 25 nt, Artificial Sequence
  Description of Artificial Sequence Synthetic probe
guggguaaagacaaauggaaaugug

SEQ ID NO: 1066 - RNA, 25 nt, Artificial Sequence
  Description of Artificial Sequence Synthetic probe
caauggacuauacaagcuggaaauu

SEQ ID NO: 1067 - RNA, 25 nt, Artificial Sequence
  Description of Artificial Sequence Synthetic probe
gaacuaugguguguggcucccaagc

SEQ ID NO: 1068 - RNA, 25 nt, Artificial Sequence
  Description of Artificial Sequence Synthetic probe
auggacaaugcgggagacauguuau

SEQ ID NO: 1069 - RNA, 25 nt, Artificial Sequence
  Description of Artificial Sequence Synthetic probe
acaaaucagacuauaacauggaacc

SEQ ID NO: 1070 - RNA, 25 nt, Artificial Sequence
  Description of Artificial Sequence Synthetic probe
augcacauggccuuacaaucgcuua

SEQ ID NO: 1071 - RNA, 25 nt, Artificial Sequence
  Description of Artificial Sequence Synthetic probe
aaaacaaaaggccugucaagcaauu

SEQ ID NO: 1072 - RNA, 25 nt, Artificial Sequence
  Description of Artificial Sequence Synthetic probe
agguaguaccagcaacaacaguauc

SEQ ID NO: 1073 - RNA, 25 nt, Artificial Sequence
  Description of Artificial Sequence Synthetic probe
agaaacuuacgaacaaucaaucacc

SEQ ID NO: 1074 - RNA, 25 nt, Artificial Sequence
  Description of Artificial Sequence Synthetic probe
gauaugaagcugcuauguuuuaugc

SEQ ID NO: 1075 - RNA, 25 nt, Artificial Sequence
  Description of Artificial Sequence Synthetic probe
gggaaugcuguguauacauugaaug

SEQ ID NO: 1076 - RNA, 25 nt, Artificial Sequence
  Description of Artificial Sequence Synthetic probe
gaggaugcaaaccuaauguauuuac

SEQ ID NO: 1077 - RNA, 25 nt, Artificial Sequence
  Description of Artificial Sequence Synthetic probe
agccacuagaggaugcuaaaauagc

SEQ ID NO: 1078 - RNA, 25 nt, Artificial Sequence
  Description of Artificial Sequence Synthetic probe
guuuaugcaaggguccauuauuuca

SEQ ID NO: 1079 - RNA, 25 nt, Artificial Sequence
  Description of Artificial Sequence Synthetic probe
gucauuauuugcaaugagccuaaug

SEQ ID NO: 1080 - RNA, 25 nt, Artificial Sequence
  Description of Artificial Sequence Synthetic probe
auugcauagucauauauggcccacc

SEQ ID NO: 1081 - RNA, 25 nt, Artificial Sequence
  Description of Artificial Sequence Synthetic probe
ugauagagcaaaggauggaggcaac

SEQ ID NO: 1082 - RNA, 25 nt, Artificial Sequence
  Description of Artificial Sequence Synthetic probe
uuaucuaugucagccuggauaaggu

SEQ ID NO: 1083 - RNA, 25 nt, Artificial Sequence
  Description of Artificial Sequence Synthetic probe
gcauuacaaacgagcacaaagaaaa

SEQ ID NO: 1084 - RNA, 25 nt, Artificial Sequence
  Description of Artificial Sequence Synthetic probe
uaaaagauugugggaccauggcacg

SEQ ID NO: 1085 - RNA, 25 nt, Artificial Sequence
  Description of Artificial Sequence Synthetic probe
gaccaugaaguauuagaugauagug

SEQ ID NO: 1086 - RNA, 25 nt, Artificial Sequence
  Description of Artificial Sequence Synthetic probe
acgacaaacgcaacuacaacauagu

SEQ ID NO: 1087 - RNA, 25 nt, Artificial Sequence
  Description of Artificial Sequence Synthetic probe
agcaauacauauggagagacaccug

SEQ ID NO: 1088 - RNA, 25 nt, Artificial Sequence
  Description of Artificial Sequence Synthetic probe
ccauuuugcauguacuaccauauac

SEQ ID NO: 1089 - RNA, 25 nt, Artificial Sequence
  Description of Artificial Sequence Synthetic probe
uuuccccaaugguagcagaaaauuu

SEQ ID NO: 1090 - RNA, 25 nt, Artificial Sequence
  Description of Artificial Sequence Synthetic probe
gauuggguuugugcauuguuuggcg

SEQ ID NO: 1091 - RNA, 25 nt, Artificial Sequence
  Description of Artificial Sequence Synthetic probe
aaugaguugguacggguguuuaaaa

SEQ ID NO: 1092 - RNA, 25 nt, Artificial Sequence
  Description of Artificial Sequence Synthetic probe
gcaaaagcaacguuaauggcaaaau

SEQ ID NO: 1093 - RNA, 25 nt, Artificial Sequence
  Description of Artificial Sequence Synthetic probe
cugugcaaauguagaacuaaacagu

SEQ ID NO: 1094 - RNA, 25 nt, Artificial Sequence
  Description of Artificial Sequence Synthetic probe
uggcgguucacagaacagugugugu

SEQ ID NO: 1095 - RNA, 25 nt, Artificial Sequence
  Description of Artificial Sequence Synthetic probe
aggagauuacuggacaguuauccgg

SEQ ID NO: 1096 - RNA, 25 nt, Artificial Sequence
  Description of Artificial Sequence Synthetic probe
ucaggcaaacgagucacaaguuaaa

SEQ ID NO: 1097 - RNA, 25 nt, Artificial Sequence
  Description of Artificial Sequence Synthetic probe
aucaaaacaacacacacagccauag

SEQ ID NO: 1098 - RNA, 25 nt, Artificial Sequence
  Description of Artificial Sequence Synthetic probe
gaaaguuucuagucagcccgcgaag

SEQ ID NO: 1099 - RNA, 25 nt, Artificial Sequence
  Description of Artificial Sequence Synthetic probe
aaacaaagaggcugugcaucaguua

SEQ ID NO: 1100 - RNA, 25 nt, Artificial Sequence
  Description of Artificial Sequence Synthetic probe
uguuucaggcccaagaauuacaggc

SEQ ID NO: 1101 - RNA, 25 nt, Artificial Sequence
  Description of Artificial Sequence Synthetic probe
ucaggcggaacaggagacagcacgg

SEQ ID NO: 1102 - RNA, 25 nt, Artificial Sequence
  Description of Artificial Sequence Synthetic probe
gaaaaugcagaugauacaggaucug

SEQ ID NO: 1103 - RNA, 25 nt, Artificial Sequence
  Description of Artificial Sequence Synthetic probe
agauaauguuucggaugaugaggau

SEQ ID NO: 1104 - RNA, 25 nt, Artificial Sequence
  Description of Artificial Sequence Synthetic probe
cuagcaacggcgauggacugugaag

SEQ ID NO: 1105 - RNA, 25 nt, Artificial Sequence
  Description of Artificial Sequence Synthetic probe
aagccugguuugcccguguugugcg

SEQ ID NO: 1106 - RNA, 25 nt, Artificial Sequence
  Description of Artificial Sequence Synthetic probe
cgcguuguacagcagauguuaaugg

SEQ ID NO: 1107 - RNA, 25 nt, Artificial Sequence
  Description of Artificial Sequence Synthetic probe
cuggcaguggaaagcaguggagaca

SEQ ID NO: 1108 - RNA, 25 nt, Artificial Sequence
  Description of Artificial Sequence Synthetic probe
uugcagguguucaaguguaguacaa

SEQ ID NO: 1109 - RNA, 25 nt, Artificial Sequence
  Description of Artificial Sequence Synthetic probe
cguguuacagaauugaagcuccgug

SEQ ID NO: 1110 - RNA, 25 nt, Artificial Sequence
  Description of Artificial Sequence Synthetic probe
gaccagcuaccagaaagacgggcug

SEQ ID NO: 1111 - RNA, 25 nt, Artificial Sequence
  Description of Artificial Sequence Synthetic probe
ggaggaugaaguagauaauaugcgu

SEQ ID NO: 1112 - RNA, 25 nt, Artificial Sequence
  Description of Artificial Sequence Synthetic probe
auaaagccaugcgugguaauguacc

SEQ ID NO: 1113 - RNA, 25 nt, Artificial Sequence
  Description of Artificial Sequence Synthetic probe
gcgcuaauugcuggcaacguacacg

SEQ ID NO: 1114 - RNA, 25 nt, Artificial Sequence
  Description of Artificial Sequence Synthetic probe
agaccacuugggccugaagaaaagc

SEQ ID NO: 1115 - RNA, 25 nt, Artificial Sequence
  Description of Artificial Sequence Synthetic probe
ugguacuacauuagaggcaauuacu

SEQ ID NO: 1116 - RNA, 25 nt, Artificial Sequence
  Description of Artificial Sequence Synthetic probe
auagacguuauagcaggucugugua

SEQ ID NO: 1117 - RNA, 25 nt, Artificial Sequence
  Description of Artificial Sequence Synthetic probe
guagagcagauguauauaauguagc

SEQ ID NO: 1118 - RNA, 25 nt, Artificial Sequence
  Description of Artificial Sequence Synthetic probe
ucuaugcacaauauacagguagugu

SEQ ID NO: 1119 - RNA, 25 nt, Artificial Sequence
  Description of Artificial Sequence Synthetic probe
gaagacaagagggaaagaccacgaa

SEQ ID NO: 1120 - RNA, 25 nt, Artificial Sequence
  Description of Artificial Sequence Synthetic probe
gguaaaaguauagaagaacaccaug

SEQ ID NO: 1121 - RNA, 25 nt, Artificial Sequence
  Description of Artificial Sequence Synthetic probe
uguuacucaccaggugugcacuaca

SEQ ID NO: 1122 - RNA, 25 nt, Artificial Sequence
  Description of Artificial Sequence Synthetic probe
cgccaaauaugucuuguaaaacaug

SEQ ID NO: 1123 - RNA, 25 nt, Artificial Sequence
  Description of Artificial Sequence Synthetic probe
uguuggcuuacacaaguacauccua

SEQ ID NO: 1124 - RNA, 25 nt, Artificial Sequence
  Description of Artificial Sequence Synthetic probe
caauacauugccuaacauugcaugu

SEQ ID NO: 1125 - RNA, 25 nt, Artificial Sequence
  Description of Artificial Sequence Synthetic probe
gcugacucacagguccugcagugca

SEQ ID NO: 1126 - RNA, 25 nt, Artificial Sequence
  Description of Artificial Sequence Synthetic probe
guugucccgccuaaacugacuucuu

SEQ ID NO: 1127 - RNA, 25 nt, Artificial Sequence
  Description of Artificial Sequence Synthetic probe
uccugcaguccacuggucuacacuu

SEQ ID NO: 1128 - RNA, 25 nt, Artificial Sequence
  Description of Artificial Sequence Synthetic probe
ccauuuuaaauccuaaccgaauucg

SEQ ID NO: 1129 - RNA, 25 nt, Artificial Sequence
  Description of Artificial Sequence Synthetic probe
cucuccauuuuguaccauuuuguac

SEQ ID NO: 1130 - RNA, 25 nt, Artificial Sequence
  Description of Artificial Sequence Synthetic probe
guguccuacuuuguuacacuacuaa

SEQ ID NO: 1131 - RNA, 25 nt, Artificial Sequence
  Description of Artificial Sequence Synthetic probe
uacccuguguccccugcccuacccu

SEQ ID NO: 1132 - RNA, 25 nt, Artificial Sequence
  Description of Artificial Sequence Synthetic probe
gcuccuaaucuauugcaucuccugc

SEQ ID NO: 1133 - RNA, 25 nt, Artificial Sequence
  Description of Artificial Sequence Synthetic probe
cacccacaugaguaacaauacaguu

SEQ ID NO: 1134 - RNA, 25 nt, Artificial Sequence
  Description of Artificial Sequence Synthetic probe
caguuccuguauguauguuuugugu

SEQ ID NO: 1135 - RNA, 25 nt, Artificial Sequence
  Description of Artificial Sequence Synthetic probe
uuugcauguuauguaugugugugca

SEQ ID NO: 1136 - RNA, 25 nt, Artificial Sequence
  Description of Artificial Sequence Synthetic probe
auuguuuuguguguguacuguguug

SEQ ID NO: 1137 - RNA, 25 nt, Artificial Sequence
  Description of Artificial Sequence Synthetic probe
ugucaaacacagguuaaaagguaac

SEQ ID NO: 1138 - RNA, 25 nt, Artificial Sequence
  Description of Artificial Sequence Synthetic probe
gguaauugucugugucauguaugug

SEQ ID NO: 1139 - RNA, 25 nt, Artificial Sequence
  Description of Artificial Sequence Synthetic probe
guuaaaagguaaccauugucuguug

SEQ ID NO: 1140 - RNA, 25 nt, Artificial Sequence
  Description of Artificial Sequence Synthetic probe
ggccccacguaccuccacaaagaag

SEQ ID NO: 1141 - RNA, 25 nt, Artificial Sequence
  Description of Artificial Sequence Synthetic probe
ccaaacuaaaacgcccugcaucauc

SEQ ID NO: 1142 - RNA, 25 nt, Artificial Sequence
  Description of Artificial Sequence Synthetic probe
uuacaggcagggcuacaggcuaggc

SEQ ID NO: 1143 - RNA, 25 nt, Artificial Sequence
  Description of Artificial Sequence Synthetic probe
aaaggacuauauguuuugggaggug

SEQ ID NO: 1144 - RNA, 25 nt, Artificial Sequence
  Description of Artificial Sequence Synthetic probe
caccaccuaaaggaaaggaagaucc

SEQ ID NO: 1145 - RNA, 25 nt, Artificial Sequence
  Description of Artificial Sequence Synthetic probe
gucacuucuacugcuauaacuuguc

SEQ ID NO: 1146 - RNA, 25 nt, Artificial Sequence
  Description of Artificial Sequence Synthetic probe
caccgucugcaucuuuggaggacac

SEQ ID NO: 1147 - RNA, 25 nt, Artificial Sequence
  Description of Artificial Sequence Synthetic probe
cauaagauggaugccacuauuuuag

SEQ ID NO: 1148 - RNA, 25 nt, Artificial Sequence
  Description of Artificial Sequence Synthetic probe
aagggucuaacucuggcaauacugc

SEQ ID NO: 1149 - RNA, 25 nt, Artificial Sequence
  Description of Artificial Sequence Synthetic probe
ccugugccaggugauuuauauauac

SEQ ID NO: 1150 - RNA, 25 nt, Artificial Sequence
  Description of Artificial Sequence Synthetic probe
cgagccauauggugacaguuuguuc

SEQ ID NO: 1151 - RNA, 25 nt, Artificial Sequence
  Description of Artificial Sequence Synthetic probe
uagcaguguauguaaguauccagau

SEQ ID NO: 1152 - RNA, 25 nt, Artificial Sequence
  Description of Artificial Sequence Synthetic probe
ggauuuuaauaccuugcaagcuagu

SEQ ID NO: 1153 - RNA, 25 nt, Artificial Sequence
  Description of Artificial Sequence Synthetic probe
cagcucauuaacaguguaauacagg

SEQ ID NO: 1154 - RNA, 25 nt, Artificial Sequence
  Description of Artificial Sequence Synthetic probe
cugguaaaccugguauagauaauag

SEQ ID NO: 1155 - RNA, 25 nt, Artificial Sequence
  Description of Artificial Sequence Synthetic probe
guuugaugauacugaaaccaguaac

SEQ ID NO: 1156 - RNA, 25 nt, Artificial Sequence
  Description of Artificial Sequence Synthetic probe
gcuccaucuacaucuauuauuguug

SEQ ID NO: 1157 - RNA, 25 nt, Artificial Sequence
  Description of Artificial Sequence Synthetic probe
uccuuuuguuccuauagccccuaca

SEQ ID NO: 1158 - RNA, 25 nt, Artificial Sequence
  Description of Artificial Sequence Synthetic probe
cauuaccuucguuacccacacauac

SEQ ID NO: 1159 - RNA, 25 nt, Artificial Sequence
  Description of Artificial Sequence Synthetic probe
cuauguccauugagucagguccuga

SEQ ID NO: 1160 - RNA, 25 nt, Artificial Sequence
  Description of Artificial Sequence Synthetic probe
gguauugacuuuguauaucaaccca

SEQ ID NO: 1161 - RNA, 25 nt, Artificial Sequence
  Description of Artificial Sequence Synthetic probe
cuuccacacuuucuacccauaauaa

SEQ ID NO: 1162 - RNA, 25 nt, Artificial Sequence
  Description of Artificial Sequence Synthetic probe
uugcagcaacccacguuucacuuac

SEQ ID NO: 1163 - RNA, 25 nt, Artificial Sequence
  Description of Artificial Sequence Synthetic probe
cccuuacacuauuaaugaugguuug

SEQ ID NO: 1164 - RNA, 25 nt, Artificial Sequence
  Description of Artificial Sequence Synthetic probe
aaccuuuauuaccacagucuguguc

SEQ ID NO: 1165 - RNA, 25 nt, Artificial Sequence
  Description of Artificial Sequence Synthetic probe
gaaguucaggaagacauagaauugc

SEQ ID NO: 1166 - RNA, 25 nt, Artificial Sequence
  Description of Artificial Sequence Synthetic probe
ugauauuaguccuauccagccugcu

SEQ ID NO: 1167 - RNA, 25 nt, Artificial Sequence
  Description of Artificial Sequence Synthetic probe
aacuuuuaccugcaccggauccuga

SEQ ID NO: 1168 - RNA, 25 nt, Artificial Sequence
  Description of Artificial Sequence Synthetic probe
ggcguugauacagaugaaacuauaa

SEQ ID NO: 1169 - RNA, 25 nt, Artificial Sequence
  Description of Artificial Sequence Synthetic probe
gucaucaccacagaaauuaguaaca

SEQ ID NO: 1170 - RNA, 25 nt, Artificial Sequence
  Description of Artificial Sequence Synthetic probe
ccuugguuuauauagccgugccaca

SEQ ID NO: 1171 - RNA, 25 nt, Artificial Sequence
  Description of Artificial Sequence Synthetic probe
gcaguguaacaaguaguacaccuau

SEQ ID NO: 1172 - RNA, 25 nt, Artificial Sequence
  Description of Artificial Sequence Synthetic probe
acauuuguuaccucuacugacagca

SEQ ID NO: 1173 - RNA, 25 nt, Artificial Sequence
  Description of Artificial Sequence Synthetic probe
cuauuaguacacacaccuaugaaga

SEQ ID NO: 1174 - RNA, 25 nt, Artificial Sequence
  Description of Artificial Sequence Synthetic probe
ggucauguauuguuuucuaguccaa

SEQ ID NO: 1175 - RNA, 25 nt, Artificial Sequence
  Description of Artificial Sequence Synthetic probe
ccuacauucacugaaccaucuauaa

SEQ ID NO: 1176 - RNA, 25 nt, Artificial Sequence
  Description of Artificial Sequence Synthetic probe
caucuguacaaucaguuucuacaca

SEQ ID NO: 1177 - RNA, 25 nt, Artificial Sequence
  Description of Artificial Sequence Synthetic probe
acaacaucugcaaauaauacuccug

SEQ ID NO: 1178 - RNA, 25 nt, Artificial Sequence
  Description of Artificial Sequence Synthetic probe
auuccaucagcaacaggguuugaug

SEQ ID NO: 1179 - RNA, 25 nt, Artificial Sequence
  Description of Artificial Sequence Synthetic probe
caacauuuauugagucuggcgcacc

SEQ ID NO: 1180 - RNA, 25 nt, Artificial Sequence
  Description of Artificial Sequence Synthetic probe
cccuuagaaccaucuauaguuucua

SEQ ID NO: 1181 - RNA, 25 nt, Artificial Sequence
  Description of Artificial Sequence Synthetic probe
uaguauuaccacguccaccauucgu

SEQ ID NO: 1182 - RNA, 25 nt, Artificial Sequence
  Description of Artificial Sequence Synthetic probe
cauuguccacucguccucccacuag

SEQ ID NO: 1183 - RNA, 25 nt, Artificial Sequence
  Description of Artificial Sequence Synthetic probe
gcucuggugguagggcaggcuaugu

SEQ ID NO: 1184 - RNA, 25 nt, Artificial Sequence
  Description of Artificial Sequence Synthetic probe
ggagguuuggguauagguacaggug

SEQ ID NO: 1185 - RNA, 25 nt, Artificial Sequence
  Description of Artificial Sequence Synthetic probe
uuuuaaaauauggcagccuaggggu

SEQ ID NO: 1186 - RNA, 25 nt, Artificial Sequence
  Description of Artificial Sequence Synthetic probe
uagcuugucgcaaugagauacagac

SEQ ID NO: 1187 - RNA, 25 nt, Artificial Sequence
  Description of Artificial Sequence Synthetic probe
auaacuguacauguagauuggcuac

SEQ ID NO: 1188 - RNA, 25 nt, Artificial Sequence
  Description of Artificial Sequence Synthetic probe
uguuuuguauucacugucaugcaca

SEQ ID NO: 1189 - RNA, 25 nt, Artificial Sequence
  Description of Artificial Sequence Synthetic probe
aucuauugggucaccauuuaaagug

SEQ ID NO: 1190 - RNA, 25 nt, Artificial Sequence
  Description of Artificial Sequence Synthetic probe
uaugcgcagguguuggugcuggugc

SEQ ID NO: 1191 - RNA, 25 nt, Artificial Sequence
  Description of Artificial Sequence Synthetic probe
cagugcuuaggccgcucuugcuauc

SEQ ID NO: 1192 - RNA, 25 nt, Artificial Sequence
  Description of Artificial Sequence Synthetic probe
aacacccaacacaagccaauauugc

SEQ ID NO: 1193 - RNA, 25 nt, Artificial Sequence
  Description of Artificial Sequence Synthetic probe
ggugucaugucauugugauauuugu

SEQ ID NO: 1194 - RNA, 25 nt, Artificial Sequence
  Description of Artificial Sequence Synthetic probe
cagugaugaaacacaacgucaacaa

SEQ ID NO: 1195 - RNA, 25 nt, Artificial Sequence
  Description of Artificial Sequence Synthetic probe
guauguucaaauuucaucuaccugg

SEQ ID NO: 1196 - RNA, 25 nt, Artificial Sequence
  Description of Artificial Sequence Synthetic probe
caacuuguacugcaccuauaauaca

SEQ ID NO: 1197 - RNA, 25 nt, Artificial Sequence
  Description of Artificial Sequence Synthetic probe
cggggacucgucacugcaacugagu

SEQ ID NO: 1198 - RNA, 25 nt, Artificial Sequence
  Description of Artificial Sequence Synthetic probe
ugcggggacaacaauccguggacag

SEQ ID NO: 1199 - RNA, 25 nt, Artificial Sequence
  Description of Artificial Sequence Synthetic probe
aacaccaaguaccccaacaaccuuu

SEQ ID NO: 1200 - RNA, 25 nt, Artificial Sequence
  Description of Artificial Sequence Synthetic probe
uacaaccaccacagaaacgacgacg

SEQ ID NO: 1201 - RNA, 25 nt, Artificial Sequence
  Description of Artificial Sequence Synthetic probe
gcaguguccgugggugccaaagaca

SEQ ID NO: 1202 - RNA, 25 nt, Artificial Sequence
  Description of Artificial Sequence Synthetic probe
augcaccgaaaccuccaagaccucc

SEQ ID NO: 1203 - RNA, 25 nt, Artificial Sequence
  Description of Artificial Sequence Synthetic probe
ggguuauauuauuggugugauggag

SEQ ID NO: 1204 - RNA, 25 nt, Artificial Sequence
  Description of Artificial Sequence Synthetic probe
guacaauuguagaaggacaaguaga

SEQ ID NO: 1205 - RNA, 25 nt, Artificial Sequence
  Description of Artificial Sequence Synthetic probe
cuauggauuauacaaacuggaagga

SEQ ID NO: 1206 - RNA, 25 nt, Artificial Sequence
  Description of Artificial Sequence Synthetic probe
uggguauacaauaacagugcaauac

SEQ ID NO: 1207 - RNA, 25 nt, Artificial Sequence
  Description of Artificial Sequence Synthetic probe
ucuagaaauguggcgugcagaacca

SEQ ID NO: 1208 - RNA, 25 nt, Artificial Sequence
  Description of Artificial Sequence Synthetic probe
agauggauggacauuacaacaaaca

SEQ ID NO: 1209 - RNA, 25 nt, Artificial Sequence
  Description of Artificial Sequence Synthetic probe
cauuggaggcauuaaacaaaacaca

SEQ ID NO: 1210 - RNA, 25 nt, Artificial Sequence
  Description of Artificial Sequence Synthetic probe
cugggaauaacucauauaggccacc

SEQ ID NO: 1211 - RNA, 25 nt, Artificial Sequence
  Description of Artificial Sequence Synthetic probe
gacucgaauggaauguguuuuguuu

SEQ ID NO: 1212 - RNA, 25 nt, Artificial Sequence
  Description of Artificial Sequence Synthetic probe
gaccuaaacgcacaaauugaacauu

SEQ ID NO: 1213 - RNA, 25 nt, Artificial Sequence
  Description of Artificial Sequence Synthetic probe
cuagaucuauacgaagcugauagua

SEQ ID NO: 1214 - RNA, 25 nt, Artificial Sequence
  Description of Artificial Sequence Synthetic probe
ggccauauuuacauaguagauuggu

SEQ ID NO: 1215 - RNA, 25 nt, Artificial Sequence
  Description of Artificial Sequence Synthetic probe
caaauacaaaugcaggaacagaucc

SEQ ID NO: 1216 - RNA, 25 nt, Artificial Sequence
  Description of Artificial Sequence Synthetic probe
guggguaugauagaugauguaacac

SEQ ID NO: 1217 - RNA, 25 nt, Artificial Sequence
  Description of Artificial Sequence Synthetic probe
guucuuaaguggauguguaauaucc

SEQ ID NO: 1218 - RNA, 25 nt, Artificial Sequence
  Description of Artificial Sequence Synthetic probe
auagaauagaugaugguggagauug

SEQ ID NO: 1219 - RNA, 25 nt, Artificial Sequence
  Description of Artificial Sequence Synthetic probe
gcauauucgauuuuggagaaauggu

SEQ ID NO: 1220 - RNA, 25 nt, Artificial Sequence
  Description of Artificial Sequence Synthetic probe
cagguuugucuaauauuagugaggu

SEQ ID NO: 1221 - RNA, 25 nt, Artificial Sequence
  Description of Artificial Sequence Synthetic probe
gaagugcuaccugugcauuauauug

SEQ ID NO: 1222 - RNA, 25 nt, Artificial Sequence
  Description of Artificial Sequence Synthetic probe
cagaaacacauaugguaauagaacc

SEQ ID NO: 1223 - RNA, 25 nt, Artificial Sequence
  Description of Artificial Sequence Synthetic probe
ccaaacuaaugucacagcuguuaaa

SEQ ID NO: 1224 - RNA, 25 nt, Artificial Sequence
  Description of Artificial Sequence Synthetic probe
gcuuauacugcugcuaauuagguuu

SEQ ID NO: 1225 - RNA, 25 nt, Artificial Sequence
  Description of Artificial Sequence Synthetic probe
caucaguugcagaaggauuaaaagu

SEQ ID NO: 1226 - RNA, 25 nt, Artificial Sequence
  Description of Artificial Sequence Synthetic probe
uguauuauaggaaugggaguaacac

SEQ ID NO: 1227 - RNA, 25 nt, Artificial Sequence
  Description of Artificial Sequence Synthetic probe
guauagaggacaaugaggaaaauag

SEQ ID NO: 1228 - RNA, 25 nt, Artificial Sequence
  Description of Artificial Sequence Synthetic probe
guaacaguagucaaucaaguggggu

SEQ ID NO: 1229 - RNA, 25 nt, Artificial Sequence
  Description of Artificial Sequence Synthetic probe
caugucacguagaagacagcggcua

SEQ ID NO: 1230 - RNA, 25 nt, Artificial Sequence
  Description of Artificial Sequence Synthetic probe
auacagaguguguuuuaccaaaacg

SEQ ID NO: 1231 - RNA, 25 nt, Artificial Sequence
  Description of Artificial Sequence Synthetic probe
gaaagugcugggcaagaugguguag

SEQ ID NO: 1232 - RNA, 25 nt, Artificial Sequence
  Description of Artificial Sequence Synthetic probe
uacaugcugugucugcaguaaaacg

SEQ ID NO: 1233 - RNA, 25 nt, Artificial Sequence
  Description of Artificial Sequence Synthetic probe
aaugaacaggcagaacaugaggcag

SEQ ID NO: 1234 - RNA, 25 nt, Artificial Sequence
  Description of Artificial Sequence Synthetic probe
gcauaugauaguggaacagaucuaa

SEQ ID NO: 1235 - RNA, 25 nt, Artificial Sequence
  Description of Artificial Sequence Synthetic probe
gggauguacaggcugguuugaagua

SEQ ID NO: 1236 - RNA, 25 nt, Artificial Sequence
  Description of Artificial Sequence Synthetic probe
acaacccugcaauggaggacccuga

SEQ ID NO: 1237 - RNA, 25 nt, Artificial Sequence
  Description of Artificial Sequence Synthetic probe
gaccuucguacucuacagcaaaugc

SEQ ID NO: 1238 - RNA, 25 nt, Artificial Sequence
  Description of Artificial Sequence Synthetic probe
gcacacuacggcuaugcauucauag

SEQ ID NO: 1239 - RNA, 25 nt, Artificial Sequence
  Description of Artificial Sequence Synthetic probe
uagaucugcaaccugaaacaacuga

SEQ ID NO: 1240 - RNA, 25 nt, Artificial Sequence
  Description of Artificial Sequence Synthetic probe
guggagacaaagcaacuauaaaaga

SEQ ID NO: 1241 - RNA, 25 nt, Artificial Sequence
  Description of Artificial Sequence Synthetic probe
cugugacccaaguguaacgucaugc

SEQ ID NO: 1242 - RNA, 25 nt, Artificial Sequence
  Description of Artificial Sequence Synthetic probe
auuaugggucguuggacagggcgcu

SEQ ID NO: 1243 - RNA, 25 nt, Artificial Sequence
  Description of Artificial Sequence Synthetic probe
cauguuaaugcaaacaagcgauuuc

SEQ ID NO: 1244 - RNA, 25 nt, Artificial Sequence
  Description of Artificial Sequence Synthetic probe
ugucaaacgccauuauguccugaag

SEQ ID NO: 1245 - RNA, 25 nt, Artificial Sequence
  Description of Artificial Sequence Synthetic probe
augggaaaacauuagaagagagggu

SEQ ID NO: 1246 - RNA, 25 nt, Artificial Sequence
  Description of Artificial Sequence Synthetic probe
auggcguguguauuaugugccuacg

SEQ ID NO: 1247 - RNA, 25 nt, Artificial Sequence
  Description of Artificial Sequence Synthetic probe
cgaagagagguauacaaguuucuau

SEQ ID NO: 1248 - RNA, 25 nt, Artificial Sequence
  Description of Artificial Sequence Synthetic probe
gcaugaaauaaggcugcagugugug

SEQ ID NO: 1249 - RNA, 25 nt, Artificial Sequence
  Description of Artificial Sequence Synthetic probe
ugugugaggugcuggaagaaucggu

SEQ ID NO: 1250 - RNA, 25 nt, Artificial Sequence
  Description of Artificial Sequence Synthetic probe
acacgaccccggacccugcacgaau

SEQ ID NO: 1251 - RNA, 25 nt, Artificial Sequence
  Description of Artificial Sequence Synthetic probe
cacggccauguuugaggauccagca

SEQ ID NO: 1252 - RNA, 25 nt, Artificial Sequence
  Description of Artificial Sequence Synthetic probe
uauauagaacacaguguagcuaacg

SEQ ID NO: 1253 - RNA, 25 nt, Artificial Sequence
  Description of Artificial Sequence Synthetic probe
gucaguaucuguuuugcaaacaugu

SEQ ID NO: 1254 - RNA, 25 nt, Artificial Sequence
  Description of Artificial Sequence Synthetic probe
aauacacuauguaggccaaguaucu

SEQ ID NO: 1255 - RNA, 25 nt, Artificial Sequence
  Description of Artificial Sequence Synthetic probe
ugugucugcaacuuugguguuuugg

SEQ ID NO: 1256 - RNA, 25 nt, Artificial Sequence
  Description of Artificial Sequence Synthetic probe
guaccgcacccuguauuacucacag

SEQ ID NO: 1257 - RNA, 25 nt, Artificial Sequence
  Description of Artificial Sequence Synthetic probe
ggcccuuuucagcagaacaguuaau

SEQ ID NO: 1258 - RNA, 25 nt, Artificial Sequence
  Description of Artificial Sequence Synthetic probe
gccuagugccauuauuuaaaccaaa

SEQ ID NO: 1259 - RNA, 25 nt, Artificial Sequence
  Description of Artificial Sequence Synthetic probe
cauuuuguacaugcaaccgaauucg

SEQ ID NO: 1260 - RNA, 25 nt, Artificial Sequence
  Description of Artificial Sequence Synthetic probe
guguacuauguguauugugcauaca

SEQ ID NO: 1261 - RNA, 25 nt, Artificial Sequence
  Description of Artificial Sequence Synthetic probe
gugugucauuauuguggcuuuuguu

SEQ ID NO: 1262 - RNA, 25 nt, Artificial Sequence
  Description of Artificial Sequence Synthetic probe
gucuguaauaaacaugaaugagugc

SEQ ID NO: 1263 - RNA, 25 nt, Artificial Sequence
  Description of Artificial Sequence Synthetic probe
uuuguguaacuguguuuguguguug

SEQ ID NO: 1264 - RNA, 25 nt, Artificial Sequence
  Description of Artificial Sequence Synthetic probe
guaaaaggcgguaguguguuguugu

SEQ ID NO: 1265 - RNA, 25 nt, Artificial Sequence
  Description of Artificial Sequence Synthetic probe
accuccaccucuacaccagcaaaac

SEQ ID NO: 1266 - RNA, 25 nt, Artificial Sequence
  Description of Artificial Sequence Synthetic probe
gucaaagccugcuguagcuaccucu

SEQ ID NO: 1267 - RNA, 25 nt, Artificial Sequence
  Description of Artificial Sequence Synthetic probe
ugucaacgggaacagccaccaacag

SEQ ID NO: 1268 - RNA, 25 nt, Artificial Sequence
  Description of Artificial Sequence Synthetic probe
caccagccuagaagauaaauauaga

SEQ ID NO: 1269 - RNA, 25 nt, Artificial Sequence
  Description of Artificial Sequence Synthetic probe
aauaugaaugcuaaccuacuggagg

SEQ ID NO: 1270 - RNA, 25 nt, Artificial Sequence
  Description of Artificial Sequence Synthetic probe
caaaauuacuuugucugcagagguu

SEQ ID NO: 1271 - RNA, 25 nt, Artificial Sequence
  Description of Artificial Sequence Synthetic probe
cuaacaugacuauuaguacugcuac

SEQ ID NO: 1272 - RNA, 25 nt, Artificial Sequence
  Description of Artificial Sequence Synthetic probe
ugauuacgucugaggcacaguuauu

SEQ ID NO: 1273 - RNA, 25 nt, Artificial Sequence
  Description of Artificial Sequence Synthetic probe
uuuaaaggguagcaaugguagagaa

SEQ ID NO: 1274 - RNA, 25 nt, Artificial Sequence
  Description of Artificial Sequence Synthetic probe
uuggggaaacaauaccugcagaguu

SEQ ID NO: 1275 - RNA, 25 nt, Artificial Sequence
  Description of Artificial Sequence Synthetic probe
accuuuagacauuguacaauccacc

SEQ ID NO: 1276 - RNA, 25 nt, Artificial Sequence
  Description of Artificial Sequence Synthetic probe
gcuauggacuuuaagguguugcagg

SEQ ID NO: 1277 - RNA, 25 nt, Artificial Sequence
  Description of Artificial Sequence Synthetic probe
gccucuugcauuaauuaauacaccu

SEQ ID NO: 1278 - RNA, 25 nt, Artificial Sequence
  Description of Artificial Sequence Synthetic probe
aaguccacacaaguuaccacagggg

SEQ ID NO: 1279 - RNA, 25 nt, Artificial Sequence
  Description of Artificial Sequence Synthetic probe
acauuggacuaaaggugcugugugu

SEQ ID NO: 1280 - RNA, 25 nt, Artificial Sequence
  Description of Artificial Sequence Synthetic probe
uauaucaguugauggcaagcaaaca

SEQ ID NO: 1281 - RNA, 25 nt, Artificial Sequence
  Description of Artificial Sequence Synthetic probe
uauuuauaauccggaccaggaacgg

SEQ ID NO: 1282 - RNA, 25 nt, Artificial Sequence
  Description of Artificial Sequence Synthetic probe
cauucccaaaguuagugcauaucaa

SEQ ID NO: 1283 - RNA, 25 nt, Artificial Sequence
  Description of Artificial Sequence Synthetic probe
gugacuaaggacaauaccaaaacaa

SEQ ID NO: 1284 - RNA, 25 nt, Artificial Sequence
  Description of Artificial Sequence Synthetic probe
uccuccuuugcauuauggccugugu

SEQ ID NO: 1285 - RNA, 25 nt, Artificial Sequence
  Description of Artificial Sequence Synthetic probe
ccuuuguuccucagucuccuuauga

SEQ ID NO: 1286 - RNA, 25 nt, Artificial Sequence
  Description of Artificial Sequence Synthetic probe
ccauuuuauucagguccugacauag

SEQ ID NO: 1287 - RNA, 25 nt, Artificial Sequence
  Description of Artificial Sequence Synthetic probe
cccuuuagguaaugugugggaaaca

SEQ ID NO: 1288 - RNA, 25 nt, Artificial Sequence
  Description of Artificial Sequence Synthetic probe
cuaguaacaccacuaauguaacugc

SEQ ID NO: 1289 - RNA, 25 nt, Artificial Sequence
  Description of Artificial Sequence Synthetic probe
acacuuaccuauaaagccuuccaca

SEQ ID NO: 1290 - RNA, 25 nt, Artificial Sequence
  Description of Artificial Sequence Synthetic probe
cuagccagucaguugcuacaccuuc

SEQ ID NO: 1291 - RNA, 25 nt, Artificial Sequence
  Description of Artificial Sequence Synthetic probe
acuauacaaacacguagaggcacac

SEQ ID NO: 1292 - RNA, 25 nt, Artificial Sequence
  Description of Artificial Sequence Synthetic probe
accugcaacauuaguaucugcugau

SEQ ID NO: 1293 - RNA, 25 nt, Artificial Sequence
  Description of Artificial Sequence Synthetic probe
gcagcuccuagauuauauagaaaag

SEQ ID NO: 1294 - RNA, 25 nt, Artificial Sequence
  Description of Artificial Sequence Synthetic probe
auuugcuguucacgguucugguaca

SEQ ID NO: 1295 - RNA, 25 nt, Artificial Sequence
  Description of Artificial Sequence Synthetic probe
gcaauauuuuaauuagcacacccac

SEQ ID NO: 1296 - RNA, 25 nt, Artificial Sequence
  Description of Artificial Sequence Synthetic probe
guacccauauaaccaauccguuauu

SEQ ID NO: 1297 - RNA, 25 nt, Artificial Sequence
  Description of Artificial Sequence Synthetic probe
accucuaguacuguacaugucagua

SEQ ID NO: 1298 - RNA, 25 nt, Artificial Sequence
  Description of Artificial Sequence Synthetic probe
agggauuccuaauuuuacugggucu

SEQ ID NO: 1299 - RNA, 25 nt, Artificial Sequence
  Description of Artificial Sequence Synthetic probe
gaguccaguguuauagaaucuggug

SEQ ID NO: 1300 - RNA, 25 nt, Artificial Sequence
  Description of Artificial Sequence Synthetic probe
acuccggcgcgaccaccuauuguug

SEQ ID NO: 1301 - RNA, 25 nt, Artificial Sequence
  Description of Artificial Sequence Synthetic probe
ggcuauguuccauuggggucuaggc

SEQ ID NO: 1302 - RNA, 25 nt, Artificial Sequence
  Description of Artificial Sequence Synthetic probe
uaguacuguuacuacuaugguugcc

SEQ ID NO: 1303 - RNA, 25 nt, Artificial Sequence
  Description of Artificial Sequence Synthetic probe
cugugcuguguauauauuuacaugc

SEQ ID NO: 1304 - RNA, 25 nt, Artificial Sequence
  Description of Artificial Sequence Synthetic probe
guuuugguuuguuauagccacaucc

SEQ ID NO: 1305 - RNA, 25 nt, Artificial Sequence
  Description of Artificial Sequence Synthetic probe
ccucuguguuuuccaguuguauauu

SEQ ID NO: 1306 - RNA, 25 nt, Artificial Sequence
  Description of Artificial Sequence Synthetic probe
gucauguugucccgcuuuugcuauc

SEQ ID NO: 1307 - RNA, 25 nt, Artificial Sequence
  Description of Artificial Sequence Synthetic probe
ugcuuuuguguuuguuugcuugugu

SEQ ID NO: 1308 - RNA, 25 nt, Artificial Sequence
  Description of Artificial Sequence Synthetic probe
ugcuacgcauauauauugcaaccau

SEQ ID NO: 1309 - RNA, 25 nt, Artificial Sequence
  Description of Artificial Sequence Synthetic probe
gugaaguguaccugccauacauugc

SEQ ID NO: 1310 - RNA, 25 nt, Artificial Sequence
  Description of Artificial Sequence Synthetic probe
caaaugaguuuuccauaaagugcug

SEQ ID NO: 1311 - RNA, 25 nt, Artificial Sequence
  Description of Artificial Sequence Synthetic probe
caguaguguacagguuaguuuggga

SEQ ID NO: 1312 - RNA, 25 nt, Artificial Sequence
  Description of Artificial Sequence Synthetic probe
cauaucauuggacaaguacagacaa

SEQ ID NO: 1313 - RNA, 25 nt, Artificial Sequence
  Description of Artificial Sequence Synthetic probe
caguagaaguagaaguaucaacaac

SEQ ID NO: 1314 - RNA, 25 nt, Artificial Sequence
  Description of Artificial Sequence Synthetic probe
acaucagcgacacagacaauaccga

SEQ ID NO: 1315 - RNA, 25 nt, Artificial Sequence
  Description of Artificial Sequence Synthetic probe
gaaucagaauuugacuccuccagag

SEQ ID NO: 1316 - RNA, 25 nt, Artificial Sequence
  Description of Artificial Sequence Synthetic probe
accaggaaaacgacccagacuacgg

SEQ ID NO: 1317 - RNA, 25 nt, Artificial Sequence
  Description of Artificial Sequence Synthetic probe
accaagacgccgcaguaucccacag

SEQ ID NO: 1318 - RNA, 25 nt, Artificial Sequence
  Description of Artificial Sequence Synthetic probe
aauacaacacccacaagaccaccac

SEQ ID NO: 1319 - RNA, 25 nt, Artificial Sequence
  Description of Artificial Sequence Synthetic probe
cuacacagacuuugaacaagaggcc

SEQ ID NO: 1320 - RNA, 25 nt, Artificial Sequence
  Description of Artificial Sequence Synthetic probe
gggguagacuauagagguauauauu

SEQ ID NO: 1321 - RNA, 25 nt, Artificial Sequence
  Description of Artificial Sequence Synthetic probe
guaugcaauauguagccuggaaaua

SEQ ID NO: 1322 - RNA, 25 nt, Artificial Sequence
  Description of Artificial Sequence Synthetic probe
cauuaagagacacaugcgaggaacu

SEQ ID NO: 1323 - RNA, 25 nt, Artificial Sequence
  Description of Artificial Sequence Synthetic probe
gcacuggaaucauuaaguacaacaa

SEQ ID NO: 1324 - RNA, 25 nt, Artificial Sequence
  Description of Artificial Sequence Synthetic probe
cauuacuguacuaaaccaccagaug

SEQ ID NO: 1325 - RNA, 25 nt, Artificial Sequence
  Description of Artificial Sequence Synthetic probe
agaaaacaauggagacgcuuuccca

SEQ ID NO: 1326 - RNA, 25 nt, Artificial Sequence
  Description of Artificial Sequence Synthetic probe
aauguuucuuuacaaggacgugguc

SEQ ID NO: 1327 - RNA, 25 nt, Artificial Sequence
  Description of Artificial Sequence Synthetic probe
ccuaugcuagaugcuaaauuacgau

SEQ ID NO: 1328 - RNA, 25 nt, Artificial Sequence
  Description of Artificial Sequence Synthetic probe
guccaccauuacuaauuacaaccaa

SEQ ID NO: 1329 - RNA, 25 nt, Artificial Sequence
  Description of Artificial Sequence Synthetic probe
augcuaaacuuggguuguuggauga

SEQ ID NO: 1330 - RNA, 25 nt, Artificial Sequence
  Description of Artificial Sequence Synthetic probe
guuugguacuuuguggaccgccaaa

SEQ ID NO: 1331 - RNA, 25 nt, Artificial Sequence
  Description of Artificial Sequence Synthetic probe
caguggauaaagcacauauguagua

SEQ ID NO: 1332 - RNA, 25 nt, Artificial Sequence
  Description of Artificial Sequence Synthetic probe
aaguaacagaugauagccaaauugc

SEQ ID NO: 1333 - RNA, 25 nt, Artificial Sequence
  Description of Artificial Sequence Synthetic probe
cacaguuuacaggauagucaauuug

SEQ ID NO: 1334 - RNA, 25 nt, Artificial Sequence
  Description of Artificial Sequence Synthetic probe
auauuagugauguguauggagacac

SEQ ID NO: 1335 - RNA, 25 nt, Artificial Sequence
  Description of Artificial Sequence Synthetic probe
cacaggagcaaauguuaauucaacc

SEQ ID NO: 1336 - RNA, 25 nt, Artificial Sequence
  Description of Artificial Sequence Synthetic probe
gcaggacuuguuuaaaaguagcaau

SEQ ID NO: 1337 - RNA, 25 nt, Artificial Sequence
  Description of Artificial Sequence Synthetic probe
acaaugaaacgccaacacaacaauu

SEQ ID NO: 1338 - RNA, 25 nt, Artificial Sequence
  Description of Artificial Sequence Synthetic probe
gaggacucuguaauacauauggaua

SEQ ID NO: 1339 - RNA, 25 nt, Artificial Sequence
  Description of Artificial Sequence Synthetic probe
cucacaaaacaguaccuauaguaac

SEQ ID NO: 1340 - RNA, 25 nt, Artificial Sequence
  Description of Artificial Sequence Synthetic probe
ggugcgggaauacacaaaauggagg

SEQ ID NO: 1341 - RNA, 25 nt, Artificial Sequence
  Description of Artificial Sequence Synthetic probe
guagaugaagagguacagggacgug

SEQ ID NO: 1342 - RNA, 25 nt, Artificial Sequence
  Description of Artificial Sequence Synthetic probe
uacauuggaaacucuggaaacacca

SEQ ID NO: 1343 - RNA, 25 nt, Artificial Sequence
  Description of Artificial Sequence Synthetic probe
uuuuaucagaccuacaagacagcgg

SEQ ID NO: 1344 - RNA, 25 nt, Artificial Sequence
  Description of Artificial Sequence Synthetic probe
ccauuaagggauauuaguaaucagc

SEQ ID NO: 1345 - RNA, 25 nt, Artificial Sequence
  Description of Artificial Sequence Synthetic probe
uacaaacagcacaugcagauaaaca

SEQ ID NO: 1346 - RNA, 25 nt, Artificial Sequence
  Description of Artificial Sequence Synthetic probe
gacgcagaaacagucaacaauuguu

SEQ ID NO: 1347 - RNA, 25 nt, Artificial Sequence
  Description of Artificial Sequence Synthetic probe
agaugaugaaagugacgaggaggau

SEQ ID NO: 1348 - RNA, 25 nt, Artificial Sequence
  Description of Artificial Sequence Synthetic probe
ugguuugaaguagaggcaauuguag

SEQ ID NO: 1349 - RNA, 25 nt, Artificial Sequence
  Description of Artificial Sequence Synthetic probe
cgcaucaaguaacuaacugcaaugg

SEQ ID NO: 1350 - RNA, 25 nt, Artificial Sequence
  Description of Artificial Sequence Synthetic probe
ccaaagaggaccugcguguuguaca

SEQ ID NO: 1351 - RNA, 25 nt, Artificial Sequence
  Description of Artificial Sequence Synthetic probe
guuuguggugcaguuggacauucag

SEQ ID NO: 1352 - RNA, 25 nt, Artificial Sequence
  Description of Artificial Sequence Synthetic probe
aauacacguaccuuguugugagugu

SEQ ID NO: 1353 - RNA, 25 nt, Artificial Sequence
  Description of Artificial Sequence Synthetic probe
agacaagcuaaacaacauacguguu

SEQ ID NO: 1354 - RNA, 25 nt, Artificial Sequence
  Description of Artificial Sequence Synthetic probe
accucaaacagaaauugaccuacag

SEQ ID NO: 1355 - RNA, 25 nt, Artificial Sequence
  Description of Artificial Sequence Synthetic probe
ugcaagacguuguauuagaacuaac

SEQ ID NO: 1356 - RNA, 25 nt, Artificial Sequence
  Description of Artificial Sequence Synthetic probe
ggagacaaacaucuagagaaccuag

SEQ ID NO: 1357 - RNA, 25 nt, Artificial Sequence
  Description of Artificial Sequence Synthetic probe
uggaccgggucauguuuggggugcu

SEQ ID NO: 1358 - RNA, 25 nt, Artificial Sequence
  Description of Artificial Sequence Synthetic probe
acgauuucaucuaauagcacauggu

SEQ ID NO: 1359 - RNA, 25 nt, Artificial Sequence
  Description of Artificial Sequence Synthetic probe
agaugucaaaguccguuaacuccgg

SEQ ID NO: 1360 - RNA, 25 nt, Artificial Sequence
  Description of Artificial Sequence Synthetic probe
uggagcuacacuagaaaguauaacu

SEQ ID NO: 1361 - RNA, 25 nt, Artificial Sequence
  Description of Artificial Sequence Synthetic probe
cagugugcagaguauguuuauuguu

SEQ ID NO: 1362 - RNA, 25 nt, Artificial Sequence
  Description of Artificial Sequence Synthetic probe
guguauagggaugauuuuccuuaug

SEQ ID NO: 1363 - RNA, 25 nt, Artificial Sequence
  Description of Artificial Sequence Synthetic probe
acacgugcugagguauauaauuuug

SEQ ID NO: 1364 - RNA, 25 nt, Artificial Sequence
  Description of Artificial Sequence Synthetic probe
cacuugagugagguauuagaaauac

SEQ ID NO: 1365 - RNA, 25 nt, Artificial Sequence
  Description of Artificial Sequence Synthetic probe
ucaacaauccacaggaacguccacg

SEQ ID NO: 1366 - RNA, 25 nt, Artificial Sequence
  Description of Artificial Sequence Synthetic probe
cagcuuauucuguguggacauaucc

SEQ ID NO: 1367 - RNA, 25 nt, Artificial Sequence
  Description of Artificial Sequence Synthetic probe
uacuuuuauauauugggagugaccg

SEQ ID NO: 1368 - RNA, 25 nt, Artificial Sequence
  Description of Artificial Sequence Synthetic probe
guuuguuaugccaaacuaugucuug

SEQ ID NO: 1369 - RNA, 25 nt, Artificial Sequence
  Description of Artificial Sequence Synthetic probe
cuuucaaugcuuaagugcaguuuug

SEQ ID NO: 1370 - RNA, 25 nt, Artificial Sequence
  Description of Artificial Sequence Synthetic probe
ucauauauacaugcagugcaguugc

SEQ ID NO: 1371 - RNA, 25 nt, Artificial Sequence
  Description of Artificial Sequence Synthetic probe
uuuugccuauacuugcauaugugac

SEQ ID NO: 1372 - RNA, 25 nt, Artificial Sequence
  Description of Artificial Sequence Synthetic probe
uuaauccuuucccuuccugcacugc

SEQ ID NO: 1373 - RNA, 25 nt, Artificial Sequence
  Description of Artificial Sequence Synthetic probe
cauuuugugcauguaaccgauuucg

SEQ ID NO: 1374 - RNA, 25 nt, Artificial Sequence
  Description of Artificial Sequence Synthetic probe
caguacugccuccauuuuacuuuac

SEQ ID NO: 1375 - RNA, 25 nt, Artificial Sequence
  Description of Artificial Sequence Synthetic probe
cugccuauuaugcauaccuauguaa

SEQ ID NO: 1376 - RNA, 25 nt, Artificial Sequence
  Description of Artificial Sequence Synthetic probe
ugucccuaaauugcccuacccugcc

SEQ ID NO: 1377 - RNA, 25 nt, Artificial Sequence
  Description of Artificial Sequence Synthetic probe
uuggguguaucuaugaguaaggugc

SEQ ID NO: 1378 - RNA, 25 nt, Artificial Sequence
  Description of Artificial Sequence Synthetic probe
cuugucaguuuccuguuucuguaua

SEQ ID NO: 1379 - RNA, 25 nt, Artificial Sequence
  Description of Artificial Sequence Synthetic probe
guuaugugucauguuuguguacaug

SEQ ID NO: 1380 - RNA, 25 nt, Artificial Sequence
  Description of Artificial Sequence Synthetic probe
uacccgugcaccauccaccaaacgc

SEQ ID NO: 1381 - RNA, 25 nt, Artificial Sequence
  Description of Artificial Sequence Synthetic probe
gcccagacuaaaacguucggccccu

SEQ ID NO: 1382 - RNA, 25 nt, Artificial Sequence
  Description of Artificial Sequence Synthetic probe
cacuaacugcagagauaaugacaua

SEQ ID NO: 1383 - RNA, 25 nt, Artificial Sequence
  Description of Artificial Sequence Synthetic probe
ggaauauguacgucauguugaagaa

SEQ ID NO: 1384 - RNA, 25 nt, Artificial Sequence
  Description of Artificial Sequence Synthetic probe
gcacugaaguaacuaaggaagguac

SEQ ID NO: 1385 - RNA, 25 nt, Artificial Sequence
  Description of Artificial Sequence Synthetic probe
aguuauuuguuaccgugguugauac

SEQ ID NO: 1386 - RNA, 25 nt, Artificial Sequence
  Description of Artificial Sequence Synthetic probe
cucuauaguuaccucagaaucacaa

SEQ ID NO: 1387 - RNA, 25 nt, Artificial Sequence
  Description of Artificial Sequence Synthetic probe
uacugcaguuauccaaaguagugca

SEQ ID NO: 1388 - RNA, 25 nt, Artificial Sequence
  Description of Artificial Sequence Synthetic probe
gucccggaugaccuuuauauuaaag

SEQ ID NO: 1389 - RNA, 25 nt, Artificial Sequence
  Description of Artificial Sequence Synthetic probe
uagggcuggaaaacuuggcgaggcu

SEQ ID NO: 1390 - RNA, 25 nt, Artificial Sequence
  Description of Artificial Sequence Synthetic probe
acgugagcagauguuuguuagacac

SEQ ID NO: 1391 - RNA, 25 nt, Artificial Sequence
  Description of Artificial Sequence Synthetic probe
aaugcagcugcuacugauuguccuc

SEQ ID NO: 1392 - RNA, 25 nt, Artificial Sequence
  Description of Artificial Sequence Synthetic probe
cacagccagggucugauaacaggga

SEQ ID NO: 1393 - RNA, 25 nt, Artificial Sequence
  Description of Artificial Sequence Synthetic probe
acugaaaccaguaacagauaucccg

SEQ ID NO: 1394 - RNA, 25 nt, Artificial Sequence
  Description of Artificial Sequence Synthetic probe
aucaggcuuacaguauagggucuuu

SEQ ID NO: 1395 - RNA, 25 nt, Artificial Sequence
  Description of Artificial Sequence Synthetic probe
uagcuauuuuauuuugcgucgcaga

SEQ ID NO: 1396 - RNA, 25 nt, Artificial Sequence
  Description of Artificial Sequence Synthetic probe
uggauggugcugauuuuauguugca

SEQ ID NO: 1397 - RNA, 25 nt, Artificial Sequence
  Description of Artificial Sequence Synthetic probe
cuccacuaacuccuuuuaauaccau

SEQ ID NO: 1398 - RNA, 25 nt, Artificial Sequence
  Description of Artificial Sequence Synthetic probe
caucuaugucuaguccauuuauucc

SEQ ID NO: 1399 - RNA, 25 nt, Artificial Sequence
  Description of Artificial Sequence Synthetic probe
gguccagacauugcaucuucuguaa

SEQ ID NO: 1400 - RNA, 25 nt, Artificial Sequence
  Description of Artificial Sequence Synthetic probe
cacuccucuugugucauuggaaccu

SEQ ID NO: 1401 - RNA, 25 nt, Artificial Sequence
  Description of Artificial Sequence Synthetic probe
guguccauaccauuaaauacuggau

SEQ ID NO: 1402 - RNA, 25 nt, Artificial Sequence
  Description of Artificial Sequence Synthetic probe
cuuugccaccacacguaccaguaau

SEQ ID NO: 1403 - RNA, 25 nt, Artificial Sequence
  Description of Artificial Sequence Synthetic probe
agaguccucugcacucacauacguc

SEQ ID NO: 1404 - RNA, 25 nt, Artificial Sequence
  Description of Artificial Sequence Synthetic probe
gacgaugcugauacuauacaugauu

SEQ ID NO: 1405 - RNA, 25 nt, Artificial Sequence
  Description of Artificial Sequence Synthetic probe
cucccuauaguauuaaugauggacu

SEQ ID NO: 1406 - RNA, 25 nt, Artificial Sequence
  Description of Artificial Sequence Synthetic probe
caacagcagcaacaauuugaauuac

SEQ ID NO: 1407 - RNA, 25 nt, Artificial Sequence
  Description of Artificial Sequence Synthetic probe
uuaagucccauacagccuguccagg

SEQ ID NO: 1408 - RNA, 25 nt, Artificial Sequence
  Description of Artificial Sequence Synthetic probe
ggcuaaaguacauuacuaccaagac

SEQ ID NO: 1409 - RNA, 25 nt, Artificial Sequence
  Description of Artificial Sequence Synthetic probe
aaaggcuacacuucguacucgcagu

SEQ ID NO: 1410 - RNA, 25 nt, Artificial Sequence
  Description of Artificial Sequence Synthetic probe
acauagugacauaucgccugcuccu

SEQ ID NO: 1411 - RNA, 25 nt, Artificial Sequence
  Description of Artificial Sequence Synthetic probe
acccugaggacacauugcaguuuca

SEQ ID NO: 1412 - RNA, 25 nt, Artificial Sequence
  Description of Artificial Sequence Synthetic probe
cuccucauagacuuguaacauauga

SEQ ID NO: 1413 - RNA, 25 nt, Artificial Sequence
  Description of Artificial Sequence Synthetic probe
gucgcaacacccaacaaguuaaggu

SEQ ID NO: 1414 - RNA, 25 nt, Artificial Sequence
  Description of Artificial Sequence Synthetic probe
caaugucacgucuagcacacccauu

SEQ ID NO: 1415 - RNA, 25 nt, Artificial Sequence
  Description of Artificial Sequence Synthetic probe
ccuuuguuauuucuacugacagugg

SEQ ID NO: 1416 - RNA, 25 nt, Artificial Sequence
  Description of Artificial Sequence Synthetic probe
gcacacauaguuaugaaaacauacc

SEQ ID NO: 1417 - RNA, 25 nt, Artificial Sequence
  Description of Artificial Sequence Synthetic probe
cuggacauuuaauauuuuccucucc

SEQ ID NO: 1418 - RNA, 25 nt, Artificial Sequence
  Description of Artificial Sequence Synthetic probe
auccguacuccgcccuccugcaccu

SEQ ID NO: 1419 - RNA, 25 nt, Artificial Sequence
  Description of Artificial Sequence Synthetic probe
auuuaaaucccuccuuuacugagcc

SEQ ID NO: 1420 - RNA, 25 nt, Artificial Sequence
  Description of Artificial Sequence Synthetic probe
ccugcaauacuuaauguuuccucua

SEQ ID NO: 1421 - RNA, 25 nt, Artificial Sequence
  Description of Artificial Sequence Synthetic probe
uauuaccaccucugcagauacuaca

SEQ ID NO: 1422 - RNA, 25 nt, Artificial Sequence
  Description of Artificial Sequence Synthetic probe
caauucccacuccaucugguuuuga

SEQ ID NO: 1423 - RNA, 25 nt, Artificial Sequence
  Description of Artificial Sequence Synthetic probe
auagacgccggugcaccagccccau

SEQ ID NO: 1424 - RNA, 25 nt, Artificial Sequence
  Description of Artificial Sequence Synthetic probe
guaccccaccgucugaggcuauacc

SEQ ID NO: 1425 - RNA, 25 nt, Artificial Sequence
  Description of Artificial Sequence Synthetic probe
auuacgauaugguagcuuaggggug

SEQ ID NO: 1426 - RNA, 25 nt, Artificial Sequence
  Description of Artificial Sequence Synthetic probe
caucugcuacacaacuuuaccaaac

SEQ ID NO: 1427 - RNA, 25 nt, Artificial Sequence
  Description of Artificial Sequence Synthetic probe
cacauggugguaugguauuguaaau

SEQ ID NO: 1428 - RNA, 25 nt, Artificial Sequence
  Description of Artificial Sequence Synthetic probe
caagacuaacuguauacugguucug

SEQ ID NO: 1429 - RNA, 25 nt, Artificial Sequence
  Description of Artificial Sequence Synthetic probe
gugucuguggggucggcucuacgaa

SEQ ID NO: 1430 - RNA, 25 nt, Artificial Sequence
  Description of Artificial Sequence Synthetic probe
gcugguguugguguugcugcuuugg

SEQ ID NO: 1431 - RNA, 25 nt, Artificial Sequence
  Description of Artificial Sequence Synthetic probe
gccauuggugcuaucuauuucuaua

SEQ ID NO: 1432 - RNA, 25 nt, Artificial Sequence
  Description of Artificial Sequence Synthetic probe
acuguauguaaaccacaagccaaua

SEQ ID NO: 1433 - RNA, 25 nt, Artificial Sequence
  Description of Artificial Sequence Synthetic probe
gcaaauaaguacugguguuauguca

SEQ ID NO: 1434 - RNA, 25 nt, Artificial Sequence
  Description of Artificial Sequence Synthetic probe
acauacacaacggaaacacaacgac

SEQ ID NO: 1435 - RNA, 25 nt, Artificial Sequence
  Description of Artificial Sequence Synthetic probe
gugacaaaguaggaauuguuacugu

SEQ ID NO: 1436 - RNA, 25 nt, Artificial Sequence
  Description of Artificial Sequence Synthetic probe
cuaaaguuucaccuaucgugcauuu

SEQ ID NO: 1437 - RNA, 25 nt, Artificial Sequence
  Description of Artificial Sequence Synthetic probe
uaacuguacauacaaagggcggaac

SEQ ID NO: 1438 - RNA, 25 nt, Artificial Sequence
  Description of Artificial Sequence Synthetic probe
guauacagacugcgccguggacagu

SEQ ID NO: 1439 - RNA, 25 nt, Artificial Sequence
  Description of Artificial Sequence Synthetic probe
gagacaacacccaguacuccacaaa

SEQ ID NO: 1440 - RNA, 25 nt, Artificial Sequence
  Description of Artificial Sequence Synthetic probe
cgacgacucgauuuaccagacucca

SEQ ID NO: 1441 - RNA, 25 nt, Artificial Sequence
  Description of Artificial Sequence Synthetic probe
cgaaaguacacaggggacaaagcga

SEQ ID NO: 1442 - RNA, 25 nt, Artificial Sequence
  Description of Artificial Sequence Synthetic probe
ccuagugaucaaauauccacuacug

SEQ ID NO: 1443 - RNA, 25 nt, Artificial Sequence
  Description of Artificial Sequence Synthetic probe
cuaaaacacaauuaugggagguaca

SEQ ID NO: 1444 - RNA, 25 nt, Artificial Sequence
  Description of Artificial Sequence Synthetic probe
gacuaugugggguuguauuauauac

SEQ ID NO: 1445 - RNA, 25 nt, Artificial Sequence
  Description of Artificial Sequence Synthetic probe
auguacuuugguagcaggagaaguu

SEQ ID NO: 1446 - RNA, 25 nt, Artificial Sequence
  Description of Artificial Sequence Synthetic probe
gacaaugauaaagcaaacacaaugg

SEQ ID NO: 1447 - RNA, 25 nt, Artificial Sequence
  Description of Artificial Sequence Synthetic probe
cuuagaagugugguuaucagagcca

SEQ ID NO: 1448 - RNA, 25 nt, Artificial Sequence
  Description of Artificial Sequence Synthetic probe
cauuagagacauuaaaugcaucacc

SEQ ID NO: 1449 - RNA, 25 nt, Artificial Sequence
  Description of Artificial Sequence Synthetic probe
caucaaagacuaaagcguuucaagu

SEQ ID NO: 1450 - RNA, 25 nt, Artificial Sequence
  Description of Artificial Sequence Synthetic probe
ugggaauaucacauuugugccacca

SEQ ID NO: 1451 - RNA, 25 nt, Artificial Sequence
  Description of Artificial Sequence Synthetic probe
gcuauaauguauacagccagacaaa

SEQ ID NO: 1452 - RNA, 25 nt, Artificial Sequence
  Description of Artificial Sequence Synthetic probe
ugaacauuggaaacuaauacgcaug

SEQ ID NO: 1453 - RNA, 25 nt, Artificial Sequence
  Description of Artificial Sequence Synthetic probe
aauccuagacauauacgaagcugau

SEQ ID NO: 1454 - RNA, 25 nt, Artificial Sequence
  Description of Artificial Sequence Synthetic probe
aauuaggcuuaauagaggaagagga

SEQ ID NO: 1455 - RNA, 25 nt, Artificial Sequence
  Description of Artificial Sequence Synthetic probe
gcacaguagacuaacaguauuugaa

SEQ ID NO: 1456 - RNA, 25 nt, Artificial Sequence
  Description of Artificial Sequence Synthetic probe
gcaaagauucacgauggccauauuu

SEQ ID NO: 1457 - RNA, 25 nt, Artificial Sequence
  Description of Artificial Sequence Synthetic probe
gggcauuaguacaauuaaaaugucc

SEQ ID NO: 1458 - RNA, 25 nt, Artificial Sequence
  Description of Artificial Sequence Synthetic probe
gaugguaacgacauuucaauagaug

SEQ ID NO: 1459 - RNA, 25 nt, Artificial Sequence
  Description of Artificial Sequence Synthetic probe
gaugcuaaacuagguaugauagaug

SEQ ID NO: 1460 - RNA, 25 nt, Artificial Sequence
  Description of Artificial Sequence Synthetic probe
auguuacuguguggcccagcaaaua

SEQ ID NO: 1461 - RNA, 25 nt, Artificial Sequence
  Description of Artificial Sequence Synthetic probe
aaagcgugguaugacaaugggacaa

SEQ ID NO: 1462 - RNA, 25 nt, Artificial Sequence
  Description of Artificial Sequence Synthetic probe
agauuaacaguguuacagcauagcu

SEQ ID NO: 1463 - RNA, 25 nt, Artificial Sequence
  Description of Artificial Sequence Synthetic probe
gaugugcaagggacaacaccagaau

SEQ ID NO: 1464 - RNA, 25 nt, Artificial Sequence
  Description of Artificial Sequence Synthetic probe
aagucaagcaugugccuuauauugg

SEQ ID NO: 1465 - RNA, 25 nt, Artificial Sequence
  Description of Artificial Sequence Synthetic probe
auguaugauuaucgagccaccaaaa

SEQ ID NO: 1466 - RNA, 25 nt, Artificial Sequence
  Description of Artificial Sequence Synthetic probe
auacacaccuacaauguuuaacgug

SEQ ID NO: 1467 - RNA, 25 nt, Artificial Sequence
  Description of Artificial Sequence Synthetic probe
aagucccuccguagcagaaaguuua

SEQ ID NO: 1468 - RNA, 25 nt, Artificial Sequence
  Description of Artificial Sequence Synthetic probe
auugguguauaacaggguauggaau

SEQ ID NO: 1469 - RNA, 25 nt, Artificial Sequence
  Description of Artificial Sequence Synthetic probe
gaagcuuauggaguaaguuuuaugg

SEQ ID NO: 1470 - RNA, 25 nt, Artificial Sequence
  Description of Artificial Sequence Synthetic probe
cauaacaguaauacuaaagcaacgc

SEQ ID NO: 1471 - RNA, 25 nt, Artificial Sequence
  Description of Artificial Sequence Synthetic probe
acggauguagacaguuguaauacug

SEQ ID NO: 1472 - RNA, 25 nt, Artificial Sequence
  Description of Artificial Sequence Synthetic probe
uaaaugacucggagucuaguggggu

SEQ ID NO: 1473 - RNA, 25 nt, Artificial Sequence
  Description of Artificial Sequence Synthetic probe
cacaccagguagaaagccaaaaugg

SEQ ID NO: 1474 - RNA, 25 nt, Artificial Sequence
  Description of Artificial Sequence Synthetic probe
caaauguguguguaucguggaaaua

SEQ ID NO: 1475 - RNA, 25 nt, Artificial Sequence
  Description of Artificial Sequence Synthetic probe
guggacgauauaaaugcugugugug

SEQ ID NO: 1476 - RNA, 25 nt, Artificial Sequence
  Description of Artificial Sequence Synthetic probe
agcguuguuuaauguacaggaaggg

SEQ ID NO: 1477 - RNA, 25 nt, Artificial Sequence
  Description of Artificial Sequence Synthetic probe
cgauagugguacagauuuaauagag

SEQ ID NO: 1478 - RNA, 25 nt, Artificial Sequence
  Description of Artificial Sequence Synthetic probe
cgaagaacaggagauaauauuucag

SEQ ID NO: 1479 - RNA, 25 nt, Artificial Sequence
  Description of Artificial Sequence Synthetic probe
guuugagguagaagcgguaauagaa

SEQ ID NO: 1480 - RNA, 25 nt, Artificial Sequence
  Description of Artificial Sequence Synthetic probe
uaccauugugugcccuagcugugca

SEQ ID NO: 1481 - RNA, 25 nt, Artificial Sequence
  Description of Artificial Sequence Synthetic probe
gacguacgaacccuacagcagcugc

SEQ ID NO: 1482 - RNA, 25 nt, Artificial Sequence
  Description of Artificial Sequence Synthetic probe
uuuguguaucaacaguacaacaacc

SEQ ID NO: 1483 - RNA, 25 nt, Artificial Sequence
  Description of Artificial Sequence Synthetic probe
guuacacuuguggcaccacgguucg

SEQ ID NO: 1484 - RNA, 25 nt, Artificial Sequence
  Description of Artificial Sequence Synthetic probe
ccacagcuaauuacuacauuguaac

SEQ ID NO: 1485 - RNA, 25 nt, Artificial Sequence
  Description of Artificial Sequence Synthetic probe
ucagacgaggaugaaauaggcuugg

SEQ ID NO: 1486 - RNA, 25 nt, Artificial Sequence
  Description of Artificial Sequence Synthetic probe
auccugaaccaacugaccuauucug

SEQ ID NO: 1487 - RNA, 25 nt, Artificial Sequence
  Description of Artificial Sequence Synthetic probe
caacccaacgcuaagagaauauauu

SEQ ID NO: 1488 - RNA, 25 nt, Artificial Sequence
  Description of Artificial Sequence Synthetic probe
ccuguaacaacgccaugagaggaaa

SEQ ID NO: 1489 - RNA, 25 nt, Artificial Sequence
  Description of Artificial Sequence Synthetic probe
ccccgacguagacaaacacaagugu

SEQ ID NO: 1490 - RNA, 25 nt, Artificial Sequence
  Description of Artificial Sequence Synthetic probe
guuucauaauauuucgggucguugg

SEQ ID NO: 1491 - RNA, 25 nt, Artificial Sequence
  Description of Artificial Sequence Synthetic probe
auggagacacauuagaacaaacacu

SEQ ID NO: 1492 - RNA, 25 nt, Artificial Sequence
  Description of Artificial Sequence Synthetic probe
gugugcuuacgauugcuaucuaaaa

SEQ ID NO: 1493 - RNA, 25 nt, Artificial Sequence
  Description of Artificial Sequence Synthetic probe
gaauaguguauagagauggaaaucc

SEQ ID NO: 1494 - RNA, 25 nt, Artificial Sequence
  Description of Artificial Sequence Synthetic probe
aaucgaauugaaaugcguugaaugc

SEQ ID NO: 1495 - RNA, 25 nt, Artificial Sequence
  Description of Artificial Sequence Synthetic probe
aggcguuggagacaucugugcauga

SEQ ID NO: 1496 - RNA, 25 nt, Artificial Sequence
  Description of Artificial Sequence Synthetic probe
ccacggacauugcaugauuuguguc

SEQ ID NO: 1497 - RNA, 25 nt, Artificial Sequence
  Description of Artificial Sequence Synthetic probe
aggacuauguuccaggacgcagagg

SEQ ID NO: 1498 - RNA, 25 nt, Artificial Sequence
  Description of Artificial Sequence Synthetic probe
caaguacaugcacacuuucuacuua

SEQ ID NO: 1499 - RNA, 25 nt, Artificial Sequence
  Description of Artificial Sequence Synthetic probe
acuacugugcaauccaagaaugugu

SEQ ID NO: 1500 - RNA, 25 nt, Artificial Sequence
  Description of Artificial Sequence Synthetic probe
cgcccuuguuaauaaaacagcuuuu

SEQ ID NO: 1501 - RNA, 25 nt, Artificial Sequence
  Description of Artificial Sequence Synthetic probe
aacaauacuugcauaacuuuggugg

SEQ ID NO: 1502 - RNA, 25 nt, Artificial Sequence
  Description of Artificial Sequence Synthetic probe
acgccaaauaguuagucaucauccu

SEQ ID NO: 1503 - RNA, 25 nt, Artificial Sequence
  Description of Artificial Sequence Synthetic probe
ccuagacuacuaacacaacuuacaa

SEQ ID NO: 1504 - RNA, 25 nt, Artificial Sequence
  Description of Artificial Sequence Synthetic probe
uccccaucuuguuuccuccuacacg

SEQ ID NO: 1505 - RNA, 25 nt, Artificial Sequence
  Description of Artificial Sequence Synthetic probe
ucgguuaccuugguuuaaccuuacc

SEQ ID NO: 1506 - RNA, 25 nt, Artificial Sequence
  Description of Artificial Sequence Synthetic probe
guccauuuuauccuuuaaauccucc

SEQ ID NO: 1507 - RNA, 25 nt, Artificial Sequence
  Description of Artificial Sequence Synthetic probe
ccugaauguccaguuuugcauuugc

SEQ ID NO: 1508 - RNA, 25 nt, Artificial Sequence
  Description of Artificial Sequence Synthetic probe
agguguguuuguuccuucauuuugu

SEQ ID NO: 1509 - RNA, 25 nt, Artificial Sequence
  Description of Artificial Sequence Synthetic probe
cauuauuacacauugcccuacuuac

SEQ ID NO: 1510 - RNA, 25 nt, Artificial Sequence
  Description of Artificial Sequence Synthetic probe
gucccuuuauuguuucuuuguccuu

SEQ ID NO: 1511 - RNA, 25 nt, Artificial Sequence
  Description of Artificial Sequence Synthetic probe
guuugucugcuguauguguguauuu

SEQ ID NO: 1512 - RNA, 25 nt, Artificial Sequence
  Description of Artificial Sequence Synthetic probe
guaugugugcauguuguauguuuug

SEQ ID NO: 1513 - RNA, 25 nt, Artificial Sequence
  Description of Artificial Sequence Synthetic probe
gucuuccagaaaauaguguuguuug

SEQ ID NO: 1514 - RNA, 25 nt, Artificial Sequence
  Description of Artificial Sequence Synthetic probe
ccccaucaccaaaacguguuaagcg

SEQ ID NO: 1515 - RNA, 25 nt, Artificial Sequence
  Description of Artificial Sequence Synthetic probe
agcuagaccuaagcccacuauaggc

SEQ ID NO: 1516 - RNA, 25 nt, Artificial Sequence
  Description of Artificial Sequence Synthetic probe
gaaagguuuucugcagaucuugauc

SEQ ID NO: 1517 - RNA, 25 nt, Artificial Sequence
  Description of Artificial Sequence Synthetic probe
aaaguuuuggccuguagaucuuaag

SEQ ID NO: 1518 - RNA, 25 nt, Artificial Sequence
  Description of Artificial Sequence Synthetic probe
uuaaacaggacccuuaugacaaacu

SEQ ID NO: 1519 - RNA, 25 nt, Artificial Sequence
  Description of Artificial Sequence Synthetic probe
ugcugcuguaacuugucaaaaggac

SEQ ID NO: 1520 - RNA, 25 nt, Artificial Sequence
  Description of Artificial Sequence Synthetic probe
uugacacauaccguuuuguucaauc

SEQ ID NO: 1521 - RNA, 25 nt, Artificial Sequence
  Description of Artificial Sequence Synthetic probe
uaaagaauaugccagacauguggag

SEQ ID NO: 1522 - RNA, 25 nt, Artificial Sequence
  Description of Artificial Sequence Synthetic probe
cuaauguauacacaccuaccaguuu

SEQ ID NO: 1523 - RNA, 25 nt, Artificial Sequence
  Description of Artificial Sequence Synthetic probe
ugugcuucuacuacuucuucuauuc

SEQ ID NO: 1524 - RNA, 25 nt, Artificial Sequence
  Description of Artificial Sequence Synthetic probe
aggcaguuauuuauauuccccuucc

SEQ ID NO: 1525 - RNA, 25 nt, Artificial Sequence
  Description of Artificial Sequence Synthetic probe
gugaucaacuuccugaaucacuaua

SEQ ID NO: 1526 - RNA, 25 nt, Artificial Sequence
  Description of Artificial Sequence Synthetic probe
gauaacaaaagugaaguaccauugg

SEQ ID NO: 1527 - RNA, 25 nt, Artificial Sequence
  Description of Artificial Sequence Synthetic probe
ggcuauggacuuuaaauuguugcag

SEQ ID NO: 1528 - RNA, 25 nt, Artificial Sequence
  Description of Artificial Sequence Synthetic probe
uacuacugugguucagggcgauugu

SEQ ID NO: 1529 - RNA, 25 nt, Artificial Sequence
  Description of Artificial Sequence Synthetic probe
gauaccaaagauacacgugauaaug

SEQ ID NO: 1530 - RNA, 25 nt, Artificial Sequence
  Description of Artificial Sequence Synthetic probe
cugaaaacucucauguagcaucugc

SEQ ID NO: 1531 - RNA, 25 nt, Artificial Sequence
  Description of Artificial Sequence Synthetic probe
guagguguugaaaucggucggggcc

SEQ ID NO: 1532 - RNA, 25 nt, Artificial Sequence
  Description of Artificial Sequence Synthetic probe
ccuaacucucaacgcuuggucuggg

SEQ ID NO: 1533 - RNA, 25 nt, Artificial Sequence
  Description of Artificial Sequence Synthetic probe
ccuuccagauaacacaguauaugau

SEQ ID NO: 1534 - RNA, 25 nt, Artificial Sequence
  Description of Artificial Sequence Synthetic probe
gugucugcauaucaauacagaguau

SEQ ID NO: 1535 - RNA, 25 nt, Artificial Sequence
  Description of Artificial Sequence Synthetic probe
aaaggugguaaugguagacaggaug

SEQ ID NO: 1536 - RNA, 25 nt, Artificial Sequence
  Description of Artificial Sequence Synthetic probe
uauuuucuaccacgcaggcaguucc

SEQ ID NO: 1537 - RNA, 25 nt, Artificial Sequence
  Description of Artificial Sequence Synthetic probe
cugaugaguaugucacccguaccag

SEQ ID NO: 1538 - RNA, 25 nt, Artificial Sequence
  Description of Artificial Sequence Synthetic probe
cuaccuccaccuucgguagcuaagg

SEQ ID NO: 1539 - RNA, 25 nt, Artificial Sequence
  Description of Artificial Sequence Synthetic probe
ccuuuaccaccauacagucuauuaa

SEQ ID NO: 1540 - RNA, 25 nt, Artificial Sequence
  Description of Artificial Sequence Synthetic probe
guugaacccacuuauucuacuacac

SEQ ID NO: 1541 - RNA, 25 nt, Artificial Sequence
  Description of Artificial Sequence Synthetic probe
gacccgauauaguuuuaccuaauac

SEQ ID NO: 1542 - RNA, 25 nt, Artificial Sequence
  Description of Artificial Sequence Synthetic probe
gccugggauguuccuguaaauacag

SEQ ID NO: 1543 - RNA, 25 nt, Artificial Sequence
  Description of Artificial Sequence Synthetic probe
caccuuuucaaauguaacuguuccu

SEQ ID NO: 1544 - RNA, 25 nt, Artificial Sequence
  Description of Artificial Sequence Synthetic probe
ugucauuaacacggucggcaucuag

SEQ ID NO: 1545 - RNA, 25 nt, Artificial Sequence
  Description of Artificial Sequence Synthetic probe
ccaacacugcauuuacaauuccuaa

SEQ ID NO: 1546 - RNA, 25 nt, Artificial Sequence
  Description of Artificial Sequence Synthetic probe
acagaugaagcaccuacuaguacug

SEQ ID NO: 1547 - RNA, 25 nt, Artificial Sequence
  Description of Artificial Sequence Synthetic probe
ggcugcuacugaugauauauaugau

SEQ ID NO: 1548 - RNA, 25 nt, Artificial Sequence
  Description of Artificial Sequence Synthetic probe
aauugcaaccucuuguuucuuccca

SEQ ID NO: 1549 - RNA, 25 nt, Artificial Sequence
  Description of Artificial Sequence Synthetic probe
ccuauaccacaugcugaagauauug

SEQ ID NO: 1550 - RNA, 25 nt, Artificial Sequence
  Description of Artificial Sequence Synthetic probe
aacauccagacgcagcacuguaagg

SEQ ID NO: 1551 - RNA, 25 nt, Artificial Sequence
  Description of Artificial Sequence Synthetic probe
cccggacuuuauggauauaguucgu

SEQ ID NO: 1552 - RNA, 25 nt, Artificial Sequence
  Description of Artificial Sequence Synthetic probe
auuaacuuuugaccccucaucagag

SEQ ID NO: 1553 - RNA, 25 nt, Artificial Sequence
  Description of Artificial Sequence Synthetic probe
cugcuuaugauccaauugauacuac

SEQ ID NO: 1554 - RNA, 25 nt, Artificial Sequence
  Description of Artificial Sequence Synthetic probe
guccauccacauuuguuacauauga

SEQ ID NO: 1555 - RNA, 25 nt, Artificial Sequence
  Description of Artificial Sequence Synthetic probe
acaaguucgggugucuaacgcugac

SEQ ID NO: 1556 - RNA, 25 nt, Artificial Sequence
  Description of Artificial Sequence Synthetic probe
accuagauuguacaguagggcuaau

SEQ ID NO: 1557 - RNA, 25 nt, Artificial Sequence
  Description of Artificial Sequence Synthetic probe
auccaacaguacgucguguggcugg

SEQ ID NO: 1558 - RNA, 25 nt, Artificial Sequence
  Description of Artificial Sequence Synthetic probe
ccaaacaggugaaauuucugguaau

SEQ ID NO: 1559 - RNA, 25 nt, Artificial Sequence
  Description of Artificial Sequence Synthetic probe
guagcucuaguuuuauaaauccugc

SEQ ID NO: 1560 - RNA, 25 nt, Artificial Sequence
  Description of Artificial Sequence Synthetic probe
accccaaccucuucuguucaaauua

SEQ ID NO: 1561 - RNA, 25 nt, Artificial Sequence
  Description of Artificial Sequence Synthetic probe
caggauuugaaauaucuaccucuag

SEQ ID NO: 1562 - RNA, 25 nt, Artificial Sequence
  Description of Artificial Sequence Synthetic probe
gauucuaguguuauaacaucuggag

SEQ ID NO: 1563 - RNA, 25 nt, Artificial Sequence
  Description of Artificial Sequence Synthetic probe
ccuacagauccaucuauaguuacau

SEQ ID NO: 1564 - RNA, 25 nt, Artificial Sequence
  Description of Artificial Sequence Synthetic probe
ccaccaguaguuauugaaccuguug

SEQ ID NO: 1565 - RNA, 25 nt, Artificial Sequence
  Description of Artificial Sequence Synthetic probe
uauaguagauguaucgccugcuaaa

SEQ ID NO: 1566 - RNA, 25 nt, Artificial Sequence
  Description of Artificial Sequence Synthetic probe
auugcaguggaccagccuaggaaua

SEQ ID NO: 1567 - RNA, 25 nt, Artificial Sequence
  Description of Artificial Sequence Synthetic probe
cccaucgugcugcucgucguaaacg

SEQ ID NO: 1568 - RNA, 25 nt, Artificial Sequence
  Description of Artificial Sequence Synthetic probe
gcaauacuguccauacaauaauugc

SEQ ID NO: 1569 - RNA, 25 nt, Artificial Sequence
  Description of Artificial Sequence Synthetic probe
uccacuguuacuacuauaugcccau

SEQ ID NO: 1570 - RNA, 25 nt, Artificial Sequence
  Description of Artificial Sequence Synthetic probe
ugguuaucaccuccucauaugagug

SEQ ID NO: 1571 - RNA, 25 nt, Artificial Sequence
  Description of Artificial Sequence Synthetic probe
gugugcauauacaugguuacuagua

SEQ ID NO: 1572 - RNA, 25 nt, Artificial Sequence
  Description of Artificial Sequence Synthetic probe
ucccgcuucugcaaucugucuauau

SEQ ID NO: 1573 - RNA, 25 nt, Artificial Sequence
  Description of Artificial Sequence Synthetic probe
aacccuuguauuuguguguuguguu

SEQ ID NO: 1574 - RNA, 25 nt, Artificial Sequence
  Description of Artificial Sequence Synthetic probe
ugcaaauguaacacaagccaauacu

SEQ ID NO: 1575 - RNA, 25 nt, Artificial Sequence
  Description of Artificial Sequence Synthetic probe
gguauaugaguguguaaugguuguu

SEQ ID NO: 1576 - RNA, 25 nt, Artificial Sequence
  Description of Artificial Sequence Synthetic probe
uaacauauacaagcgaaacacaacg

SEQ ID NO: 1577 - RNA, 25 nt, Artificial Sequence
  Description of Artificial Sequence Synthetic probe
gaaacagaggaucagccaaaacagg

SEQ ID NO: 1578 - RNA, 25 nt, Artificial Sequence
  Description of Artificial Sequence Synthetic probe
ugaaaauauuuccucuaccuggcau

SEQ ID NO: 1579 - RNA, 25 nt, Artificial Sequence
  Description of Artificial Sequence Synthetic probe
ucccuugcaguaacacuacgccuau

SEQ ID NO: 1580 - RNA, 25 nt, Artificial Sequence
  Description of Artificial Sequence Synthetic probe
auccaggcaacaacccgcgacggca

SEQ ID NO: 1581 - RNA, 25 nt, Artificial Sequence
  Description of Artificial Sequence Synthetic probe
ugugacaacccagucguccguuugc

SEQ ID NO: 1582 - RNA, 25 nt, Artificial Sequence
  Description of Artificial Sequence Synthetic probe
gucuaccagcgugucaguggacuac

SEQ ID NO: 1583 - RNA, 25 nt, Artificial Sequence
  Description of Artificial Sequence Synthetic probe
aagcgaccaagacaguguggauaca

SEQ ID NO: 1584 - RNA, 25 nt, Artificial Sequence
  Description of Artificial Sequence Synthetic probe
gcaacuaucauaccccuccgcaacg

SEQ ID NO: 1585 - RNA, 25 nt, Artificial Sequence
  Description of Artificial Sequence Synthetic probe
accagugacgagcaaguauccacug

SEQ ID NO: 1586 - RNA, 25 nt, Artificial Sequence
  Description of Artificial Sequence Synthetic probe
gcaagguuauugauuguuaugacuc

SEQ ID NO: 1587 - RNA, 25 nt, Artificial Sequence
  Description of Artificial Sequence Synthetic probe
acagacaagugggaagugcauuaua

SEQ ID NO: 1588 - RNA, 25 nt, Artificial Sequence
  Description of Artificial Sequence Synthetic probe
gaggaacagguguacuauguaaaau

SEQ ID NO: 1589 - RNA, 25 nt, Artificial Sequence
  Description of Artificial Sequence Synthetic probe
guggacuuuuggggacuauauuaua

SEQ ID NO: 1590 - RNA, 25 nt, Artificial Sequence
  Description of Artificial Sequence Synthetic probe
ugauguaggacagugguguaaaacc

SEQ ID NO: 1591 - RNA, 25 nt, Artificial Sequence
  Description of Artificial Sequence Synthetic probe
gcauuacacaagcuggacauuuaua

SEQ ID NO: 1592 - RNA, 25 nt, Artificial Sequence
  Description of Artificial Sequence Synthetic probe
ccaucugcagcaaggaaaacacaau

SEQ ID NO: 1593 - RNA, 25 nt, Artificial Sequence
  Description of Artificial Sequence Synthetic probe
uguuucuugcauuguccauugcuca

SEQ ID NO: 1594 - RNA, 25 nt, Artificial Sequence
  Description of Artificial Sequence Synthetic probe
aggugcuguuugccauaguucuugg

SEQ ID NO: 1595 - RNA, 25 nt, Artificial Sequence
  Description of Artificial Sequence Synthetic probe
accguacuuccacuguaaugcccug

SEQ ID NO: 1596 - RNA, 25 nt, Artificial Sequence
  Description of Artificial Sequence Synthetic probe
caaggcaugugaagcuauugaacug

SEQ ID NO: 1597 - RNA, 25 nt, Artificial Sequence
  Description of Artificial Sequence Synthetic probe
cagcaagagagaacaauauacauac

SEQ ID NO: 1598 - RNA, 25 nt, Artificial Sequence
  Description of Artificial Sequence Synthetic probe
cuuucgcagcguuuaaguguguuac

SEQ ID NO: 1599 - RNA, 25 nt, Artificial Sequence
  Description of Artificial Sequence Synthetic probe
gaagaugcagacagugauggacacc

SEQ ID NO: 1600 - RNA, 25 nt, Artificial Sequence
  Description of Artificial Sequence Synthetic probe
gcagauuagauuugaacgaggaaga

SEQ ID NO: 1601 - RNA, 25 nt, Artificial Sequence
  Description of Artificial Sequence Synthetic probe
gguggccauauuuaaauagcagauu

SEQ ID NO: 1602 - RNA, 25 nt, Artificial Sequence
  Description of Artificial Sequence Synthetic probe
ggcaccuaguacaaauuaaaugucc

SEQ ID NO: 1603 - RNA, 25 nt, Artificial Sequence
  Description of Artificial Sequence Synthetic probe
gauacauauaugcgaaaugcuuugg

SEQ ID NO: 1604 - RNA, 25 nt, Artificial Sequence
  Description of Artificial Sequence Synthetic probe
gaucguaaauuagcuaugcuagacg

SEQ ID NO: 1605 - RNA, 25 nt, Artificial Sequence
  Description of Artificial Sequence Synthetic probe
ucacuuuuggcuagaaccuuuaaca

SEQ ID NO: 1606 - RNA, 25 nt, Artificial Sequence
  Description of Artificial Sequence Synthetic probe
aauugcauugugcugugugggccag

SEQ ID NO: 1607 - RNA, 25 nt, Artificial Sequence
  Description of Artificial Sequence Synthetic probe
caguggauaaaauggagaugugaua

SEQ ID NO: 1608 - RNA, 25 nt, Artificial Sequence
  Description of Artificial Sequence Synthetic probe
agauaguaauaguaacgccgcugca

SEQ ID NO: 1609 - RNA, 25 nt, Artificial Sequence
  Description of Artificial Sequence Synthetic probe
uagcguguuugaccugucagaaaug

SEQ ID NO: 1610 - RNA, 25 nt, Artificial Sequence
  Description of Artificial Sequence Synthetic probe
auuagugaaguuauaggggaaacgc

SEQ ID NO: 1611 - RNA, 25 nt, Artificial Sequence
  Description of Artificial Sequence Synthetic probe
ccagauacgugcauguuaauugaac

SEQ ID NO: 1612 - RNA, 25 nt, Artificial Sequence
  Description of Artificial Sequence Synthetic probe
aggacuuagcacauuacuacaugua

SEQ ID NO: 1613 - RNA, 25 nt, Artificial Sequence
  Description of Artificial Sequence Synthetic probe
cauggggaguaguaauauuagcauu

SEQ ID NO: 1614 - RNA, 25 nt, Artificial Sequence
  Description of Artificial Sequence Synthetic probe
uaauacaacccuaugugcuauaugc

SEQ ID NO: 1615 - RNA, 25 nt, Artificial Sequence
  Description of Artificial Sequence Synthetic probe
uccaacuguagcagaaggauuuaaa

SEQ ID NO: 1616 - RNA, 25 nt, Artificial Sequence
  Description of Artificial Sequence Synthetic probe
guagcgacagcaguaacauggaugu

SEQ ID NO: 1617 - RNA, 25 nt, Artificial Sequence
  Description of Artificial Sequence Synthetic probe
uguuuguagcgacagucaaauagac

SEQ ID NO: 1618 - RNA, 25 nt, Artificial Sequence
  Description of Artificial Sequence Synthetic probe
cuggaaauggggauagcaauggcag

SEQ ID NO: 1619 - RNA, 25 nt, Artificial Sequence
  Description of Artificial Sequence Synthetic probe
gagacucagguaaccguggagaaua

SEQ ID NO: 1620 - RNA, 25 nt, Artificial Sequence
  Description of Artificial Sequence Synthetic probe
gaagguuaauaacagugccagacag

SEQ ID NO: 1621 - RNA, 25 nt, Artificial Sequence
  Description of Artificial Sequence Synthetic probe
caguaaauguuaaccacccaaaagu

SEQ ID NO: 1622 - RNA, 25 nt, Artificial Sequence
  Description of Artificial Sequence Synthetic probe
acaguagugagaaagcggcggcagg

SEQ ID NO: 1623 - RNA, 25 nt, Artificial Sequence
  Description of Artificial Sequence Synthetic probe
cgaaaguuugggugcaguauagaaa

SEQ ID NO: 1624 - RNA, 25 nt, Artificial Sequence
  Description of Artificial Sequence Synthetic probe
ugcacgggaaaugcauguuuuaaaa

SEQ ID NO: 1625 - RNA, 25 nt, Artificial Sequence
  Description of Artificial Sequence Synthetic probe
gccuuguuuaaugugcaggaagccc

SEQ ID NO: 1626 - RNA, 25 nt, Artificial Sequence
  Description of Artificial Sequence Synthetic probe
caggugacaaaauuucagaugacga

SEQ ID NO: 1627 - RNA, 25 nt, Artificial Sequence
  Description of Artificial Sequence Synthetic probe
guuuauggacacacuauccuuugug

SEQ ID NO: 1628 - RNA, 25 nt, Artificial Sequence
  Description of Artificial Sequence Synthetic probe
guagaaaccucgcaagacggauugc

SEQ ID NO: 1629 - RNA, 25 nt, Artificial Sequence
  Description of Artificial Sequence Synthetic probe
auccuuugcuacuagcuagacgagc

SEQ ID NO: 1630 - RNA, 25 nt, Artificial Sequence
  Description of Artificial Sequence Synthetic probe
uuaccugacuccgacuccgagaaug

SEQ ID NO: 1631 - RNA, 25 nt, Artificial Sequence
  Description of Artificial Sequence Synthetic probe
gaaguugaccuugugugcuacgagc

SEQ ID NO: 1632 - RNA, 25 nt, Artificial Sequence
  Description of Artificial Sequence Synthetic probe
gacauuguuuuagauuuggaaccac

SEQ ID NO: 1633 - RNA, 25 nt, Artificial Sequence
  Description of Artificial Sequence Synthetic probe
aaugcauggaccaaaagcaacacuu

SEQ ID NO: 1634 - RNA, 25 nt, Artificial Sequence
  Description of Artificial Sequence Synthetic probe
agacagcaacgacaagcgcguagug

SEQ ID NO: 1635 - RNA, 25 nt, Artificial Sequence
  Description of Artificial Sequence Synthetic probe
aggacagugucgugggugucggacc

SEQ ID NO: 1636 - RNA, 25 nt, Artificial Sequence
  Description of Artificial Sequence Synthetic probe
cuaaaaccucuauguccaacagaua

SEQ ID NO: 1637 - RNA, 25 nt, Artificial Sequence
  Description of Artificial Sequence Synthetic probe
gcugcugauacgcuguuauagaugc

SEQ ID NO: 1638 - RNA, 25 nt, Artificial Sequence
  Description of Artificial Sequence Synthetic probe
cugaaaccaagacaccguuacauga

SEQ ID NO: 1639 - RNA, 25 nt, Artificial Sequence
  Description of Artificial Sequence Synthetic probe
uccguguauggagaaacauuagagg

SEQ ID NO: 1640 - RNA, 25 nt, Artificial Sequence
  Description of Artificial Sequence Synthetic probe
cuguacaccguaugcagcgugucug

SEQ ID NO: 1641 - RNA, 25 nt, Artificial Sequence
  Description of Artificial Sequence Synthetic probe
cugcaagaaagagagguauuugaau

SEQ ID NO: 1642 - RNA, 25 nt, Artificial Sequence
  Description of Artificial Sequence Synthetic probe
caugauauucgcaucaauugugugu

SEQ ID NO: 1643 - RNA, 25 nt, Artificial Sequence
  Description of Artificial Sequence Synthetic probe
gagcacaacauugaauauuccucug

SEQ ID NO: 1644 - RNA, 25 nt, Artificial Sequence
  Description of Artificial Sequence Synthetic probe
cuacacaacgaccauacaaacugcc

SEQ ID NO: 1645 - RNA, 25 nt, Artificial Sequence
  Description of Artificial Sequence Synthetic probe
aacggcauggcacgcuuugaggauc

SEQ ID NO: 1646 - RNA, 25 nt, Artificial Sequence
  Description of Artificial Sequence Synthetic probe
uaaagguaguugaaaagaaaagggc

SEQ ID NO: 1647 - RNA, 25 nt, Artificial Sequence
  Description of Artificial Sequence Synthetic probe
gucgugcuaaaacagguuucuuuua

SEQ ID NO: 1648 - RNA, 25 nt, Artificial Sequence
  Description of Artificial Sequence Synthetic probe
guaucugucuugcaaauauguaacc

SEQ ID NO: 1649 - RNA, 25 nt, Artificial Sequence
  Description of Artificial Sequence Synthetic probe
guguagcccuuauuguauaagccaa

SEQ ID NO: 1650 - RNA, 25 nt, Artificial Sequence
  Description of Artificial Sequence Synthetic probe
gguguuugcaauauauuuuguuggc

SEQ ID NO: 1651 - RNA, 25 nt, Artificial Sequence
  Description of Artificial Sequence Synthetic probe
uuacucaccuguauuucugugccaa

SEQ ID NO: 1652 - RNA, 25 nt, Artificial Sequence
  Description of Artificial Sequence Synthetic probe
gguauguacacugccuuacccugua

SEQ ID NO: 1653 - RNA, 25 nt, Artificial Sequence
  Description of Artificial Sequence Synthetic probe
caaaacgacuuuucagcaaaacagu

SEQ ID NO: 1654 - RNA, 25 nt, Artificial Sequence
  Description of Artificial Sequence Synthetic probe
cuagccuuuuguccuuauuuaaacc

SEQ ID NO: 1655 - RNA, 25 nt, Artificial Sequence
  Description of Artificial Sequence Synthetic probe
cauuuuaugcaugcaaccgaauucg

SEQ ID NO: 1656 - RNA, 25 nt, Artificial Sequence
  Description of Artificial Sequence Synthetic probe
caaacuccauuuuagugcuguacgc

SEQ ID NO: 1657 - RNA, 25 nt, Artificial Sequence
  Description of Artificial Sequence Synthetic probe
guuuguaugcacuauaguaacacac

SEQ ID NO: 1658 - RNA, 25 nt, Artificial Sequence
  Description of Artificial Sequence Synthetic probe
gugguguuccuuacuguuuaauguu

SEQ ID NO: 1659 - RNA, 25 nt, Artificial Sequence
  Description of Artificial Sequence Synthetic probe
ccuugggcagugugugucagguuag

SEQ ID NO: 1660 - RNA, 25 nt, Artificial Sequence
  Description of Artificial Sequence Synthetic probe
aacaugcaugguuacuuuuacgcgu

SEQ ID NO: 1661 - RNA, 25 nt, Artificial Sequence
  Description of Artificial Sequence Synthetic probe
gcuauguguauguaugacuguaugu

SEQ ID NO: 1662 - RNA, 25 nt, Artificial Sequence
  Description of Artificial Sequence Synthetic probe
uguaugguugugcuuguacuguaug

SEQ ID NO: 1663 - RNA, 25 nt, Artificial Sequence
  Description of Artificial Sequence Synthetic probe
uuccucuucuucaccagcuaaacgu

SEQ ID NO: 1664 - RNA, 25 nt, Artificial Sequence
  Description of Artificial Sequence Synthetic probe
cuaaaaggcgggcggcuccuaccuc

SEQ ID NO: 1665 - RNA, 25 nt, Artificial Sequence
  Description of Artificial Sequence Synthetic probe
uagacccaaggcuaguguaucugcc

SEQ ID NO: 1666 - RNA, 25 nt, Artificial Sequence
  Description of Artificial Sequence Synthetic probe
agcuuuucugcagaccuggaucagu

SEQ ID NO: 1667 - RNA, 25 nt, Artificial Sequence
  Description of Artificial Sequence Synthetic probe
auccccuggcuaaauauaaguuuug

SEQ ID NO: 1668 - RNA, 25 nt, Artificial Sequence
  Description of Artificial Sequence Synthetic probe
auccccaccaguugcaacuagcuua

SEQ ID NO: 1669 - RNA, 25 nt, Artificial Sequence
  Description of Artificial Sequence Synthetic probe
caaucaauaccuucgccauguggag

SEQ ID NO: 1670 - RNA, 25 nt, Artificial Sequence
  Description of Artificial Sequence Synthetic probe
cauuaacuaaauaugaugcccguga

SEQ ID NO: 1671 - RNA, 25 nt, Artificial Sequence
  Description of Artificial Sequence Synthetic probe
caugacuauuaaugcagcuaaaagc

SEQ ID NO: 1672 - RNA, 25 nt, Artificial Sequence
  Description of Artificial Sequence Synthetic probe
gauuaccucugaggcccaauuauuu

SEQ ID NO: 1673 - RNA, 25 nt, Artificial Sequence
  Description of Artificial Sequence Synthetic probe
uccucccaguucuguauauguugcu

SEQ ID NO: 1674 - RNA, 25 nt, Artificial Sequence
  Description of Artificial Sequence Synthetic probe
uuguauuggaaggguggcaauggca

SEQ ID NO: 1675 - RNA, 25 nt, Artificial Sequence
  Description of Artificial Sequence Synthetic probe
gcagguaauguuggggaagccauuc

SEQ ID NO: 1676 - RNA, 25 nt, Artificial Sequence
  Description of Artificial Sequence Synthetic probe
aagcuauuacaggaaucaaaggcug

SEQ ID NO: 1677 - RNA, 25 nt, Artificial Sequence
  Description of Artificial Sequence Synthetic probe
accccgauagaggacggugacaugg

SEQ ID NO: 1678 - RNA, 25 nt, Artificial Sequence
  Description of Artificial Sequence Synthetic probe
uuguccaccucuugcauuaguuaau

SEQ ID NO: 1679 - RNA, 25 nt, Artificial Sequence
  Description of Artificial Sequence Synthetic probe
agucuacaccagguaauacagggga

SEQ ID NO: 1680 - RNA, 25 nt, Artificial Sequence
  Description of Artificial Sequence Synthetic probe
cauuggacuaagggcgcggugugua

SEQ ID NO: 1681 - RNA, 25 nt, Artificial Sequence
  Description of Artificial Sequence Synthetic probe
auagaagauagccgggacaauauau

SEQ ID NO: 1682 - RNA, 25 nt, Artificial Sequence
  Description of Artificial Sequence Synthetic probe
gaggucucuaauuuagcagguaaua

SEQ ID NO: 1683 - RNA, 25 nt, Artificial Sequence
  Description of Artificial Sequence Synthetic probe
gucauccauuauuuaauaggcugga

SEQ ID NO: 1684 - RNA, 25 nt, Artificial Sequence
  Description of Artificial Sequence Synthetic probe
uccaucuuucuauaauccugaccag

SEQ ID NO: 1685 - RNA, 25 nt, Artificial Sequence
  Description of Artificial Sequence Synthetic probe
guuagugcauaucaguauagagugu

SEQ ID NO: 1686 - RNA, 25 nt, Artificial Sequence
  Description of Artificial Sequence Synthetic probe
ugguaccaaaacaaacaucccuaaa

SEQ ID NO: 1687 - RNA, 25 nt, Artificial Sequence
  Description of Artificial Sequence Synthetic probe
gccauccuuauuacucuguuuccaa

SEQ ID NO: 1688 - RNA, 25 nt, Artificial Sequence
  Description of Artificial Sequence Synthetic probe
ggauacauauguaaaacguaccagu

SEQ ID NO: 1689 - RNA, 25 nt, Artificial Sequence
  Description of Artificial Sequence Synthetic probe
auacagggagcuacauuugcacuau

SEQ ID NO: 1690 - RNA, 25 nt, Artificial Sequence
  Description of Artificial Sequence Synthetic probe
cccuucguaccucagucuccuucug

SEQ ID NO: 1691 - RNA, 25 nt, Artificial Sequence
  Description of Artificial Sequence Synthetic probe
ccauuuuauucagguccugauauag

SEQ ID NO: 1692 - RNA, 25 nt, Artificial Sequence
  Description of Artificial Sequence Synthetic probe
acagcuaauguuacugccccuuugg

SEQ ID NO: 1693 - RNA, 25 nt, Artificial Sequence
  Description of Artificial Sequence Synthetic probe
ccuucuacauuauccuuugcuagua

SEQ ID NO: 1694 - RNA, 25 nt, Artificial Sequence
  Description of Artificial Sequence Synthetic probe
caccuucugcacaauuaccuauuaa

SEQ ID NO: 1695 - RNA, 25 nt, Artificial Sequence
  Description of Artificial Sequence Synthetic probe
caaacacguagggguacgcaaauag

SEQ ID NO: 1696 - RNA, 25 nt, Artificial Sequence
  Description of Artificial Sequence Synthetic probe
cauuuacuacacguagaacaggugu

SEQ ID NO: 1697 - RNA, 25 nt, Artificial Sequence
  Description of Artificial Sequence Synthetic probe
ccccacaacauuaauaucugcugau

SEQ ID NO: 1698 - RNA, 25 nt, Artificial Sequence
  Description of Artificial Sequence Synthetic probe
cagguuauauaguagggcuuuucag

SEQ ID NO: 1699 - RNA, 25 nt, Artificial Sequence
  Description of Artificial Sequence Synthetic probe
cagguuuuagacgccuugcugcucc

SEQ ID NO: 1700 - RNA, 25 nt, Artificial Sequence
  Description of Artificial Sequence Synthetic probe
cuauacacgguacuggcaacgaacc

SEQ ID NO: 1701 - RNA, 25 nt, Artificial Sequence
  Description of Artificial Sequence Synthetic probe
cuggaauacauagcuaugaggaaau

SEQ ID NO: 1702 - RNA, 25 nt, Artificial Sequence
  Description of Artificial Sequence Synthetic probe
cugguaauauuuugauuagcacucc

SEQ ID NO: 1703 - RNA, 25 nt, Artificial Sequence
  Description of Artificial Sequence Synthetic probe
ugauccuccaguaauugaggcucca

SEQ ID NO: 1704 - RNA, 25 nt, Artificial Sequence
  Description of Artificial Sequence Synthetic probe
guaguacuacuauaacaaacccacu

SEQ ID NO: 1705 - RNA, 25 nt, Artificial Sequence
  Description of Artificial Sequence Synthetic probe
cccacaucuaguacuguacauguaa

SEQ ID NO: 1706 - RNA, 25 nt, Artificial Sequence
  Description of Artificial Sequence Synthetic probe
ggggcugguguucccaauuuuacug

SEQ ID NO: 1707 - RNA, 25 nt, Artificial Sequence
  Description of Artificial Sequence Synthetic probe
ugugguggagucaguugggccuaca

SEQ ID NO: 1708 - RNA, 25 nt, Artificial Sequence
  Description of Artificial Sequence Synthetic probe
acuauaguugaugucacuccugcac

SEQ ID NO: 1709 - RNA, 25 nt, Artificial Sequence
  Description of Artificial Sequence Synthetic probe
guguauauauugccaugcuuugugg

SEQ ID NO: 1710 - RNA, 25 nt, Artificial Sequence
  Description of Artificial Sequence Synthetic probe
ugcgcuuugcuuuuguguuugucug

SEQ ID NO: 1711 - RNA, 25 nt, Artificial Sequence
  Description of Artificial Sequence Synthetic probe
guaaucgccauauauugcaaccauu

SEQ ID NO: 1712 - RNA, 25 nt, Artificial Sequence
  Description of Artificial Sequence Synthetic probe
auuguaacacugggaaagguaacgu

SEQ ID NO: 1713 - RNA, 25 nt, Artificial Sequence
  Description of Artificial Sequence Synthetic probe
gcuaagcauauauauugcacccauu

SEQ ID NO: 1714 - RNA, 25 nt, Artificial Sequence
  Description of Artificial Sequence Synthetic probe
ugaaguguaauugccauacauugcu

SEQ ID NO: 1715 - RNA, 25 nt, Artificial Sequence
  Description of Artificial Sequence Synthetic probe
caaaugaguuguccauaaaguguug

SEQ ID NO: 1716 - RNA, 25 nt, Artificial Sequence
  Description of Artificial Sequence Synthetic probe
accuaguguacagguuauuuuggga

SEQ ID NO: 1717 - RNA, 25 nt, Artificial Sequence
  Description of Artificial Sequence Synthetic probe
ggacaaguacagauaauaaagacag

SEQ ID NO: 1718 - RNA, 25 nt, Artificial Sequence
  Description of Artificial Sequence Synthetic probe
ugauaaaacuacgccuguaauccau

SEQ ID NO: 1719 - RNA, 25 nt, Artificial Sequence
  Description of Artificial Sequence Synthetic probe
aacaacgccaacaguagaaguccac

SEQ ID NO: 1720 - RNA, 25 nt, Artificial Sequence
  Description of Artificial Sequence Synthetic probe
gaaucagaaccugacuccuccagag

SEQ ID NO: 1721 - RNA, 25 nt, Artificial Sequence
  Description of Artificial Sequence Synthetic probe
accaggaaaacgacccagagcaagu

SEQ ID NO: 1722 - RNA, 25 nt, Artificial Sequence
  Description of Artificial Sequence Synthetic probe
accgagaguauuuacuguccugacu

SEQ ID NO: 1723 - RNA, 25 nt, Artificial Sequence
  Description of Artificial Sequence Synthetic probe
auuacacagacuuugaacaggaggc

SEQ ID NO: 1724 - RNA, 25 nt, Artificial Sequence
  Description of Artificial Sequence Synthetic probe
gguggauuacagaggcauauauuau

SEQ ID NO: 1725 - RNA, 25 nt, Artificial Sequence
  Description of Artificial Sequence Synthetic probe
auaauggagagugugggugguguaa

SEQ ID NO: 1726 - RNA, 25 nt, Artificial Sequence
  Description of Artificial Sequence Synthetic probe
uuguauggaauaugugguguggaaa

SEQ ID NO: 1727 - RNA, 25 nt, Artificial Sequence
  Description of Artificial Sequence Synthetic probe
acaugugaugaacuguggcgcacgg

SEQ ID NO: 1728 - RNA, 25 nt, Artificial Sequence
  Description of Artificial Sequence Synthetic probe
cacuggaagcaauaaguaacacaau

SEQ ID NO: 1729 - RNA, 25 nt, Artificial Sequence
  Description of Artificial Sequence Synthetic probe
cauuaauguacuaaaccaccagaug

SEQ ID NO: 1730 - RNA, 25 nt, Artificial Sequence
  Description of Artificial Sequence Synthetic probe
ccauuagauaacaaugguaauccug

SEQ ID NO: 1731 - RNA, 25 nt, Artificial Sequence
  Description of Artificial Sequence Synthetic probe
cagauacguguuggagauacauaga

SEQ ID NO: 1732 - RNA, 25 nt, Artificial Sequence
  Description of Artificial Sequence Synthetic probe
cuagacaaugccaaauuagguuugc

SEQ ID NO: 1733 - RNA, 25 nt, Artificial Sequence
  Description of Artificial Sequence Synthetic probe
uugguacuguguggaccaccaaaua

SEQ ID NO: 1734 - RNA, 25 nt, Artificial Sequence
  Description of Artificial Sequence Synthetic probe
ugccaguggauaaagcauauaugua

SEQ ID NO: 1735 - RNA, 25 nt, Artificial Sequence
  Description of Artificial Sequence Synthetic probe
aguaacagaugauagccaaauugcc

SEQ ID NO: 1736 - RNA, 25 nt, Artificial Sequence
  Description of Artificial Sequence Synthetic probe
gcaacacaguuuacaagacaaucaa

SEQ ID NO: 1737 - RNA, 25 nt, Artificial Sequence
  Description of Artificial Sequence Synthetic probe
cacaagagcaaauguuaauucaacc

SEQ ID NO: 1738 - RNA, 25 nt, Artificial Sequence
  Description of Artificial Sequence Synthetic probe
ggggaguaauuguaaugaugcuaau

SEQ ID NO: 1739 - RNA, 25 nt, Artificial Sequence
  Description of Artificial Sequence Synthetic probe
uguguguacuaucauaugcaaugcu

SEQ ID NO: 1740 - RNA, 25 nt, Artificial Sequence
  Description of Artificial Sequence Synthetic probe
guuguaacgauuggauaugugcaau

SEQ ID NO: 1741 - RNA, 25 nt, Artificial Sequence
  Description of Artificial Sequence Synthetic probe
gagugccauauacagaguuggugcg

SEQ ID NO: 1742 - RNA, 25 nt, Artificial Sequence
  Description of Artificial Sequence Synthetic probe
guaguaacguacaaggaagauuaca

SEQ ID NO: 1743 - RNA, 25 nt, Artificial Sequence
  Description of Artificial Sequence Synthetic probe
caccaacacaccaauugcaggaacu

SEQ ID NO: 1744 - RNA, 25 nt, Artificial Sequence
  Description of Artificial Sequence Synthetic probe
cacucgguaucaaauauggauauag

SEQ ID NO: 1745 - RNA, 25 nt, Artificial Sequence
  Description of Artificial Sequence Synthetic probe
uggaggcucgcaaaacaguaauugu

SEQ ID NO: 1746 - RNA, 25 nt, Artificial Sequence
  Description of Artificial Sequence Synthetic probe
acgaaaagggaaaugggugcgggag

SEQ ID NO: 1747 - RNA, 25 nt, Artificial Sequence
  Description of Artificial Sequence Synthetic probe
uuggaaacaucacaacagguagaau

SEQ ID NO: 1748 - RNA, 25 nt, Artificial Sequence
  Description of Artificial Sequence Synthetic probe
ggcuaauauuaucagaagacagcgg

SEQ ID NO: 1749 - RNA, 25 nt, Artificial Sequence
  Description of Artificial Sequence Synthetic probe
gguagucccuuaagugauauuagua

SEQ ID NO: 1750 - RNA, 25 nt, Artificial Sequence
  Description of Artificial Sequence Synthetic probe
aaguacaaacagcacaugcagaugc

SEQ ID NO: 1751 - RNA, 25 nt, Artificial Sequence
  Description of Artificial Sequence Synthetic probe
uggaugguuucagguagaagcaauu

SEQ ID NO: 1752 - RNA, 25 nt, Artificial Sequence
  Description of Artificial Sequence Synthetic probe
cgcaucaucuaaauaacugcaaugg

SEQ ID NO: 1753 - RNA, 25 nt, Artificial Sequence
  Description of Artificial Sequence Synthetic probe
uacgugugguacaacagcugcuuau

SEQ ID NO: 1754 - RNA, 25 nt, Artificial Sequence
  Description of Artificial Sequence Synthetic probe
uuggacauucagaguaccaaagagg

SEQ ID NO: 1755 - RNA, 25 nt, Artificial Sequence
  Description of Artificial Sequence Synthetic probe
uaccuuguuguaagugugaguuggu

SEQ ID NO: 1756 - RNA, 25 nt, Artificial Sequence
  Description of Artificial Sequence Synthetic probe
uauauuagaacuugcaccgcaaacg

SEQ ID NO: 1757 - RNA, 25 nt, Artificial Sequence
  Description of Artificial Sequence Synthetic probe
guaaaguaccaacguugcaagaggu

SEQ ID NO: 1758 - RNA, 25 nt, Artificial Sequence
  Description of Artificial Sequence Synthetic probe
agaaucuacaguauaaccaugcaug

SEQ ID NO: 1759 - RNA, 25 nt, Artificial Sequence
  Description of Artificial Sequence Synthetic probe
ggagacauacgaguagacaagcuac

SEQ ID NO: 1760 - RNA, 25 nt, Artificial Sequence
  Description of Artificial Sequence Synthetic probe
uggaccgggucauguuugcaguguu

SEQ ID NO: 1761 - RNA, 25 nt, Artificial Sequence
  Description of Artificial Sequence Synthetic probe
cacugugaacauaaaagacgauuuc

SEQ ID NO: 1762 - RNA, 25 nt, Artificial Sequence
  Description of Artificial Sequence Synthetic probe
auaaauauucaguguauggggcaac

SEQ ID NO: 1763 - RNA, 25 nt, Artificial Sequence
  Description of Artificial Sequence Synthetic probe
gcaguauguaggguauguuuauugu

SEQ ID NO: 1764 - RNA, 25 nt, Artificial Sequence
  Description of Artificial Sequence Synthetic probe
caucugagcgagguauuacaaauac

SEQ ID NO: 1765 - RNA, 25 nt, Artificial Sequence
  Description of Artificial Sequence Synthetic probe
ucagcaauacacaggaacguccacg

SEQ ID NO: 1766 - RNA, 25 nt, Artificial Sequence
  Description of Artificial Sequence Synthetic probe
gccuguagauauccauggauuccau

SEQ ID NO: 1767 - RNA, 25 nt, Artificial Sequence
  Description of Artificial Sequence Synthetic probe
guacauauaaaaggcagccuguugu

SEQ ID NO: 1768 - RNA, 25 nt, Artificial Sequence
  Description of Artificial Sequence Synthetic probe
cugaacacagcaguucucuauacua

SEQ ID NO: 1769 - RNA, 25 nt, Artificial Sequence
  Description of Artificial Sequence Synthetic probe
ggcacacauaccaauacuuuuacuu

SEQ ID NO: 1770 - RNA, 25 nt, Artificial Sequence
  Description of Artificial Sequence Synthetic probe
cuacauccauaaauuugugcaaccg

SEQ ID NO: 1771 - RNA, 25 nt, Artificial Sequence
  Description of Artificial Sequence Synthetic probe
ugucugguaguguaaguuauacagu

SEQ ID NO: 1772 - RNA, 25 nt, Artificial Sequence
  Description of Artificial Sequence Synthetic probe
gccaguauaacuacuuuugcauuca

SEQ ID NO: 1773 - RNA, 25 nt, Artificial Sequence
  Description of Artificial Sequence Synthetic probe
ccucccuuguaauaaaacugcuuuu

SEQ ID NO: 1774 - RNA, 25 nt, Artificial Sequence
  Description of Artificial Sequence Synthetic probe
caauaguuuggcaaccaacguaucu

SEQ ID NO: 1775 - RNA, 25 nt, Artificial Sequence
  Description of Artificial Sequence Synthetic probe
ucguacuggcgcaccuuaguuaguc

SEQ ID NO: 1776 - RNA, 25 nt, Artificial Sequence
  Description of Artificial Sequence Synthetic probe
cccacauaguuggcaccaguaacag

SEQ ID NO: 1777 - RNA, 25 nt, Artificial Sequence
  Description of Artificial Sequence Synthetic probe
gucguugguacuauuugcuuuuaga

SEQ ID NO: 1778 - RNA, 25 nt, Artificial Sequence
  Description of Artificial Sequence Synthetic probe
uggccggguugugugcgaccgcuuu

SEQ ID NO: 1779 - RNA, 25 nt, Artificial Sequence
  Description of Artificial Sequence Synthetic probe
aacuauaccguguggccauuuugua

SEQ ID NO: 1780 - RNA, 25 nt, Artificial Sequence
  Description of Artificial Sequence Synthetic probe
ccuaagguguguuacauuauaugca

SEQ ID NO: 1781 - RNA, 25 nt, Artificial Sequence
  Description of Artificial Sequence Synthetic probe
cugugacuaacauauguccuuguuu

SEQ ID NO: 1782 - RNA, 25 nt, Artificial Sequence
  Description of Artificial Sequence Synthetic probe
uauguccguguccuuugugguugca

SEQ ID NO: 1783 - RNA, 25 nt, Artificial Sequence
  Description of Artificial Sequence Synthetic probe
guguauguuugcaaguaugugugua

SEQ ID NO: 1784 - RNA, 25 nt, Artificial Sequence
  Description of Artificial Sequence Synthetic probe
guguaugugcauguauguguaugug

SEQ ID NO: 1785 - RNA, 25 nt, Artificial Sequence
  Description of Artificial Sequence Synthetic probe
ugugucauguugguguugguauguu

SEQ ID NO: 1786 - RNA, 25 nt, Artificial Sequence
  Description of Artificial Sequence Synthetic probe
uguuguuugucugugugguuguaua

SEQ ID NO: 1787 - RNA, 25 nt, Artificial Sequence
  Description of Artificial Sequence Synthetic probe
accacaucuaccucuaaacacaaac

SEQ ID NO: 1788 - RNA, 25 nt, Artificial Sequence
  Description of Artificial Sequence Synthetic probe
uuacaggcagguguucgcagacggc

SEQ ID NO: 1789 - RNA, 25 nt, Artificial Sequence
  Description of Artificial Sequence Synthetic probe
auucccauuaggacgcaaauuucug

SEQ ID NO: 1790 - RNA, 25 nt, Artificial Sequence
  Description of Artificial Sequence Synthetic probe
aaaaguuuaguucugaacuggacca

SEQ ID NO: 1791 - RNA, 25 nt, Artificial Sequence
  Description of Artificial Sequence Synthetic probe
ccuaugauggucuuaacuuuuggaa

SEQ ID NO: 1792 - RNA, 25 nt, Artificial Sequence
  Description of Artificial Sequence Synthetic probe
accuacaaucagcagcaauuacaug

SEQ ID NO: 1793 - RNA, 25 nt, Artificial Sequence
  Description of Artificial Sequence Synthetic probe
caucugcuagucuuguagauacaua

SEQ ID NO: 1794 - RNA, 25 nt, Artificial Sequence
  Description of Artificial Sequence Synthetic probe
guaccagcuguguaugauucuaaua

SEQ ID NO: 1795 - RNA, 25 nt, Artificial Sequence
  Description of Artificial Sequence Synthetic probe
auuguccacuacuacagacucuacu

SEQ ID NO: 1796 - RNA, 25 nt, Artificial Sequence
  Description of Artificial Sequence Synthetic probe
gaaacuccuaguaguuauguguaug

SEQ ID NO: 1797 - RNA, 25 nt, Artificial Sequence
  Description of Artificial Sequence Synthetic probe
guauauuaagggcacugacauucgu

SEQ ID NO: 1798 - RNA, 25 nt, Artificial Sequence
  Description of Artificial Sequence Synthetic probe
guaccuuuggauauaugucaaucug

SEQ ID NO: 1799 - RNA, 25 nt, Artificial Sequence
  Description of Artificial Sequence Synthetic probe
gguacauuacaagaaacgaaaagcg

SEQ ID NO: 1800 - RNA, 25 nt, Artificial Sequence
  Description of Artificial Sequence Synthetic probe
gaauugguaaauacuccuauugagg

SEQ ID NO: 1801 - RNA, 25 nt, Artificial Sequence
  Description of Artificial Sequence Synthetic probe
ccuaccaauguacaacaaggggacu

SEQ ID NO: 1802 - RNA, 25 nt, Artificial Sequence
  Description of Artificial Sequence Synthetic probe
auguugcaguggacuguaaacaaac

SEQ ID NO: 1803 - RNA, 25 nt, Artificial Sequence
  Description of Artificial Sequence Synthetic probe
ugaaaauuccccguuuuccucuaau

SEQ ID NO: 1804 - RNA, 25 nt, Artificial Sequence
  Description of Artificial Sequence Synthetic probe
ccugagucuacauuauauaauccag

SEQ ID NO: 1805 - RNA, 25 nt, Artificial Sequence
  Description of Artificial Sequence Synthetic probe
ccauccauauuuuaagguuccuaug

SEQ ID NO: 1806 - RNA, 25 nt, Artificial Sequence
  Description of Artificial Sequence Synthetic probe
guacaucuagguuauuaacuguagg

SEQ ID NO: 1807 - RNA, 25 nt, Artificial Sequence
  Description of Artificial Sequence Synthetic probe
caauugauacaaccuuugccauaac

SEQ ID NO: 1808 - RNA, 25 nt, Artificial Sequence
  Description of Artificial Sequence Synthetic probe
caguugccuuuaacacccucuacuc

SEQ ID NO: 1809 - RNA, 25 nt, Artificial Sequence
  Description of Artificial Sequence Synthetic probe
cugauguuguauuaccaucuacaac

SEQ ID NO: 1810 - RNA, 25 nt, Artificial Sequence
  Description of Artificial Sequence Synthetic probe
uggaacacgccuguaaauacugguc

SEQ ID NO: 1811 - RNA, 25 nt, Artificial Sequence
  Description of Artificial Sequence Synthetic probe
uacuaauacuaccauuccucuuggu

SEQ ID NO: 1812 - RNA, 25 nt, Artificial Sequence
  Description of Artificial Sequence Synthetic probe
uggcuucugcugcauccacuacaua

SEQ ID NO: 1813 - RNA, 25 nt, Artificial Sequence
  Description of Artificial Sequence Synthetic probe
cguucccacauaucaguuccuucau

SEQ ID NO: 1814 - RNA, 25 nt, Artificial Sequence
  Description of Artificial Sequence Synthetic probe
caccugauacugacaauacuacagu

SEQ ID NO: 1815 - RNA, 25 nt, Artificial Sequence
  Description of Artificial Sequence Synthetic probe
ggacccuauggauaacuuauaugau

SEQ ID NO: 1816 - RNA, 25 nt, Artificial Sequence
  Description of Artificial Sequence Synthetic probe
aaccauugguugccccugagcaggc

SEQ ID NO: 1817 - RNA, 25 nt, Artificial Sequence
  Description of Artificial Sequence Synthetic probe
uaguaacauuaccccugcugacagc

SEQ ID NO: 1818 - RNA, 25 nt, Artificial Sequence
  Description of Artificial Sequence Synthetic probe
gaccauguuuacacgccgagguaca

SEQ ID NO: 1819 - RNA, 25 nt, Artificial Sequence
  Description of Artificial Sequence Synthetic probe
aacaguacguuuuagcagaguaggc

SEQ ID NO: 1820 - RNA, 25 nt, Artificial Sequence
  Description of Artificial Sequence Synthetic probe
uacuacucuuacauaugaaccugcu

SEQ ID NO: 1821 - RNA, 25 nt, Artificial Sequence
  Description of Artificial Sequence Synthetic probe
aacgcacccuucaucauuuguaaca

SEQ ID NO: 1822 - RNA, 25 nt, Artificial Sequence
  Description of Artificial Sequence Synthetic probe
guauuugcaacacauggcacuggua

SEQ ID NO: 1823 - RNA, 25 nt, Artificial Sequence
  Description of Artificial Sequence Synthetic probe
uguuuguaaguaccccuacaucagg

SEQ ID NO: 1824 - RNA, 25 nt, Artificial Sequence
  Description of Artificial Sequence Synthetic probe
uauaauagaagugccacaaacaggu

SEQ ID NO: 1825 - RNA, 25 nt, Artificial Sequence
  Description of Artificial Sequence Synthetic probe
cuaacccugcauuuacagacccgac

SEQ ID NO: 1826 - RNA, 25 nt, Artificial Sequence
  Description of Artificial Sequence Synthetic probe
cuaccacuacaccggcaguuuuaga

SEQ ID NO: 1827 - RNA, 25 nt, Artificial Sequence
  Description of Artificial Sequence Synthetic probe
guaccaacauuuacaggcaccucug

SEQ ID NO: 1828 - RNA, 25 nt, Artificial Sequence
  Description of Artificial Sequence Synthetic probe
caguguuauuacaucugggacacca

SEQ ID NO: 1829 - RNA, 25 nt, Artificial Sequence
  Description of Artificial Sequence Synthetic probe
gaacccuccauugugcaauuggugg

SEQ ID NO: 1830 - RNA, 25 nt, Artificial Sequence
  Description of Artificial Sequence Synthetic probe
ggaaaaccuaauacuguuguggaug

SEQ ID NO: 1831 - RNA, 25 nt, Artificial Sequence
  Description of Artificial Sequence Synthetic probe
gguacuacacuugcagacaaaauau

SEQ ID NO: 1832 - RNA, 25 nt, Artificial Sequence
  Description of Artificial Sequence Synthetic probe
caguaacuguuauagugugcauuug

SEQ ID NO: 1833 - RNA, 25 nt, Artificial Sequence
  Description of Artificial Sequence Synthetic probe
gugguuauuacacagucuuacucuu

SEQ ID NO: 1834 - RNA, 25 nt, Artificial Sequence
  Description of Artificial Sequence Synthetic probe
cauuugagguguuugcuguauaccu

SEQ ID NO: 1835 - RNA, 25 nt, Artificial Sequence
  Description of Artificial Sequence Synthetic probe
gcauguauauauguugcacuguccc

SEQ ID NO: 1836 - RNA, 25 nt, Artificial Sequence
  Description of Artificial Sequence Synthetic probe
cccacacuguacacuauauguauau

SEQ ID NO: 1837 - RNA, 25 nt, Artificial Sequence
  Description of Artificial Sequence Synthetic probe
gggguauaugacauuauaagugugu

SEQ ID NO: 1838 - RNA, 25 nt, Artificial Sequence
  Description of Artificial Sequence Synthetic probe
gaaacuguuaaacuaccaucuagug

SEQ ID NO: 1839 - RNA, 25 nt, Artificial Sequence
  Description of Artificial Sequence Synthetic probe
uguuucagaagcacaacgugacaag

SEQ ID NO: 1840 - RNA, 25 nt, Artificial Sequence
  Description of Artificial Sequence Synthetic probe
aagacggagccuuuguuguggugac

SEQ ID NO: 1841 - RNA, 25 nt, Artificial Sequence
  Description of Artificial Sequence Synthetic probe
ucaguagaagugcaggccaaaacaa

SEQ ID NO: 1842 - RNA, 25 nt, Artificial Sequence
  Description of Artificial Sequence Synthetic probe
agcccucugagcccgacaacguguc

SEQ ID NO: 1843 - RNA, 25 nt, Artificial Sequence
  Description of Artificial Sequence Synthetic probe
uacugaaucuguugccgaccuacag

SEQ ID NO: 1844 - RNA, 25 nt, Artificial Sequence
  Description of Artificial Sequence Synthetic probe
guaccacugacggaaaaguauccac

SEQ ID NO: 1845 - RNA, 25 nt, Artificial Sequence
  Description of Artificial Sequence Synthetic probe
uauuacgaaagguuuaugcaggaug

SEQ ID NO: 1846 - RNA, 25 nt, Artificial Sequence
  Description of Artificial Sequence Synthetic probe
aaacccaagggcguguggauuacug

SEQ ID NO: 1847 - RNA, 25 nt, Artificial Sequence
  Description of Artificial Sequence Synthetic probe
uguagugugggguacaauuuacuuu

SEQ ID NO: 1848 - RNA, 25 nt, Artificial Sequence
  Description of Artificial Sequence Synthetic probe
gggacaagaguaacucaaugcauua

SEQ ID NO: 1849 - RNA, 25 nt, Artificial Sequence
  Description of Artificial Sequence Synthetic probe
aguaaugaacuauggcauacaaagc

SEQ ID NO: 1850 - RNA, 25 nt, Artificial Sequence
  Description of Artificial Sequence Synthetic probe
agccuugcuaaaacugcauauagug

SEQ ID NO: 1851 - RNA, 25 nt, Artificial Sequence
  Description of Artificial Sequence Synthetic probe
uaacuauuggaauugugugcgacug

SEQ ID NO: 1852 - RNA, 25 nt, Artificial Sequence
  Description of Artificial Sequence Synthetic probe
guauuuacauaguagacuaaccgug

SEQ ID NO: 1853 - RNA, 25 nt, Artificial Sequence
  Description of Artificial Sequence Synthetic probe
uaacccuguagaagacaauaggugg

SEQ ID NO: 1854 - RNA, 25 nt, Artificial Sequence
  Description of Artificial Sequence Synthetic probe
guuuagauagaaaacacagacaccu

SEQ ID NO: 1855 - RNA, 25 nt, Artificial Sequence
  Description of Artificial Sequence Synthetic probe
uucagcaagucacuuuugguuagag

SEQ ID NO: 1856 - RNA, 25 nt, Artificial Sequence
  Description of Artificial Sequence Synthetic probe
aaggcacgccaaaacgaaauuguau

SEQ ID NO: 1857 - RNA, 25 nt, Artificial Sequence
  Description of Artificial Sequence Synthetic probe
uugcauguuccagacagcuguaugc

SEQ ID NO: 1858 - RNA, 25 nt, Artificial Sequence
  Description of Artificial Sequence Synthetic probe
caccuacuacccaacuuaaaguauu

SEQ ID NO: 1859 - RNA, 25 nt, Artificial Sequence
  Description of Artificial Sequence Synthetic probe
gauagugaaaaccaggauccuaaau

SEQ ID NO: 1860 - RNA, 25 nt, Artificial Sequence
  Description of Artificial Sequence Synthetic probe
caagacaaccggcguauacagugcc

SEQ ID NO: 1861 - RNA, 25 nt, Artificial Sequence
  Description of Artificial Sequence Synthetic probe
ucacuaaauguaagcaguacacagg

SEQ ID NO: 1862 - RNA, 25 nt, Artificial Sequence
  Description of Artificial Sequence Synthetic probe
agcaaagucgccauuacaggaauua

SEQ ID NO: 1863 - RNA, 25 nt, Artificial Sequence
  Description of Artificial Sequence Synthetic probe
cagacaguauagaaagcaguccuuu

SEQ ID NO: 1864 - RNA, 25 nt, Artificial Sequence
  Description of Artificial Sequence Synthetic probe
uaaacaaacaggugacacagucuca

SEQ ID NO: 1865 - RNA, 25 nt, Artificial Sequence
  Description of Artificial Sequence Synthetic probe
ucacuaaauuuuguguguccguggu

SEQ ID NO: 1866 - RNA, 25 nt, Artificial Sequence
  Description of Artificial Sequence Synthetic probe
cggacacuacaacagcuguuuaugg

SEQ ID NO: 1867 - RNA, 25 nt, Artificial Sequence
  Description of Artificial Sequence Synthetic probe
cuguguuguaaguguaacaaggcac

SEQ ID NO: 1868 - RNA, 25 nt, Artificial Sequence
  Description of Artificial Sequence Synthetic probe
uguuagagcuauguccauacaauga

SEQ ID NO: 1869 - RNA, 25 nt, Artificial Sequence
  Description of Artificial Sequence Synthetic probe
cauggaccaaagcccaccgugcagg

SEQ ID NO: 1870 - RNA, 25 nt, Artificial Sequence
  Description of Artificial Sequence Synthetic probe
caccuaacaacaaaacgaagauuac

SEQ ID NO: 1871 - RNA, 25 nt, Artificial Sequence
  Description of Artificial Sequence Synthetic probe
guguaugcaacuacauuagaaacca

SEQ ID NO: 1872 - RNA, 25 nt, Artificial Sequence
  Description of Artificial Sequence Synthetic probe
ggaacuacgauauuacucggaaucg

SEQ ID NO: 1873 - RNA, 25 nt, Artificial Sequence
  Description of Artificial Sequence Synthetic probe
ugaccuauguguaguguauagagac

SEQ ID NO: 1874 - RNA, 25 nt, Artificial Sequence
  Description of Artificial Sequence Synthetic probe
acaacggacagagguauaugaauuu

SEQ ID NO: 1875 - RNA, 25 nt, Artificial Sequence
  Description of Artificial Sequence Synthetic probe
ggcgcuauuucacaacccugaggaa

SEQ ID NO: 1876 - RNA, 25 nt, Artificial Sequence
  Description of Artificial Sequence Synthetic probe
uuguguuuugccuaugcuugcaaca

SEQ ID NO: 1877 - RNA, 25 nt, Artificial Sequence
  Description of Artificial Sequence Synthetic probe
auguauuacucaucugcaggugugc

SEQ ID NO: 1878 - RNA, 25 nt, Artificial Sequence
  Description of Artificial Sequence Synthetic probe
gccaaguuucuauccuaccuauaaa

SEQ ID NO: 1879 - RNA, 25 nt, Artificial Sequence
  Description of Artificial Sequence Synthetic probe
ggcaggucaugaacuaaaugucucu

SEQ ID NO: 1880 - RNA, 25 nt, Artificial Sequence
  Description of Artificial Sequence Synthetic probe
ccgcccuguaauaauuuauaugcuu

SEQ ID NO: 1881 - RNA, 25 nt, Artificial Sequence
  Description of Artificial Sequence Synthetic probe
cacaccacauuacucauuuguacuu

SEQ ID NO: 1882 - RNA, 25 nt, Artificial Sequence
  Description of Artificial Sequence Synthetic probe
ugguauguacaucccgcccgcccac

SEQ ID NO: 1883 - RNA, 25 nt, Artificial Sequence
  Description of Artificial Sequence Synthetic probe
ggcauaacccuuaauucuuuuggca

SEQ ID NO: 1884 - RNA, 25 nt, Artificial Sequence
  Description of Artificial Sequence Synthetic probe
caacuuuugaaccacacuaccuaug

SEQ ID NO: 1885 - RNA, 25 nt, Artificial Sequence
  Description of Artificial Sequence Synthetic probe
gcauguaccacaggauuccauuuug

SEQ ID NO: 1886 - RNA, 25 nt, Artificial Sequence
  Description of Artificial Sequence Synthetic probe
gcagcacacuuguauauauauguuc

SEQ ID NO: 1887 - RNA, 25 nt, Artificial Sequence
  Description of Artificial Sequence Synthetic probe
auugcccuacccauauuuguggcuu

SEQ ID NO: 1888 - RNA, 25 nt, Artificial Sequence
  Description of Artificial Sequence Synthetic probe
guuaagggugguguuuagguggcgu

SEQ ID NO: 1889 - RNA, 25 nt, Artificial Sequence
  Description of Artificial Sequence Synthetic probe
guaugguuucugugugguuuacuaa

SEQ ID NO: 1890 - RNA, 25 nt, Artificial Sequence
  Description of Artificial Sequence Synthetic probe
gugugcguguuguguguauuugugu

SEQ ID NO: 1891 - RNA, 25 nt, Artificial Sequence
  Description of Artificial Sequence Synthetic probe
cgcccugccuauguauguguuugug

SEQ ID NO: 1892 - RNA, 25 nt, Artificial Sequence
  Description of Artificial Sequence Synthetic probe
ccccauccucuuccgcuuccucguc

SEQ ID NO: 1893 - RNA, 25 nt, Artificial Sequence
  Description of Artificial Sequence Synthetic probe
caaacccagaccaggccuuaaaagg

SEQ ID NO: 1894 - RNA, 25 nt, Artificial Sequence
  Description of Artificial Sequence Synthetic probe
ucuuuggauuuggaucaguuugcau

SEQ ID NO: 1895 - RNA, 25 nt, Artificial Sequence
  Description of Artificial Sequence Synthetic probe
cuaaagaagacccuuuggcaaaaua

SEQ ID NO: 1896 - RNA, 25 nt, Artificial Sequence
  Description of Artificial Sequence Synthetic probe
ggauucuacaauuuuagaacagugg

SEQ ID NO: 1897 - RNA, 25 nt, Artificial Sequence
  Description of Artificial Sequence Synthetic probe
uuuaagcaguacauuaggcaugggg

SEQ ID NO: 1898 - RNA, 25 nt, Artificial Sequence
  Description of Artificial Sequence Synthetic probe
ugcacaaacauuuacuccagcaaac

SEQ ID NO: 1899 - RNA, 25 nt, Artificial Sequence
  Description of Artificial Sequence Synthetic probe
ccaauuuaaccauuagcacugcugu

SEQ ID NO: 1900 - RNA, 25 nt, Artificial Sequence
  Description of Artificial Sequence Synthetic probe
gguucuaugauaaccucugauucuc

SEQ ID NO: 1901 - RNA, 25 nt, Artificial Sequence
  Description of Artificial Sequence Synthetic probe
guuauauuuauucagcuacucccag

SEQ ID NO: 1902 - RNA, 25 nt, Artificial Sequence
  Description of Artificial Sequence Synthetic probe
ggugcuggccgcgacccuauuagua

SEQ ID NO: 1903 - RNA, 25 nt, Artificial Sequence
  Description of Artificial Sequence Synthetic probe
agacaaggcuuauauuaaggguacu

SEQ ID NO: 1904 - RNA, 25 nt, Artificial Sequence
  Description of Artificial Sequence Synthetic probe
cuggugugguuggugaugccauucc

SEQ ID NO: 1905 - RNA, 25 nt, Artificial Sequence
  Description of Artificial Sequence Synthetic probe
agcagauacauauggcaauucuaug

SEQ ID NO: 1906 - RNA, 25 nt, Artificial Sequence
  Description of Artificial Sequence Synthetic probe
cuguguguaaauacccugauuacuu

SEQ ID NO: 1907 - RNA, 25 nt, Artificial Sequence
  Description of Artificial Sequence Synthetic probe
gcuacuaaaucagauguuccauugg

SEQ ID NO: 1908 - RNA, 25 nt, Artificial Sequence
  Description of Artificial Sequence Synthetic probe
ugugucuacugucauugaggauggc

SEQ ID NO: 1909 - RNA, 25 nt, Artificial Sequence
  Description of Artificial Sequence Synthetic probe
auuauaggcugcgcuccuccuauug

SEQ ID NO: 1910 - RNA, 25 nt, Artificial Sequence
  Description of Artificial Sequence Synthetic probe
guggacaacaaacaaacucaguuau

SEQ ID NO: 1911 - RNA, 25 nt, Artificial Sequence
  Description of Artificial Sequence Synthetic probe
uaauccagacacagaucguuuggug

SEQ ID NO: 1912 - RNA, 25 nt, Artificial Sequence
  Description of Artificial Sequence Synthetic probe
uuggucuuccugauccuaauuuguu

SEQ ID NO: 1913 - RNA, 25 nt, Artificial Sequence
  Description of Artificial Sequence Synthetic probe
uacacgugcugaaauaccuaaggua

SEQ ID NO: 1914 - RNA, 25 nt, Artificial Sequence
  Description of Artificial Sequence Synthetic probe
aacccgcaccggcauauauuauuau

SEQ ID NO: 1915 - RNA, 25 nt, Artificial Sequence
  Description of Artificial Sequence Synthetic probe
cgcauugucaacacagaagaauaua

SEQ ID NO: 1916 - RNA, 25 nt, Artificial Sequence
  Description of Artificial Sequence Synthetic probe
guauuuaccaccugcaccaguguca

SEQ ID NO: 1917 - RNA, 25 nt, Artificial Sequence
  Description of Artificial Sequence Synthetic probe
uauacauauuuguuacgcaaacgcc

SEQ ID NO: 1918 - RNA, 25 nt, Artificial Sequence
  Description of Artificial Sequence Synthetic probe
gguggggauuacuacuuuguggccg

SEQ ID NO: 1919 - RNA, 25 nt, Artificial Sequence
  Description of Artificial Sequence Synthetic probe
gacacacaacaugcuauuguuauac

SEQ ID NO: 1920 - RNA, 25 nt, Artificial Sequence
  Description of Artificial Sequence Synthetic probe
gcccuuuauuccacacacaucuauu

SEQ ID NO: 1921 - RNA, 25 nt, Artificial Sequence
  Description of Artificial Sequence Synthetic probe
uguuaccuacuucacccacugugug

SEQ ID NO: 1922 - RNA, 25 nt, Artificial Sequence
  Description of Artificial Sequence Synthetic probe
ccuauucauacggguccugauguug

SEQ ID NO: 1923 - RNA, 25 nt, Artificial Sequence
  Description of Artificial Sequence Synthetic probe
caucuuaugcuaauguuacuauccc

SEQ ID NO: 1924 - RNA, 25 nt, Artificial Sequence
  Description of Artificial Sequence Synthetic probe
ccuucauugucuuccucuguuucuu

SEQ ID NO: 1925 - RNA, 25 nt, Artificial Sequence
  Description of Artificial Sequence Synthetic probe
cauuuucuccuuugucuacacaacu

SEQ ID NO: 1926 - RNA, 25 nt, Artificial Sequence
  Description of Artificial Sequence Synthetic probe
caaaccacaccuaugcuucgcucuc

SEQ ID NO: 1927 - RNA, 25 nt, Artificial Sequence
  Description of Artificial Sequence Synthetic probe
ugaaacagguuuuaugcagccuaca

SEQ ID NO: 1928 - RNA, 25 nt, Artificial Sequence
  Description of Artificial Sequence Synthetic probe
ccuuuacuuuccccuucuacuaaua

SEQ ID NO: 1929 - RNA, 25 nt, Artificial Sequence
  Description of Artificial Sequence Synthetic probe
auaaguaguauugcaccugcugagg

SEQ ID NO: 1930 - RNA, 25 nt, Artificial Sequence
  Description of Artificial Sequence Synthetic probe
auauuauuaaacugcaccgcccugc

SEQ ID NO: 1931 - RNA, 25 nt, Artificial Sequence
  Description of Artificial Sequence Synthetic probe
cuacugauguugcaccagauccuga

SEQ ID NO: 1932 - RNA, 25 nt, Artificial Sequence
  Description of Artificial Sequence Synthetic probe
gauacaucauuguccuuugaggaac

SEQ ID NO: 1933 - RNA, 25 nt, Artificial Sequence
  Description of Artificial Sequence Synthetic probe
uuaguaagcccucuacauuuguuac

SEQ ID NO: 1934 - RNA, 25 nt, Artificial Sequence
  Description of Artificial Sequence Synthetic probe
gguuaagguuacuaauccagacuuu

SEQ ID NO: 1935 - RNA, 25 nt, Artificial Sequence
  Description of Artificial Sequence Synthetic probe
guuuauauagcagggcauuuucaca

SEQ ID NO: 1936 - RNA, 25 nt, Artificial Sequence
  Description of Artificial Sequence Synthetic probe
guaaggaacccauuagcaguacacc

SEQ ID NO: 1937 - RNA, 25 nt, Artificial Sequence
  Description of Artificial Sequence Synthetic probe
guauuugccuccaauguuacuacug

SEQ ID NO: 1938 - RNA, 25 nt, Artificial Sequence
  Description of Artificial Sequence Synthetic probe
auauauuuaccaguaccccuacguc

SEQ ID NO: 1939 - RNA, 25 nt, Artificial Sequence
  Description of Artificial Sequence Synthetic probe
cauuuauugaggcaccacaaucagg

SEQ ID NO: 1940 - RNA, 25 nt, Artificial Sequence
  Description of Artificial Sequence Synthetic probe
acaagcacuaacauugaaaaucccu

SEQ ID NO: 1941 - RNA, 25 nt, Artificial Sequence
  Description of Artificial Sequence Synthetic probe
auuacuuccucuucuacaacaacuc

SEQ ID NO: 1942 - RNA, 25 nt, Artificial Sequence
  Description of Artificial Sequence Synthetic probe
auucaggcucuacuauaccuaccuu

SEQ ID NO: 1943 - RNA, 25 nt, Artificial Sequence
  Description of Artificial Sequence Synthetic probe
uccggccaggccuccaauuauuauu

SEQ ID NO: 1944 - RNA, 25 nt, Artificial Sequence
  Description of Artificial Sequence Synthetic probe
gacggccugguguuguagauauugc

SEQ ID NO: 1945 - RNA, 25 nt, Artificial Sequence
  Description of Artificial Sequence Synthetic probe
uuauuccuaagguaaagggcacuac

SEQ ID NO: 1946 - RNA, 25 nt, Artificial Sequence
  Description of Artificial Sequence Synthetic probe
aauuauauuccacaugcaaagcugc

SEQ ID NO: 1947 - RNA, 25 nt, Artificial Sequence
  Description of Artificial Sequence Synthetic probe
acaaugguggcugcacgugcacggc

SEQ ID NO: 1948 - RNA, 25 nt, Artificial Sequence
  Description of Artificial Sequence Synthetic probe
ccacaucaccuuuaacuacauuuac

SEQ ID NO: 1949 - RNA, 25 nt, Artificial Sequence
  Description of Artificial Sequence Synthetic probe
aaucccaauauguguuugcagcagc

SEQ ID NO: 1950 - RNA, 25 nt, Artificial Sequence
  Description of Artificial Sequence Synthetic probe
uguauauaguuacucgcaaccauug

SEQ ID NO: 1951 - RNA, 25 nt, Artificial Sequence
  Description of Artificial Sequence Synthetic probe
gucauuggguauuaugacaguguaa

SEQ ID NO: 1952 - RNA, 25 nt, Artificial Sequence
  Description of Artificial Sequence Synthetic probe
uuaaaguaccaucaagugugacagu

SEQ ID NO: 1953 - RNA, 25 nt, Artificial Sequence
  Description of Artificial Sequence Synthetic probe
cacaccaacgucaaaaguuuauuga

SEQ ID NO: 1954 - RNA, 25 nt, Artificial Sequence
  Description of Artificial Sequence Synthetic probe
guaauacaaaagcaggcauuguuac

SEQ ID NO: 1955 - RNA, 25 nt, Artificial Sequence
  Description of Artificial Sequence Synthetic probe
uguuuaaagaagugucaucuaccug

SEQ ID NO: 1956 - RNA, 25 nt, Artificial Sequence
  Description of Artificial Sequence Synthetic probe
gcaacuaaaacugcguuuauaguuc

SEQ ID NO: 1957 - RNA, 25 nt, Artificial Sequence
  Description of Artificial Sequence Synthetic probe
ggaacugcaggcccaaacaccggag

SEQ ID NO: 1958 - RNA, 25 nt, Artificial Sequence
  Description of Artificial Sequence Synthetic probe
caccugcgaccaccaaauacacugu

SEQ ID NO: 1959 - RNA, 25 nt, Artificial Sequence
  Description of Artificial Sequence Synthetic probe
gacuccuccacagucaccccgcugu

SEQ ID NO: 1960 - RNA, 25 nt, Artificial Sequence
  Description of Artificial Sequence Synthetic probe
caccacaacaacgaaaacgacagcg

SEQ ID NO: 1961 - RNA, 25 nt, Artificial Sequence
  Description of Artificial Sequence Synthetic probe
cgaccaauaccuauuccgccuccgc

SEQ ID NO: 1962 - RNA, 25 nt, Artificial Sequence
  Description of Artificial Sequence Synthetic probe
cacccucuacuacaacuguugaaca

SEQ ID NO: 1963 - RNA, 25 nt, Artificial Sequence
  Description of Artificial Sequence Synthetic probe
guaucuaguaccuacagcaccccgu

SEQ ID NO: 1964 - RNA, 25 nt, Artificial Sequence
  Description of Artificial Sequence Synthetic probe
gagguauauauguguggcaauguaa

SEQ ID NO: 1965 - RNA, 25 nt, Artificial Sequence
  Description of Artificial Sequence Synthetic probe
cguggacuauacagguauuuauuac

SEQ ID NO: 1966 - RNA, 25 nt, Artificial Sequence
  Description of Artificial Sequence Synthetic probe
uggacuauacauguuggacauaugu

SEQ ID NO: 1967 - RNA, 25 nt, Artificial Sequence
  Description of Artificial Sequence Synthetic probe
guuugaugggaauaaggacaauaca

SEQ ID NO: 1968 - RNA, 25 nt, Artificial Sequence
  Description of Artificial Sequence Synthetic probe
augcuaugaacuauggggcgaggcc

SEQ ID NO: 1969 - RNA, 25 nt, Artificial Sequence
  Description of Artificial Sequence Synthetic probe
gcauuagaaucgcuaaacaaaucug

SEQ ID NO: 1970 - RNA, 25 nt, Artificial Sequence
  Description of Artificial Sequence Synthetic probe
aucaaaacaaaaggccugccaagcc

SEQ ID NO: 1971 - RNA, 25 nt, Artificial Sequence
  Description of Artificial Sequence Synthetic probe
aucaaguaguaccagcaucggcagu

SEQ ID NO: 1972 - RNA, 25 nt, Artificial Sequence
  Description of Artificial Sequence Synthetic probe
gaaagaaacaugcaaacccuuaacc

SEQ ID NO: 1973 - RNA, 25 nt, Artificial Sequence
  Description of Artificial Sequence Synthetic probe
gacccuaugucaucguuuaaaugug

SEQ ID NO: 1974 - RNA, 25 nt, Artificial Sequence
  Description of Artificial Sequence Synthetic probe
ggaauccuguauaugcacuaaauga

SEQ ID NO: 1975 - RNA, 25 nt, Artificial Sequence
  Description of Artificial Sequence Synthetic probe
gcugcaaauuguaugcccaccauug

SEQ ID NO: 1976 - RNA, 25 nt, Artificial Sequence
  Description of Artificial Sequence Synthetic probe
gaccaguaccuaagaaauuuccuaa

SEQ ID NO: 1977 - RNA, 25 nt, Artificial Sequence
  Description of Artificial Sequence Synthetic probe
cgauaccaggguauuaacuuuaugu

SEQ ID NO: 1978 - RNA, 25 nt, Artificial Sequence
  Description of Artificial Sequence Synthetic probe
guauagaugugacaaagugcaagac

SEQ ID NO: 1979 - RNA, 25 nt, Artificial Sequence
  Description of Artificial Sequence Synthetic probe
cacuaacaaugucagcauggauuag

SEQ ID NO: 1980 - RNA, 25 nt, Artificial Sequence
  Description of Artificial Sequence Synthetic probe
cacuacaaacgagcacaaagaaaau

SEQ ID NO: 1981 - RNA, 25 nt, Artificial Sequence
  Description of Artificial Sequence Synthetic probe
aauuggcugauacagauagcaaugc

SEQ ID NO: 1982 - RNA, 25 nt, Artificial Sequence
  Description of Artificial Sequence Synthetic probe
uugaccaugauguaguagacgauag

SEQ ID NO: 1983 - RNA, 25 nt, Artificial Sequence
  Description of Artificial Sequence Synthetic probe
agcacguuugaacuaucgcaaaugg

SEQ ID NO: 1984 - RNA, 25 nt, Artificial Sequence
  Description of Artificial Sequence Synthetic probe
acaacuacagcacaguuuugaugau

SEQ ID NO: 1985 - RNA, 25 nt, Artificial Sequence
  Description of Artificial Sequence Synthetic probe
cauuaguagcacauauggcgaaaca

SEQ ID NO: 1986 - RNA, 25 nt, Artificial Sequence
  Description of Artificial Sequence Synthetic probe
uuauagaaccaccuaagcuacguag

SEQ ID NO: 1987 - RNA, 25 nt, Artificial Sequence
  Description of Artificial Sequence Synthetic probe
ccauugccaaauguuuagguacauu

SEQ ID NO: 1988 - RNA, 25 nt, Artificial Sequence
  Description of Artificial Sequence Synthetic probe
acuguuagcuagauuuacaugugcc

SEQ ID NO: 1989 - RNA, 25 nt, Artificial Sequence
  Description of Artificial Sequence Synthetic probe
caugugauugggguacuauugugcu

SEQ ID NO: 1990 - RNA, 25 nt, Artificial Sequence
  Description of Artificial Sequence Synthetic probe
guauguacuaccauauacaaugccu

SEQ ID NO: 1991 - RNA, 25 nt, Artificial Sequence
  Description of Artificial Sequence Synthetic probe
ugccuuauuugggguaucgccaaug

SEQ ID NO: 1992 - RNA, 25 nt, Artificial Sequence
  Description of Artificial Sequence Synthetic probe
aaacaugcugcacggacuggguaug

SEQ ID NO: 1993 - RNA, 25 nt, Artificial Sequence
  Description of Artificial Sequence Synthetic probe
gaguugguaaggguauuuaaaagug

SEQ ID NO: 1994 - RNA, 25 nt, Artificial Sequence
  Description of Artificial Sequence Synthetic probe
caaugcaaaagcaauguuuauggca

SEQ ID NO: 1995 - RNA, 25 nt, Artificial Sequence
  Description of Artificial Sequence Synthetic probe
ccaauguaggacuaaacaguauaug

SEQ ID NO: 1996 - RNA, 25 nt, Artificial Sequence
  Description of Artificial Sequence Synthetic probe
gaccuggaaacaaacgaaaaugcua

SEQ ID NO: 1997 - RNA, 25 nt, Artificial Sequence
  Description of Artificial Sequence Synthetic probe
gaugggcaaaaugacgggucacaac

SEQ ID NO: 1998 - RNA, 25 nt, Artificial Sequence
  Description of Artificial Sequence Synthetic probe
agacuguggaaggacccuuacaggu

SEQ ID NO: 1999 - RNA, 25 nt, Artificial Sequence
  Description of Artificial Sequence Synthetic probe
aggagauuacuggacaguuauccgg

SEQ ID NO: 2000 - RNA, 25 nt, Artificial Sequence
  Description of Artificial Sequence Synthetic probe
cagcaacaaccaaaacaggcaaacc

SEQ ID NO: 2001 - RNA, 25 nt, Artificial Sequence
  Description of Artificial Sequence Synthetic probe
gcagcccauuaaaagacauuacaaa

SEQ ID NO: 2002 - RNA, 25 nt, Artificial Sequence
  Description of Artificial Sequence Synthetic probe
aaacacaggcacacaaagaggcugu

SEQ ID NO: 2003 - RNA, 25 nt, Artificial Sequence
  Description of Artificial Sequence Synthetic probe
gcacaggcguuguugcagguccaag

SEQ ID NO: 2004 - RNA, 25 nt, Artificial Sequence
  Description of Artificial Sequence Synthetic probe
aauaguauuuguagucaggcggaac

SEQ ID NO: 2005 - RNA, 25 nt, Artificial Sequence
  Description of Artificial Sequence Synthetic probe
gauacaaaugauacagggucugaua

SEQ ID NO: 2006 - RNA, 25 nt, Artificial Sequence
  Description of Artificial Sequence Synthetic probe
cgggagauaauauaucagacgauga

SEQ ID NO: 2007 - RNA, 25 nt, Artificial Sequence
  Description of Artificial Sequence Synthetic probe
acaucggcaauggacagugaaggua

SEQ ID NO: 2008 - RNA, 25 nt, Artificial Sequence
  Description of Artificial Sequence Synthetic probe
uaagccuggugugcccguggugugc

SEQ ID NO: 2009 - RNA, 25 nt, Artificial Sequence
  Description of Artificial Sequence Synthetic probe
auuucagcaaauguuacugggcgac

SEQ ID NO: 2010 - RNA, 25 nt, Artificial Sequence
  Description of Artificial Sequence Synthetic probe
aaagcaguggagacagccuucgcau

SEQ ID NO: 2011 - RNA, 25 nt, Artificial Sequence
  Description of Artificial Sequence Synthetic probe
ugcagguguucgaguguuguacagc

SEQ ID NO: 2012 - RNA, 25 nt, Artificial Sequence
  Description of Artificial Sequence Synthetic probe
guguuacagaauuaaagugcacugu

SEQ ID NO: 2013 - RNA, 25 nt, Artificial Sequence
  Description of Artificial Sequence Synthetic probe
uaacaccacaaccugaaauugacuu

SEQ ID NO: 2014 - RNA, 25 nt, Artificial Sequence
  Description of Artificial Sequence Synthetic probe
caauuaaaggacauaguguuggagu

SEQ ID NO: 2015 - RNA, 25 nt, Artificial Sequence
  Description of Artificial Sequence Synthetic probe
uagugaaacccagguguaauaacgc

SEQ ID NO: 2016 - RNA, 25 nt, Artificial Sequence
  Description of Artificial Sequence Synthetic probe
auugcagaaaaccaccaagacaacg

SEQ ID NO: 2017 - RNA, 25 nt, Artificial Sequence
  Description of Artificial Sequence Synthetic probe
agaaaagcaaaaggugguggacgac

SEQ ID NO: 2018 - RNA, 25 nt, Artificial Sequence
  Description of Artificial Sequence Synthetic probe
gaugucagagaccacuugggccuga

SEQ ID NO: 2019 - RNA, 25 nt, Artificial Sequence
  Description of Artificial Sequence Synthetic probe
cauuagaggccauuacuaacaaaag

SEQ ID NO: 2020 - RNA, 25 nt, Artificial Sequence
  Description of Artificial Sequence Synthetic probe
aagguauaguaggucuguguauggu

SEQ ID NO: 2021 - RNA, 25 nt, Artificial Sequence
  Description of Artificial Sequence Synthetic probe
gggacaauacgccauaugcagcaug

SEQ ID NO: 2022 - RNA, 25 nt, Artificial Sequence
  Description of Artificial Sequence Synthetic probe
guagcauuuacagaacuuaggauug

SEQ ID NO: 2023 - RNA, 25 nt, Artificial Sequence
  Description of Artificial Sequence Synthetic probe
guuguguagagcagauguguauaau

SEQ ID NO: 2024 - RNA, 25 nt, Artificial Sequence
  Description of Artificial Sequence Synthetic probe
gucuaugcacaauauucagguauug

SEQ ID NO: 2025 - RNA, 25 nt, Artificial Sequence
  Description of Artificial Sequence Synthetic probe
acgaauuaugugaagccugcaauac

SEQ ID NO: 2026 - RNA, 25 nt, Artificial Sequence
  Description of Artificial Sequence Synthetic probe
uuugaagacauaagagaaagaccac